(19) United States
(12) Patent Application Publication
(10) Pub. No.: US 2024/0253990 A1
(43) Pub. Date: Aug. 1, 2024

(54) Systems and Methods for Nitric Oxide Delivery

(71) Applicant: Third Pole, Inc., Waltham, MA (US)

(72) Inventors: Benjamin J. Apollonio, Lunenburg, MA (US); Frank Heirtzler, Londonderry, NH (US); Nathaniel G. Jackson, Lexington, MA (US); Gregory W. Hall, Belmont, MA (US)

(73) Assignee: Third Pole, Inc., Waltham, MA (US)

(21) Appl. No.: 18/427,703

(22) Filed: Jan. 30, 2024

Related U.S. Application Data
(60) Provisional application No. 63/482,219, filed on Jan. 30, 2023.
(60) Provisional application No. 63/507,071, filed on Jun. 8, 2023.

(51) Int. Cl. C01B 21/20; A61K 33/00; A61M 16/12

(52) U.S. Cl. CPC C01B 21/203; A61K 33/00; A61M 16/122; A61M 2202/0275

(57) ABSTRACT

Systems and methods for nitric oxide (NO) delivery are provided. A NO delivery system can include one or more pairs of electrodes configured to ionize a reactant gas into an NO-containing product gas, a delivery line to deliver at least a portion of the product gas into an inspiratory flow of gas, and a controller. The controller is configured to control an amount of NO in the product gas generated by the one or more pairs of electrodes using one or more parameters as input to the at least one controller. One of the parameters is a dilution value derived as a function of an inspiratory flow rate and a target inspiratory gas NO concentration level. The dilution value is used by the controller to set a flow rate of the product gas injected into the inspiratory flow and to determine a target concentration of NO in the product gas.

### RELATED APPLICATIONS

This application claims the benefit of and priority to U.S. Provisional Application Ser. No. 63/482,219, filed on Jan. 30, 2023, and U.S. Provisional Application Ser. No. 63/507,071, filed on Jun. 8, 2023, each of which is incorporated herein by reference in their entireties.

### FEDERALLY SPONSORED RESEARCH OR DEVELOPMENT

This invention was made with government support under Grant Number HL134429 awarded by the National Institutes of Health. The government has certain rights in the invention.

### FIELD

The present disclosure relates to systems and methods for generating and/or delivering nitric oxide.

### BACKGROUND

Nitric oxide has found to be useful in a number of ways for treatment of disease, particularly cardiac and respiratory ailments. Previous systems for producing NO and delivering the NO gas to a patient have several disadvantages. For example, tank-based systems required large tanks of NO gas at a high concentration and pressure. When treatment using this system is paused, NO in the circuit stalls and converts into NO₂, requiring the user to purge the manual ventilation circuit before resuming manual ventilation. Synthesizing NO from NO₂ or N₂O₄ requires the handling of toxic chemicals. Prior electric generation systems involve generating plasma in the main flow of air to be delivered to patients or pumped through a delivery tube.

### SUMMARY

The present disclosure is directed to systems and methods for nitric oxide (NO) delivery. In some embodiments, a NO delivery system includes one or more pairs of electrodes configured to ionize a reactant gas into an NO-containing product gas, a delivery line configured to deliver at least a portion of the product gas into an inspiratory flow of gas, and at least one controller. The at least one controller is configured to control an amount of NO in the product gas generated by the one or more pairs of electrodes using one or more parameters as input to the at least one controller. One of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

In some embodiments, the dilution value is a dilution factor, the dilution factor being a function of an injected inspiratory flow and a pre-injection inspiratory flow. In some embodiments, the dilution value is a dilution ratio, the dilution ratio being a function of an injected inspiratory flow and a flow of inspiratory gas downstream of the NO injection. In some embodiments, the dilution value is variable. In some embodiments, the controller is configured to select the dilution value to minimize a level of inhaled NO₂. In some embodiments, the controller is configured to select the dilution value to minimize a dilution of inspiratory flow and corresponding inhaled oxygen levels. In some embodiments, the controller is configured to select the dilution value such that the flow controller operates within an acceptable operating range. In some embodiments, the controller is configured to select the dilution value such that the NO concentration of the product gas is compatible with materials in a product gas flow pathway.

In some embodiments, the controller is configured to select a high dilution value to purge one or more product gas pathways to reset the product gas concentration within the one or more pathways to one or more known conditions. In some embodiments, one of the known conditions is a known product gas concentration. In some embodiments, the controller is configured to initiate a purge at specific time intervals. In some embodiments, the controller is configured to initiate a purge when an expected product gas NO concentration and a measured product gas NO concentration differ by a threshold amount.

In some embodiments, the flow rate of product gas injected into the inspiratory flow has a variable flow rate. In some embodiments, the controller is configured to convert the variable flow rate of product gas injected into the inspiratory flow to a constant flow rate of product gas when a detected breath frequency increases beyond a threshold.

In some embodiments, a nitric oxide (NO) delivery system is provided and includes one or more pairs of electrodes configured to ionize a reactant gas into an NO-containing product gas, a scrubber configured to remove NO₂ from the product gas, a flow controller configured to deliver at least a portion of the product gas into an inspiratory flow of gas and at least a portion of the product gas upstream of the scrubber, and at least one controller. The at least one controller is configured to control an amount of NO in the product gas generated by the one or more pairs of electrodes using one or more parameters as input to the at least one controller. One of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

In some embodiments, a nitric oxide (NO) delivery system is provided that includes one or more pairs of electrodes configured to ionize a reactant gas into an NO-containing product gas, and at least one controller. The at least one controller is configured to control an amount of NO in the product gas generated by the one or more pairs of electrodes using one or more parameters as input to the at least one controller. One of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

In some embodiments, the system further includes a delivery line configured to deliver at least a portion of the product gas into an inspiratory flow of gas. In some embodiments, the system further includes a flow controller configured to deliver at least a portion of the product gas into an inspiratory flow of gas and at least a portion of the product gas upstream of the one or more pairs of electrodes. In some embodiments, the flow controller is in electrical communication with the controller such that the controller is configured to regulate the amount of the product gas delivered to the inspiratory flow and upstream of the one or more pairs of electrodes.

In some embodiments, the dilution value is a dilution factor, the dilution factor being a function of an injected inspiratory flow and a pre-injection inspiratory flow. In some embodiments, the dilution value is a dilution ratio, the dilution ratio being a function of an injected inspiratory flow and a flow of inspiratory gas downstream of the NO injection.

### BRIEF DESCRIPTION OF THE DRAWING

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A illustrates an exemplary embodiment of an electrical NO generation system;

FIG. 1B depicts an embodiment of a NO generation and delivery system with a recirculation architecture;

FIG. 1C depicts an exemplary embodiment of a pulsed NO delivery system with a pressurized scrubber and pressurized bypass architecture;

FIG. 1D depicts an exemplary embodiment of a NO delivery system with a pressurized scrubber and pressurized bypass device with removable modules;

FIG. 1E depicts an exemplary embodiment of a NO generation and delivery system;

FIG. 2A depicts an exemplary embodiment of a NO generation system that utilizes a humidity management cartridge (HMC) to maintain an acceptable humidity level within a gas;

FIG. 2B depicts an exemplary system with a built-in, reusable dehumidification feature that includes desiccant material in a chamber;

FIG. 3 depicts an exemplary graph showing experimental results of a reactant gas humidity control system that utilizes 1,2,3-Propanetriol that targets 49% RH in a gas stream;

FIG. 4 depicts an exemplary embodiment of a NO generation system with active drying of the desiccant material;

FIG. 5 depicts an exemplary embodiment of a gas humidity reduction process that utilizes elevated pressure;

FIG. 6 depicts an exemplary embodiment of a gas humidity reduction process that utilizes a reduction in temperature;

FIG. 7 depicts an exemplary embodiment of a gas humidity reduction process that utilizes a water partial pressure difference across a membrane;

FIG. 8 depicts an exemplary embodiment of a NO generation system that includes a humidity modification process that acts on the blended mixture of fresh reactant gas and returned product gas;

FIG. 9 depicts an exemplary process, utilized by a NO generation device controller, to set the NO product gas concentration and produce product gas at the set concentration;

FIG. 10 depicts an exemplary control scheme for the product gas injection flow control;

FIG. 11 illustrates various exemplary flows in the system and defines the terms “dilution factor” and “dilution ratio;”

FIG. 12A depicts an exemplary plot of dilution factors as a function of inspired NO dose for a given system architecture;

FIG. 12B depicts an embodiment of a graph showing an exemplary dilution strategy that utilizes a specific linear relationship between target dose and dilution factor;

FIG. 13 depicts a method for determining product gas concentration using a dilution factor;

FIG. 14 depicts an exemplary embodiment of a redundant recirculation architecture with redundant NO sensors in each generator;

FIG. 15 depicts an exemplary embodiment of a NO generation system with redundant NO generators that can each dose to two independent gas streams;

FIG. 16 depicts an exemplary embodiment of a NO generation system with a rudimentary NO generation backup for dosing one gas stream;

FIG. 17A depicts an exemplary embodiment of a system with redundant NO generation channels and redundant NO sensors shared by the NO generation channels;

FIG. 17B depicts an exemplary embodiment of a NO generation system with recirculation architecture, a remote injection module and purge pathway;

FIG. 18 depicts an exemplary system that utilizes a single plasma chamber that can be configured for either recirculation architecture or a pressurized scrubber reservoir;

FIG. 19A depicts an exemplary system that can serve as both a continuous delivery system and a pulsed NO delivery system with different valve locations;

FIG. 19B depicts an exemplary embodiment of a system with the reactant gas pump located before the plasma chamber, thereby protecting the pump from particulates originating from the plasma chamber;

FIG. 20 depicts an exemplary embodiment of a system that includes particle removal before the pump;

FIG. 21 depicts an exemplary graph of an example of a plasma calibration curve;

FIG. 22 depicts an exemplary flowchart of an embodiment of how a NO generation controller can intermittently read from a sensor during battery operation;

FIG. 23 depicts an exemplary graph showing an embodiment of NO delivery to an inspiratory flow profile;

FIG. 24 depicts an exemplary graph of a ventilator flow profile that is dosed with intermittent NO pulses;

FIG. 25 depicts an exemplary NO delivery system for delivering alternating flows of NO and another gas;

FIG. 26A depicts the effects of system lag on inspired dose accuracy;

FIG. 26B depicts the effects of a predictive NO delivery system on inhaled dose accuracy;

FIG. 26C depicts an embodiment of NO delivery that utilizes leading edge delay compensation;

FIG. 26D depicts an embodiment of NO delivery that utilizes leading edge delay utilizes leading edge and trailing edge compensation;

FIG. 26E depicts an embodiment of NO delivery that triggers off the pressure wave within the inspiratory limb;

FIG. 26F depicts exemplary inputs and outputs of a NO delivery system that utilizes a fast flow sensor and a slow flow sensor to detect and accurately dose patient respirations; and

FIG. 27 depicts exemplary data from a sensor measuring EMI output from a plasma chamber;

FIG. 28A illustrates an exemplary graph of an embodiment of a relationship between electrode gap and EMI emissions for a parallel electrode pair energized with AC voltage;

FIG. 28B illustrates an exemplary embodiment of a peak detector circuit;

FIG. 29 illustrates an exemplary graph that presents experimental data using an electrical NO generator with an optical sensor that collects light output from the plasma chamber;

FIG. 30 depicts an exemplary graph showing the IR absorption spectrum for nitric oxide gas;

FIG. 31A depicts an exemplary graph showing exemplary experimental data that demonstrates the relationship between sound level and plasma activity (duty cycle) in an electric NO generator;

FIG. 31B depicts an exemplary graph of experimental data that demonstrates the relationship between acoustic noise and NO production;

FIGS. 32A and 32B depict an exemplary embodiment of a particle trap used to collect electrode particles within an electric NO generation system;

FIG. 33 depicts an exemplary embodiment of a plasma chamber with an opposed, notched electrode design;

FIGS. 34A and 34B depict an exemplary embodiment of an electrode design with smooth surfaces for low electrical field;

FIGS. 35A, 35B, and 35C depict cross sections of exemplary embodiments of electrodes;

FIG. 36 depicts an exemplary embodiment of an electrode design that eliminates sharp edges;

FIG. 37 depicts an exemplary embodiment of a full parallel design in which the electrodes are oriented parallel to the direction of reactant gas flow;

FIG. 38 depicts an exemplary embodiment of an array of electrode pairs;

FIG. 39 depicts an exemplary embodiment of a full parallel design with three electrodes;

FIG. 40 depicts an exemplary embodiment of a full parallel design with three electrodes oriented along the direction of reactant gas flow;

FIG. 41 depicts an exemplary embodiment of a 3-electrode full parallel design in an array configuration;

FIG. 42 depicts an exemplary embodiment of full parallel electrodes with six electrodes providing 5 electrode gaps;

FIG. 43 depicts an exemplary embodiment of full parallel electrodes;

FIG. 44A depicts an embodiment of a pair of parallel electrodes in a plasma chamber;

FIG. 44B depicts an embodiment of a pair of parallel electrodes with insulators;

FIG. 45A depicts an embodiment of a plasma chamber with bent electrodes;

FIG. 45B depicts an embodiment of a plasma chamber with an array of bent electrodes;

FIG. 45C depicts an embodiment of a plasma chamber with bent electrodes;

FIG. 46A depicts an embodiment of a plasma chamber with electrodes made from dissimilar materials;

FIG. 46B depicts an embodiment of a plasma chamber with electrodes with dissimilar geometry;

FIG. 46C depicts an embodiment of a plasma chamber with dissimilar electrodes;

FIG. 46D depicts an embodiment of a plasma chamber with dissimilar electrodes and consistent electrode gap;

FIG. 47A depicts an exemplary embodiment of a plasma vortex electrode assembly;

FIG. 47B depicts exemplary electrodes of a plasma vortex electrode assembly;

FIG. 48 depicts an exemplary embodiment of a scrubber having 3 stacks of grooved scrubber sheet material 4 layers tall;

FIG. 49 depicts an exemplary embodiment of a variable scrubbing system to scrub a product gas flow to achieve a target NO concentration;

FIG. 50A depicts an exemplary embodiment of a system with variable scrubbing in a recirculation architecture;

FIG. 50B depicts an exemplary embodiment of a variable product gas NO scrubber;

FIG. 51 depicts a process for calibration of inspiratory NO gas sensor using a reference sensor;

FIG. 52 depicts a process for calibration of inspiratory NO₂ gas sensor using a reference sensor;

FIG. 53 depicts an exemplary sensor output to a step change in input as can be used for expedited calibration.

FIG. 54 depicts an exemplary embodiment of a lung transport device that perfuses the lung with de-oxygenated fluid while ventilating the lung with positive pressure;

FIG. 55 depicts an exemplary embodiment of a lung transport device that delivers NO in the perfusate;

FIG. 56 depicts an exemplary embodiment of an organ transport device;

FIG. 57 depicts an exemplary embodiment of an organ transplant device wherein the chamber housing the organ is filled with an NO-containing gas;

FIG. 58 depicts an exemplary graph showing higher levels of obstruction result in a slower rise in CO2 levels during exhalation;

FIG. 59 depicts an exemplary embodiment of a NO delivery device that utilizes a blower to pressurize an airway and deliver gas;

FIG. 60 depicts an embodiment of an electrical NO generation and delivery system configured for delivery of inspiratory gas and NO to a patient;

FIG. 61 depicts an embodiment of an electrical NO generation and delivery system that utilizes an SpO₂ measurement as an input;

FIGS. 62A and 62B depict the effects of NO delivery flow waveform on inhaled dose accuracy;

FIG. 63 depicts an embodiment of a NO generation system that delivers NO to the high pressure gas within a high frequency jet ventilation (HFJV) system;

FIG. 64 depicts an exemplary embodiment of a NO generation system installed in a HFJV system;

FIG. 65 depicts an exemplary embodiment of a NO generation system that connects directly to a HFJV system;

FIG. 66 depicts an exemplary embodiment of a NO generation system that delivers high pressure NO gas to a HFJV system;

FIG. 67 depicts an exemplary embodiment of a NO generation system that delivers NO to the low-pressure portion of a HFJV system;

FIG. 68 depicts an exemplary embodiment of a NO device that monitors activity on a HFJV flow controller line;

FIG. 69 depicts an exemplary embodiment of a NO generation system dosing a HFJV system;

FIG. 70 depicts an exemplary embodiment of a NO generation system dosing a HFJV system;

FIG. 71 depicts an exemplary embodiment of a HFJV system that maintains separation between HFJV gas and NO gas until the HFJV flow controller;

FIG. 72 depicts an embodiment of a HFJV system that controls HFJV flow and NO flow with a common flow controller;

FIG. 73 depicts an exemplary embodiment of a NO delivery system operating with a HFJV system;

FIG. 74A depicts an exemplary embodiment of a jet ventilation system in combination with an NO delivery system;

FIG. 74B depicts an exemplary graph of the pulsed inspiratory pressure within the patient during treatment;

FIG. 75 depicts an exemplary NO delivery system setup applied to a ventilator circuit;

FIG. 76 depicts an exemplary embodiment of external portions of a NO delivery system;

FIG. 77 depicts an exemplary embodiment of a NO delivery and gas sampling assemblies with heating element and the sterile sleeve deployed;

FIG. 78 depicts an exemplary embodiment of the NO injector, gas sample and sterile sleeve assembly;

FIG. 79 depicts an exemplary embodiment of a NO injector and gas sample assembly provided without the sterile sleeve;

FIG. 80 depicts an exemplary embodiment of a NO injector and gas sample assembly that utilizes flow control within the controller;

FIG. 81 depicts an exemplary embodiment of a NO injector assembly;

FIG. 82 depicts an exemplary embodiment of an NO injector that utilizes a venturi to draw NO product gas into the inspiratory flow;

FIG. 83 depicts an exemplary embodiment of a remote NO injector that utilizes a compressed gas-actuated valve to modulate NO injection into the inspiratory flow;

FIG. 84 illustrates an embodiment of an external gas flow controller in the form of a balloon with a lumen;

FIG. 85 illustrates an embodiment of an external gas flow controller in the form of an annular/torroidal balloon that squeezes a gas flow tube;

FIG. 86 illustrates an embodiment of an external gas flow controller in the form of an annular/toroidal balloon that variably occludes gas flow;

FIG. 87 depicts an exemplary embodiment of a remote injection module for a NO delivery system with an external recirculation loop;

FIG. 88 depicts an exemplary embodiment of an injection module with external product gas recirculation;

FIG. 89 depicts an exemplary embodiment of an injection module with external product gas recirculation;

FIG. 90 depicts an exemplary embodiment of an injection module with built-in processor;

FIG. 91 depicts a remote injection module with built-in processor and redundant gas supply, gas return, injection flow measurement, and injection flow control;

FIG. 92A depicts a cross-section of an exemplary extrusion with redundant outbound and return product gas pathways;

FIG. 92B depicts a cross-section of a multi-lumen extrusion for pneumatic and electrical communication between a NO generator and a remote injection module with insulative lumens and electrical conductors;

FIG. 93A depicts an NO injector with flow sensor downstream of the flow controller;

FIG. 93B depicts an NO injector with flow sensor upstream of the flow controller;

FIG. 93C depicts an NO injector with flow sensor upstream of the flow controller and a bleed path back to the return flow path;

FIG. 94 depicts a remote injection module that utilizes pressure drop across a flow restriction to measure inspiratory flow;

FIG. 95 depicts a remote injection module with push/pull architecture;

FIGS. 96A, 96B, 96C, 96D, and 96E depict several pneumatic architectures for a remote injection module;

FIG. 97 depicts a NO generation system that provides inspiratory flow and NO to a patient;

FIG. 98 depicts a NO generation system with external blower accessory for delivering inspiratory flow and NO to a patient;

FIG. 99A depicts an exemplary plot showing closing of a flow controller;

FIG. 99B depicts an embodiment of a system that enables a NO delivery system to rapidly slow or arrest the gas flow through a lumen;

FIG. 100 depicts an exemplary embodiment of a NO generation and delivery system for pulsed NO delivery;

FIG. 101 depicts an exemplary embodiment of a NO generation system with a NO concentration sensor in fluid communication with the scrubber;

FIG. 102 depicts an exemplary embodiment of a ventilator cartridge;

FIG. 103 depicts an exemplary embodiment of a remote injection ventilator cartridge design with similar interface as the design depicted in FIG. 16, so that the ventilator cartridges are interchangeable;

FIG. 104 depicts an exemplary embodiment of a system that is configured to determine the size of an unknown volume;

FIG. 105 depicts an exemplary embodiment of a system architecture that can be utilized by a NO generation and/or delivery system to quantify an unknown volume;

FIG. 106A depicts an exemplary embodiment of a volume displacement component that varies the position of a piston in a cylinder with a motor and ball-screw;

FIG. 106B depicts an exemplary embodiment of a volume displacement component that varies a dead volume with a piston driven by a motor-driven cam and return spring;

FIG. 106C depicts an exemplary embodiment of an architecture for compensating for a variable dead volume;

FIG. 106D depicts an exemplary embodiment of an architecture for determining the volume of an unknown cavity;

FIG. 106E depicts an exemplary embodiment of an architecture for determining the volume of an unknown cavity;

FIG. 106F depicts an exemplary embodiment of a system with an unknown scrubber dead volume;

FIG. 107 depicts an exemplary graph showing exemplary inspiratory flow data from a Hamilton T−1 ventilator circuit;

FIG. 108 depicts an embodiment of a system with NO delivered through a tube that is independent of the inspiratory limb;

FIG. 109 depicts an exemplary graph of a method that can be used by a NO delivery system to detect treatment type;

FIG. 110 depicts an exemplary embodiment of a NO injection module that is inserted into an inspiratory limb;

FIG. 111 depicts an exemplary embodiment of an NO injection module that is built into a patient Y fitting;

FIGS. 112A and 112B depict an exemplary embodiment of a NO injection module that promotes mixing of NO into the inspiratory flow;

FIG. 113 depicts an exemplary embodiment of a NO injection module;

FIG. 114 depicts an embodiment of an inspiratory flow path in which NO is introduced to an inspiratory flow through an array of orifices;

FIG. 115 depicts an exemplary embodiment of a NO generation and delivery system that is mounted to a ventilator stand;

FIG. 116 depicts an exemplary embodiment of a system with a wireless remote user interface;

FIG. 117 depicts an exemplary embodiment of a NO generation and delivery system;

FIG. 118 depicts an exemplary graph showing exemplary flow data collected from a ventilator circuit;

FIG. 119 depicts an embodiment of a NO delivery system connected to the inspiratory limb of a ventilation circuit.

FIG. 120 depicts an exemplary graph showing the effect of adding humidity to a 403 ppm NO gas stream at room temperature; and

FIG. 121 depicts an embodiment of a sterilization process that utilizes a recirculating loop of NO.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments. Figures depicting architectures forgo the details of also depicting cabling and control elements to provide focus on the innovation.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure relates to systems and methods of nitric oxide (NO) delivery for use in various settings, for example, inside a hospital room, in an emergency room, in a doctor's office, in a clinic, and outside a hospital setting as a portable or ambulatory device or gas source during patient transport. An NO generation and/or delivery system can take many forms, including but not limited to a device configured to work with an existing medical device that utilizes a product gas, a stand-alone device, a module that can be integrated with an existing medical device, one or more types of cartridges that can perform various functions of the NO system, a compact NO inhaler, and an electronic NO tank. There are multiple applications for generated NO, for example, inhaled therapy to increase oxygen uptake, inhaled therapy to treat infection, topical treatments to treat infection, sterilization of surfaces and equipment, and generation of liquid solutions containing NO nanobubbles. The NO generation system uses a reactant gas containing a mixture of at least oxygen and nitrogen, including but not limited to ambient air, and an electrical discharge (plasma) to produce a product gas that is enriched with NO.

An NO generation device can be used with any device that can utilize NO, including but not limited to a ventilator, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a nebulizer, an extracorporeal membrane oxygenation (ECMO), a bypass system, an automated CPR system, an oxygen delivery system, an oxygen concentrator, an oxygen generation system, and an automated external defibrillator AED, MRI, a gas sterilization system, a nanobubble generator, and a patient monitor. In addition, the destination for nitric oxide produced can be any type of delivery device associated with any medical device, including but not limited to a ventilator, nasal cannula, a manual ventilation device, a face mask, inhaler, or any other delivery circuit. The NO generation capabilities can be integrated into any of these devices, or the devices can be used with a NO generation device as described herein.

In some embodiments, a NO generation device can include one or more gas sensors to measure the concentration of NO in the product gas. In some embodiments, a NOx (i.e. NO+NO₂) sensor can be utilized to measure NO. Various NO sensor locations and quantities are depicted in the figures. It should be understood that one or more product gas NO sensors can be added to any of the embodiments to inform the device controller of NO gas product gas concentration. The NO product gas concentration is utilized by the device controller as one or more of feedback to the plasma controls to obtain a target product gas NO concentration, an input into an injection flow calculation to determine the quantity of NO to inject into a patient inspiratory stream to attain an accurate delivered NO dose, and to assess an amount of NO loss occurring within the system.

References to the term “pump” are inclusive to all methods of propelling a gas, for example, through a pipe, including but not limited to gear pumps, diaphragm pumps, syringe pumps, blowers, centrifugal pumps, reciprocating piston pumps, piezoelectric pumps, and others. References to the term “valve” are inclusive of variable and binary flow control devices of all types unless specifically stated otherwise. A “flow controller” can be as simple as a binary valve and as sophisticated as a mass flow controller. It should be understood that a simple binary valve in combination with appropriate measurements (e.g., pressure, temperature, humidity, flow) can serve as a mass flow controller. Figures depicting pneumatic architecture do not necessarily show all necessary sensors in order to keep the focus on architecture.

The present disclosure includes ideas in the areas of NO generation and NO delivery. It should be noted that NO delivery concepts can be applicable to NO delivered from a multitude of sources, including NO tanks, electrically generated NO and chemically derived NO (e.g., donor molecules, N₂O₄ processes, etc.).

Gaseous NO for the aforementioned treatments can be sourced from a variety of systems using a variety of approaches. In some embodiments, gaseous NO is sourced from a cylinder of NO in a balance of an inert gas (e.g. nitrogen). In some embodiments, NO is formulated from the reduction of nitrogen dioxide gas. In some embodiments, nitrogen dioxide gas is formed from N₂O₄ and then reduced to NO. In some embodiments, NO is generated by chemical release from a polymer or other solid using heat or a light source. In some embodiments, NO is formed by focusing laser energy within a reactant gas. In some embodiments, NO is generated electrically via either electrical discharge, dielectric barrier discharge or microwave energy.

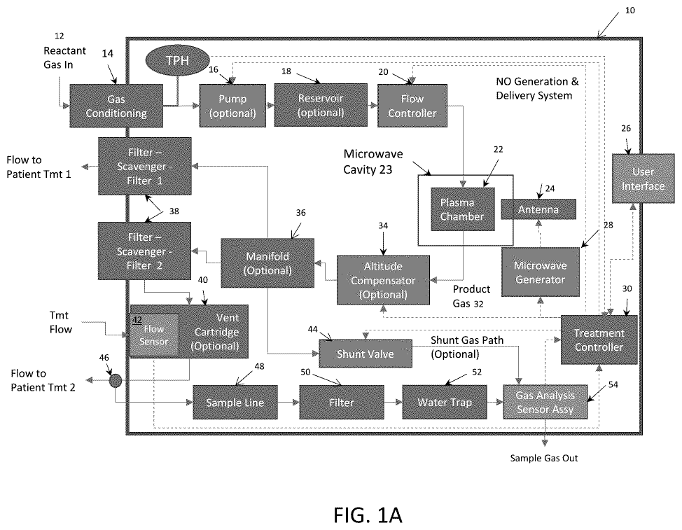

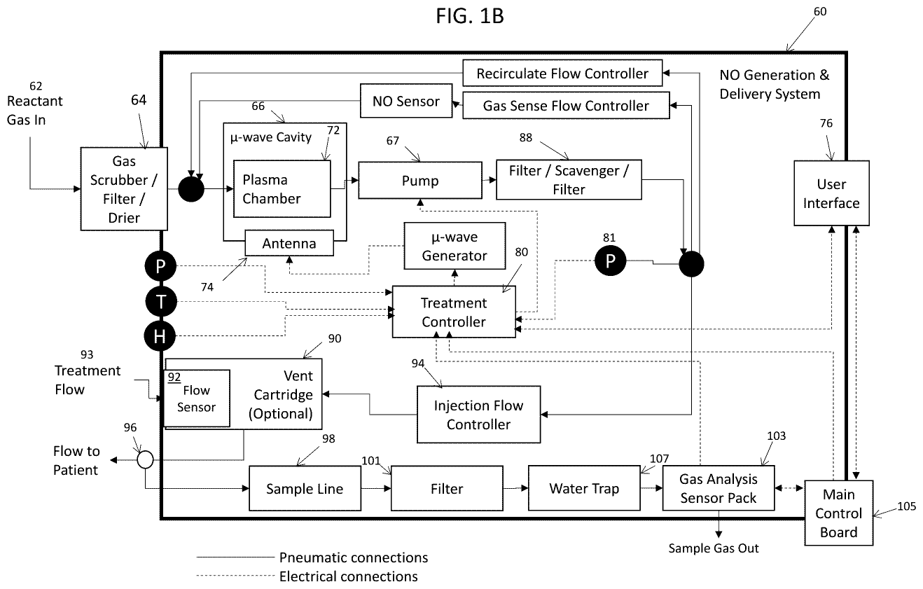

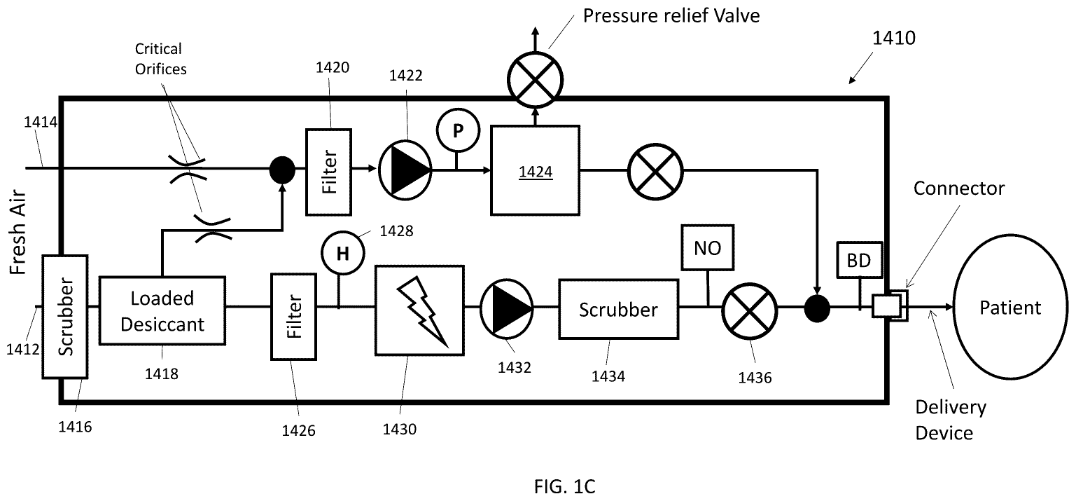

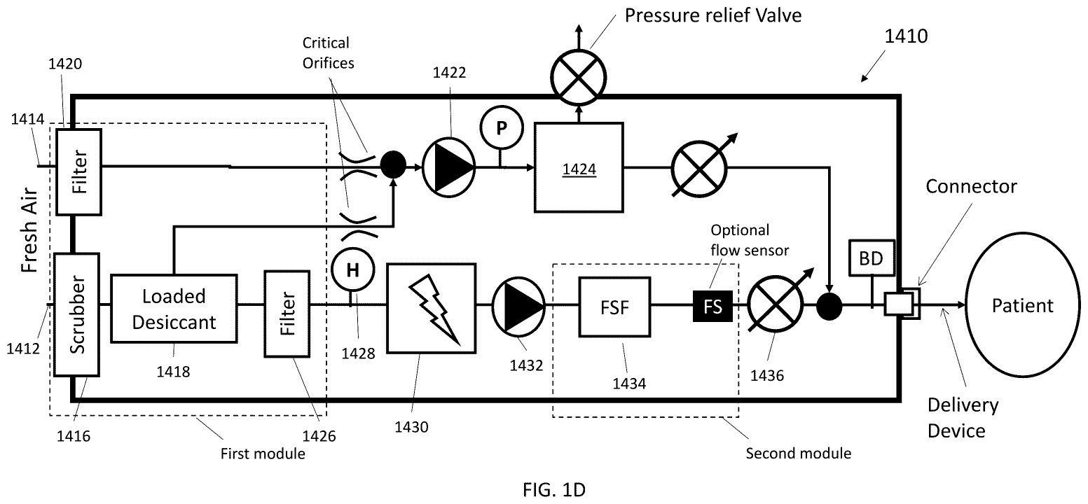

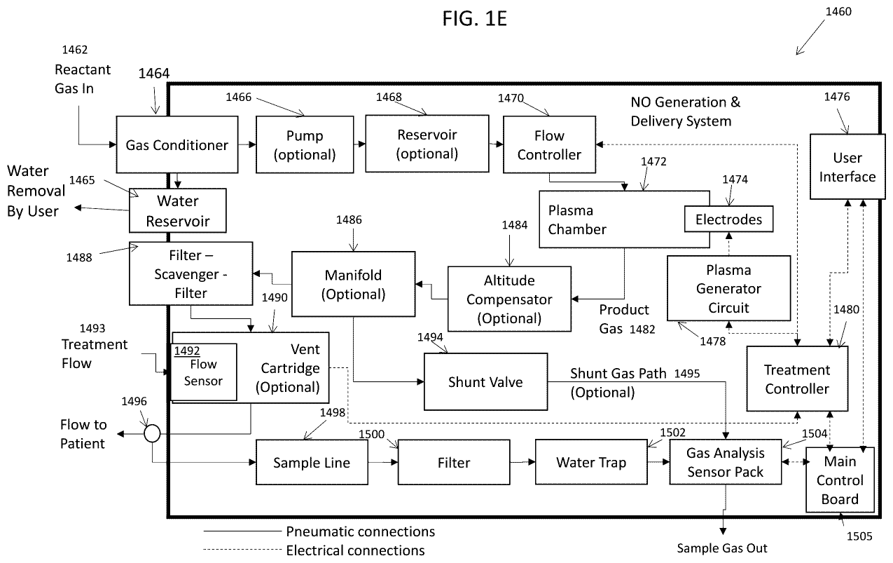

FIG. 1A illustrates an exemplary embodiment of an electrical NO generation system 10 that includes components for reactant gas intake 12 and delivery to a plasma chamber 22. In some embodiments, this NO generation system is used in conjunction with a breath detection system for utilizing the information therefrom to deliver a drug (NO) to a patient based on detected respiratory events. Reactant gas enters the system through a gas conditioning cartridge 14 that includes one or more of a chemical scrubber (e.g. VOCs, ammonia), particulate filter, and one or more humidity adjustment mechanisms. A temperature, pressure and/or humidity (TPH) sensor can characterize the physical properties of the reactant gas. This information is transferred to the treatment controller for input into the NO generators calculation of microwave activity (e.g. frequency, power level) and reactant gas flow rate to achieve a target level of NO production. The system is configured to produce, with the use of a microwave generation circuit 28, microwave cavity 23, and one or more microwave source antennas 24, a product gas 32 containing a desired amount of NO from the reactant gas. The system includes a treatment controller 30 in electrical communication with the microwave generator 28 that is configured to control the concentration of NO in the product gas 32 using one or more control parameters relating to conditions within the system and/or conditions relating to a separate device for delivering the product gas to a patient and/or conditions relating to the patient receiving the product gas.

The controller 30 is also in communication with a user interface 26 that allows a user to interact with the system, enter a target NO dose level, view information about the system and NO production, and/or control parameters related to NO production.

The controller 30 is also in communication with a respiratory sensor. The respiratory sensor is in fluid communication with the inspiratory gas pathway and is utilized to capture respiratory data so that the controller can detect respiratory events.

In some embodiments, the NO system pneumatic path includes a pump pushing or pulling air through a manifold 36. In other embodiments, pressurized reactant gas is provided to the inlet of the NO generator. The manifold is configured with one or more valves (e.g., three-way valves, binary valves, check valves, and/or proportional orifices). The treatment controller 30 controls pump power, gas flow rate, the frequency of plasma pulses, the power in the plasma, and/or the direction of the gas flow post-electrical discharge. By configuring valves, the treatment controller can direct gas to the manual respiration pathway, the ventilator pathway, or the gas sensor chamber for direct measurement of NO, NO₂, and O₂ levels in the product gas. In some embodiments, respiratory gas (i.e., the treatment flow) can be directed through a ventilator cartridge that measures the mass flow of the respiratory gas and can merge the respiratory gas with NO product gas.

The output from the NO generation system in the form of the product gas 32 enriched with the NO produced in the plasma chamber 22 can either be directed to a respiratory or other device for NO delivery to a patient or can be directed to a plurality of components provided for self-test or calibration of the NO generation system. In some embodiments, the system collects gases to sample in two ways: 1) gases are collected from a patient inspiratory circuit near the patient and pass through a sample line 48, a filter 50, and a water trap 52, or 2) gases are shunted directly from the pneumatic circuit as they exit the plasma chamber 322. In some embodiments, product gases are shunted with a shunt valve 44 to the gas sensors after being scrubbed but before dilution into a patient airstream. In some embodiments (not shown), shunted product gases are diluted with air prior to exposure to gas sensors to provide NO/NO₂/O₂ concentrations in the range of the gas sensors. This dilution brings product gas concentration (e.g. 50 to 5000 ppm) down to inhaled concentrations (e.g. 0.1 to 300 ppm) for the gas concentration to be within the inhaled gas sensor measurement range. In some embodiments, product gases are collected from an inspiratory air stream near the device and/or within the device post-dilution. Within the gas analysis portion of the device, the product gas passes through one or more sensors to measure one or more of temperature, humidity, concentrations, pressure, and flow rate of various gasses therein. Sampled gas is then optionally scrubbed for NO and/or NO₂ (not shown) prior to release to atmosphere. In other embodiments, sampled gas is released to house vacuum or introduced to the reactant gas pathway (not shown).

FIG. 1B depicts an embodiment of a NO generation and delivery system 60 with a recirculation architecture. Reactant gas 62 enters the system through a gas conditioner 64 that includes one or more of a chemical scrubber, a particulate filter, and a humidifier/dehumidifier. In some embodiments, reactant gas is dried completely or nearly completely to eliminate changes in NO production calibration related to reactant gas water content. Reactant gas passes through a junction where the recirculated gas enters the pathway and enters a plasma chamber. The plasma chamber is located within a microwave cavity that forms a plasma ball within the reactant gas flow path. In other embodiments (not shown), NO is generated within the plasma chamber by electrical discharges between two or more electrodes. A pump 67 is used to pull gas through the plasma chamber. In some embodiments, the pump operates at a constant flow rate (e.g., 3 slpm). Alternatively, the pump 67 can be positioned downstream of a filter/scrubber/filter 88. Gas enters the recirculation loop to make up for gas injected into the patient inspiratory pathway. Gas exiting the pump passes through a NO₂ scrubber to eliminate NO₂ formed by the NO generation process and NO₂ formed from oxidation of NO as it travels around the recirculation loop. The NO₂ scrubber includes one or more filters before and/or after it to prevent migration of scrubber material and capture particles released from the scrubber and potentially other parts of the system. After exiting the scrubber/filter stage 88, the gas enters a node that is maintained at constant pressure, as measured by pressure sensor 81. There are two or more flow paths exiting the constant pressure node. One flow path leads to a flow controller that controls product gas injection into the patient inspiratory stream. In some embodiments, constant concentration and pressure NO is introduced to the patient inspiratory stream in proportion to the inspiratory stream mass flow rate. In other embodiments, NO is intruded in a pulsatile manner to dose specific respiratory events (e.g. beginning of inhalation, middle of inhalation, end of inhalation). The node is maintained at constant pressure to facilitate and improve the accuracy of the injection flow controller. A second flow path and flow controller can be utilized to maintain a target pressure at the node and return product gas to the beginning of the recirculation loop (pre-plasma chamber). A third and optional flow path includes a flow controller that regulates the product gas flow through one or more gas sensors used to monitor the product gas NO and/or NO₂ levels within the recirculation loop. A microwave antenna or stub 74 within the resonant microwave cavity 72 is energized by a microwave generator 78 that produces microwaves of varying pulse frequency, pulse duration, and/or power level based on desired treatment conditions received from a treatment controller 80. A user interface 76 receives desired treatment conditions (dose, treatment mode, etc.) from the user and communicates them to the main control board 105. In some embodiments, the main control board 105 may include a controller, processor and/or other computer hardware/software for controlling other elements/components of the system, including other controllers. The main control board 405 relays to the treatment controller 80 a target dose and monitors measured NO concentrations from the gas analysis sensor pack 103. The main control board 105 monitors the system for error conditions and can generate alarms, as required. Reactant gas 62 is converted into product gas 82 when it passes through the plasma chamber 72 and is at least partially converted into nitric oxide and nitrogen dioxide. Ambient gas measurements made by pressure, temperature and/or humidity sensors on the left edge of the image are used as inputs to the treatment controller for plasma compensation to improve dose accuracy. In some embodiments, the treatment controller varies one or more of the duty cycle, the frequency and/or the power of the microwave pulses as humidity increases from 10% to 50%, for example, to maintain a constant production level of NO. In some embodiments, the treatment controller varies one or more of the microwave pulse duty cycle, pulse frequency and/or power as pressure increases in the plasma chamber to maintain a constant production level of NO. In some embodiments, the vent cartridge 90 includes a mass flow sensor 92 that measures the treatment gas flow 93. The treatment gas flow measurements from the flow sensor 92 serve as an input into the reactant gas flow controller 70 via the treatment controller 80. In some embodiments, the inspiratory mass flow sensor is utilized as the respiratory sensor for breath detection. After product gas 82 is introduced to the treatment flow, it passes through inspiratory tubing and an optional humidifier (not shown) or moisture exchanger (not shown). Near the patient, a fitting 96 is used to pull a fraction of inspired gas from the inspiratory flow, through a sample line 98, filter 101, water trap 107 and humidity exchange tubing (e.g. Nafion) to prepare the gas sample and convey it to gas sensors 104. Gas sensors may measure one or more of nitrogen, oxygen, nitric oxide, nitrogen dioxide, helium, and carbon dioxide. Sample gas exits the gas analysis sensor pack 104 to ambient air. In some embodiments, the sample gas is scrubbed for one or more of NO, NO₂, and medicines prior to release to the environment. In some embodiments, the system 60 can optionally direct gas through a shunt valve 94 and shunt gas path 95 directly to the gas sensor pack and out of the system. In some embodiments involving the shunt valve 94, the manifold 86 includes a valve (not shown) to block flow to the filter-scavenger-filter when the shunt valve 94 is open.

FIG. 1C depicts a pulsed NO delivery system 1410 with a pressurized scrubber and pressurized bypass architecture. Reactant gas (i.e., air) enters the system into either a desiccant pathway 1412 or a bypass pathway 1414. Gas passing through the desiccant pathway is scrubbed for VOCs and harmful chemicals using a scrubber 1416 prior to passing through desiccant 1418 where it is either partially or completely dried. A portion of the dried reactant gas exits the drying component and is introduced to the bypass gas flow. The ratio of the blending is set in this embodiment by critical orifices, however other embodiments utilize proportional valves and/or pump speed to set the flow rate in each channel. In this embodiment, the mix ratio (desiccated vs. non-desiccated gas) is the same for all environmental conditions, resulting in some variation in purge gas humidity. The main objective of drying the purge gas (i.e. gas in the bypass channel) is to prevent condensation within the system when ambient conditions are humid. The mix ratio is selected to ensure that purge gas does not condense at worst case ambient humidity levels and purge gas pressures. Gas in the bypass pathway flows through a particle filter 1420 and a pump 1422 before it is accumulated in a purge reservoir 1424, the exit of which is controlled by a valve. In the event that patient respirations are slow and too much gas accumulates in the bypass reservoir, a pressure relief valve can be opened (passively or actively) based on the pressure within the bypass reservoir. Active pressure relief is controlled by the system controller, based on the pressure as measured by a pressure sensor. Other embodiments use a permanently open orifice to continuously bleed pressure from the purge reservoir to prevent over-pressurization. Other embodiments use a proportional flow valve at the exit of the accumulator (not shown).

On the desiccated flow path, reactant gas passes through a particle filter 1426 to remove particulate that could be altered in the plasma chamber or clog the flow path and/or components downstream. A humidity sensor 1428 measures the humidity of the reactant gas to provide an indication when the desiccant stage has been exhausted. In some embodiments, this humidity measurement is also utilized as an input to calculating the plasma parameters (e.g. duty cycle, frequency, power) to achieve accurate amounts of NO production. Reactant gas then flows into a plasma chamber 1430 where nitric oxide is formed in the reactant gas due to elevated temperatures from making a plasma in the gas. The plasma is formed by an arc discharge between two electrodes in some embodiments. In other embodiments, the energy from microwave antennas is focused in a small area where a plasma ball forms. Product gas (reactant gas+NO) exits the plasma chamber and flows through a pump 1432 that pressurizes a NO₂ scrubber 1434 with product gas. An optional NO concentration sensor is in fluid communication with the pressurized scrubber to report to the system controller the concentration of NO gas to be delivered. The exit of the pressurized scrubber is controlled by one or more valves 1436. In some embodiments, the valve is a proportional valve.

The NO generation and delivery system depicted in FIG. 1C operates by delivering a pulse of NO followed by a pulse of purge gas. This approach leaves the delivery device devoid of NO-containing gas between breaths, thereby decreasing the amount of time that NO can oxidize into NO₂. In some embodiments, the delivery device is a nasal cannula. In other embodiments, the delivery device is a tube connected to a mask. The delivery device connects to the generator with a connector. A sensor labeled “BD” in the figure measures pressure within the delivery device to detect breaths. In some embodiments, the BD sensor measures gas properties in the same lumen in which drug (NO) is delivered. In other embodiments, the BD sensor is in fluid communication with or located in a dedicated lumen for breath detection.

Not shown in FIG. 1C is the treatment controller that collects data from the various sensors and controls the plasma chamber, the pump and the valves to conduct the patient treatment. In some embodiments, the treatment controller is a hardware circuit. In other embodiments, the treatment controller is a software-driven microprocessor. For example, the treatment controller utilizes the indicated product gas NO concentration from a NO sensor as an input into determining the flow rate and duration of a pulse of gas to be delivered in order to deliver a target dose. In some embodiments, the treatment controller utilizes the NO sensor to close the loop on NO generation in the plasma chamber, increasing or decreasing plasma activity (i.e. frequency, duty cycle, energy, etc.) to achieve a target product gas concentration. Measurement of product gas NO concentration enables a NO generation system to compensate for variations in environment (pressure, temperature, humidity) and the system (electrode wear, scrubber age, etc) over time.

FIG. 1D depicts an embodiment of a pressurized scrubber, pressurized bypass device with removable modules. In some embodiments, the modules are disposable. A first module treats incoming gas (typically air) into the system by removing particles with a particle filter, volatile organic compounds with a scrubber and excess water with a desiccant. A second module includes a filter-scrubber-filter (labeled FSF) for removal of particles and NO₂ gas as well as an optional flow sensor. Inclusion of a flow sensor within the removable module enables easy replacement of a flow sensor, sparing the user from having to recalibrate the flow sensor over time. In some embodiments, the first and second removable modules are combined into a common assembly.

FIG. 1E depicts an embodiment of a NO generation and delivery system 1460. Reactant gas 1462 enters the system through a gas conditioner 1464. The gas conditioner does one or more of filter for particulates, add or remove water, and scrub for VOCs. In some embodiments, the gas conditioner is completely passive (no active components). In other embodiments, the gas conditioner is active (e.g. a variable chiller to condense water or a means to pressurize gas to a known level to condense out a predictable amount of water), controlled by the device controller based on a humidity measurement (humidity sensor not shown). In some embodiments, water removed from reactant gas by the gas conditioner collects in a water reservoir 1465 for subsequent removal by a user. In some embodiments, water removed from reactant gas by the gas conditioner is added back to the product gas (not shown). This can eliminate the need for a user to drain the water reservoir when the reservoir is full.

A pump 1466 is used to propel gas through the system. Whether or not a system includes a pump can depend on the pressure of the reactant gas supply. If reactant gas is pressurized, a pump may not be required. If reactant gas is at or near atmospheric pressure, a pump or other means to move reactant gas through the system is required. A reservoir 1468 after the pump attenuates rapid changes in pressure and/or flow from a pump. Coupled with a flow controller 1470, the reservoir, when pressurized, can enable a system to provide flow rates to the plasma chamber 1472 that are greater than the pump 1466 flow rate. This enables the use of a smaller, lighter, quieter and more efficient pump. Electrodes 1474 within the plasma chamber 1472 are energized by a plasma generation circuit 1478 that produces high voltage inputs based on desired treatment conditions received from a treatment controller 1480. The treatment controller utilizes sensor measurements of reactant gas pressure (sensor marked “P”) and/or reactant gas flow rate (based on flow controller setting or flow rate sensor (not shown)) as inputs in determining plasma parameters to produce a target amount of NO. A user interface 1476 receives desired treatment conditions (dose, treatment mode, etc.) from the user and communicates them to the main control board 1505. The main control board 1505 relays to the treatment controller 1480 the target dose and monitors measured NO concentrations from the gas analysis sensor pack 1504. The main control board 1505 monitors the system for error conditions and generates alarms, as required. The reactant gas 1462 is converted into product gas 1482 when it interacts with the plasma as it passes through the plasma chamber 1472 and is partially converted into nitric oxide and nitrogen dioxide. An altitude compensator 1484, typically consisting of one or more valves (for example, proportional, binary, 3-way), is optionally used to provide a backpressure within the plasma chamber 1472 for additional controls in nitric oxide production. In some embodiments, the altitude compensator setting is varied by the treatment controller based on the plasma chamber pressure sensor measurement. Product gases pass through a manifold 1486, as needed, to reach a filter-scavenger-filter 1488 assembly that removes nitrogen dioxide from the product gas. From the filter-scavenger-filter 1488, product gas is introduced to a patient treatment flow directly, or indirectly through a vent cartridge 1490. In some embodiments, the vent cartridge 1490 includes a flow sensor 1492 that measures the treatment flow 1493. The treatment flow measurements from the flow sensor 1492 serve as an input into the reactant gas flow controller 1470 via the treatment controller 1480. After product gas 1482 is introduced to the treatment flow, it passes through inspiratory tubing. Near the patient, a fitting 1496 is used to pull a fraction of inspired gas from the inspiratory flow, through a sample line 1498, filter 1500, water trap 1502 and moisture exchange tubing (e.g. Nafion) to prepare the gas sample and convey it to gas sensors 1504. Sample gas exits the gas analysis sensor pack 1504 to ambient air. In some embodiments, the sample gas that is exhausted to the environment is first scrubbed of NO and/or NO₂ prior to release (not shown). In some embodiments, the system 1460 can optionally direct gas through a shunt valve 1494 and shunt gas path 1495 directly to the gas sensor pack and out of the system. In some embodiments involving the shunt valve 1494, the manifold 1486 includes a valve (not shown) to block flow to the filter-scavenger-filter when the shunt valve 1494 is open.

### Gas Humidity Management

Multiple solutions to humidity management, which involves the addition or removal of water, are presented herein. These solutions may be used individually or in combination to achieve a target level of humidity management. In some embodiments, humidity management involves the removal of water from gas (e.g., reactant gas, product gas) within the system. In some embodiments, humidity management is also utilized to increase the humidity of a gas. This can be necessary to protect a gas sensor (e.g., electrochemical sensor) or a material (e.g., soda lime scrubber) from being dried out during device operation.

Nitric oxide generation systems involve one or more gas flows through the system (e.g., sample gas flow, reactant gas flow). The humidity within these gases must often be managed to be within a particular range, the range driven by one or more of prevention of condensation, gas-contacting sensor requirements, scrubber material requirements (e.g. water content) and NO production limitations. In some embodiments, 25% of the incoming gas water content must be removed to prevent condensation within the system. In some embodiments, upwards of 50% of the water content must be removed in order to ensure predictable and consistent NO generation from electrical discharges, for example. For example, ambient air at 40° C. and 95% relative humidity contains 48.5 g/m\hat{ }3 of water. One embodiment of an electrical NO generation system requires less than 12 g/m\hat{ }3 of water within the reactant gas to reliably initiate plasma. Hence, 75% of the reactant gas water content must be removed prior to NO generation. Even when electrical breakdown is certain within humid reactant gas, NO production can decrease as much as 50% in the presence of humidity. This is because water within the reactant gas increases the resistance between electrodes and absorbs thermal energy that could otherwise be utilized in generating NO. In some embodiments, all of the water (i.e. >99%) is removed from the reactant gas prior to NO generation to ensure consistent and efficient NO generation.

Various methods have been conceived to manage the humidity level within a gas stream to a target range. In some embodiments, desiccant material (e.g., silica, polyglycol, 1,2,3-propanetriol) is utilized to drive the humidity of sample gas to a target range prior to the gas analysis sensors. In some embodiments, the type of desiccant selected is based on the target humidity range. For example, molecular sieve material can be utilized to remove 100% of water from a gas stream. Molecular sieve material can also be utilized to achieve a target level of humidity between ambient levels and zero by varying the ratio of 100% dried gas and ambient gas in a blend of gases. In some embodiments, a humidity management material (e.g., 1,2,3-Propanetriol) is utilized to achieve a target humidity level in a reactant gas stream (e.g., 49% relative humidity). In some embodiments, a humidity management material can be loaded with water at the beginning of use. When exposed to excessively humid gas stream, the humidity management material pulls water out of the gas stream. When exposed to excessively dry gas, the humidity management material releases water into the gas stream to increase the gas stream humidity. Humidity management materials can be utilized in addition to or instead of a water separator to manage water content within a gas stream within a nitric oxide device. In some embodiments, desiccant is utilized to dehumidify a stream of gas sampled from a humidified inspiratory limb so that the gas can be passed to one or more gas sensors for gas analysis. In some embodiments, dry desiccant is utilized to at least partially dry the reactant gas entering a NO generation device to prevent condensation within the device. Humidity management material is typically in the form of beads, sheets, tubes or fibers that are arranged to form gas passages with high surface area. Beads can be useful because of their high surface area and the tortuous path gas must pass through, ensuring high bead-gas interaction. In some embodiments, humidity management material is formed into grids, channels, foams or other shapes to provide high gas-humidity management material interaction while not presenting too much flow restriction.

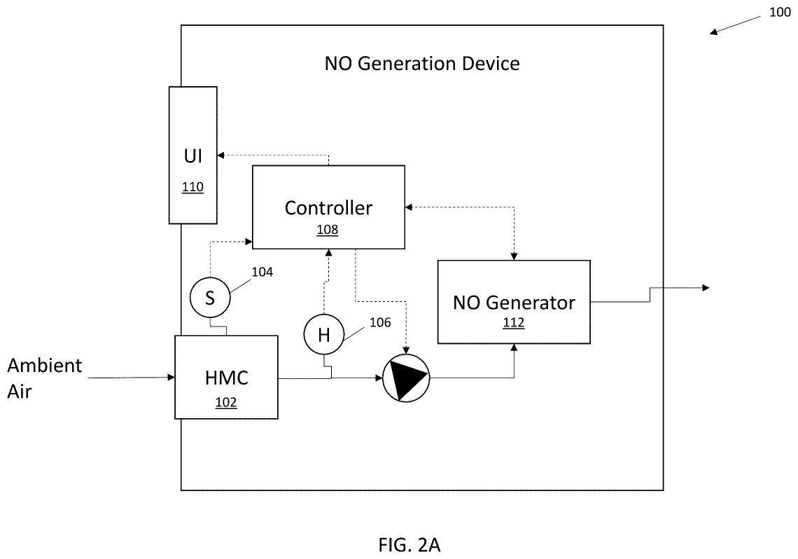

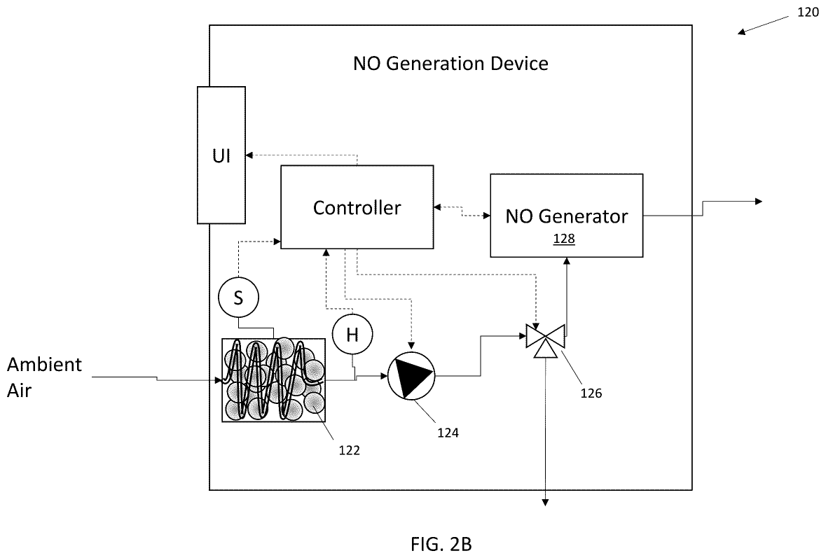

In some embodiments, humidity management material can be packaged within a removable cartridge. FIG. 2A depicts an exemplary embodiment of a NO generation device 100 that utilizes a humidity management cartridge (HMC) 102 to maintain an acceptable humidity level within a gas. Ambient air enters the device through an HMC that is removably attached to the NO generator enclosure 112. A sensor 104 is utilized to detect the presence of the HMC. There are multiple technologies for this sensor including but not limited to optical, capacitive, electrical contacts, mechanical button press, etc. Gas exiting the HMC is measured for water content using a sensor 106. Both sensors can be configured to communicate with a controller component 108. The controller component 108 communicates with a user interface (UI) 110, as needed, to inform the user of device status, including but not limited to HMC installation status, HMC status (e.g. viable or needs replacement) and reactant gas humidity status. HMC status may be based on one or more of a duration of time that the HMC has been installed, the measured humidity of the gas exiting the HMC, an expiration date of the HMC, and humidity levels of the gas entering the HMC (e.g., ambient humidity). In some embodiments, the controller also utilizes the gas water content information as an input into determining NO generation settings (e.g., plasma duty, frequency, power, etc.). For example, the controller can compensate for elevated humidity within the reactant gas by increasing plasma duty cycle in order to achieve a target production level of NO despite production quenching effects.

In some embodiments, the gas stream that passes through an HMC serves as a reactant gas for a plasma chamber or a purge gas. In some embodiments, the gas is an inspiratory limb sample gas collected for analysis (not shown). When the water content of reactant gas falls outside of the acceptable range, this can be indicative of the HMC requiring replacement. Depending on the ambient air conditions, an HMC may require replacement due to either drying out (i.e., no remaining moisture content to humidify an incoming gas stream) or excessive moisture (i.e., unable to adsorb additional water from the incoming gas, saturation). Gas water content falling outside of the acceptable range of water content can also be an indication of HMC failure (e.g., cracked housing, exhausted humidity management material). When gas downstream of the HMC falls out of the target range, a NO generation device may do one or more of the following: stop treatment and warn the user that the HMC requires replacement.

In some embodiments, the HMC is a single-use component that is disposed of after use. In some embodiments, the HMC is a reusable component. In one exemplary embodiment, an HMC is filled with a humidity management material (e.g., a desiccant or 2-way humidity exchange material). After removal from the NO generation system, the HMC is placed in an environment with low relative humidity (e.g., low water content, high temperature) to drive water out of the humidity management material. In some embodiments, a HMC drying device is used to remove water from an HMC. The drying device elevates the temperature of the HMC. In some embodiments, the drying device conveys a stream of gas through the HMC to expedite the drying process. A humidity sensor downstream of the HMC is utilized to measure the humidity of the gas exiting the HMC. A controller within the HMC drying device can utilize the gas humidity measurement to determine when the HMC is sufficiently dry that it can be put back into service.

In some embodiments, a humidity management material is utilized to manage water content in the reactant gas and/or purge gas for an NO generation system. Over time, the humidity management material can absorb water to a point that it has no additional capacity for water. In some embodiments, a humidity sensor downstream of the desiccant is utilized to detect when the desiccant is reaching capacity. For example, in some embodiments, a molecular sieve material is nearly 100% efficient at water removal. In some embodiments, when the humidity in the gas downstream of the desiccant begins to increase and/or crosses a predetermined threshold, this change is measured by a sensor and reported to a controller within the device. In some embodiments, the device prompts the user to replace desiccant material when the gas humidity downstream of the desiccant material reaches and/or crosses a threshold.

In some embodiments, the system monitors one or more of ambient humidity, pressure and temperature with one or more sensors. The system controller utilizes one or more of the ambient humidity, ambient temperature, ambient pressure, and reactant gas flow rate (measured or known by design) to calculate the quantity of water that has entered the system over a period of time. Knowing the capacity of the humidity management material, the system can predict when the desiccant will be fully utilized. In some embodiments, the system controller generates warnings and/or alarms as the desiccant is filling to alert the user as to when the desiccant will require replacement.

### Reusable Humidity Management

In some embodiments, the HMC can be a reusable component. For example, an HMC can be removed and placed in a hot dry environment to drive water out of saturated desiccant material. In some embodiments, the HMC is exposed to humidity at a target humidity (e.g., 30%, or 50%) so that the HMC will lose or take-on water, as needed, to restore a target water content level. In some embodiments, gas is passed through the HMC as it is restored to expedite the humidity transfer process.

### Permanent Humidity Management

In some embodiments, an NO generation and/or delivery device includes a permanent solution to reactant gas management. This solution capitalizes on the fact that that a NO generation device is predominantly used in a humidity-controlled environment with access to unlimited electrical power.

FIG. 2B depicts an exemplary system 120 with a built-in, reusable dehumidification feature that includes humidity management material 122 (e.g., beads of 1,2,3-Propanetriol) in a chamber. Ambient air enters the system and passes through humidity exchange tubing (e.g., perforated tubing, Nafion tubing) in close proximity to the humidity management material. The air then passes through a pump 124 and an optional 3-way valve 126. Depending on the valve setting, as dictated by the device controller in this example, gas can either be directed to the environment or through a plasma chamber. When the design does not include the 3-way valve, all gas is passed through the plasma chamber/NO generator 128. In systems that include a 3-way valve, gas is directed toward the environment when the NO generation system is not in use.

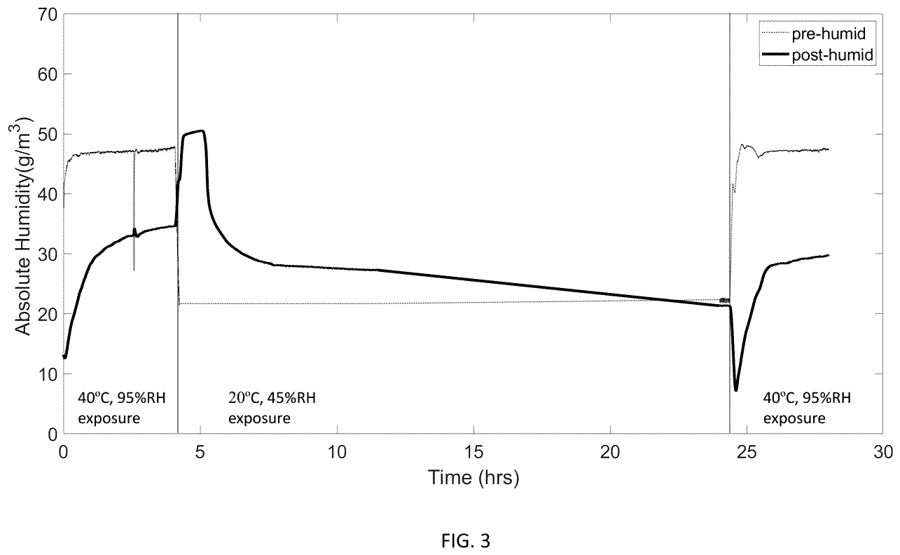

The system is typically utilized in a controlled environment (e.g., hospital, ambulance), where temperatures are roughly 20° C. and 20-50% relative humidity (RH). When the device is taken outside (e.g., to transport a patient), it may be exposed to ambient air with higher water content. The humidity management material removes water from the incoming gas when the incoming gas is higher than the target humidity of the humidity management material. When the system is returned to a controlled environment, the drier incoming gas will remove water from the humidity management material. The quantity of humidity management material can be sized according to the expected exterior conditions and duration of transport to ensure adequate humidity management when the system is outside. For example, humidity management material can be sized to last 2 hours when a system is exposed to 40° C. 95% RH. FIG. 3 depicts an exemplary graph showing experimental results of a reactant gas humidity control system that utilizes 1,2,3-Propanetriol that targets 49% RH in a gas stream. The experiment begins with high humidity gas (40° C., 95% Relative humidity) entering the system to simulate device operation in a high-humidity external environment (e.g. patient transport between hospitals). During the initial 4-hours, the humidity management material removes water from the gas stream to limit reactant gas absolute humidity to less than 35 g/m\hat{ }3. After 4 hours, the device receives drier ambient air (20° C., 50% RH) to simulate the hospital environment. Within the hospital, the humidity control material releases water into the gas stream to reset. The post-humidity management material gas stream humidity increases to 50 g/m\hat{ }3 as water is released for roughly 2 hours. After these 2 hours, the humidity management material is effectively reset. At roughly 24.5 hours, the humidity management material is again exposed to 40° C., 95% Relative humidity gas.

As the humidity management material releases water, there is a risk that gas humidity becomes too high for NO generation and/or the internal pressure within the system (e.g. condensation conditions). In some embodiments, the release of water from the humidity management material is rate-limited so that reactant gas humidity levels remain within the acceptable range. This can be done by blending gas flow through the humidity management material with ambient gas. In some embodiments, the humidity management material is reset by flowing ambient air through it while an independent flow of ambient air is delivered to the NO generation pathway.

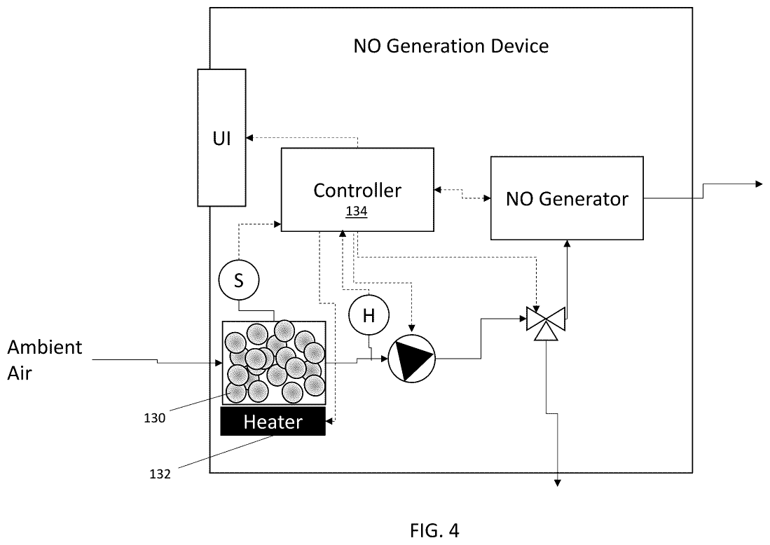

In some embodiments, as depicted in FIG. 4, the drying of the humidity management material can be done actively when the system is plugged into an external power source. As shown in FIG. 4, incoming ambient gas passes through a bed of humidity management material 130, in direct contact. A heater 132, controlled by a system controller 134, is utilized to expedite drying of the desiccant material. In some embodiments, the heater is only utilized when the system is connected to external power. In some embodiments, the system uses battery power to power the heater.

### Gas Compression to Remove Water

For gas at a given temperature and pressure, there is a maximal amount of water that the gas can hold in the vapor state. Any amount of water beyond that cannot be maintained as a gas and will condense out of the gas. Every NO generation system varies pressure of gas within it as the gas courses through the system. The highest pressure within the system is known, so the maximum amount of water content that can be allowed within the gas without condensation is also known. In some embodiments, gas within the system (e.g., reactant gas or product gas) is compressed to a specific amount to remove water within the gas to prevent condensation elsewhere in the system. The amount that the gas is compressed correlates with a maximal amount of water content that can be utilized by the system without condensation. In the compression process, excess water content condenses out of the gas thereby limiting the water content within the gas downstream. In some embodiments, the maximal pressure within a NO generation system is located after a pump and before a scrubber, where the pressure reaches a maximum level within the system. An exemplary system compresses incoming reactant gas to a level that exceeds the pressure elsewhere in the system to force excess water out of the gas before the gas flows into downstream parts of the system. In some embodiments, reactant gas is pumped into a reservoir and pressurized beyond a minimum value that is related to the minimum pressure required to condense excess water from the reactant gas.

In some embodiments, water removed from a flow of reactant gas and/or product gas is collected in a reservoir and drained by a user. In some embodiments, water collected from reactant gas and product gas is combined with water collected from the inspired gas sample flow. This reduces the number of use steps to maintain the water reservoir. In some embodiments, the common reservoir also decreases the dead volume of the sample gas flow path, enabling NO₂ measurements that are more reflective of the NO₂ concentration at the inspiratory limb due to decreased transit time. The lack of a water trap within the gas sample line also ensures that the dead volume of the gas sample path is consistent over time, thereby preventing variation in transit time and NO₂ measurements due to variation in water level within the water trap.

In some embodiments, a controller varies the amount of water removed from the reactant and/or product gas during device operation. In some embodiments, the amount of water removed is related to one or more of the ambient humidity, the system operating temperature, the age of the scrubber (e.g. older soda lime scrubbers are more dry, hence less water removal may be necessary), the product gas injection flow rate into the inspiratory limb (explanation: higher product gas injection in a recirculating architecture results in higher quantities of ambient make-up air being added to the system. Higher ambient air content within a recirculation loop can require a greater amount of humidity removal level to prevent condensation within the system).

FIG. 5 depicts an embodiment of a gas humidity removal process that involves pressurizing the gas to force condensation. Ambient air enters the process at step 140. The gas is then pressurized (e.g., with a pump) in step 142 and cooled (e.g., active cooling or passive cooling) in step 144. As the pressurized gas cools, water condenses out of the gas and is either collected or expelled from the system (step 146). The pressurized gas then flows through an expansion valve (step 148) and optional warming stage (step 150) prior to flowing on to downstream, humidity-sensitive process steps (e.g., plasma) and components (e.g., scrubber and sensors). The optional warming stage is a mitigation that ensures that the relative humidity in the gas flow is less than 100% to prevent possible condensation.

### Gas Cooling to Remove Water

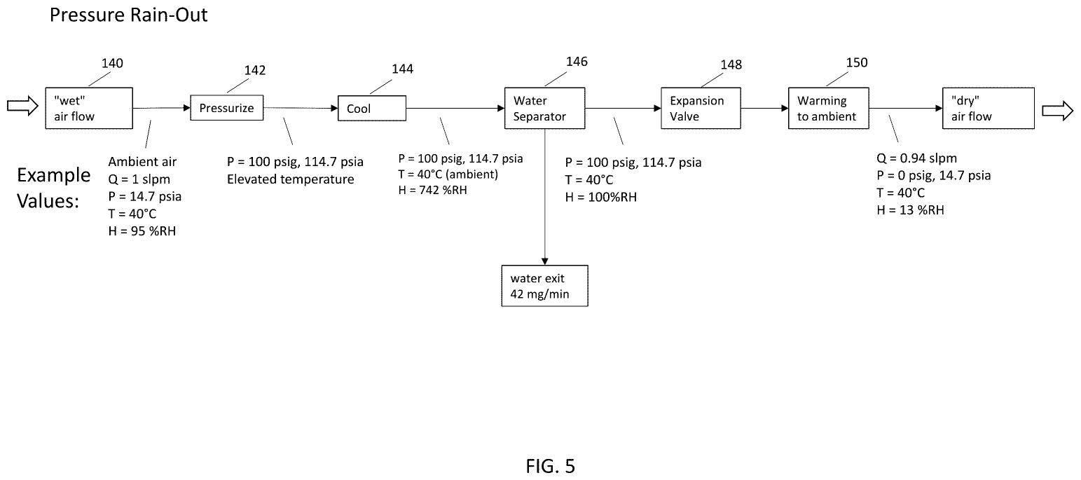

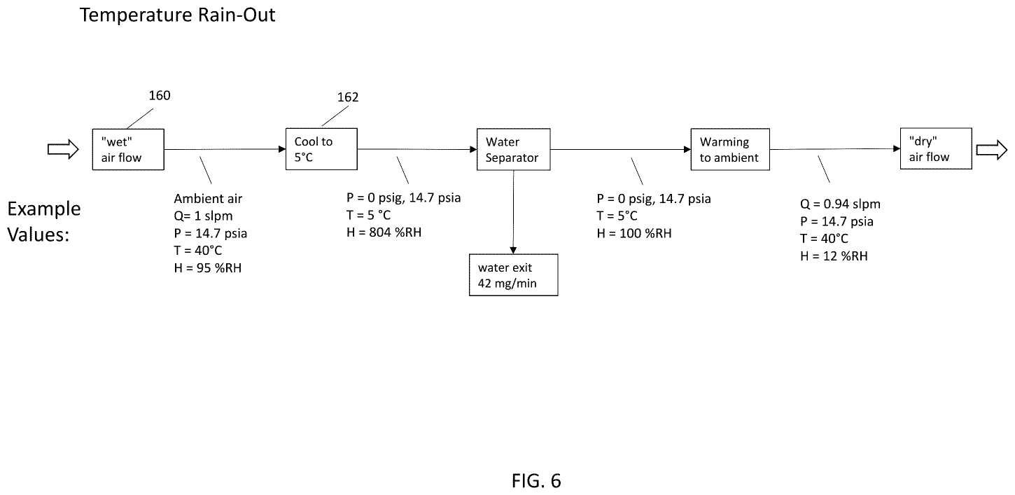

FIG. 6 depicts an embodiment of a gas humidity removal process that involves cooling ambient air to force condensation. Ambient air enters the process in step 160 and passes through a chilling step 162 (e.g. a cold heat exchanger, thermoelectric device, vortex cooling tube, etc.) that reduces the gas temperature and results in water condensation. Condensed water is one or more of collected, expelled, or reintroduced to the gas flow later in the process steps. After water has condensed from the gas flow, the gas flow is optionally warmed back to ambient conditions prior to subsequent process steps.

### Membrane Humidity Management

In some embodiments, humidity transfer material is utilized to remove water from a gas stream. In some embodiments, the transfer material is in the form of a tube. In some embodiments, the transfer material is in the form of a sheet or membrane. In some embodiments, a vacuum is pulled on one side of the membrane, which promotes water transfer from a high-pressure side to the low-pressure side. A sweep flow on the low-pressure side ensures that the low pressure side can remove water from the system.

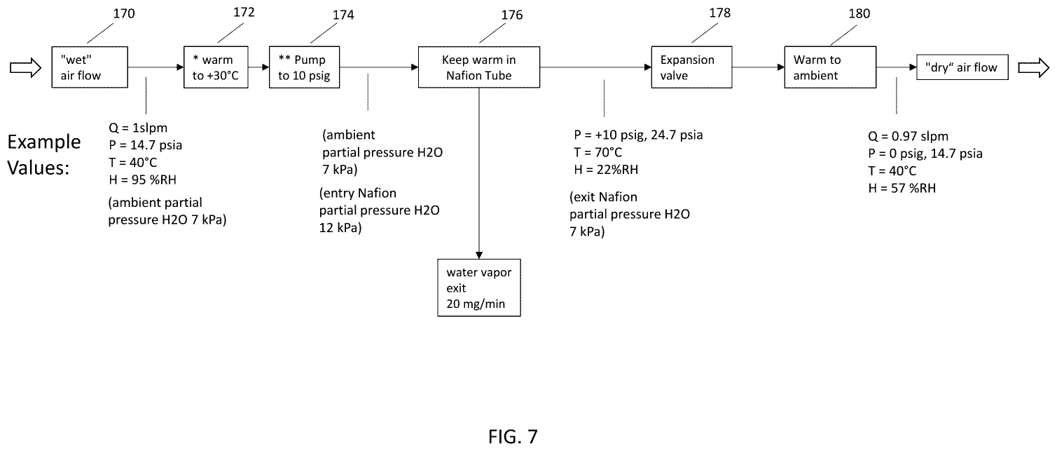

FIG. 7 depicts an embodiment of a gas humidity removal process utilized in a NO generation system. Humid ambient air enters the system in step 170. The gas is warmed (step 172) and pressurized (step 174) to increase the partial pressure of water in the air relative to ambient conditions. Warming the gas first is done to ensure that there is no condensation after the gas is pressurized. Prevention of condensation is key to this design since liquid water disables water transport through Nafion tubing. Water travels across the Nafion membrane as vapor (step 176). The combination of elevated temperature and pressure increases water transport out of the treated gas. In some embodiments, the water vapor is released to atmosphere. In some embodiments, the water vapor condenses and is collected in a water trap. The heated, pressurized gas is then released through an expansion valve (step 178), which reduces the gas temperature as a result in the decrease in pressure. The gas is then optionally warmed back to ambient temperature (step 180) before continuing to NO generation steps within the system.

Some NO₂ scrubber materials (e.g., soda lime) contain water. When utilized in a recirculation architecture, water introduced to the product gas in the scrubber can present a condensation problem elsewhere in the system where gas temperature and gas pressure conditions will vary. In some embodiments, water levels within the gas entering the plasma chamber are managed after incoming reactant gas flow and returning product gas flow are merged. This enables a NO delivery system to ensure non-condensing humidity levels within the system despite varying ambient water content levels and varying water added to the product gas from a water-containing scrubber (e.g., soda lime).

During NO generation to dose a dynamic inspiratory flow (e.g., a ventilator flow waveform), the mixture of fresh reactant gas and returned product gas will fluctuate in real time. This can result in fluctuating humidity levels within the gas entering the plasma chamber. In some embodiments, the humidity of the gas entering the plasma chamber is measured to inform the system controller so that it can modify the plasma control parameters (e.g., power, frequency, duty cycle, dithering) as required to produce the target quantity of NO.

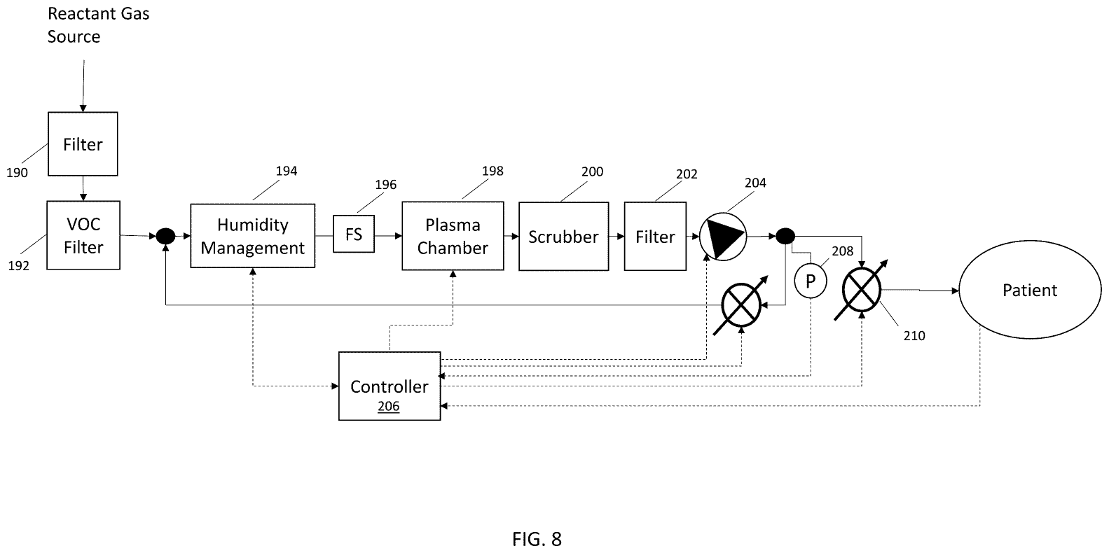

FIG. 8 depicts an exemplary embodiment of a NO generation system with a recirculation loop architecture that removes water from gas after incoming ambient gas and returned product gas flows are merged. Ambient air enters the system and is filtered for particulate and VOCs using a filter 190 and a VOC filter 192 before entering the recirculation loop. The incoming air is merged with returning product gas before flowing through a humidity management component 194. In some embodiments, a passive humidity management device (e.g. a sponge, accumulator, humidity management material, etc.) acts as a low-pass humidity filter to even out the humidity fluctuations in the reactant gas entering the plasma chamber over time to decrease NO production variances associated with reactant gas humidity variances. In some embodiments, the humidity management component is active (e.g., pressure-based or temperature-based forced condensation), driving the gas humidity (up or down) to a target range that satisfies acceptable water content levels for downstream components (i.e. pump, plasma chamber, scrubber, NO sensor) and to prevent condensation within the system. The humidity-adjusted gas flows through a flow sensor 196, a plasma chamber 198, a scrubber 200, a filter 202, and a pump 204 (not necessarily in that order) prior to reaching a node that is maintained at a constant pressure by the device controller. The pump speed is modulated by a controller 206 to maintain a target flow rate through the plasma chamber, as indicated by the flow sensor. The product gas pressure at the node is modulated by the controller 206 to maintain a constant pre-injector pressure, as indicated by a pressure sensor 208 (marked with a “P”). A flow controller 210 is modulated by the controller 206 to deliver a desired quantity of NO to the patient.

In some embodiments, the passive humidity management component includes (at least in part) a gas-permeable material (e.g. open-cell foam, sponge, mesh, textile, bed of beads, etc.) that can absorb and release water molecules. This material acts like a low-pass filter, homogenizing the humidity level within the mixture of fresh and returned gas as it enters the plasma chamber, thereby making the humidity level within the plasma chamber more consistent over time. In some embodiments, the humidity level within the plasma chamber is sufficiently consistent over time that compensation for gas humidity levels in the plasma chamber are not required.

### VOC Management

The quality and safety of medicinal gas provided by a NO generation and or delivery system can be affected by volatile organic compounds (VOCs) in the ambient air. For example, volatile organic compounds have the potential to turn into harmful compounds when exposed to an electrical discharge. Other volatile organic compounds can combust in the presence of an electrical discharge, thereby generating additional energy within the plasma chamber and increasing NO production. Unchecked, uncontrolled increases in NO production can result in losses in NO dose accuracy at the patient. Some embodiments of a NO generation and/or delivery system include a VOC scrubber (e.g., activated carbon) to remove VOCs from the incoming gas stream. VOC scrubbers have a finite service life, however, and it is therefore desirable to know when a VOC scrubber has reached or is about to reach its end of service life. In some embodiments of a NO generation and/or delivery device, the system includes a VOC sensor in the incoming gas stream (i.e., reactant gas) located downstream of the VOC scrubber. When the VOC levels within the post-scrubber gas exceed a threshold, the system controller, having received information from the VOC sensor, can prompt the user to one or more of replace the VOC scrubber and relocate the device to a cleaner air source. In some embodiments, a NO generation and/or delivery system utilizes a photo ionization detector (PID) for measurement of VOCs within a reactant gas stream within the system.

A PID sensor can also be utilized to measure NOx levels within a product gas stream downstream of a plasma chamber. In some embodiments, a PID sensor is located downstream of a plasma chamber and a NO₂ scrubber, so that the PID signal is indicative of the level of NO (i.e., the only remaining VOC) within the gas stream.

### Architecture

### Recirculation Architecture Control

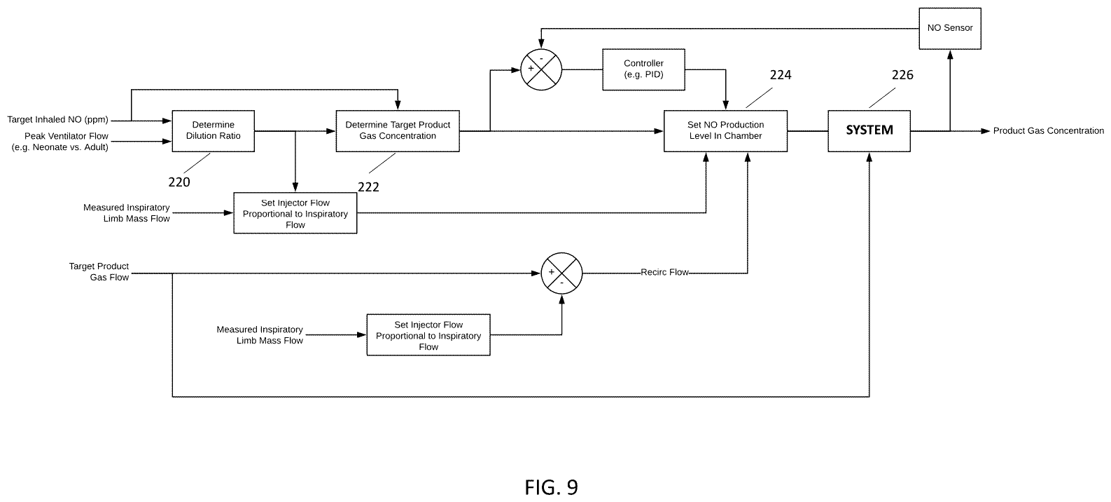

FIG. 9 depicts an exemplary process utilized by a NO generation device controller to set the NO product gas concentration and produce product gas at the set concentration. The process receives a target inhaled NO concentration from an external source (e.g. a user, patient, other device) and peak expected ventilator/inspiratory flow (e.g. corresponding to a particular patient type or treatment). A dilution ratio is determined (step 220) as will be described below. The dilution ratio is utilized in setting the flow rate through the NO injection flow controller. The dilution ratio is also utilized by a NO device controller to determine a target product gas concentration (step 222). NO production level (i.e., degree of plasma activity) is set in step 224 and can be determined based on the target product gas concentration (from step 222), the measured NO concentration in injected gas, the measured inspiratory flow rate, and the recirculated flow rate (i.e. portion of reactant gas already containing NO). The control outputs result in operation of the system 226, which may include a plasma reactor/chamber, pumps, valves, and/or other elements, to generate an actual product gas with concentration that may differ from the ideal target.

Over time, the NO production can vary due to a myriad of factors including but not limited to environmental conditions, electrode wear, scrubber age, inaccuracies in production, inaccuracies in product gas injection, drift in one or more sensors, and other factors. In some embodiments (as shown), the actual concentration of the product gas is measured by one or more NO gas sensors. If the measured product gas concentration differs from the target, plasma control parameters are adjusted accordingly to bring the concentration back to target. In some embodiments, additional NO sensors provide redundant measurements to ensure an accurate measurement can be obtained. In some embodiments, the controller calculates the average between the two or more sensors. In some embodiments, the controller does not use readings from a NO concentration sensor when that sensor presents an error condition or an out-of-range value (e.g. sensor railed). In some embodiments, the controller utilizes a voting scheme with three or more sensors, utilizing two or more sensor measurements that are in agreement and not using outlier measurements from other sensors.

In some embodiments, the range of possible NO product gas concentrations exceeds the ability of one sensor to provide an accurate measurement. In some embodiments, multiple product gas NO sensors with varying range are utilized to provide accurate NO measurements across a broad range of product gas concentrations. For example, one embodiment includes a first NO gas concentration sensor for product gas concentrations between 50 and 500 ppm NO and a second NO gas concentration sensor for product gas concentrations between 500 and 2500 ppm. In some embodiments, NO measurements are made by more than one methodology (e.g. electrochemical, infrared spectroscopy, chemiluminescent, etc.), as appropriate for the given range of NO to be measured. The controller utilizes the product gas concentration sensor reading from the appropriate sensor for the current concentration of product gas.

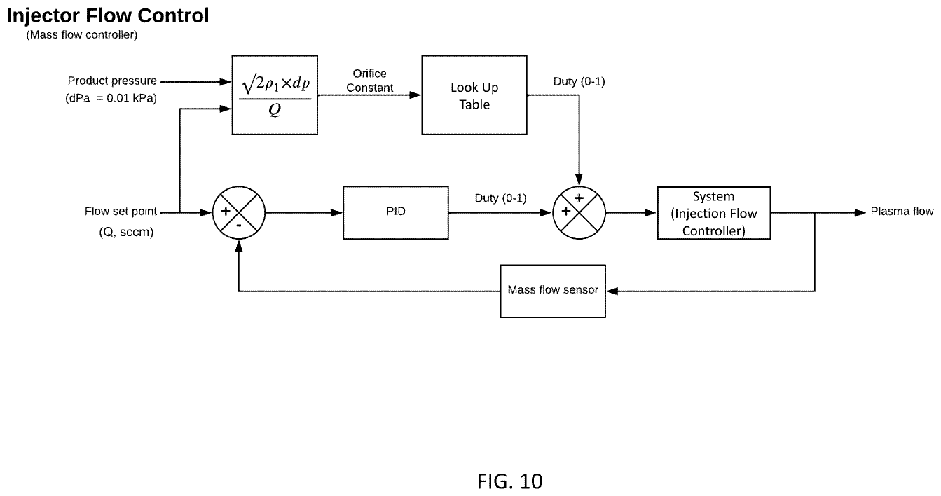

Constant concentration product gas is injected into the inspiratory flow at a rate proportional to the measured inspiratory limb flow rate in accordance with the dilution ratio. FIG. 10 depicts an exemplary control scheme for the product gas injection flow control. An NO injection flow set point is derived as a function of the inspiratory flow and target inspiratory gas dilution level, typically expressed as either a “dilution factor” or “dilution ratio” which are mathematically related and defined below. The NO injection flow set point and product gas pressure are utilized to calculate a target orifice size based on the mass density of gas (p), pressure difference (dp) across the orifice and the target flow rate (Q). One or more of these values serve as input into a look-up table or an equation that determines an operating point of a valve (i.e. feed-forward). The actual mass flow exiting the flow controller is measured by a mass flow sensor, the output of which serves as feedback to a closed loop (e.g. PID) controller. The closed loop controller is combined with the feed-forward control to produce both fast and accurate response of the flow controller.

### Inspiratory Gas Dilution Level

NO product gas concentration and the amount required to inject are inversely related. For example, a high NO product gas concentration results in less product gas being injected into the inspiratory limb to achieve a target inhaled concentration. For any desired inhaled concentration, a range of product gas concentrations and dilution values are possible. In general, low product gas concentrations can result in less NO₂ formation within the product gas by oxidation. On the other hand, low product gas concentrations require greater injected flow which results in higher dilution of the inspiratory flow. Inspiratory flow dilution can result in lower inhaled oxygen levels (NO product gas has low (21%) to no oxygen, whereas inspiratory limb gas can be 100% oxygen). Furthermore higher levels of inspiratory flow dilution result in greater gas volume within the inspiratory limb which can result in potential ventilator faults. In some embodiments, a NO delivery system will operate with a specific relationship between product gas concentration and dilution ratio. In some embodiments, the delivery system operates with a specific relationship between peak NO production (the mathematical product of peak inspiratory flow rate & target NO concentration) and dilution ratio. This relationship is typically stored within the NO delivery system controller as a look-up table or mathematical formula. In one simple embodiment, the dilution ratio is always 10:1 (inspiratory flow: injected NO flow), for example.

In some embodiments, tank-based NO delivery systems operate with a fixed concentration of NO gas in a balance of nitrogen. The level of inhaled gas dilution from tank-sourced gas is directly proportional to the target inhaled concentration (e.g., 80 ppm inhaled concentration requires the inhaled gas to be 9 parts inhaled gas to 1 part 800 ppm tank gas, or a dilution of 10%). Hence, tank-sourced treatments require a higher level of inspiratory gas dilution for a higher dose. Given that there is no oxygen within tank-sourced NO gas (if there was, it would oxidize the NO and form NO₂), higher concentrations of inhaled NO involve a significant reduction in FiO2. Supplementary oxygen is needed to deliver high doses of NO (e.g. NO concentrations exceeding 80 ppm) from a tank to a patient while maintaining adequate oxygen levels in the inspired gas.

NO generation technology (e.g., electrical, N₂O₄, NO donor molecules, etc.) allows for the concentration of the product gas to be varied. Tank-based NO systems operate with a fixed concentration of NO gas, a feature that limits dose control to one variable: NO gas flow rate. Systems that can vary NO concentration within a product gas can control delivered dose with two variables: NO concentration and injected product gas flow rate.

A dilution factor and a dilution ratio are related values, as shown in FIG. 11. The dilution factor is defined as the pre-injection inspiratory flow rate divided by the injected NO product gas flow rate. Maximizing dilution factor results in high concentration NO product gas flowing at a low flow rate, thereby minimizing dilution of FiO₂. Minimizing dilution factor involves flowing lower concentration, slower-to-oxidize NO product gas at a higher flow rate, thereby reducing transit time of the NO to the patient resulting in lowest inhaled NO₂. In some embodiments, the tradeoff between FiO₂ and NO₂ levels is optimized for one or more of patient condition, patient size, NO dose, inspiratory flow rate levels, respiratory device (e.g. ventilator, jet ventilator, etc.), treatment duration, supplemental oxygen requirements, and other parameters.

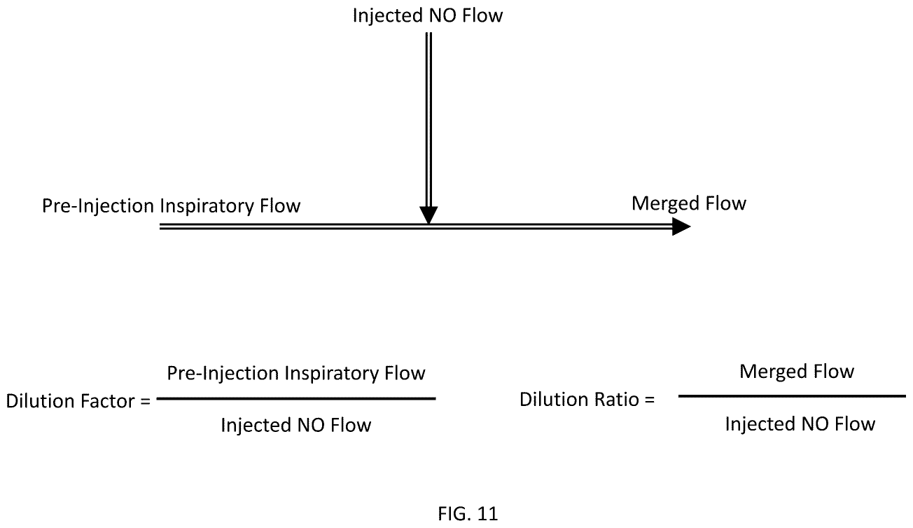

FIG. 12A depicts an exemplary graph showing dilution factor as a function of inhaled concentration (i.e., inspired NO dose) for a given system architecture. The exemplary dilution factor limits were generated from a collection of constraints including maximum product gas concentration (2400 ppm), minimum product gas concentration (50 ppm), maximum product gas injection flow rate (2.5 lpm), minimum product gas injection flow rate (0.02 lpm), maximum NO production rate (5080 ppm.lpm), injector dead volume (1.5 mL) and allowable NO₂ from oxidation (0.5-lppm depending on dose) that results when low injector flows age in the injector dead volume. For each level of delivered dose, there is a range of possible dilution factors that satisfy the constraints. The shaded area labeled “Adult operating space” represents the range of dilution factors that can be used for the range of inhaled concentration in consideration of the various constraints. Neonate and low-flow rate treatments can utilize the dilution factor space for labeled Adult and the dilution factor space labeled “Neonate only.”

The dotted line 230 represents a limit to ventilator dilution to prevent adding too much volume to the ventilator circuit. In some embodiments, this value is a dilution factor of 9 (i.e., 10% dilution of the inspiratory flow). At low doses, the maximum dilution ratio is constrained by the minimum injector flow and minimum dosable flow (2 lpm ventilator flow/0.02 lpm injector flow). At higher doses, the maximum becomes constrained by NO₂ formation and/or maximum product gas concentration. For example, the exemplary system could support a maximum dilution factor of 100 up to 24 ppm (2400 ppm/100), but NO₂ formation limits it to 95 at 20 ppm. Above 24 ppm, the maximum dilution factor is limited by maximum product gas concentration (2400 ppm/dose) up to approximately 40 ppm, at which point the maximum production rate (5080 ppm) becomes the limiting constraint. At very low doses, the minimum dilution factor (100) is a function of the minimum injection rate (0.5 ppm*2 slpm=1 ppm*slpm), the minimum injector concentration (50 ppm) and the minimum injector flow (0.02 slpm). At medium doses it's constrained by the maximum ventilator flow divided by the maximum injector flow (120/2.5=48). Above 40 ppm it becomes constrained by the maximum production rate (5080 ppm*lpm) which constrains the maximum dosable flow (e.g., 5080 ppm-lpm/80 ppm=63.5 lpm) and therefore the dilution factor (63.5 lpm/2.5 lpm=25.4).

In some embodiments, the controller in a NO generation and/or delivery system varies the dilution factor in relation to one or more of target NO dose, patient type (e.g., adult, neonate), patient condition (e.g. hypoxia, infection, lung transplant), treatment type (e.g., ventilator, anesthesia, manual resuscitator, CPAP), FiO₂, minute volume, inspiratory flow rate, inspiratory pressure, respiratory device volume limitations (e.g., added volume that triggers a system self-test in a ventilator), acceptable NO₂ limit and other factors.

FIG. 12B depicts an embodiment of a graph showing an exemplary dilution level strategy that utilizes a specific linear relationship between target dose and dilution factor for adult and neonate patients, respectively. In some embodiments, the relationship between target dose and dilution factor is more complex. In some exemplary embodiments, a NO generation and/or delivery device minimizes inspiratory flow dilution (i.e., high dilution factor) for hypoxic patients to maximize FiO₂. In some exemplary embodiments, a NO generation and/or delivery device maximizes inspiratory flow dilution (i.e. minimum dilution factor, within acceptable limits for the respiratory device) when treating an infection because high NO concentration is more important than oxygenation in this case. In some embodiments, inspiratory flow dilution is maximized to minimize inhaled NO₂ levels.

In some embodiments, the dilution ratio is selected by a controller in the system according to the capability of the injection flow controller. In other words, the dilution ratio and or product gas concentration is selected to result in a range of injected NO flow rates that are within an acceptable range for (i.e., compatible with) the flow controller (i.e., within the dynamic range of the flow controller). For example, a flow controller operating range is between 1 and 5 lpm. A ventilator flow to be dosed has a waveform that varies from 5 to 60 lpm. The controller selects a dilution factor of 12:1 (5×12=60) to ensure that product gas flow rates do not exceed the flow rate capability of the flow controller. This results in NO in the vent circuit diluted to 5 slpm/(60 slpm+5 slpm)=1/13 of the product gas concentration. In system embodiments that enable the connection of multiple types of remote injection apparatus, adjustment of the dilution ratio to accommodate injection flow controller capability can be necessary. In some embodiments, the controller is configured to select a dilution value such that the NO concentration of the product gas is compatible with materials in a product gas flow pathway.

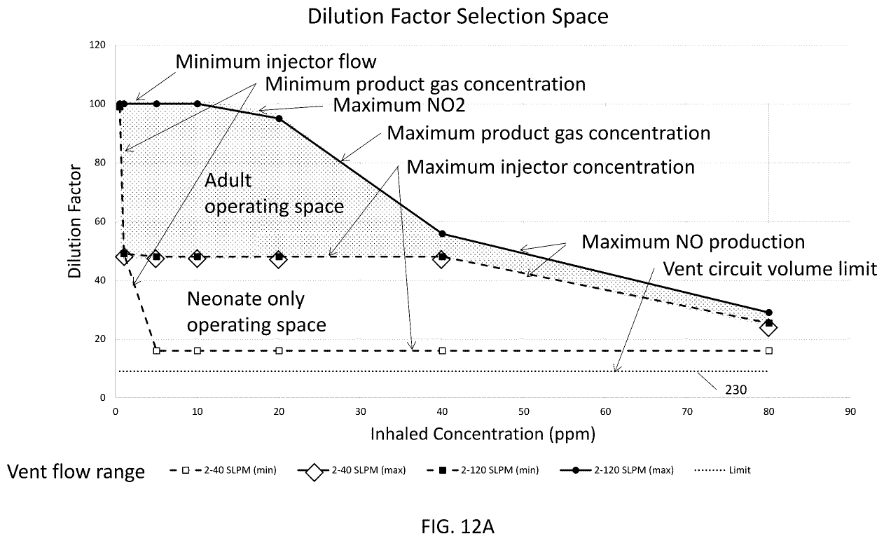

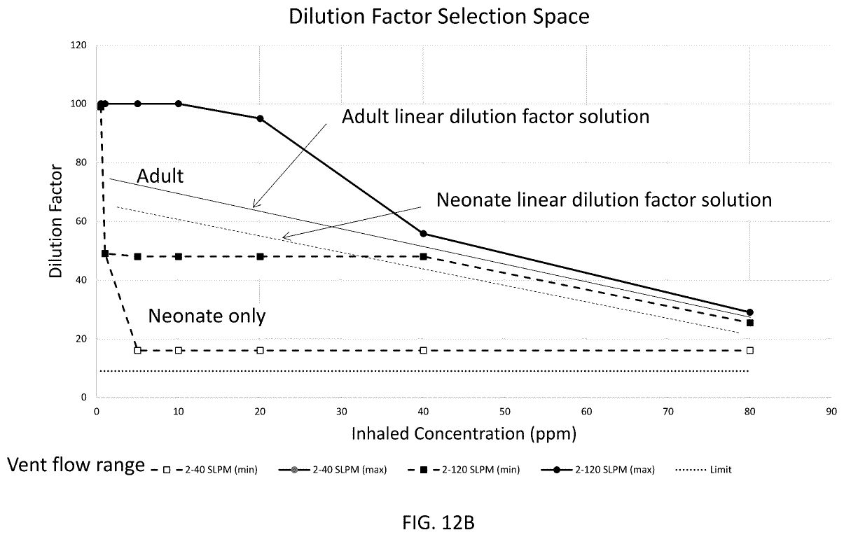

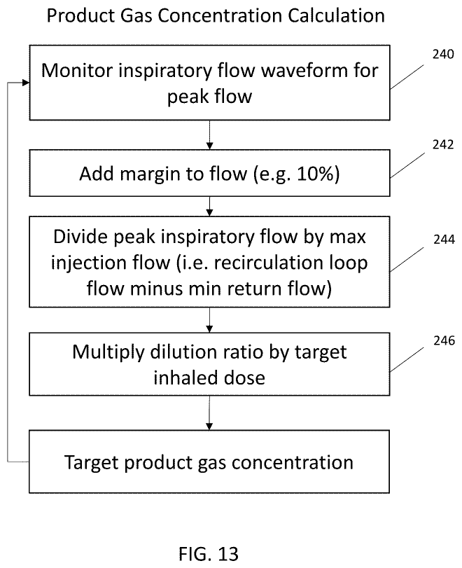

FIG. 13 depicts an embodiment of a method for how a NO delivery system can determine the target product gas NO concentration. The system begins by monitoring the patient respiratory waveform to determine the peak inspiratory flow rates (step 240). In some embodiments, this information comes from a inspiratory flow sensor. In some embodiments, this information is received directly from a ventilator. Margin is added to the peak inspiratory flow value to ensure that adequate product gas will be available in the event of minor deviations in inspiratory waveform (step 242). The margin can account for variation in treatment and provide headroom. The margin can vary from 5 to 100% of the peak inspiratory flow rate. The peak inspiratory flow rate with margin is divided by the maximum flow rate of NO product gas that can be delivered by the NO delivery system to derive the dilution factor and/or dilution ratio (step 244). In some embodiments that utilize a recirculation loop, the maximum injected flow rate is less than the recirculation flow rate, making the return gas flow always greater than zero. This can improve system stability and decrease product gas aging within the return leg of an external recirculation loop. In some embodiments, this minimum return gas flow rate is in the range of 0.1 lpm to 2 lpm. The dilution factor is then multiplied by the target inhaled dose (step 246) to provide the target product gas NO concentration. The target inhaled dose is typically provided by a user. In some embodiments, the target inhaled dose is fixed for a device. The system continues to monitor the inspiratory flow waveform as it generates NO product gas at the target product gas concentration. In the event that patient behavior or ventilation settings change and the peak inspiratory flow rate changes, the NO delivery system controller can change the target NO product gas concentration. In some embodiments, the product gas concentration is varied in real time. In some embodiments, the product gas concentration is updated periodically or only after the peak inspiratory flow changes by a threshold amount. These approaches of selecting a product gas concentration apply to all types of NO generation where a system can vary the product gas concentration including but not limited to systems that derive NO electrically or chemically (e.g. N₂O₄, NO₂, or NO donor molecule approaches).

In one example, the peak flow can equal 100 slpm, and the peak flow with margin can equal 110 slmp. The recirculation loop flow can be 51 spm, and the minimum return flow can be 0.5 slpm. The peak injection flow equals the difference between the recirculation loop flow and the minimum return flow (4.5 slpm). The dilution factor can be calculated as 100/4.5, which equals 24.4. The dilution ratio will be 25.4 The target dose is 40 ppm, and the target product gas concentration is 1018 ppm (25.4 multiplied by 40 ppm).

### Recirculation Architecture NO Measurement Alternatives

Some architectures of an NO delivery system divide the NO product gas flow into two or more flows, including a flow to the patient and one or more of a flow to an NO concentration sensor, a return path to the system (AKA Recirculation architecture), and ejection out of the system (AKA Dump architecture). When a recirculation architecture is utilized, the quantity of NO delivered to the patient and the quantity returned to the system are in constant flux as the system modulates NO delivery to accurately dose the patient inspiratory flow. As a result, the mix of fresh reactant gas and returned product gas in the plasma chamber can vary. This is due to the loss of NO due to reaction with oxygen in the product gas (oxidation), interaction with other materials in the system (e.g. tubing and scrubbing material), in accuracies in NO production within the plasma chamber, and/or inaccuracies in gas flow control within the system.

In some embodiments, the NO concentration of product gas within the recirculation architecture is measured so that the concentration of product gas upstream of the injector is known by the controller that controls product gas injection. When the NO concentration within the recirculation system is higher than a target value, the controller can respond by injecting less product gas into the inspiratory flow so that a target NO mass flow level (ppm.slpm) is still delivered to the inspiratory flow. Similarly, when the NO concentration within the recirculation system is lower than a target value, the controller can respond by injecting more product gas into the inspiratory limb. Another way of describing these changes in the quantity of injected product gas is to say that the dilution ratio (ratio of inspiratory flow rate to injected gas flow rate) is modulated in the presence of varying product gas NO concentration to achieve a target NO dose delivery (e.g., ppm.lpm, or mg/hr).

Some embodiments of a NO generation system have redundant NO generators to ensure reliable delivery or to simultaneously provide NO to more than one application. In one embodiment of a system with redundant NO generators, each generator has one or more product gas NO sensors dedicated solely to that generator.

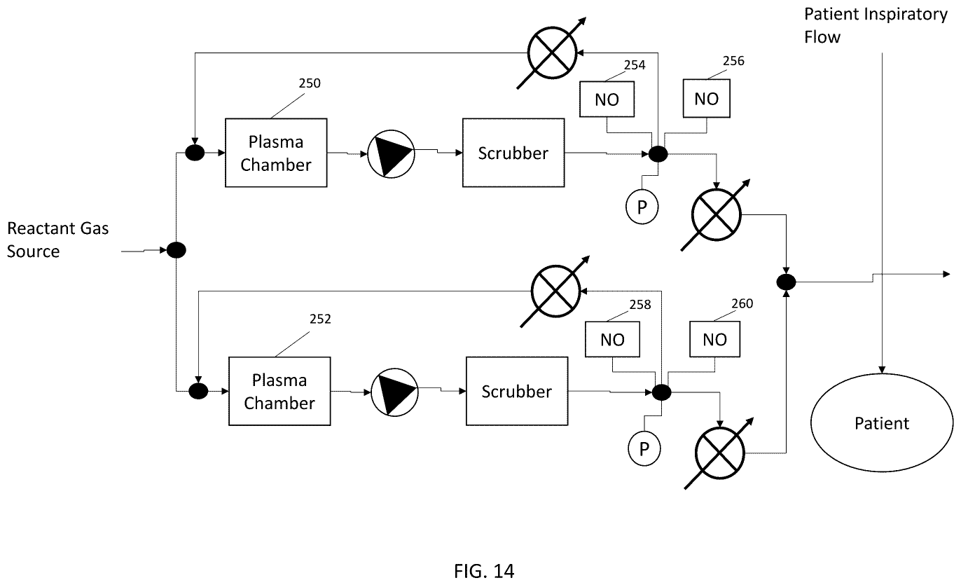

FIG. 14 depicts an exemplary embodiment of a redundant recirculation architecture with redundant NO sensors in each generator. In some embodiments, each NO generation loop is controlled by a common controller (e.g., a microprocessor, not shown). In some embodiments, each NO generation loop is controlled by its own controller (not shown). When more than one controller is utilized, the two or more controllers can either communicate directly or indirectly, for example, through a separate controller that communicates with each of the NO generation controllers. Communication between the controllers is utilized to ensure continuous and/or accurate NO generation. For example, a NO generator can fail and its controller can communicate to the other NO generator a fault condition. In response to this fault condition, the second NO generator can automatically begin generating and delivering NO to substitute for the initial failed NO generator. In another example, a central controller can direct NO production by turning on and off individual NO generators. NO generation selection can be made based on the status of each NO generator (e.g., a fault condition or an exhausted scrubber) or in an effort to equalize wear on each NO generator to prolong overall system service life.

Reactant gas enters the system and flows through one of the plasma chambers 250, 252 where nitrogen and oxygen in the reactant gas are ionized by a plasma, forming a product gas with measurable amounts of nitric oxide and nitrogen dioxide. The product gas flows through a scrubber that removes nitrogen dioxide (and some NO, depending on the chemistry). The product gas then flows through a pump and on to a node. The node is maintained at a constant pressure and NO concentration, as measured by sensors 254, 256 (in a first recirculation loop) and sensors 258, 260 (in a second recirculation loop) in fluid communication with the node. The sensors communicate with a controller (not shown) that controls the system. The controller controls the pressure at the node by modulating the return flow controller and pump speed. In some embodiments, the pump speed is constant and only the return flow controller is utilized to maintain a target pressure at the node. The concentration of NO at the node is modulated by the controller by modulating plasma activity in the plasma chamber (e.g. frequency, duty cycle, power level, etc.). The controller also modulates the injection flow controller to deliver a target amount of NO to the inspiratory flow to achieve a prescribed dose of NO to the patient. In some cases, the dose of NO is described as a target concentration (e.g. ppm) in the inspired gas. In other cases, the dose of NO is prescribed as a quantity of NO per unit time (e.g. mg/hr). The depicted system includes a duplicate NO generation recirculation loop with redundant NO sensors. In some embodiments, the two recirculation loops are controlled by a common controller. In some embodiments, added safety is provided by having redundant controllers as well.

Placement of one or more NO sensors post-scrubber can be useful because there is essentially no NO₂. Thus, a NOx sensor that measures both NO and NO₂ may be used as an NO sensor in this location because the NOx (NO+NO₂) reading would be equivalent to an NO reading since the NO₂ contents are at or near zero. In some embodiments, the hNO sensor is located in the return flow pathway of the recirculation loop, after the bifurcation to inject NO flow (not shown). This provides the advantage of decreasing dead volume between the scrubber and NO injector, to reduce transit time and to minimize injected NO₂ levels.

In some embodiments, open loop control of NO production within the recirculation loop is sufficient to maintain a target concentration at the node with acceptable accuracy. Open loop control requires characterization of the system through calibration to know the amount of NO generated under all foreseeable environmental, service life and clinical scenarios. In some embodiments, the rate of change of the NO concentration under open loop control is slow enough that continuous monitoring of the NO product gas concentration is not necessary. In some embodiments, slow drifts in product gas NO concentration can be detected at sensors measuring the inhaled NO concentration at the patient. For example, these drifts in NO product gas concentration can be adequately measured and tracked with slow electrochemical sensors having a t-90 response time on the order of 30 seconds.

In some embodiments, the concentration within the recirculation loop varies too much for open loop control alone. For example, the rate of NO loss to a scrubber may vary with scrubber age and chemistry. The rate at which the NO concentration at the node deviates from an acceptable range will vary with the control scheme and NO generator design. When the NO product gas concentration varies too greatly in real time, some embodiments utilize one or more NO sensors that measure product gas concentration directly.

In some embodiments, the NO generation system purges the product gas pathway (e.g., a recirculation loop, linear gas pathway) with non-NO containing gas (e.g., air) to reset the product gas concentration within the pathway to known conditions (i.e., zero NO within the loop). In some embodiments, the controller can be used to purge the product gas pathway using one or more control schemes. In some embodiments, the device controller can initiate a purge at specific time intervals. In some embodiments, the device controller can initiate a purge when specific conditions are detected (e.g. NO loss term exceeds a threshold, error between expected inhaled dose and dose indicated by inspiratory gas sensors differ by a threshold). In some embodiments device purge is accomplished by turning off the plasma and flowing product gas around the recirculation loop until all NO has oxidized into NO₂ and has been scrubbed by a scrubber (e.g. soda lime). In other embodiments, a product gas pathway is purged by turning off the plasma and continuing to deliver gas to an inspiratory limb until the gas within the NO device no longer has NO or NO₂ in it. In other embodiments, the NO product gas pathway is purged by turning off the plasma and opening an outlet (e.g. a valve) in the product gas pathway so that product gas within the pathway is directed out of the device (e.g. to ambient air, to a house vacuum) with optional scrubbing of NO, NO₂ or NOx and replaced with non-NO/NO₂ containing gas (e.g. reactant gas). In some embodiments, purging is completed after a set amount of time. In some embodiments, purging is completed after a set volume of gas has been passed through the product gas pathway. In some embodiments, purging is completed after product gas concentration indicated by one or more of a product gas NO sensor and a product gas NO₂ sensor indicates that there is no remaining NO and NO₂, respectively. In some embodiments that have redundant NO generation channels, the controller purges the recirculation loop of one NO generation channel while it treats a patient with product gas from another NO generation channel.

In some embodiments, the NO generation system utilizes a product gas NO sensor as feedback to a closed-loop system to modulate plasma activity to regulate product gas NO concentration to the target level.

Some types of NO sensors (e.g., some examples of electrochemical sensor) can be damaged by continuous exposure to high concentration NO gas. In some embodiments, a system with redundant NO sensors alternates gas sensor use to provide down time to each sensor to reset. Gas flow to each of the sensors is controlled by one or more valves or pumps so that the sensors are exposed to non-NO containing gas while they reset. In some embodiments, the non-NO containing gas is flowed past the NO sensor to expedite the sensor reset. In some embodiments, a NO sensor exposure schedule is managed by a controller (e.g., microprocessor) in the system. In some embodiments, the controller provides a purge flow to a NO gas sensor for a set amount of time, that amount of time being related to an amount of time required to return the sensor to acceptable conditions (e.g. initial conditions). In some embodiments, the controller monitors the output of a NO sensor during the resetting process to assess when the process is complete (e.g., when the no-load output signal returns to within an acceptable range of a prior reference (e.g., zero value). In some embodiments, changes in the zero value and/or the time to reach a stable zero value are tracked by a controller for one or more resetting processes. This information can be utilized by a controller to detect when a sensor is at or near the end of its service life, for example. In some embodiments, the sensor exposure schedule and associated valve and pump functions are managed by a hardware timing circuit.

In some embodiments, the purge gas utilized to reset a NO sensor is released to the atmosphere. In some embodiments, the purge gas is scrubbed of NO and/or NO₂ prior to release into the atmosphere. In some embodiments, the purge gas is introduced to a gas flow path (e.g. incoming reactant gas flow path, or product gas flow path) within the system after purging the NO sensor. In some embodiments, the product gas NO sensor is located on the return leg of the recirculation loop to minimize dead volume between the plasma chamber and injector. A reduction in outbound path dead volume can reduce inhaled NO₂ levels.

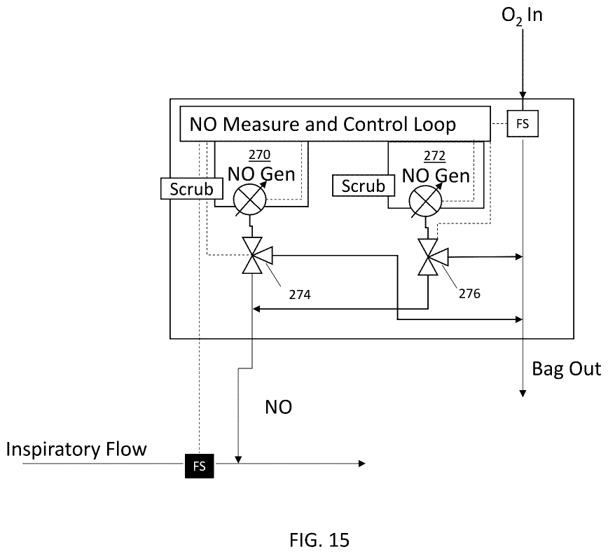

FIG. 15 depicts an embodiment of a NO generation system with redundant NO generators 270, 272. NO generators can be inclusive of all components required to generate NO, including but not limited to high voltage transformer, electrodes, plasma chamber, gas pump, NO concentration sensor, pressure sensor, etc. The system is designed such that product gas from each NO generator can be delivered to more than one output (e.g. a patient inspiratory flow and a manual resuscitation bag flow). The controller controls the configuration of 3-way valves 274, 276 (or equivalent pneumatic components) that select the destination of a product gas flow. In one scenario, each product gas flow flows to an independent destination. The two product gas flows from two independent NO generators can dose two independent inspiratory gas flows simultaneously with appropriate product gas flow and concentration for their respective dosed gas flows. In some applications, the two product gas flows can be merged into a common destination gas flow to increase the dose further (e.g. high dose applications like infection treatment). Each NO generator can serve as a backup for the other NO generator. In the event that one NO generator fails, gas from the other NO generator can be delivered in an equivalent concentration and flow rate resulting in no lapse or compromise in treatment.

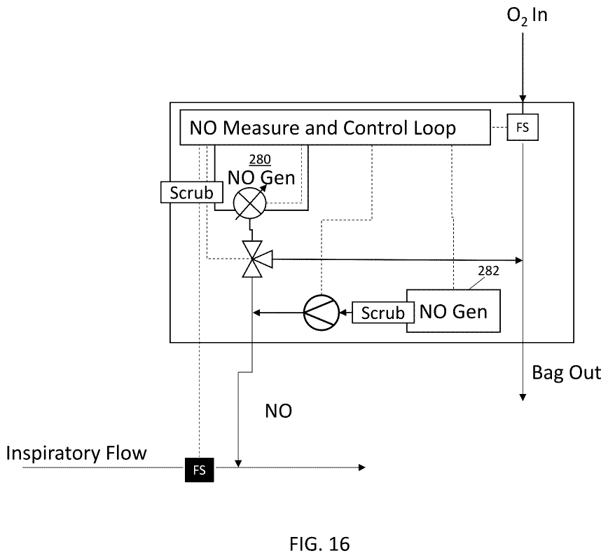

FIG. 16 depicts an embodiment of an NO generation system with rudimentary backup NO generation. A first NO generator 280 is capable of accurately dosing either an inspiratory gas flow or a bag flow based on flow sensor inputs and the selection valve setting, as determined by the controller. In the event that the primary NO generator fails, or in the case of the need for supplemental NO, a backup NO generator 282 can be activated by the controller or manually to deliver NO to the inspiratory flow. In some embodiments (shown), the backup NO generator provides constant flow rates of NO product gas. In some embodiments, the rudimentary backup NO generator utilizes a critical orifice to maintain a constant flow rate. In some embodiments, the backup generator is a linear architecture (i.e. no recirculation). In some embodiments, the output of the backup generator can be directed to either a first treatment (e.g. a ventilator) or a second treatment (e.g. a manual resuscitation bag). In some embodiments, the destination of the backup generator output is user selectable. In some embodiments, the flow controller in a remote injection module defaults to open when it fails so as to not obstruct the output of a backup NO generator to delivery NO to patient through a external recirculation loop. In some embodiments, the return flow controller in an external recirculation loop is normally closed without power to prioritize flow to the patient through the injection flow controller.

In some embodiments, the backup generator delivers a variable concentration of NO. In some embodiments, the concentration of NO delivered by the backup generator is selected to match the concentration of NO inhaled by the patient during treatment with the primary NO generator. In some embodiments, the backup generator initially produces NO (i.e. ppm.lpm) at a similar rate to the mean production that had been delivered by the primary NO generator. This ensures continuity in minute volume of NO to the patient. In some embodiments of a backup NO generator, one or more of the flow rate, NO concentration, and NO production can be varied by a user).

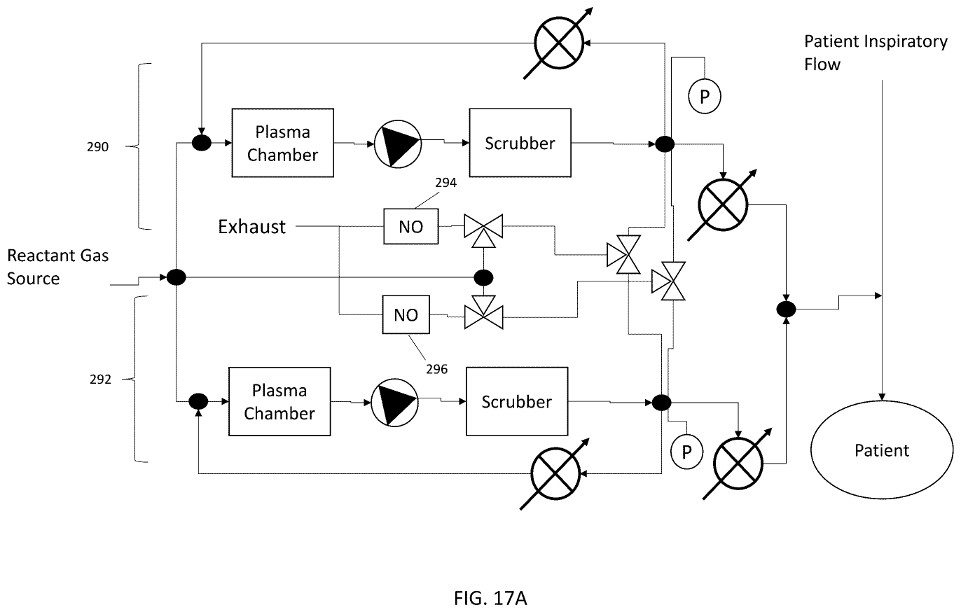

As shown in FIG. 17A, in some embodiments a system can be configured with redundant NO generation channels 290, 292 and redundant NO sensors 294, 296. Gas flow from the constant pressure node is directed by the device controller to flow to the NO sensors. Three-way valves (or equivalent pneumatic components) are utilized to direct product gas from either NO production channel or fresh reactant gas through each of the sensors. In some embodiments, a pump pulls sample gas from the constant pressure node. This approach can provide the ability to control the gas flow rate through each of the sensors. This approach enables each NO sensor to measure each NO concentration in each channel at all times while also enabling the ability to purge the sensors. This approach also provides the ability to compare the outputs of the NO sensors from a common gas source to check calibration. In the event that the signal form each of the sensors is not in sufficient agreement (e.g., within 5%), the system controller can generate an alarm and/or terminate treatment.

When a product gas NO sensor is used intermittently, the system typically remains stable between product gas NO readings. Hence, the system may operate without measuring product gas concentration for extended periods of time (e.g. many minutes). Changes in ambient conditions (e.g. pressure, temperature, and humidity) and patient treatment settings (e.g. ventilator mode, NO dose, breath rate, inspiratory flow rates) can influence steady-state operating conditions. Hence, when there is a change in either ambient conditions or patient treatment settings (e.g. based on sensor measurements, user inputs, or communicated information from adjunct equipment), the NO delivery system may break the periodic product gas sensor reading pattern and check the product gas NO concentration at an earlier than scheduled time point. Following the new time point, the system controller will either resume the old sensor reading schedule or begin a new sensor reading schedule in relation to the time of the latest sensor reading.

In some embodiments, low concentration NO sensors are utilized to measure the concentration of product gas. The product gas is diluted with gas (e.g., air) to bring the concentration down to a level that can be measured by the low concentration NO sensors. This allows for the low concentration electrochemical sensors to be utilized continuously. In some embodiments, the low concentration sensors utilized to measure diluted product gas are also utilized to measure the concentration of NO in inspiratory gas by the same system.

In some embodiments, NO concentration within a recirculation loop is measured intermittently to detect drift in the loop concentration. For example, the loop concentration can be measured every 30 minutes. This allows the NO sensor to be isolated from the gas flow except during gas measurement to prolong the service life of the sensor.

FIG. 17B depicts an electrical NO generation system with purge flow. Reactant gas (e.g., air) enters the system on the left side of the image. In some embodiments, the reactant gas is prepared with one or more of VOC scrubbing, NO₂ scrubbing, NO scrubbing, NOx scrubbing, particulate filtration and humidity adjustment prior to entering a recirculation loop. The flow passes through a plasma chamber and a pump (not shown). In some embodiments the humidity of the incoming reactant gas is measured with one or more humidity sensors (not shown). Pressure of the product gas exiting the pump is measured by a pressure sensor. The product gas passes through a scrubber (shown) or a reducer (not shown) that removes NO₂ from the product gas. The concentration of NO is then measured with a gas sensor (e.g., a NO sensor, NOx sensor). The flow rate of the product gas is measured by a flow sensor that is utilized by a system controller (not shown) as input to a closed loop control on pump speed. In some embodiments, the scrubber dampens pulsatility in the product gas stemming from the pump. The product gas flows through a three-way valve or equivalent binary valve/manifold connection that either directs gas to a remote injection module or through an internal shunt pathway.

Gas that flows out to the remote injection module travels through a lumen. At the remote injection module, the flow bifurcates with a portion of the flow going to the patient inspiratory flow and the remaining flow returning to the NO device. Gas flowing to the inspiratory flow travels through a flow sensor and flow controller. A pressure sensor upstream of the injection flow controller provides input into the flow controller control as does inspiratory flow measurements. The injection flow controller is controlled by a controller (not shown) that is either located in the remote injection module or the main NO device.

Returning gas flow from the return injection flow controller reenters the main No device and passes through a three-way valve (or equivalent) that can also receive flow from the shunt flow. The next bifurcation in the flow path provides an option to send product gas through a flow controller and flow sensor (not necessarily in that order) that injects flow into a bag (i.e. manual resuscitator) flow in proportion to flow measured in the bag flow by a flow sensor, controlled by the NO device controller. Pressure in the product gas is measured in the return leg as input to both the bag flow controller and the return flow controller. A return gas flow controller is modulated by the device controller to maintain a constant pressure upstream of the active injection flow control (i.e., the bag or the inspiratory injection flow controller). A three-wave valve (or equivalent) directs returning product gas towards a purge gas flow path or to merge with incoming reactant gas flow. In some embodiments, the three-way valve is binary in its states. In other embodiments, the three-way valve is variable and can send a portion of the returning gas to the purge flow. In some embodiments, the purge gas flow flows through a scrubber (not shown) to remove NO and NO₂ from the gas before it is released to atmosphere.

### Photoacoustic Sensor

Electrochemical sensors can operate in limited humidity conditions and have a finite life. In some embodiments, a photoacoustic NO₂ sensor is utilized in a NO generation system to measure concentrations of NO₂ in a gas (e.g., product gas, inspired gas). Photoacoustic sensors utilize a light source and filter to provide light at a particular absorption band for a target gas molecule (e.g., NO₂ absorbs light in the range of 250 to 650 nm with a maximum at 403 nm). The light is pulsed into a chamber of finite volume. The gas absorbs the light energy and expands due to the increased temperature. The gas expansion produces a pressure signal that is measured by a pressure sensor (e.g., microphone, piezoelectric pressure sensor, etc.). This selective absorption and expansion within a finite volume enables measurement of nitrogen dioxide concentration. In some embodiments, a photoacoustic sensor is utilized to measure NO₂ and/or NO concentration in gas within the system (e.g., reactant gas, product gas, inspired gas).

### Reconfigurable Architecture

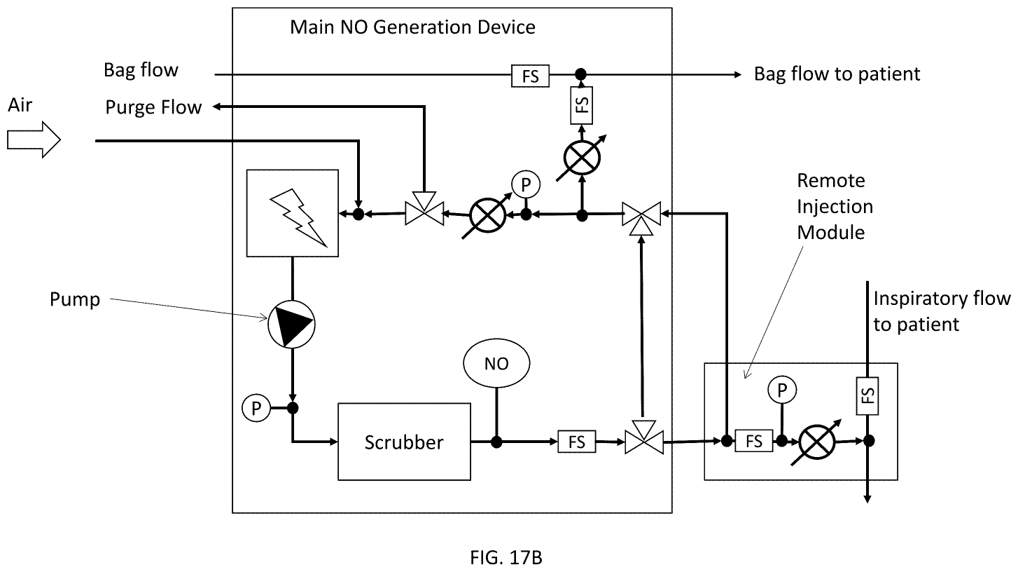

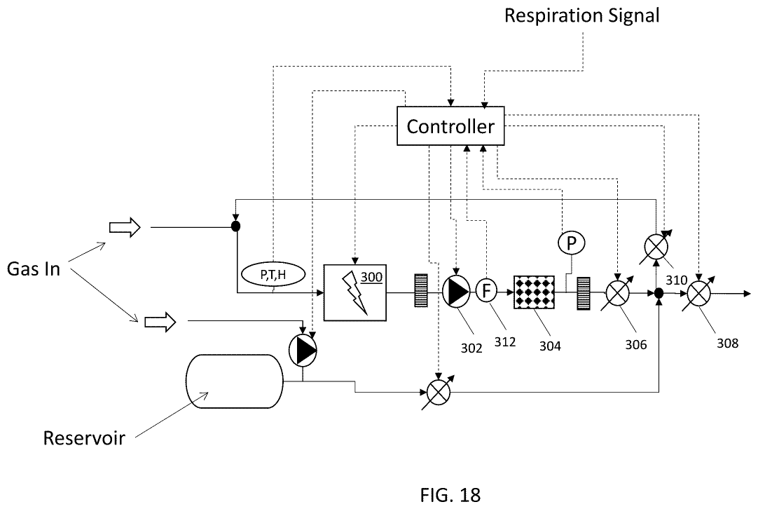

In some embodiments, a NO generation system can be utilized for both continuous NO delivery and pulsed NO delivery with gas purging. FIG. 18 depicts an exemplary system that utilizes a single plasma chamber 300 that can be configured for either recirculation architecture or a pressurized scrubber reservoir. Reactant gas enters the system and may be sourced from a cylinder, house supply, ambient air, or other source. The reactant gas may be filtered, scrubbed, and/or desiccated (not shown). The reactant gas flows into the plasma chamber 300 where a portion of the flow is converted to NO and possibly some NO₂. This NO-containing product gas is then optionally filtered for particulate prior to entering a pump 302. The pump pushes the product gas into an NO₂ scrubber 304. Exiting the scrubber, the gas is filtered for particulates before and flows through a valve 306 (i.e., binary or variable flow controller). At this point, product gas either flows out to the patient through a second flow controller 308 or returns to a location at or before the plasma chamber through another flow controller 310. A second inlet into the system sources purge gas from the same or a different source. The purge gas may be filtered, scrubbed, and/or desiccated (not shown). The purge gas is pressurized into a reservoir by a pump. The pressurized purge gas is controllably released from the reservoir with a purge flow controller. Purge gases exit through the same flow controller as the product gas.

In a recirculation mode, product gas flows either through the patient flow controller or through the return flow controller. The delivery system can be purged either with gas from the purge reservoir or reactant gas that has passed through the system with the plasma turned off.

In pulsed mode, the flow controller downstream from the scrubber is normally closed to make product gas accumulate within the scrubber. Product gas is then released from the system in boluses that are optionally chased by a bolus of purge gas.

The system is controlled by a controller that receives sensor information about the reactant gas, product gas, and gas into which the NO is introduced (not shown, e.g., inspiratory gas). In some embodiments, sensor types include but are not limited to measurements of humidity, pressure, temperature, flow, NO concentration, NO₂ concentration, O₂ concentration, and He concentration.

The amount of NO that exits the system and the timing thereof is controlled by the controller. The controller receives respiratory signals to determine NO output flow requirements. In some embodiments, respiratory signals are measured by the NO device (e.g. a pressure sensor in a delivery lumen, a flow sensor in an inspiratory line). In other embodiments, the NO device receives respiratory signals and/or trigger information from an external device (e.g., ventilator, CPAP machine, etc.)

In some embodiments, the controller utilizes sensor measurements to assess the operation of the system and respond to variations in the system to control NO generation and delivery. A reactant gas sensor measures one or more of pressure, temperature and humidity. The controller utilizes this information to generate a target amount of NO. NO production is varied by increasing or decreasing one or more of plasma discharge frequency, duty cycle, dithering and power. Conditions indicated by the sensors that decrease NO production are met with changes to plasma control parameters that increase NO production. For example, the controller can compensate for an indicated increase in humidity by increasing one or more of plasma frequency, duty cycle or power. In another example, decreased pressure within the plasma chamber indicates that there are fewer O₂ and N₂ molecules within the plasma chamber. The controller can respond to lower plasma chamber pressure by increasing one or more of plasma frequency, duty cycle, and power.

The flow rate of gas through the plasma chamber is optionally measured by a flow sensor 312, labeled “F” in FIG. 18. In some embodiments, flow is measured by a sensor (shown). In other embodiments, the flow rate is measured indirectly (e.g. pump rotational rate, rate of pressure rise in a scrubber or reservoir, etc.). The amount of NO generated within the plasma chamber varies with flow rate through the chamber. The relationship between reactant gas flow rate and NO production is related to the cross-sectional area of the plasma chamber (larger cross-section yields more uniform gas velocity and less dependence on flow rate). Gas flow nozzles can focus gas flow in a particular area, such as an electrode gas, but can be highly dependent on gas flow rate. This information can be stored within the controller in the form of an equation or look-up table, for example. For example, the controller can detect that the product gas flow rate is lower than a target value. The controller responds by increasing voltage to the gas pump to achieve the target flow rate. In some embodiments, the target product gas flow rate is in the range of 0.1 to 10 lpm. For example, target product gas flow rate can be between 3-5 lpm. In some embodiments, the target product gas flow rate is in the range of 3 to 5 lpm. Faster product gas flow rates hasten the transit time from scrubber to injector. Faster product gas flow rates also provide capacity for higher injector flow rates which decreases residence time within the injector. The limit to higher injector flow rates comes from increased back pressure and the need for higher/heavier/more power-consuming pumping capability to circulate gas within the recirculation loop.

In some embodiments, a controller detects that a scrubber or purge gas reservoir are not reaching a target pressure level between NO bolus deliveries. The controller then responds by increasing the pump voltage to increase pump speed and average gas flow rate through the system. In some embodiments, the controller utilizes the rate of change in pressure within a reservoir (e.g., bypass gas reservoir, NO₂ scrubber housing) to infer the gas flow rate into or out of the reservoir. In some embodiments, the controller either calculates or infers from prior characterization data the volume of gas that has left a reservoir based on the change in pressure within that reservoir.

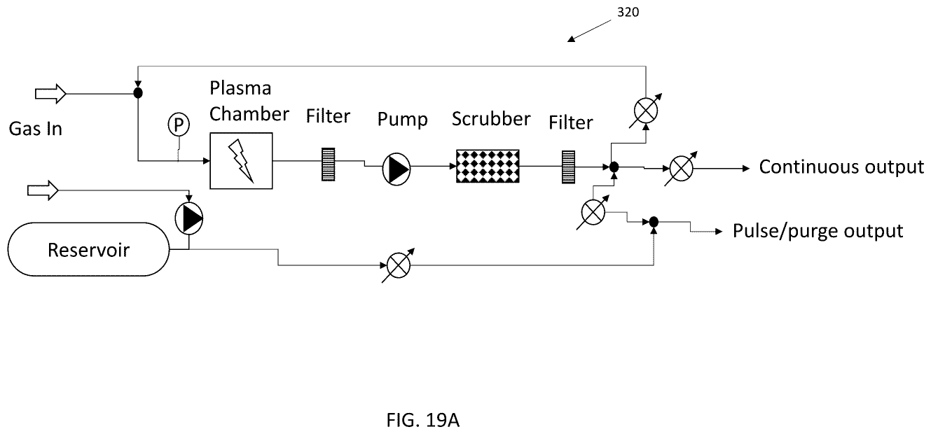

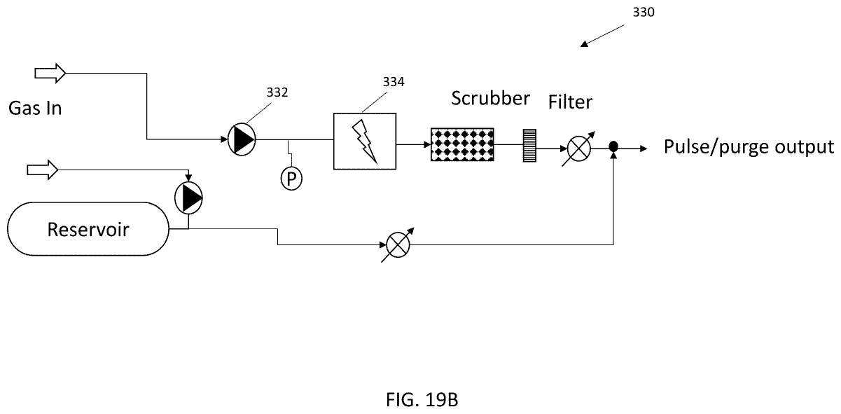

FIG. 19A depicts an exemplary system 320 that can also serve as both a continuous delivery system or a pulsed NO delivery system with different valve locations. A system that can provide both pulsed and continuous treatment enables a wide variety of treatments to be administered with one NO generation device. This type of system is typically controlled by a controller (e.g., microprocessor, FPGA, etc.) (not shown). In the embodiment depicted, pressure within the plasma chamber varies with altitude from roughly 14.7 psia at sea level down to 10 psia at 10,000 ft elevation. Measurement of plasma chamber pressure with a sensor (not shown) enables the controller to adjust plasma activity in response to changes in atmospheric pressure in order to maintain a target NO production level.

FIG. 19B depicts an exemplary embodiment of a system 330 with a reactant gas pump 332 located before a plasma chamber 334, thereby protecting the pump from particulates originating from the plasma chamber. In this embodiment, the plasma chamber pressure varies within a range dictated by the internal pressure of the scrubber. In some embodiments, pressure within the scrubber varies from 20 psia to 26 psia, for example. The controller measures the plasma chamber pressure with a pressure sensor (labeled “P”) and adjusts plasma chamber activity in order to maintain a target level of NO production. For example, in some embodiments, as the plasma chamber increases, the controller decreases the duty cycle of the plasma in order to maintain a consistent level of NO production. The relationship between plasma duty cycle, pressure, and NO production is captured in one or more look up tables or equations utilized by the controller.

### Fixed Duration Electrical Discharges

In some embodiments, an electrical NO generation device modulates NO production by varying the frequency of electrical breakdowns while holding the duration of electrical discharges to a fixed duration (e.g. 500 usec). The same principal can be applied to microwave NO generation devices as well. This method can provide an adequate NO production range while improving NO production accuracy, particularly at low production levels, because the occurrence and timing of electrical breakdown in the plasma chamber is more consistent than a fixed frequency/variable duration approach. In some embodiments, frequency modulation also reduces NO₂ production within the plasma chamber because longer duty cycles are associated with lower NO₂.

Pump after Scrubber

Electrical discharges between electrodes can often create electrode particles in the gas downstream of the discharge. The particles can be the result of vaporized electrode material and sputtered electrode material. These particles can be extremely small in diameter (e.g., <5 nm) and up to 50 micron, and difficult to remove entirely from a gas stream. These metallic particles can present a variety of challenges to device longevity (e.g., decreasing voltage required for electrical creepage across surfaces, fouling pumps and valves).

In some embodiments, pneumatic components are protected from plasma generation particulates by locating them downstream of a scrubber and/or particle filter. Locating the pump after the scrubber can decrease the maximum pressure within the system. Lower maximum pressure decreases the rate of NO to NO₂ conversion and permits higher levels of water to be in the product gas without the risk of condensation. Furthermore, locating pneumatic components after a scrubber can decrease the exposure of said components to corrosive NO₂.

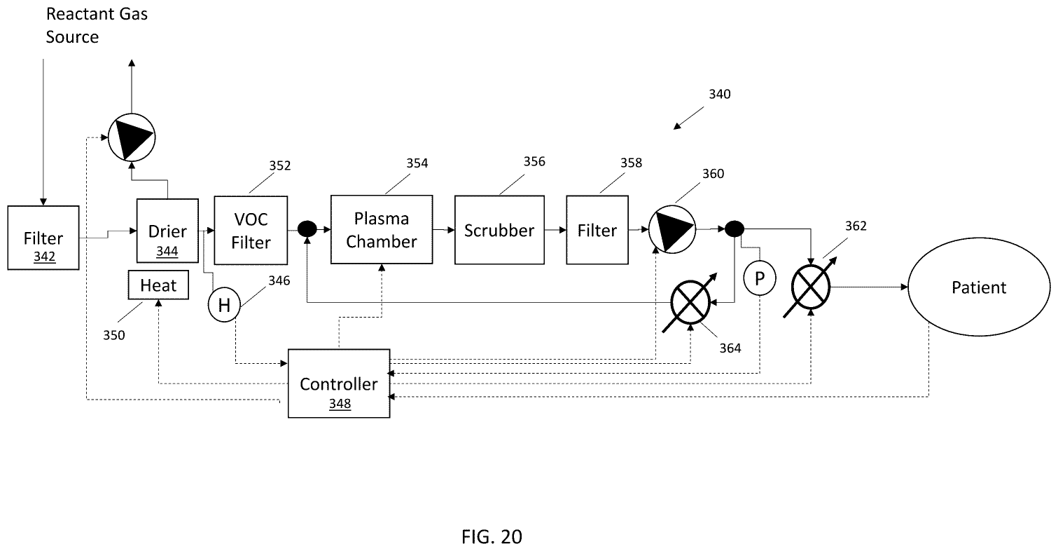

FIG. 20 depicts an exemplary embodiment of a system 340 that includes particle removal before the pump. Reactant gas enters the system through a particle filter 342. The gas then passes through an active drier 344. Humidity of the gas exiting the drier is measured by a humidity sensor 346 and reported to a device controller 348. A heater 350 below the drier is modulated by the controller 348 based on the humidity of the gas exiting the drier. A pump above the drier, also controlled by the controller, is utilized to dry out the drier with additional gas and return that gas to the atmosphere.

After the drying stage, gas passes through a volatile organic compound (VOC) filter 352 (e.g., activated carbon) to remove one or more of VOCs, semi-VOCs, and/or other harmful substances. Gas then enters the plasma chamber 354 and passes through an optional filter (not shown), a scrubber 356, and a filter 358. Gas then passes through a pump 360 that draws gas through the system. Located after the pump is an intersection between several pneumatic pathways, referred to as a “node.” In some embodiments, the node is maintained at constant pressure and NO concentration. Pressure in the node is measured via pressure sensor and reported to the device controller. Two flow controllers can direct gas from the node. A first flow controller 362 modulates NO product gas flow to a patient inspiratory stream. A second flow controller 364 directs excess gas back to a location upstream of the plasma chamber, forming a recirculation loop. The return flow controller is modulated to maintain a constant pressure at the node.

The concentration of NO at the node is held constant by modulating plasma activity within the plasma chamber. The controller directs the plasma chamber to make varying amounts of NO as a function of one or more of the target patient dose setting, the inspiratory flow rate, the amount of NO expected to be lost to oxidation, the amount of NO expected to be lost to interaction with the system (e.g., scrubber), and the amount of returned product gas vs. fresh reactant gas entering the plasma chamber. At a high level, the plasma chamber is generating NO in the fresh reactant gas and generating NO to make up for lost NO from transiting the recirculation loop.

### NO Generation

### Recirculation Loop Concentration Management

In some embodiments (not shown), the concentration of NO at the node shown in FIG. 20 is measured with a NO sensor to serve as feedback to the controller. A product gas NO sensor can provide compensation for variation in the system performance, as can occur from one or more of electrode wear, scrubber to scrubber variance, reactant gas water content, inaccuracies in injected NO volume, inaccuracy in calculated NO losses, and other factors. In some embodiments, the concentration of NO within a recirculation architecture can drift over time. In some embodiments, a product gas NO concentration sensor is utilized by the system controller to quantify the drift in product gas concentration. In some embodiments, the system controller uses a plasma chamber calibration to predict an amount of NO produced at current reactant gas conditions and plasma parameters (open loop control). Then the controller utilizes the NO loss calculation to calculate an expected amount of NO at a product gas NO sensor. When the calculated prediction and NO sensor measurements disagree more than a threshold amount, it is an indication that the NO sensor may have failed or is out of calibration and the controller can prompt the user to address the issue. In some embodiments with back-up NO generation capability, a controller will switch NO production to a back-up NO generation channel in the event that a product gas NO concentration sensor fault is detected.

In some embodiments, the recirculation loop is periodically purged of product gas to prevent drift in product gas concentration. In some embodiments, the plasma is turned off for a period of time (e.g., 30 seconds) periodically (e.g., every 15 minutes) to reset the gas concentration within the recirculation loop to zero. In some embodiments, the dilution ratio (i.e., the ratio of the inspiratory mass flow to the injected NO product gas flow) is altered to increase the quantity of gas exiting the recirculation loop and fresh gas entering the recirculation loop. In some embodiments, this is accompanied with a reduction in the NO product gas concentration in order to maintain a target inhaled NO concentration. In some embodiments, the NO product gas concentration is not altered because the volume of the recirculation loop is small enough that the transient shift in inhaled NO concentration would be negligible at the patient. By increasing the turnover of gas within the recirculation loop via changing the dilution ratio, NO concentration within the loop can be more accurately known since the NO within the loop is fresher and NO loss effects are less.

### Generation and Delivery of Low Doses of NO

In some embodiments, a NO generation system increases the gas turn-over within the recirculation loop by intermittently producing NO. The device controller first directs the plasma chamber to bring the NO concentration within a recirculation loop to a target value and then stops NO generation (i.e., plasma activity) while continuing gas flow through the loop. The concentration of NO within the loop decreases over time due to a combination of NO oxidation and/or interaction with system components (e.g., the scrubber). In some embodiments, the NO concentration decreases in a predictable manner that can be calculated by the controller. As the concentration decreases, the product gas injection flow rates are increased to deliver the correct moles of NO to accurately dose the inspiratory flow. In other words, the dilution ratio (i.e. quantity of NO mass flow required to dose a specific inspiratory mass flow) is altered to achieve a target inhaled NO concentration. When the concentration of NO within the recirculation loop nears or reaches a minimum value below which the system would not be able to accurately dose the patient, the controller re-ignites the plasma chamber to load it with NO again. This minimum NO concentration within the recirculation loop is related to the NO mass flow within the recirculation loop (i.e. ppm.lpm, or ulpm). The recirculation loop NO mass flow must always exceed NO mass flow required at the patient. When the NO mass flow within the loop falls to an amount that barely satisfies the patient production demand (e.g. 25% of the patient max production level), the system recharges the recirculation loop with NO. In a specific example, a patient is being dosed with 5 ppm NO while on a ventilator set at 2 lpm bias flow and 15 lpm peak flow. The maximum production level that the patient requires is 5 ppm*15 lpm, or 75 ppm.lpm of NO. The flow rate through the recirculation loop is a constant 3 lpm. The maximum inspiratory limb dilution is set to 9:1. Hence, the maximum injected NO flow rate is 1/10 of the maximum inspiratory flow rate (15 lpm), or 1.5 lpm. Thus, the concentration within the recirculation loop must exceed 50 ppm (750 ppm.lpm/1.5 lpm) to accurately dose the patient. In order to not fall below the minimum amount of NO within the recirculation loop, the NO system operates with a 25% safety margin, making the minimum NO concentration within the recirculation loop 62.5 ppm. When the NO concentration within the recirculation loop falls to 62.5 ppm, the system turns on the plasma and increases the concentration of gas within the loop to a high level (e.g. 2000 ppm) and then turns off the plasma and allows the NO concentration to decrease again over time. This approach enables a NO generation system to operate with known NO concentration within the recirculation loop, the known NO concentration provided by one or more of an NO sensor or a NO oxidation computational model.

### Target Flow Pressure Compensation: Increasing Product Gas Pressure

Inspiratory flow pressures are typically very low. For example, CPAP has a maximum pressure of 30 cm H₂O, or 0.42 psi. The pressure in the recirculation loop is maintained at a target pressure that is sufficient to deliver accurate amounts of NO to the inspiratory flow. In one exemplary embodiment, the product gas pressure within the NO generation system is 2.2 psi (15 kPa). In some embodiments, the product gas pressure within the NO generation system is between 1 and 30 psi. Although higher NO pressure facilitates injection of NO into a breathing system, the higher pressure also proportionally increases the rate of NO loss to oxidation.

In some applications, (e.g., jet ventilation), NO product gas is introduced to a high pressure region of the system. In high pressure applications, some embodiments of a NO generation system increase the pressure of the NO product gas, accordingly. The controller of the NO generation device determines when to increase the pressure of the product gas in response to one or more of measurements of target flow pressure via pressure sensor, input from the user, and/or direct communication (electric or wireless) with the concomitant therapy device. Increased NO product gas pressure will increase the rate of NO loss due to oxidation. In some embodiments, the device controller accounts for the additional NO loss by increasing NO production at the plasma chamber (i.e. increasing the frequency, power, or duty cycle of plasma pulses). In some embodiments, the amount of NO loss is either measured with an NO or NOx sensor or calculated.

In some embodiments, the pressure of the target (e.g. inspiratory) flow is measured by a pressure sensor within a ventilator cartridge, injector module, or gas sampling module.

### Target Flow Pressure Compensation: Increasing Product Gas Concentration

In some embodiments, high pressure in a target inspiratory flow (e.g., high flow nasal cannula treatment) results in reduced pressure drop from product gas to target flow across the injection flow controller. In some embodiments, the NO product gas injection mass flow controller opens more to compensate for the reduced pressure drop. In some applications, increased target gas flow pressure can result in reduced mass flow of product gas into the target flow when the product gas pressure is near that of the target flow. In some embodiments, the decrease in product gas mass flow through the flow controller is compensated for by increasing the product gas concentration to ensure that a target number of moles of NO are delivered to the target flow (e.g. inspiratory flow).

Product Gas Measurement with NOx Sensor

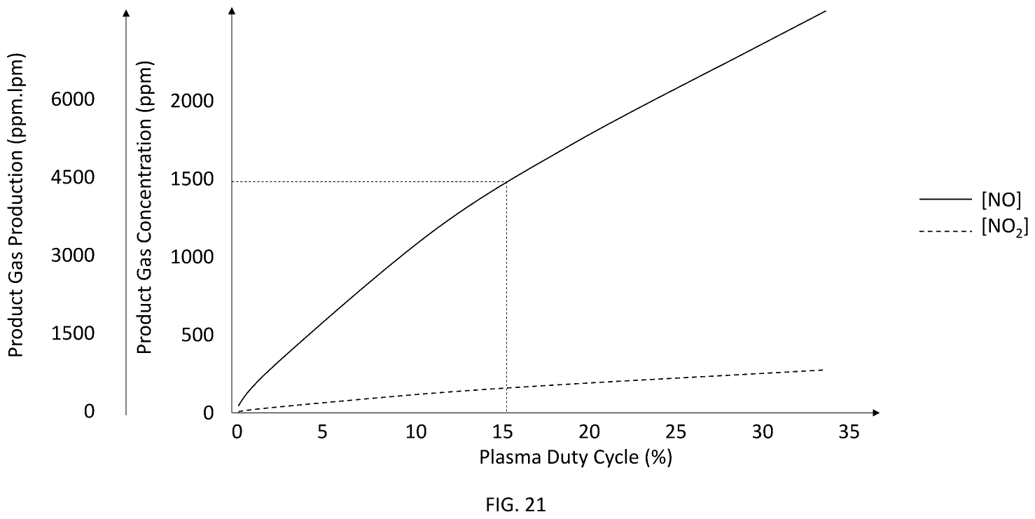

In some embodiments, a NOx sensor (i.e., a sensor that measures NO and NO₂) is utilized to monitor NO production. In systems that have been characterized for NO to NO₂ ratio, the quantity of NO and NO in a NOx measurement can be calculated. Characterization for NO to NO₂ ratio involves measuring the NO and NO₂ levels across a range of NO production levels and typically performed at the time of manufacture and after system servicing (e.g. electrode replacement). FIG. 21 depicts an exemplary graph of an example of a calibration curve for a specific reactant gas flow rate. A NO generation system is operated over a range of plasma duty cycles at a constant reactant gas flow rate. The level of NO and NO₂ production at each duty cycle is captured. Similar characterization can be performed for various levels of reactant gas humidity, reactant gas flow rate, reactant gas temperature, reactant gas pressure, electrode wear status, system operating temperature, and other factors for the controller to estimate the amount of NO and NO₂ production for a given set of input conditions and plasma settings. Although duty cycle is presented as the independent plasma control variable, characterization of a system can be done as a function of one or more plasma control variables including plasma frequency, plasma duty cycle and plasma power. The Y axis is in units of gas concentration. In some embodiments, the Y-axis portrays NO production (e.g., ppm.lpm), the mathematical product of product gas concentration and product gas flow rate.

The calibration curve is used by a NO generation controller as follows. The controller determines a target product gas concentration based on many factors, including but not limited to patient treatment parameters (e.g. patient inspiratory flow rate, target inhaled concentration), reactant gas parameters (e.g. temperature, pressure, humidity), and system parameters (e.g. scrubber age, NO loss measured or calculated, quantity of NO returning from a recirculation loop, quantity of fresh gas added to recirculation loop). The controller then utilizes the calibration curve in either a tabular or mathematical equation form to relate the target product gas concentration to the plasma settings (e.g. duty cycle) required to generate that production. In FIG. 21, a target NO product gas concentration of 1500 ppm is highlighted. The controller correlates the target NO product gas concentration to 15% duty cycle and commands electrical discharges to occur accordingly.

In some embodiments, the NOx sensor is located downstream of a NO₂ scrubber. This enables the NOx sensor to serve as a NO sensor since NO₂ has been removed from the gas stream. Best results are obtained when a highly effective NO₂ scrubber is utilized (e.g. soda lime, MOF, ascorbic acid, etc.).

Some versions of NO product gas sensors (e.g., NOx sensors) draw considerable electrical power (e.g., 10 W). Operating the product gas sensor continuously during battery-powered NO generation can hasten battery drainage and shorten battery life. In some embodiments, when the system is operating off battery power, the NO product gas sensor is powered intermittently or not at all to save battery power. The sensor may be used more frequently or even continuously when the NO device is powered by external power (e.g., AC power, external battery). This same principle can be applied to other sensors that are not required continuously for device operation.

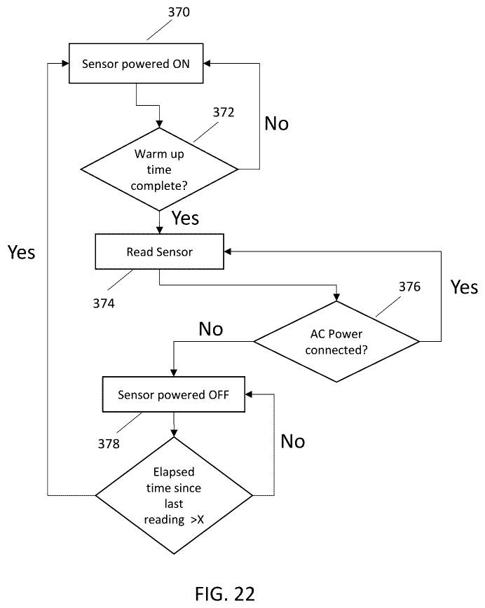

FIG. 22 depicts an exemplary flowchart of an embodiment of how a NO generation controller can intermittently read from a sensor during battery operation. When the sensor is powered on (step 370), there is a warm-up period required (step 372) before the sensor can be read. Once the warm-up period is complete, the sensor can be read (step 374). If the system is connected to external power (decision made at step 376), the system can leave the sensor on and continue reading from it. If the system is operating from limited or internal power, the sensor can be depowered (step 378). After the particular period of time elapses, the sensor can be powered on again and read. The period of time that the sensor is off is a function of how much deviation can be expected by the measurand and how long it takes for the sensor to warm-up.

### Pulsed Delivery

In some embodiments of an NO delivery system, NO gas is delivered in a pulsatile manner to one or more breaths. In some embodiments, the user sets one or more of the following parameters in the system: NO pulse duration, pulse flow rate, frequency (e.g., every breath, alternative breaths, time period between pulses, etc.), NO concentration (within the concentrated pulse or at the patient), and NO dose (e.g., mg/hr). The system then delivers NO according to these settings, independent of patient respiratory rate, tidal volume, and inspiratory flow rate. In some embodiments, the NO delivery system monitors inspiratory flow with a flow sensor and delivers a pulse of NO during inspiratory events.

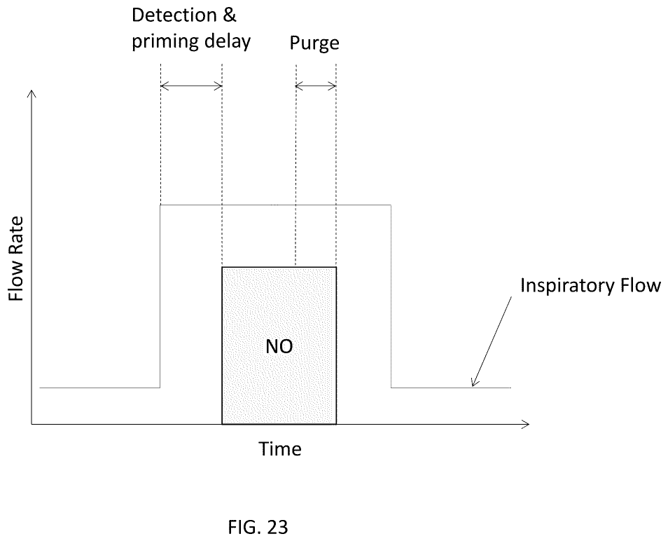

FIG. 23 depicts an exemplary graph showing an embodiment of an inspiratory flow profile (e.g., square wave). The NO delivery device detects the inspiratory event. The NO delivery device then primes the NO delivery lumen and delivers NO to the patient during the inspiration. After introducing the target amount of NO (typically measured in moles) to the NO delivery device, the NO system then purges the delivery device of NO before the end of the inspiratory event, thereby leaving the delivery device devoid of NO and NO₂.

### Discontinuous NO Delivery

NO oxidizes into NO₂ over time. One approach to reducing inhaled NO₂ levels is to minimize transit time of NO from its source to the patient. In some embodiments, NO is delivered in an alternating fashion with a non-NO containing gas. For example, the following approaches all deliver the same inhaled concentration to a patient:
1) Continuous: 100 ppm NO at 1 lpm;
2) Intermittent with higher flow rate: 100 ppm NO at 2 lpm, N₂ at 2 lpm, alternating with a 50% duty cycle.

Intermittent delivery enables a higher overall flow rate for decreased transit time while maintaining the same amount of NO delivered. This is particularly useful when delivering NO through a long tube to a patient inspiratory limb or directly to the patient because it decreases the transit time of the gas. It should be noted that this approach delivers more gas overall to the target, which can affect dilution ratio in some applications.

In some embodiments, NO is delivered in alternating fashion with another gas (e.g., oxygen). By sequentially alternating gases, interaction between the two gases is minimized while still delivering the same overall quantity of NO to the patient. Alternating NO and oxygen delivery can result in less NO₂ formation.

Intermittent delivery can be applied to treatments that require continuously variable NO output (e.g., ventilator flows). In an exemplary embodiment, the NO and O₂ delivery are alternated at a frequency of 100 Hz. The NO pulse frequency is selected to be high enough that the ventilator flow profile is accurately dosed and both gases reach all recruited locations of the lung.

FIG. 24 depicts a ventilator flow profile that is dosed with intermittent NO pulses. Black portions of the graph depict NO delivery and white portions depict another inspiratory gas (e.g., air, oxygen, heliox). The frequency of NO pulses in FIG. 24 is 44 Hz, however other frequencies could be equally effective. This approach allows the NO to interact less with the inspiratory flow. This can be beneficial when the inspiratory flow is 100% oxygen which rapidly oxidizes NO into NO₂.

In some embodiments, NO is delivered in a pulsatile manner intermittently, followed by a purge gas. The intermittent NO pulses are delivered with rapid flow rates and short transit times to minimize NO oxidation during delivery. In some embodiments, NO pulses are delivered at a constant frequency (e.g., every 50 msec NO or 100 msec) with the quantity of NO in each pulse scaled to the amount of inspiratory gas flowed over the same time period.

In some embodiments, the NO is diluted with another gas during delivery so there is a faster flow rate in the delivery tube and shorter transit time. In some embodiments, the dilution gas has low or no oxygen to minimize NO oxidation during transit. In some embodiments, the dilution gas is sourced from the ventilator circuit so that there is less overall dilution/volume change to the ventilator circuit.

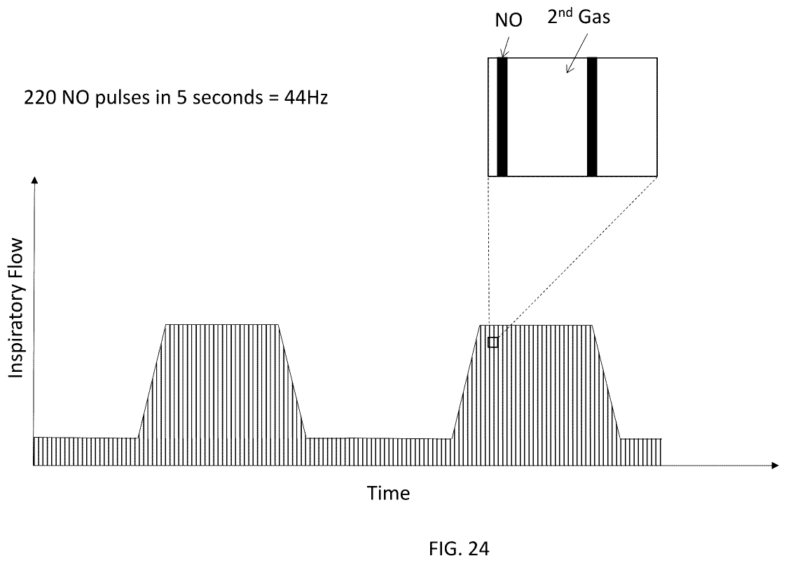

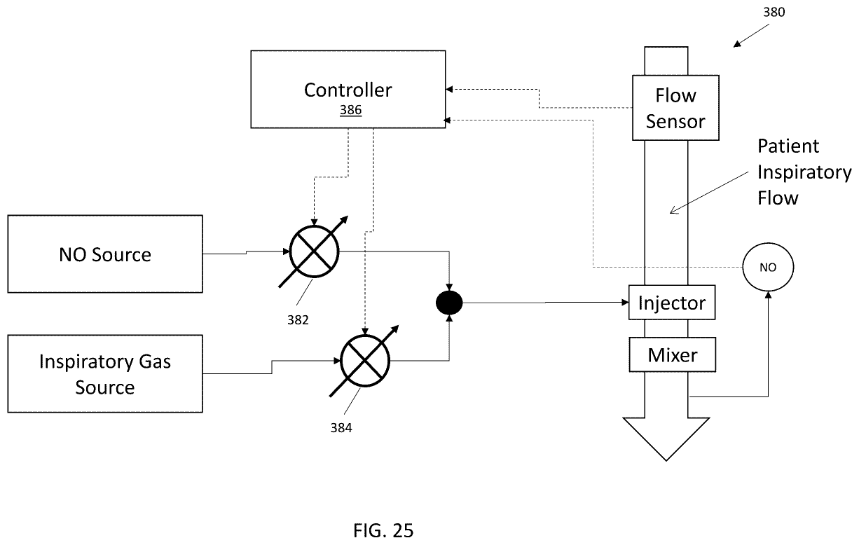

FIG. 25 depicts an exemplary NO delivery system 380 for delivering alternating flows of NO and another gas. Two flow controllers 382, 384 for NO and another gas are controlled by a common controller 386. The controller receives mass flow information about the patient inspiratory flow either directly (shown) or indirectly from another device (not shown). The controller calculates the amount of NO required to accurately provide either a target mixed concentration or a target NO mass per unit time (e.g. mg/hr). The controller then controls the gas flow controllers to provide flow in proportion to the inspiratory flow in an alternating fashion. In some embodiments, the flow of two gases is controlled by a single three-way valve (not shown). After NO gas is introduced to the inspiratory limb, the gas passes through an optional mixer (e.g., static, dynamic). An optional NO sensor in the inspiratory limb is utilized to measure the diluted NO concentration.

The train of alternating pulses of NO-containing gas and another gas can vary in duty cycle. In some embodiments, the NO duty cycle is 50%. In some embodiments, the NO duty cycle is between 1 and 99%, depending on the quantity of NO to deliver, the frequency of NO pulses, and the concentration of source NO gas.

### System Lag Effects on Inhaled NO Concentration

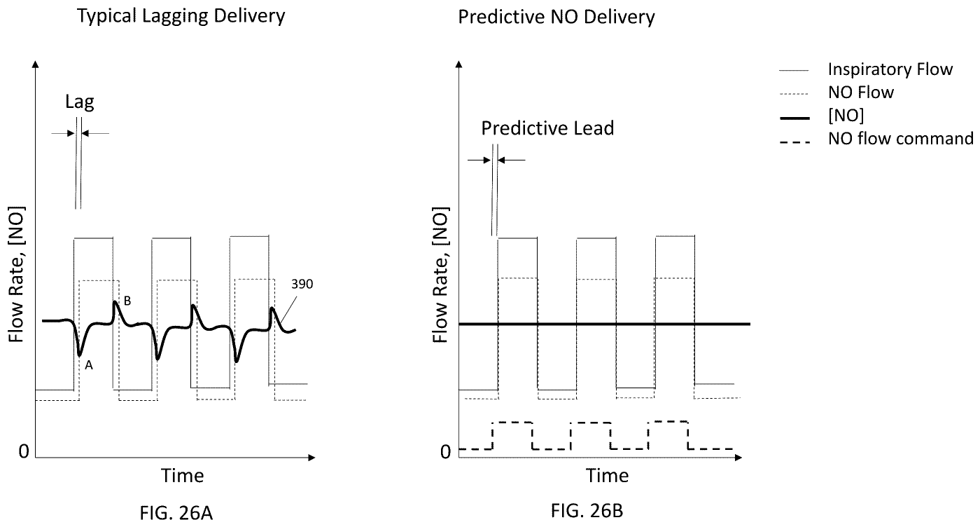

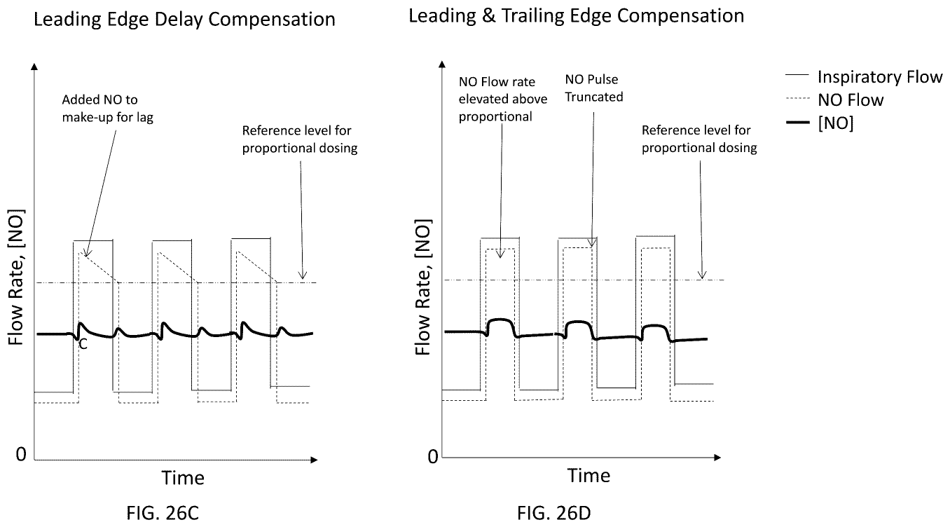

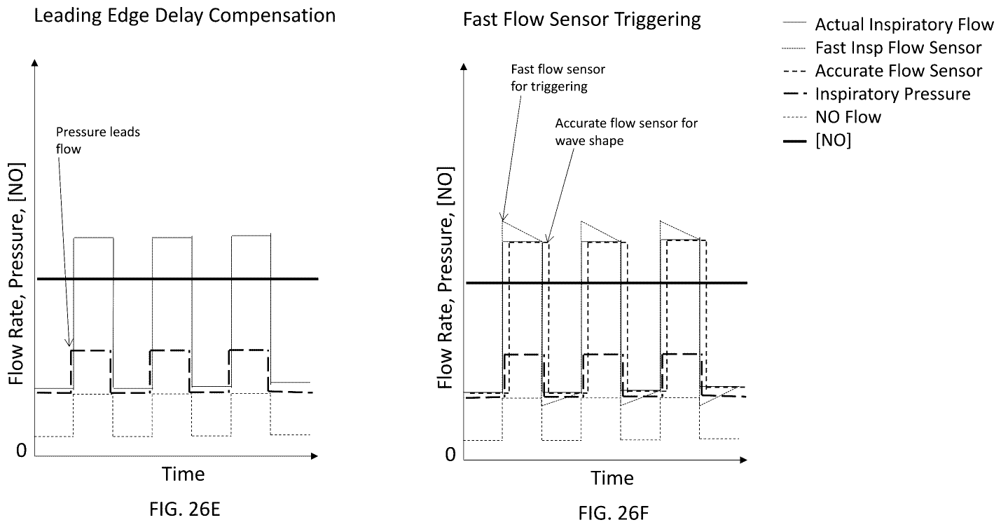

In some embodiments, the controller in a NO delivery system compensates for inaccuracies in NO delivery (e.g. system lag) to ensure that a target number of moles of NO are delivered to a patient within an inspiratory event. FIG. 26A depicts an exemplary graph showing inhaled concentration (line 390) when a NO delivery system has inherent lag. When the inspiratory flow rate increases during inhalation, the concentration of gas within the inspiratory limb can drop due to delay in detecting the inspiration and delivering NO. The drop in inspiratory gas concentration is annotated with an “A”. Lag in decreasing NO delivery can result in overdosing the inspiratory flow, as shown at point “B”.

### Predictive NO Delivery

FIG. 26B depicts an exemplary graph showing the inhaled NO concentration for a NO delivery system that predicts inspiration. In this embodiment, the NO delivery device controller either receives the ventilator waveform from a user or ventilator or measures and characterizes the periodic ventilator waveform with an appropriate sensor. Once the ventilator waveform is characterized, the NO delivery system compensates for system lag by commanding NO delivery ahead of future inspiratory events so that actual NO delivery occurs in unison with the inspiratory flow. It should be noted that the lag behind a rising edge can differ from the lag behind a falling edge of an inspiratory event. The rising edge lag is related to the NO delivery system's ability to detect the inspiratory event (e.g. flow sensor response time and resolution), calculate the amount of NO to deliver and related flow controller setting, actuate the flow controller and establish flow to deliver the required quantity of NO. Establishing flow can be delayed when the system has inherent flow restriction (e.g. some NO₂ scrubbers) or considerable dead volume. The falling edge lag is related to similar factors but is typically a different value. For example reducing flow from a high level to a low level can involve depressurizing gas between the flow controller and the inspiratory flow. The duration of NO delivery lag on the trailing edge of an inspiratory event is related to the dead volume of the NO system between the flow controller and the inspiratory flow as that volume of gas must bleed down in pressure, overdosing the inspiratory flow as it does so. In one example, a system with a rising edge lag of 10 msec and a falling edge lag of 15 msec commands NO delivery 10 msec ahead of the inspiratory event and commands reduced NO flow 15 msec before the end of the inspiratory event. As can be seen in the figure, shifting the phase of the NO command signal an appropriate amount of time ahead of the inspiratory waveform can result in constant inhaled NO concentrations. In some embodiments, the amount of system lag during the rise and fall of an inspiratory event is characterized for a given NO delivery system as it varies with inspiratory flow slew rate and magnitude. The NO delivery controller modulates the predictive lead of the NO delivery command signal based on inspiratory flow and NO delivery system characteristics, accordingly.

### Compensatory NO Delivery: Leading Edge

In some applications, the inspiratory waveform is not periodic and/or the system does not have a predictive algorithm. In these cases, a NO delivery system with inherent delivery delays can still improve upon the inhaled NO dose accuracy by compensating for the rising edge lag. FIG. 26C depicts an exemplary graph showing the way in which a system can add additional NO (i.e., overdose) to the inspiratory event to make up for the delayed introduction of NO to the inspiratory flow. A linear decay in NO overdosing is depicted, however it should be understood that other NO overdosing approaches are possible (e.g., exponential decay, step-wise decay, etc.). In some embodiments, this approach is utilized when the location of NO injection is near the patient (e.g. mask or cannula delivery), where the dose inspiratory gas has little to no transit time before entering the patient. By comparison, the downward deviation in inhaled dose is less than the unmitigated scenario (depicted in FIG. 26A, point A) when NO compensation is deployed.

### Compensatory NO Delivery: Trailing Edge

A NO delivery system may also compensate for inherent system delays by truncating the pulse of NO delivered into an inspiratory event. FIG. 26D depicts an exemplary graph showing inputs and output of a NO delivery system that truncates the NO pulse to prevent overdosing the slow ventilator flow (i.e., bias flow) after an inspiratory event. This is automatically done in predictive NO delivery, as described above, whereby the NO flow rate is decreased prior to the end of inspiration in a periodic ventilatory treatment. When the timing of the end of inspiration is not known, a NO delivery system can deliver a target quantity of NO into the breath in less time than the inspiratory event. In some embodiments, the duration of the inspiratory event is inferred from the duration of one or more prior inspiratory events. In some embodiments, NO is delivered at a maximal rate (e.g. a pulse) into an inspiratory event until a target quantity (e.g. moles) of NO has been delivered to the patient. This approach is most effective when the point of NO injection is near the patient (e.g. the patient Wye connector, nasal cannula, mask) so that the gas dosed during peak flow rates is the same gas that is inhaled by the patient. In some embodiments, NO is delivered at a constant rate or a rate proportional to the bias flow of a ventilator setting between inspiratory events. This approach eliminates overdosing of the inspiratory flow at the end of inhalation due to system lag, thereby conserving NO.

### Pressure-Wave Triggering

FIG. 26E depicts exemplary inputs and output of a NO delivery that triggers inspiratory event dosing off the pressure wave in the inspiratory flow. A change in pressure occurs before flow begins, thereby providing an indication of an upcoming inspiratory event before flow takes place. In a spontaneously breathing patient, the pressure event is a drop in inspiratory limb pressure as the patient draws gas into their lungs. In a ventilated patient, the pressure event is a rise in pressure as the ventilator pushes inspiratory gas to the patient. As shown in FIG. 26E, the pressure wave occurs first, followed by the flow wave. The NO delivery device controller monitors a pressure sensor in communication with the inspiratory flow. Once the inspiratory flow is detected, the NO delivery controller initiates the release of NO to dose the impending respiratory event. In some embodiments, the controller characterizes the shape of one or more prior flow waveforms to determine the flow rate of the NO pulse to be delivered. This approach provides an advantage of faster initiation and termination of NO delivery, improving the accuracy of inspiratory flow dosing, thereby enabling more constant NO concentration within the inspiratory limb. This ultimately results in improved inhaled dose control. A similar approach can be utilized by a NO delivery controller to predict the end of the inspiratory event using a change in pressure in the inspiratory flow.

### Fast Flow Triggering

Fast-responding flow sensors can be beneficial in detecting an event but tend to be noisy, less-accurate and underdamped. In some embodiments of a NO delivery system, a fast-responding flow sensor is utilized to detect inspiratory events. In some embodiments, the flow profile of the respiratory waveform is provided by an external device (e.g. a ventilator). In some embodiments, the respiratory waveform is accurately measured with a slower-responding flow sensor (i.e., a sensor with time delay). FIG. 26F depicts exemplary inputs and output of a NO delivery system that utilizes a very-fast-response inspiratory flow sensor to monitor an inspiratory flow and detect respiratory events. There may be a typical trade-off between fast-response and lower-accuracy. For example, the signal from the fast-response flow sensor signal increases soon after the actual inspiratory event but overshoots the actual max inspiratory flow rate. A slower inspiratory flow sensor is utilized to capture the inspiration wave shape. The inspiration wave shape of one or more prior breaths is utilized to guide NO delivery to a current inspiration. The flow event-timing is established via the fast-response flow sensor. The slow-response-more-accurate flow sensor is used to control dosing level, preferably with a knowledge of the shape of some number of prior flow waveforms. This dual sensor approach enables fast system response to initiate delivery of NO to an inspiratory event while also providing accuracy during a relatively long dose after the initiation.

### Variable NO Production

The level of energy required to excite reactant gas between electrodes scales with NO production levels. In some applications, a single transformer is unable to address the entire NO production range for a NO generation system. Hence, some embodiments have two or more electrical transformers to address the range of electrical energy required for the range of NO production. In some embodiments, the system includes a low range transformer and high range transformer that are used one at a time. In some embodiments, two transformers are utilized in series like a voltage divider so that a single transformer is used for low production levels and both transformers are utilized for high production levels.

In some embodiments, the system includes a potentiometer (i.e., variable resistor) that is controlled by the controller to tune the resonant frequency of the high voltage circuit. In some embodiments, the system includes a bank of capacitors, of which a varying number of capacitors can be utilized, as dictated by the controller, to vary the high voltage circuit resonant frequency. In some embodiments, a system varies the resonant frequency to enable lower production levels of NO. In some embodiments, a system varies the high voltage circuit components (e.g. capacitors) to compensate for changes in the high voltage circuit (e.g. electrode wear). In some embodiments, the controller opens/closes a switch to disconnect/connect a bank of capacitors to re-tune the circuit and change production levels. In some embodiments, this method is utilized to enable a system to produce accurate amounts of NO at low production levels (e.g. neonate treatments) and high production levels (e.g. adult treatments).

### Plasma Initiation

Plasma initiation (e.g., breakdown of gases within an electrode gap) is facilitated by the presence of ions in the plasma chamber. In some embodiments, UV light is used to create ions within the plasma chamber to facilitate breakdown. In some embodiments, a plasma chamber includes a UV lamp. In some embodiments, the UV lamp is on continuously. In some embodiments, the UV lamp is used intermittently. For example, UV may only be used when the electrodes are cold. In some embodiments, a hot wire is used to generate ions in the reactant gas to facilitate breakdown. The thermionic emissions of the heated wire (e.g. a nichrome wire with appropriate electrical current flowing through it) emits ions. When placed upstream or near an electrode gap, the emitted ions facilitate electrical breakdown in the gap when high voltage is applied.

### Low Production

### Reactant Gas Mixture

Optimal production of NO in a nitrogen/oxygen gas mixture occurs at the stochiometric ratio of nitrogen and oxygen (i.e. 50/50). As the ratio favors oxygen or nitrogen, the amount of NO produced per standard electrical discharge decreases. This relationship can be leveraged when low levels of NO production are required. In one embodiment of a NO generation system, low levels of NO production are achieved by utilizing reactant gas with low amounts of either nitrogen or oxygen. In one example, reactant gas with 5% oxygen is utilized. Utilizing low oxygen levels in the reactant gas can mean that there is less oxygen for the NO to react with, thereby increasing the half-life of the NO gas mixture. In another example, electrical discharges are made in a gas mixture with low nitrogen levels. This can be used when low levels of NO are desired and a patient requires high levels of oxygen. By having high levels of oxygen, the product gas does not dilute other gas streams that the product gas may be mixed with. In some embodiments, the product gas with high levels of oxygen is inhaled by a patient undiluted.

In some embodiments, pressure-swing adsorption is utilized to separate oxygen and nitrogen to form a gas mixture with a target amount of oxygen and nitrogen. This gas mixture is then used as reactant gas in an electrical NO generation system.

### Shifting NO/NO₂ Ratio in NO Production

In some embodiments of an electrical NO generation system, low NO output is achieved by altering parameters within the plasma chamber to shift the NO/NO₂ ratio towards making more NO₂. This has a two-fold effect: First, less NO is generated, and second, more NO is lost within a soda lime scrubber due to the proportional scrubbing NO in the presence of product gas NO₂. This approach can enable a NO generation system to rapidly change the product gas NO concentration. For example, this can be used in a system that utilizes a recirculation architecture where product gas circulates within the system prior to being dispensed into an inspiratory flow. In one exemplary embodiment a NO generation and delivery system is treating a patient with a high dose of NO (e.g. 40 ppm) when the user decreases the target inhaled concentration to 10 ppm. The system responds by shifting plasma activity to favor generation of more NO₂, thereby decreasing the NO concentration circulating within the recirculation loop.

In some embodiments, this approach is utilized to achieve product gas concentrations that are lower than could be achieved within the plasma chamber. This enables a NO generation and delivery system to deliver lower inhaled concentrations than otherwise would be possible which can aid in providing greater dose resolution at low dose settings (e.g. during patient weaning). In some embodiments, a NO generation system alternates between NO₂ production and NO production to achieve a lower concentration NO product gas.

There are multiple ways that NO₂ production can be increased in a plasma-based NO generation system. In some embodiments of an electrical NO generator operating at constant electrical discharge frequency, the duty cycle is decreased to a level that prohibits electrical breakdown. In the absence of electrical breakdown, ozone is formed in the plasma chamber. The ozone reacts with NO in the product gas to form NO₂. In this embodiment, the generation system generates series of ozone generation discharges and NO generation discharges to produce a low concentration NO product gas. The ratio of ozone to NO generation electrical discharges can be varied as needed to vary the NO concentration in the product gas.

In some embodiments, the duty cycle is turned down to a lower duration that produces electrical breakdown but yields a higher proportion of NO₂ to NO. Higher NO₂ occurs, in part, because the electrodes are colder. In some embodiments, the electrical discharge duration is alternated between a higher value and a lower value over time. The higher value produces NO with low NO₂. When a soda lime scrubber is utilized, NO is absorbed in proportion to NO₂ absorbed. Hence, a low electrical discharge duration produces greater amounts of NO₂ so that the mixture loses more NO within the scrubber.

In some embodiments, an electrical NO generator that can vary both duty cycle and frequency can increase NO₂ production by increasing the frequency and decreasing the duty cycle. This has the effect of generating more NO₂ for a given amount of NO production.

Intentional generation of NO₂ or increased NO₂/NO ratio can be used rather than dithering between production and 0 production since the boluses of generated NO from each electrical discharge are closer together in time and space (dithering produces NO pulses that are spread out in space and time, requiring more time/volume for mixing into a homogeneous mixture). Varying NO/NO₂ ratio during NO production can result in smoother, more consistent product gas concentration as well as enabling lower net NO production (i.e. ppm.lpm output of the system).

### NO Generation Timing

The amount of time that it takes for gas to break down within an electrode gap is a function of electrode gap, voltage level, reactant gas ion content, electrode temperature, electrode wear, reactant gas humidity, reactant gas nitrogen/oxygen content, reactant gas pressure and/or other factors. These sources of variance can be challenging to account for and will change day to day and over the lifetime of a NO generation system.

Variation in breakdown time results in variation in actual on-time of the plasma which, in turn, affects NO production accuracy. For example, a NO generation system operating at 50% duty cycle and 200 Hz generates electrical discharges lasting 2.5 msec in duration. A delay in breakdown of 25 μsec results in a 1% decrease in plasma ON-time, which would result in roughly a 1% reduction in NO produced. At 5% duty cycle at 200 Hz, the electrical discharges last 250 μsec. At this lower duty cycle, the same 25 μsec delay results in a 10% deviation in duty cycle duration error which directly relates to NO production accuracy.

In some embodiments, the controller of a NO generation device measures the variation (e.g., delay) in plasma breakdown time and/or termination time to quantify the actual plasma ON-time. The controller then compensates for increases or decreases in plasma on time, as needed, to achieve a target plasma ON-time and NO production level. Compensation is achieved by extending a current or future electrical discharge or by inserting an additional discharge to a series of electrical discharges.

### Reduction of Discharge Timing Variation

In some embodiments, a NO generation system can include a UV source that emits UV light into the plasma chamber. The UV light increases the number of ions in the electrode gap, shortening breakdown time and decreasing breakdown time variance. Reduction of breakdown time variance improves the consistency and accuracy of plasma pulse duration which directly relates to NO production accuracy.

### Breakdown Detection

Some embodiments of a NO generation system detect the actual timing of the electrical discharge breakdown event. In some embodiments, the breakdown event is detected by sensing an increase in electromagnetic emissions (e.g. radiofrequency emissions with an antenna, light emissions with an optical sensor) or changes in the voltage or current within the high voltage circuit that energizes the electrodes.

By knowing the actual breakdown time, the controller can prolong a current electrical discharge, prolong a subsequent electrical discharge, and/or add an extra electrical discharge to make up for lost NO production in order to maintain a target NO production rate. In other words, despite variance in electrical breakdown timing at the onset of an electrical discharge, a NO generation controller can select a appropriate discharge termination time to ensure the electrical discharge is active for a target duration. For example, the NO generation controller can ensure that all discharges are a uniform duration, if so desired, by varying the discharge termination time to be a set amount of time after actual breakdown occurred.

In some embodiments, a NO generation system tracks the average plasma duration over time and adjusts the duration of one or more plasma pulses periodically to account for an excess or deficit in NO production as compared to a target production rate. For example, a system may adjust the length of a plasma pulse every minute to trim the NO production run rate to match a target value.

All other parameters being equal, breakdown time increases with electrode gap. Hence, breakdown time can also be used as a means to estimate electrode wear. In some embodiments, a NO generation system tracks breakdown time over the life of the device to track electrode wear. In some embodiments, the NO generation system generates an alarm and/or requires electrode replacement when breakdown time for a particular set of input conditions exceeds a threshold. In one specific example, when the controller measures electrode breakdown greater than 30 μsec, it does one or more of changes electrode use to another electrode assembly, generates a fault, and recommends electrode replacement.

In some embodiments, a NO generation system adjusts plasma settings (e.g. peak voltage, plasma AC waveform, frequency, etc.) for the current electrode gap length based on estimates of electrode gap length from breakdown time measurements.

Breakdown detection can also be used in detecting non-breakdown events (i.e. events where high voltage is applied but no breakdown occurs). Non-breakdown events can be an indicator of excessive electrode wear, cold electrodes, or other system faults. In some embodiments, a NO generation system that detects one or more non-breakdown events initiates an alarm to prompt electrode replacement. In some embodiments, the NO generation system attempts breakdown a second time. In some embodiments, a NO generation system alters plasma parameters (e.g. voltage, AC waveform, resonant frequency, etc.) in the event of a non-breakdown event to try to improve the success rate of breakdown. In some embodiments, a NO generation system prolongs a subsequent electrical discharge or adds an additional electrical discharge in a series of discharges to make-up for lost production upon the detection of a non-breakdown event.

### Plasma Termination Detection

In some embodiments, the timing of the actual termination of a plasma pulse is tracked. In some embodiments, the end of plasma is detected via electromagnetic emissions. In some embodiments, electromagnetic emissions are collected by an antenna. In some embodiments, electromagnetic interference (EMI) occurs in the signal of an existing sensor and can be quantified by processing the sensor signal. For example, a sensor signal can be filtered (typically high pass filtration) to leave only the EMI interference component. Then, the EMI signal amplitude is quantified. In some embodiments, the end of plasma is detected optically. When the plasma terminates, so too do light emissions from the plasma. This feature can be beneficial in detecting premature termination of plasma which would result in under-production of NO. A NO device controller calculates the actual duration of a plasma pulse by subtracting the breakdown time from the plasma end time. The NO device controller can compare the actual plasma duration to the target plasma duration. If the actual plasma duration is less than target duration, one or more future plasma pulses can be prolonged to make-up for lost NO production. Similarly, if the actual plasma duration is longer than the target duration, the NO device controller can shorten or skip one or more future plasma pulses to adjust the NO production rate to align with a target NO production rate.

### Plasma Termination Detection: Optical Emissions

In some embodiments, a NO generation system includes an optical sensor that can detect optical emissions from the plasma (e.g. visible spectrum, UV spectrum). The controller of the NO generation system monitors the optical sensor to determine the presence/absence of plasma, timing of plasma formation, plasma power, and changes in plasma power. In one example, a controller monitors an optical sensor at a fast frequency (e.g. 100 Hz). When the plasma is off, there is no light within the plasma chamber and the sensor reads zero. When the plasma occurs, light is emitted from the plasma and the light sensor output value climbs to a peak value. In some embodiments, the controller determines that plasma has formed when the light sensor value meets are exceeds a threshold value and marks the coincident time as the plasma formation time. In some embodiments, as is the case with slower optical sensors, the plasma formation time may be shifted earlier in time by the t90 time of the optical sensor to account for delays from sensor speed.

### Pulse Termination Detection: Electromagnetic Emissions

In some embodiments, a NO generation system utilizes an antenna to quantify electromagnetic emissions from the plasma chamber. EMI can permeate the interior of a NO generation system, resulting in EMI artifact occurring in the signal of many sensors. In some embodiments, a dedicated antenna is utilized to pick up the EMI signal. In some embodiments, the signal of an existing sensor (e.g. pressure, temperature, etc.) is utilized by the controller to quantify the magnitude of EMI artifact. For example, the signals of interest include High dv/dt and di/dt generated by plasma breakdown, and secondary voltage (indicating proportional to plasma path length). The latter can be measured using a capacitive pickup/“antenna.” The former is by nature very high frequency (10's to 100's of MHz) and can be picked up using a range of antennas/probes.

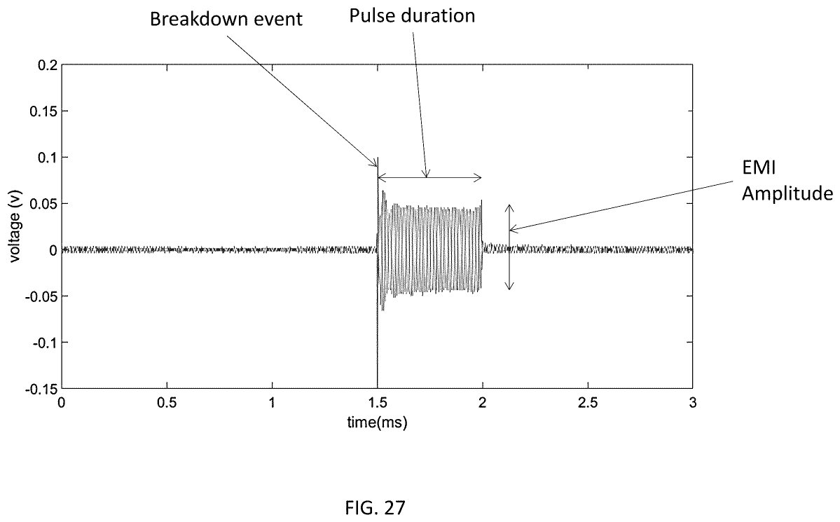

FIG. 27 depicts an exemplary graph showing data from a sensor measuring EMI output from a plasma chamber. The EMI signal can be utilized to identify the instant that breakdown occurs as well as the actual duration of the plasma pulse. The breakdown time is defined as the amount of time from the application of high voltage to the electrodes to the time that plasma forms.

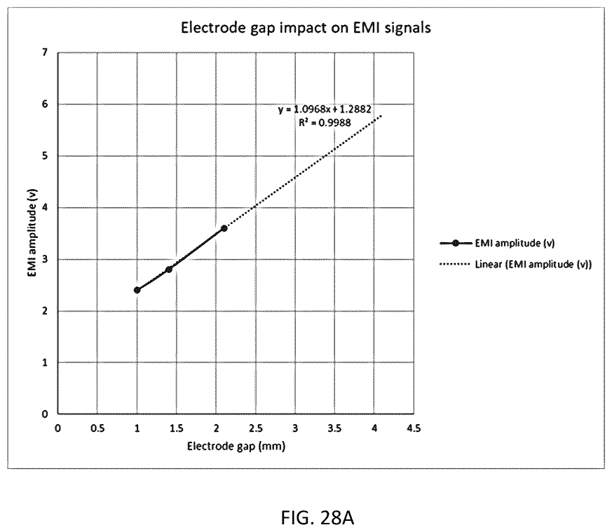

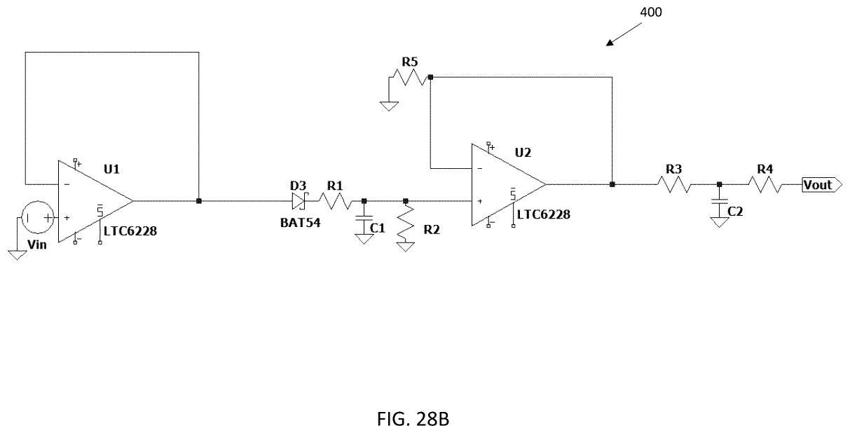

FIG. 28A demonstrates an exemplary graph of an embodiment of a relationship between electrode gap and EMI emissions for an overlapping, parallel electrode pair energized with AC voltage. The amplitude of the EMI output is directly proportional to the electrode gap. This relationship can be used by the device controller to quantify the electrode gap and infer a level of NO production. For example, when operating at approximately constant current, which should produce more or less a constant plasma cross section/volume resistivity, the voltage in the gap and NO production scales more or less linearly with gap length. This voltage can be measured via capacitive coupling in the aforementioned capacitive probe. Operating at a constant [resonant] frequency can also be helpful in this measurement technique because antenna-matching and probe impedances vary with frequency. However, in some embodiments, the system could apply a correction factor for operating frequency, since the electrical properties of said probe/antenna should be fairly well defined and repeatable.

### Peak Detector

During NO generation based on plasma breakdown of air particles, electromagnetic interference (EMI) signals are being generated and this signal has the potential to help quantify NO production. The beginning of the NO generation is signified by a large spike in the EMI signal coinciding with the breakdown event, as seen in FIG. 27. The arc length (shortest electric gap between two electrodes) with constant current is directly proportional to the amplitude of the EMI signal after the breakdown event, as seen in FIG. 28A. Arc length with constant current is also directly proportional to the NO production per unit time.

In some embodiments, to detect the beginning of the breakdown (i.e., the large spike shown in FIG. 27), the EMI signal is passed to an Analog-to-Digital converter (ADC) or an analog detector (e.g., a comparator) with a binary output for further processing. The challenge with accurately detecting and capturing the breakdown event is that it can occur within a microsecond, which makes it difficult to be detected when passed to an ADC that samples at less than 1 MSps.

A peak detector is a hardware circuit that latches in the event of a large peak in a signal. By latching, the peak detector changes a brief transient event into a simple step function that can be detected with an ADC operating at slower frequency. In other words, a peak detector has the potential to detect the EMI pulse associated with a plasma break-down event by quenching the large spike in values, thereby creating a delay in the signal to enable an ADC operating at a slower frequency (e.g., 50 to 500 KSps) to log the event.

In some embodiments, the amplitude of the peak detector waveform remains at an elevated value throughout the EMI pulse. In some embodiments, the amplitude of the peak detector waveform tracks the peaks of the EMI waveform during the NO generation pulse to provide the amplitude values of the EMI. Thus, a peak detector circuit can be used to detect the beginning of NO generation and the amplitude of the EMI signal, a mechanism to quantify NO production.

A peak detector by definition is used to determine the maximum value of an input signal. It stores this value for a period of time until the circuit comes to a reset condition. A reset condition can be reached as a function of time (e.g., emptying of a capacitor) or an external input (e.g., reset from a microprocessor).

In some embodiments, the peak detector has an asymmetrical bandwidth response to rising/falling voltage. It has the ability to acquire high frequency transients (breakdown) while having a low-pass response to falling voltage that allows an ADC to capture the signal.

For example, the falling passband can be set to remove the 50-400 kHz resonant frequency but also decay quickly enough to recover from the breakdown event and measure plasma voltage. With a minimum duty of 1.5% at 200 Hz, a minimum 75 us pulse width (13.3 kHz) is achieved. Achieving such a passband can be difficult, especially for a 50 kHz operating frequency. The settling time from the breakdown event is tuned carefully to accurately acquire the waveform amplitude at the end of the pulse.

In some embodiments, two envelope detector circuits can be employed. One would be tuned to reject the high-frequency breakdown event and capture only the amplitude of the post-breakdown signal. The other would be tuned to capture the breakdown event specifically.

In some embodiments, a peak detector circuit would employ an active reset to discharge the hold capacitor after the breakdown event is detected and allow accurate measurement of the post-breakdown signal amplitude.

And as previously mentioned, in some embodiments, a comparator would be used to turn the breakdown event into a digital trigger signal that does not require an ADC, but such an embodiment lacks the ability to measure post-breakdown amplitude by itself.

FIG. 28B illustrates an exemplary embodiment of a peak detector circuit 400. The input signal is passed through a voltage follower circuit which acts as a non-inverting buffer, to a Schottky diode which is the main peak detection component. This diode was chosen because of its very fast switching capability, fast enough to capture the breakdown event on the EMI signal. A capacitor C1 stores the current peak value until a higher peak value is acquired and this is passed through to another follower circuit and a capacitor C2 to hold the voltage signal which is to be sampled by the ADC. The operational amplifiers are also chosen for their gain-bandwidth product (e.g. 890 MHz) and slew rate (e.g. 500V/μs).

The values of the resistors and capacitors are chosen to create a bandpass filter circuit that allows the frequencies of breakdown event and desired EMI amplitude detection region to pass.

### NO Production Measurement

### Optical

The amount of light emissions from the plasma in a NO generator is related to the amount of power in the plasma and the amount of NO being produced. In some embodiments, an optical sensor is used to quantify the light emissions from the plasma. The quantity of light is then converted into a NO production value based on a calibration table or calibration function by the device processor.

Light emissions from the plasma vary with NO production. One way to capture drifts in NO production (including those associated with electrode wear and/or reactant gas humidity) is to quantify the light emissions from the plasma. In some embodiments, a photo-detection sensor is located at or near the plasma chamber to detect light emissions from the plasma. Depending on the type of sensor used, electromagnetic emissions from the plasma chamber can interfere with sensor and sensing circuit operation. In some embodiments, a light conductor (e.g., optical fiber, light pipe, etc.) is utilized to convey light from the plasma chamber to a remotely-mounted photo sensor. In this way, the sensor can be located away from the plasma chamber, thereby minimizing EMI. In some embodiments, the light conductor passes through a Faraday cage to reach the photo sensor.

In some embodiments, NO production within an electrical NO generation device is monitored with an optical sensor that receives the light output from the plasma chamber. In some embodiments, plasma light emissions are directed through a convex lens to focus the light emissions on an optical sensor to increase sensitivity. In some embodiments, a convex lens is utilized to focus the plasma light emissions on the end of an optical fiber. The optical fiber conducts the light, enabling the optical sensor to be far away from the plasma chamber and less susceptible to EMI emissions. In some embodiments, the sensing system includes a reference light source for checking calibration of the NO measurement sensor. In some embodiments, the reference light source is covered during NO generation to prevent contamination of the light source surfaces with pollutants (e.g. gases or particles) from the plasma chamber. The reference light source enables the system to quantify the effects of light source degradation, light sensor degradation, material deposition on the optics, and other failure modes. Periodically (e.g. daily, every treatment case, etc.), the system turns off the power plant, uncovers the calibration light source and turns on the calibration light source. Light travels from the source to the light receptors. The quantity of light received by the light sensor is compared to one or more values collected at an earlier time. The reference light source is utilized intermittently, thereby prolonging the service light of the light source.

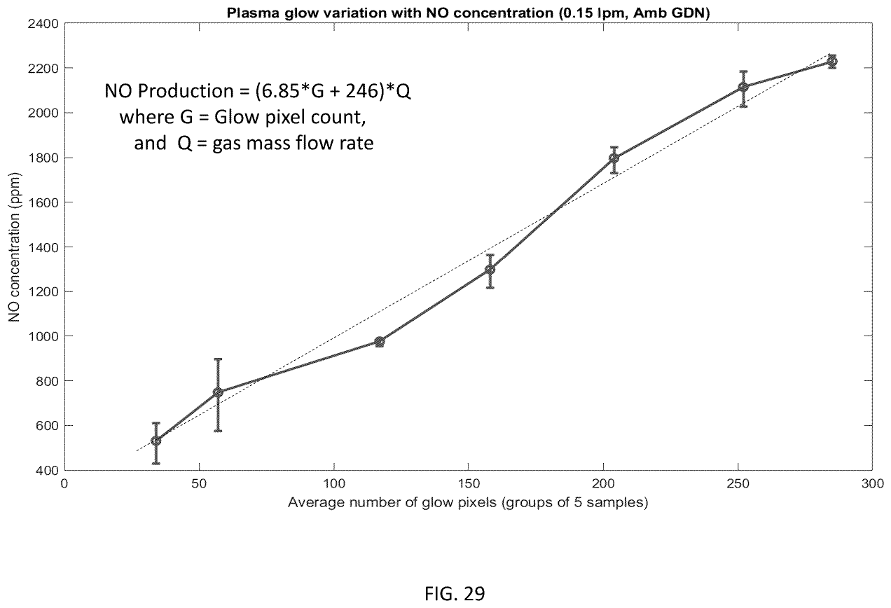

FIG. 29 illustrates an exemplary graph that presents experimental data using an electrical NO generator with an optical sensor that collects light output from the plasma chamber. The data were collected with an optical camera. Light intensity was determined by determining the count of pixels that registered light from the plasma. The flow rate of reactant gas (air) through the plasma chamber was 0.15 slpm. A linear relationship between optical light output from the plasma and NO concentration can be seen for the subject NO generation system. The relationship between optical light output and NO production is also linear because NO production is the product of NO concentration and gas flow rate and the gas flow rate was held constant (0.15 slpm). In some embodiments of a NO generation system, the system controller utilizes an optical output measurement from the plasma chamber to determine whether or not the plasma chamber is functioning (binary information). In another embodiment, the controller utilizes plasma chamber optical output measurements to quantify the amount of NO being generated. The NO generation levels are determined by utilizing the equation shown in FIG. 29 regarding the measured optical output (G) and gas flow rate (Q). In some embodiments, the system controller uses the derived NO generation measurement as an input into a closed-loop control system to achieve a target NO production level.

### Infrared Absorption

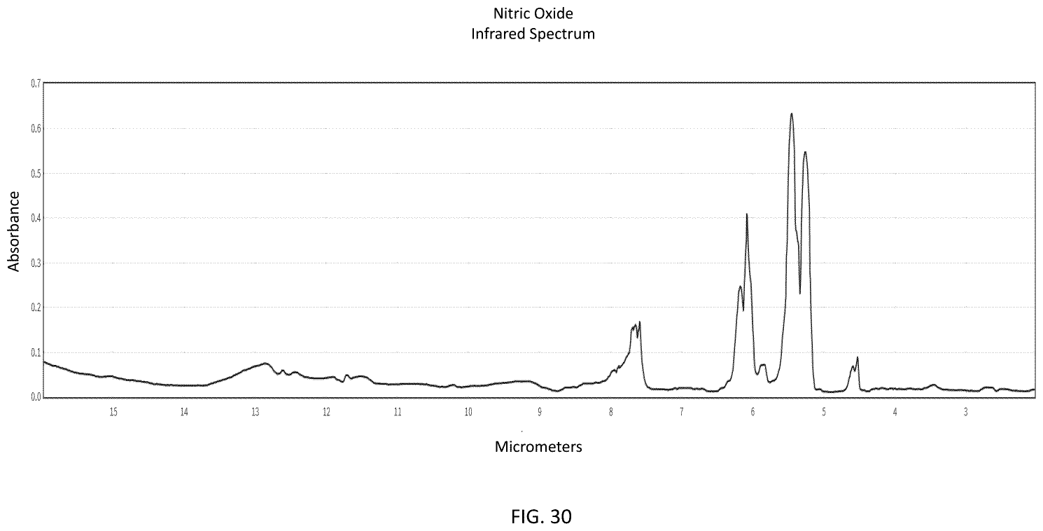

NO concentration can also be measured via infrared absorption (e.g., a Non-Dispersive Infrared (NDIR) sensor, also known as a “pyroelectric sensor”). An NDIR sensor, designed for measuring the concentration of nitric oxide (NO) works on the principle of photon absorption at particular wavelengths, particularly the IR spectrum. Operating in the IR spectrum allows for NO absorption lines that are particularly well defined, and that IR sources have low energy requirements, making an efficient measurement operating point. FIG. 30 depicts an exemplary graph showing the IR absorption spectrum for nitric oxide gas with well-defined absorption lines from 5 um-6.5 um.

In some embodiments of a NO generation and/or delivery system, a sensor system consisting of an IR source that includes the above range of wavelengths in its emissions is positioned at one end of a cavity. A pair of sensing elements are positioned at the other end of the cavity. Each sensing element has a narrow bandpass optical filter. A first sensing element, referred to as the “absorption sensor”, includes a filter that permits wavelengths that include the NO absorption spectra. The reference sensor has a filter that does not permit NO absorption wavelengths.

During operation, the source will emit a pulse of infrared energy. The reference sensor is used to quantify the strength of the IR source. This information can be used to compensate for changes in the reference source power over time. The absorption sensor detects the full magnitude of emissions from the source in the NO-band in the absence of NO. When NO is introduced to the chamber, a portion of the source emissions are absorbed by the NO. The intensity of infrared reaching the absorption sensor is diminished when NO gas is within the cavity due to absorption by the NO gas. The ratio of the absorption sensor signal with and without the presence of NO is proportional to the concentration of NO in the cavity. The ratio of light intensity (transmittance) is described by the Beer-Lambert law:

$$T = \frac{I}{{I}_{0}} {e}^{- aC}$$

where T is the transmittance,
I is the NO intensity,
I₀ is the reference intensity,
C is the NO concentration (e.g. ppm), and
a is a constant—determined by calibration.

The use of dual sensors allows for compensation related to the degradation of the source over time (e.g., due to age), which is compensated by an equivalent decrease in both the NO and reference intensity. A similar approach can be utilized within a NO generation and/or delivery system to measure the concentration of other gases, such as oxygen, nitrogen, nitrogen dioxide, and helium.

### Electrode Wear Detection

### Capacitance Approach

The air gap between electrodes in a NO generation device has a capacitance that changes with electrode gap. As electrodes wear, the gap increases. Hence, a capacitance measurement of the high voltage circuit can be indicative of electrode gap. In some embodiments, a NO generation device measures the capacitance across the electrodes either directly or indirectly (e.g., shifts in the resonant frequency). In some embodiments, the NO generation device alters NO production parameters (i.e., frequency, duty cycle, power, etc.) in response to the electrode capacitance measurement to achieve a target amount of NO production.

When the gap of the electrodes changes, the capacitance value of the electrode will change based on the capacitance formula between the two electrode surfaces.

The electrode capacitance formula is C=(€0×€1×A)/D, where €0 is the permittivity or dielectric constant of air, €1 is the permittivity or dielectric constant of electrode material (e.g., iridium), A is the surface area of the overlap between the electrodes, and D is the distance or gap between the two electrodes.

As electrodes wear, changes in A and D occur which induce changes in capacitance between the electrodes. Given that the resonance frequency is calculated as f=1/(2*pi*sqrt(LC)), where L is the circuit inductance and C is the circuit capacitance (including electrodes), the resonance frequency will change in response to change in either the inductance or the capacitance of the total circuit.

In some embodiments of a NO generation system, the system is calibrated for a relationship between the resonance frequency, duty cycle and NO production. This relationship/formula/equation is used to adjust the duty cycle based on changes in the resonant frequency to keep the NO production constant. This method detects and corrects for electrode wear.

In some embodiments, the electrodes are replaced after one or more of a set service life, a threshold amount of resonant frequency shift, a threshold amount of change in NO production. In some embodiments, electrodes and/or a plasma chamber are packaged in the form of a cartridge for simple replacement. In some embodiments, a NO system performs a resonant frequency search on a new electrode set after installation.

### Gas Pressure Method

Plasma discharges are extremely hot. When they occur, gas in the vicinity of the plasma expands creating a pressure wave that propagates up and downstream from the plasma chamber as a sound wave. As a result, electrical discharges can be detected using a pressure sensor (e.g. microphone, diaphragm sensor, etc.). The magnitude of the pressure wave correlates with the amount of NO produced.

Some embodiments of electrical NO generators include a pressure sensor in fluid communication with the plasma chamber. This sensor is typically utilized to quantify reactant gas pressure for dose control, account for effects of altitude on NO generation and can also be used to capture the acoustic pressure wave of electrical discharges. The amplitude of the pressure wave and/or the area under the pressure pulse can be utilized to estimate the power of the discharge and resulting NO produced. The actual pressure change is also a function of reactant gas water content, plasma chamber pressure, reactant gas composition (i.e., types and quantities of gas) and other parameters. In some embodiments, these parameters are measured and utilized to compensate the pressure data for their effects.

As a result, electrical discharges can be detected using a pressure sensor (e.g., microphone, diaphragm sensor, etc.). The magnitude of the pressure wave correlates with the amount of NO produced.

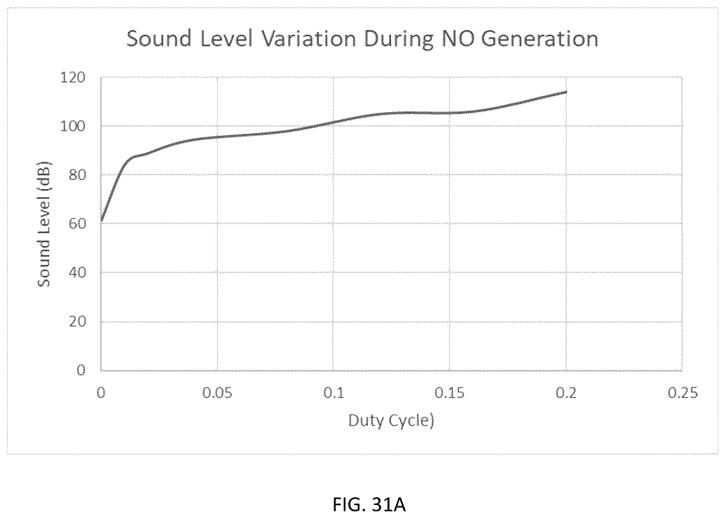

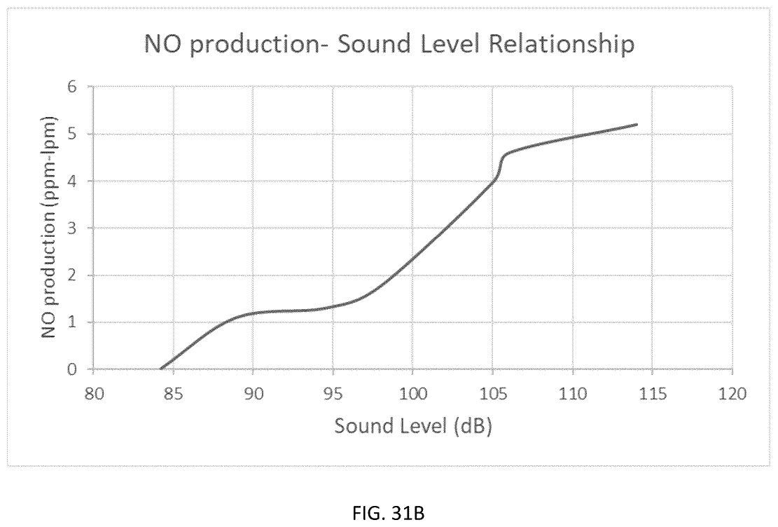

FIG. 31A depicts an exemplary graph showing exemplary experimental data from an electric NO generator. A microphone was used to quantify the sound level in decibels as the device was swept through a range of NO production levels (i.e. plasma duty cycles). In some embodiments, the presence/absence of sound emanating from the plasma chamber is utilized by the controller to determine whether or not the plasma is functioning. In some embodiments, the relationship between sound level and NO production is utilized by the controller to quantify the amount of NO production based on the magnitude of sound, as measured by a sound sensor (e.g., microphone).

FIG. 31B depicts an exemplary graph showing exemplary experimental data that demonstrate the relationship between acoustic noise and NO production. Higher levels of NO production generate more acoustic noise because electrical discharges are more frequent and/or more energetic. In one embodiment, the controller of a NO generation device monitors the sound level at a location that is in fluid communication with the plasma chamber. In some embodiments, the microphone is separated from the plasma chamber by a diaphragm to protect the sensing elements from corrosive NO and NO₂ gas (not shown). The controller enters the sound level measurement into an equation or a look-up table that yields the NO production level. In some embodiments, the NO generation system controller uses the sound level measurements as feedback to the NO generation process. If sound levels (or calculated NO production based on sound levels) are too high, the NO controller decreases plasma activity (e.g. frequency, duration, energy) to shift production toward the target value. The same but opposite approach is utilized when sound levels indicate that NO production is too low.

### Electrode Wear

High voltage electrodes tend to wear over time due to the high temperature of the plasma. The high temperature can vaporize electrode materials causing them to condense into solid particles downstream as the gas cools. These particles, albeit made from dense metals, are extremely light and can travel great distances within a gas stream. Unmitigated, electrode particles can collect on surfaces and affect the performance of system components (e.g., sensors, valves, pumps) as well as present a risk to a patient.

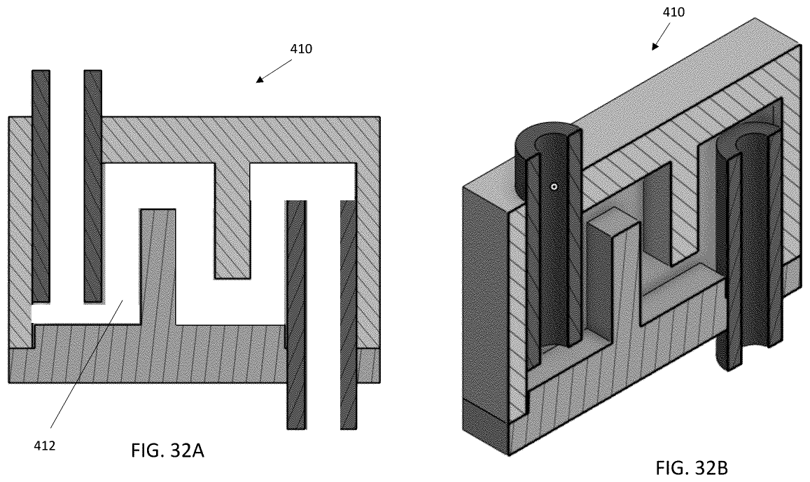

FIGS. 32A and 32B depict an exemplary embodiment of a particle trap 410 used to collect electrode particles within an electric NO generation system. The particle trap comprises a tortuous path 412 with tight turns. The heavy particles within the gas stream are unable to navigate the tight turns and collect in the corners of the particle trap. In some embodiments, the particle trap is lined with a sticky material (e.g., adhesive) or a viscous material (e.g., glycerin) to adhere to the particles and prevent further particle migration as the system is subjected to shock and vibration.

In some embodiments, the product gas flow path is designed to have a point of flow stagnation. The stagnation point is associated with flow velocities that are at or near zero. In some embodiments, the flow velocity reverses. The stagnation and/or reversal of product gas flow causes heavy electrode particles in the gas stream to fall out of the gas stream and deposit in the pathway. In some embodiments, flow is induced to spin in a cyclonic manner, promoting deposition of heavier gas/particulate species in an outer region while presenting a flow stream cleared of heavier species in the relatively stagnated center of the cyclonic activity.

### Electrode Design

The design of a high voltage electrode system for NO generation requires attention to many details. In some embodiments, high voltage leads from the transformer to the electrodes are spaced away from other conductors and grounded surfaces to minimize capacitance between electrical conductors. Minimizing capacitance between high voltage conductors and other conductive elements decreases losses that can occur in the high voltage power which can result in loss of power at the plasma and possibly prevent electrical breakdown from occurring between the electrodes.

Capacitance between the electrodes can also present a sink for electrical power. In some embodiments, the geometry of the electrode is minimized to minimize capacitance between the electrodes.

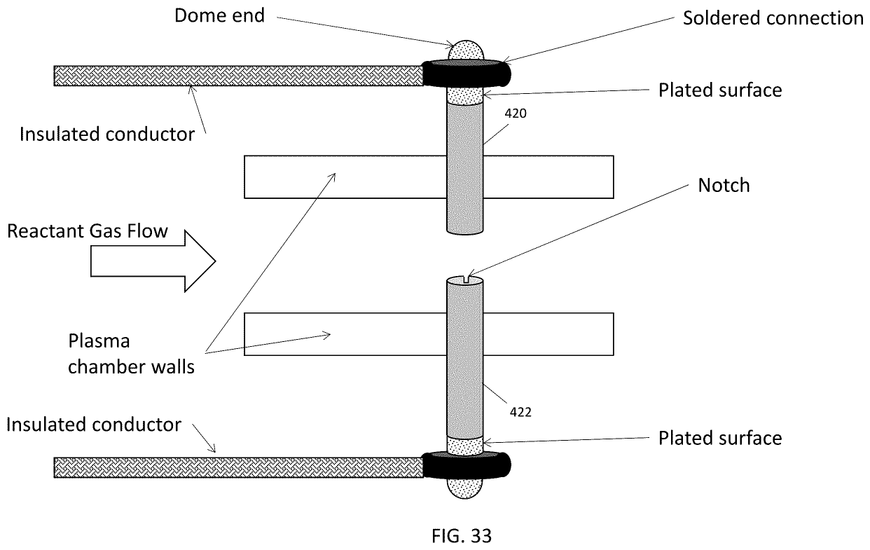

In some embodiments, a notch is added to the surface of an electrode to provide a sharp edge with high electric field to promote electrical breakdown. Contrastingly, rounded surfaces are desirable in all parts of the high voltage circuit except where breakdown is desired. FIG. 33 depicts an exemplary embodiment of a plasma chamber with an opposed electrode design. Each electrode 420, 422 has one or more notches on the surface facing the plasma chamber to facilitate electrical breakdown. The electrodes are fabricated from a high melting temperature, electrically conductive material (e.g. noble metals, titanium, iridium, stainless steel, platinum, hafnium, tungsten). Materials that serve well as electrodes are often difficult to electrically connect to. In some embodiments, the electrode material is plated with another material that can be soldered to (e.g. copper, gold, silver). In some embodiments, a material that is easily soldered to (e.g. copper) is crimped to the outer surface of the electrode to facilitate connection with the high voltage wires.

In some embodiments, the outer ends of the electrodes are radiused or domed to decrease electrical field. The electrical conductor is connected to the electrode by exposing a length of the conductor roughly equal to the circumference of the electrode. The wire is wrapped around the electrode and then soldered in place. The solder is formed with smooth, radiused edges to minimize electrical field concentrations.

In some embodiments, the plasma chamber, itself, is formed with an electrically insulative material (e.g., polymers like PEEK, or ceramics like alumina or Macor). In some embodiments, the surface of the plasma chamber is finished with a glaze, glyptal or other type of sealer to smooth the surface and reduce the potential for surface charge.

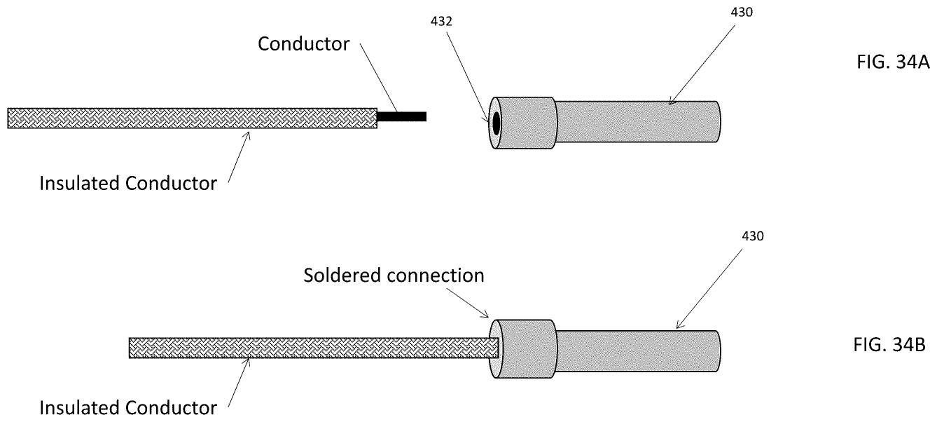

FIGS. 34A and 34B depict an exemplary embodiment of a rod-shaped electrode 430 with a solder cup 432 formed in the end. In some embodiments, the solder cup is plated with an electrically conductive material (e.g. copper, gold, silver) prior to soldering (not shown). FIG. 34A depicts the high voltage wire and electrode prior to assembly. FIG. 34B shows the high voltage wire soldered to the electrode. This design allows the wire to exit parallel to the electrode which decreases capacitance between the two wires.

When high voltage electrodes are oriented in an axial, opposed orientation, plasma emanates from the sharpest features, where the electric field is highest. For example, if the electrodes are shaped like cylinders, the circular edge at the end of the electrode is where arcing and erosion occurs. Over time, cylindrical electrodes approach a hemispherical shape at the arcing end. The rounded features of the electrode produce lower electrical field and require higher excitation voltage to breakdown gases within the gap. This effect can lead to decreased NO production when not compensated for by the device controller (e.g. with higher voltage).

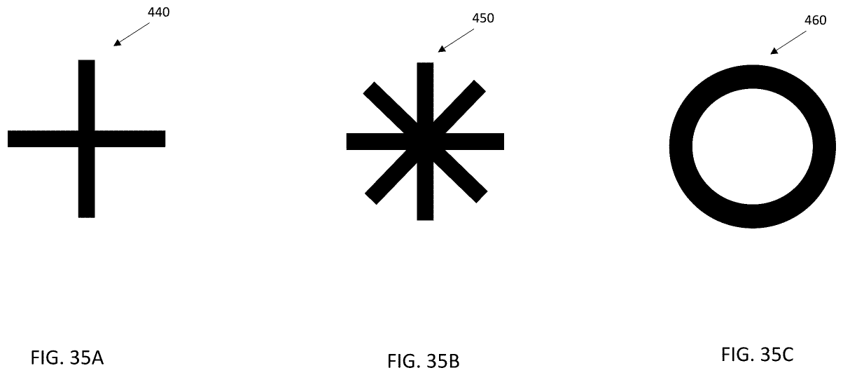

FIG. 35A depicts the cross section of an exemplary embodiment of an electrode 440 that is cross-shaped. The cross shape provides more sharp edges than a simple cylinder, increasing electrical field strength and lowering breakdown voltage. As the electrode wears, the sharp edges continue to provide high field strength and low breakdown voltage. The benefits of an electrode with a cross-shaped electrode include more consistent performance over time. FIG. 35B depicts a cross-section of an exemplary embodiment of an electrode 450 shaped like an asterisk that provides sharp features as it wears. FIG. 35C depicts a cross section of an exemplary embodiment of an electrode 460 shaped like a tube that provides sharp features as it wears.

### Electrode Wear

Electrode wear is a common issue with hot plasma systems that rely on arcing between electrodes to produce the heat to disassociate nitrogen and oxygen molecules. Systems that rely on the edge of an electrode to enhance the electric field and electrical breakdown of the reactant gas are subject to drifts in NO production over time as sharp edges wear.

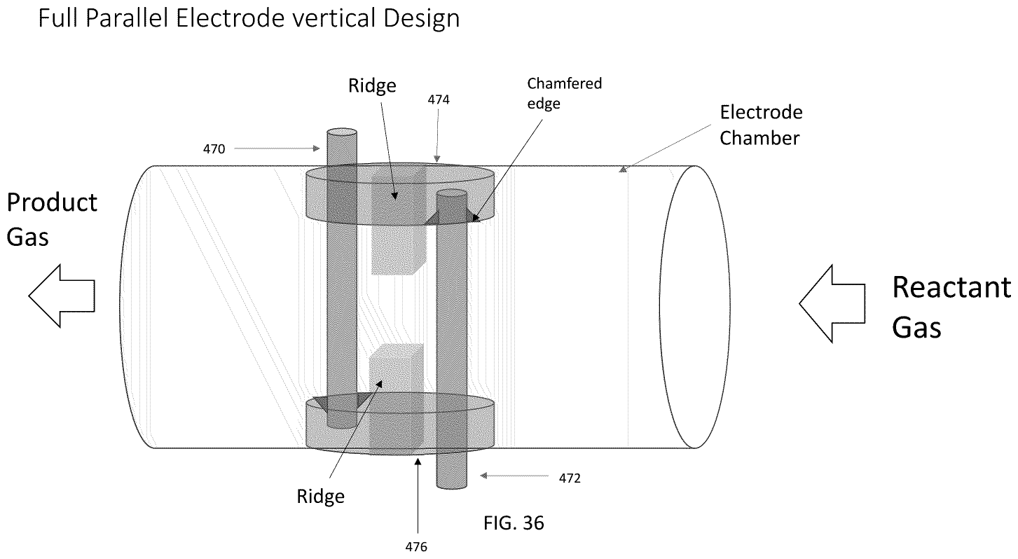

FIG. 36 depicts an exemplary embodiment of an electrode design that eliminates sharp edges, referred to as the “full parallel design.” The design consists of two, essentially parallel electrodes 470, 472 located within a reactant gas flow. In some embodiments, the plane defined by the parallel electrodes is orthogonal with the reactant gas flow. In some embodiments, the plane defined by the parallel electrodes is parallel to the reactant gas flow. In some embodiments, this relationship is oblique.

The ends of each electrode can be embedded into the wall of the chamber to bury the sharp ends of each electrode. The inner wall of the chamber is chamfered or otherwise shaped to prevent high electrical fields forming where the electrode penetrates the surface, thereby minimizing or preventing the triple-junction effect. One or more ridges or valleys between the electrode insertion points provide sufficient creepage distance to prevent electrical discharges along the wall of the chamber.

In some embodiment, the chamber is constructed from a tube-like structure with holes along the sides. The electrodes are inserted into inserts 474, 476 constructed from non-electrically conductive material (e.g., ceramic, glass, polymer). The inserts are installed into the chamber to hold the electrodes in the desired orientation with respect to the reactant gas flow. In some embodiments, the ridges and valleys are a feature of the insert. In other embodiments, the ridges and valleys are feature of the insert.

Arcing occurs between the electrodes, across the gap in the region between the ridges. The electrical field between the electrodes is essentially uniform within the gap region. Hence, breakdown can occur anywhere within the region defined by the electrodes and ridges. This approach can improve electrode longevity by distributing electrode wear over a larger surface area. Furthermore, electrode wear is slow as compared to electrode designs with sharp surfaces, thereby slowing NO production drift over the service life of the electrodes.

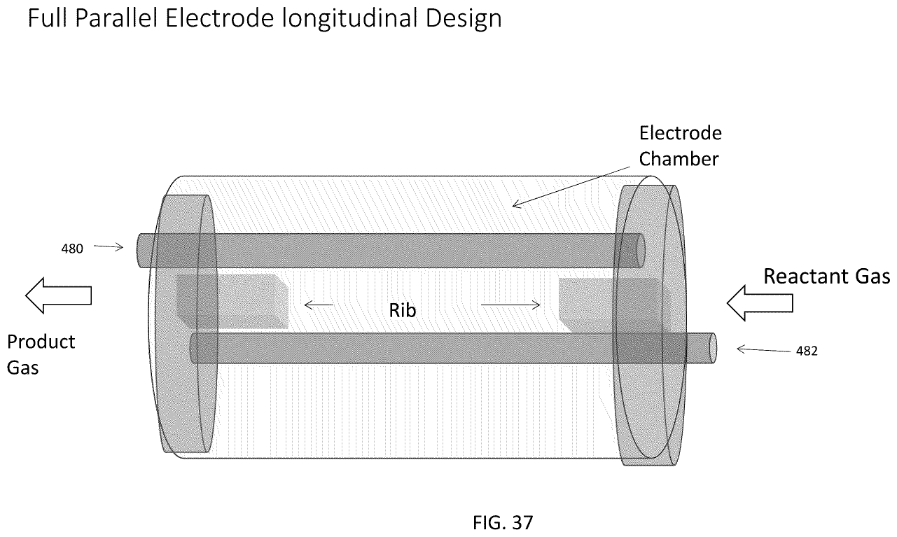

FIG. 37 depicts an embodiment of a full parallel design in which the electrodes 480, 482 are oriented parallel to the direction of reactant gas flow.

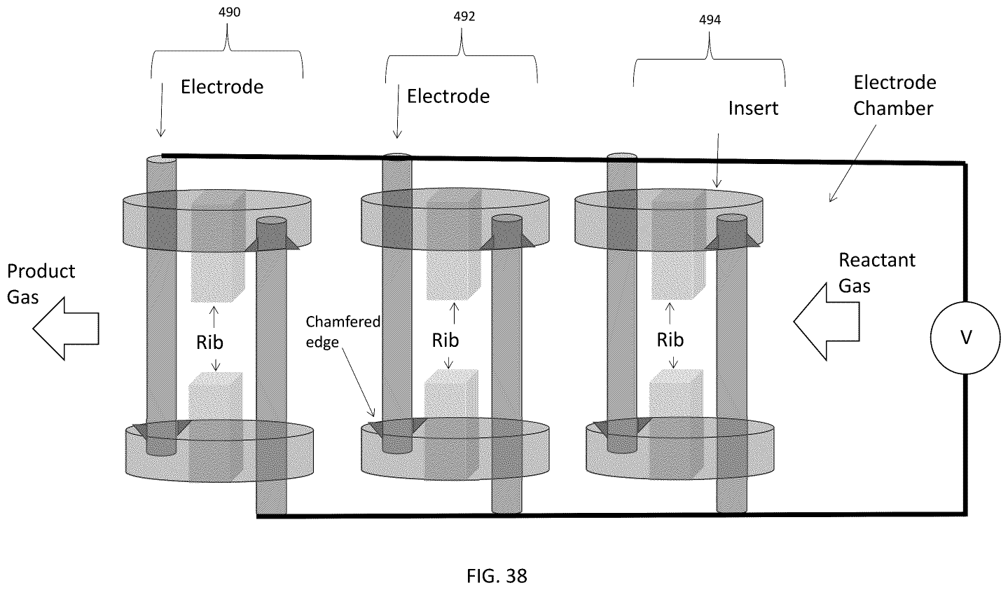

FIG. 38 depicts an embodiment that includes an array of electrode pairs. In some embodiments (shown), the electrode pairs 490, 492, 494 are all energized in unison by a high voltage supply. Once breakdown occurs between one pair, the voltage decreases rapidly, preventing breakdown at another electrode pair. In some embodiments (not shown), the high voltage to each pair of electrodes is switched by the NO device.

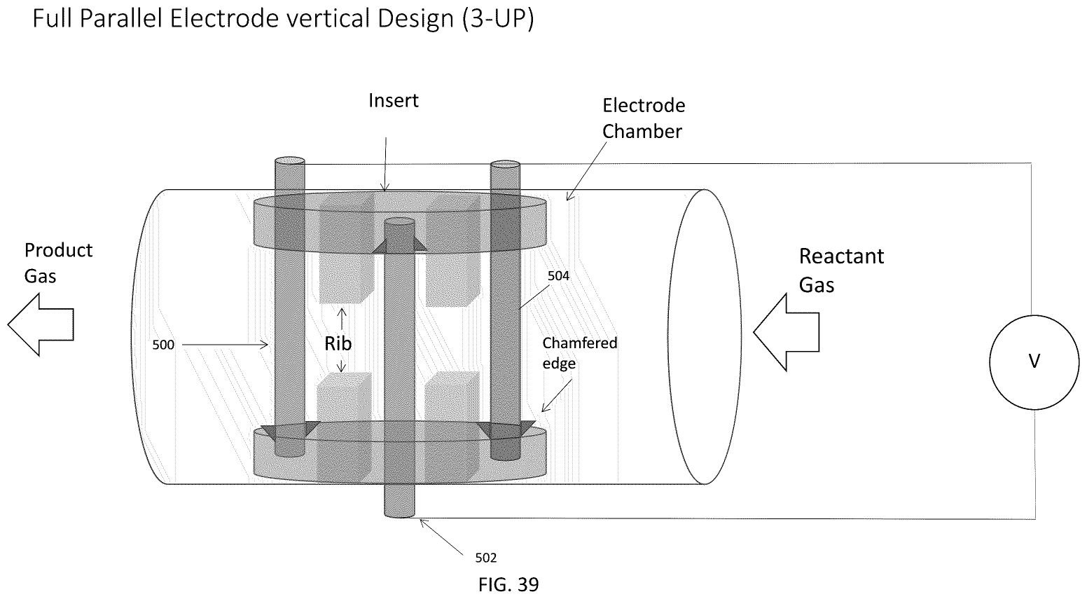

FIG. 39 depicts an embodiment of a full parallel design with three electrodes 500, 502, 504. This design can achieve twice the surface area for arcing for only a 50% increase in electrodes. The increased surface area for arcing increasing the overall longevity of the assembly.

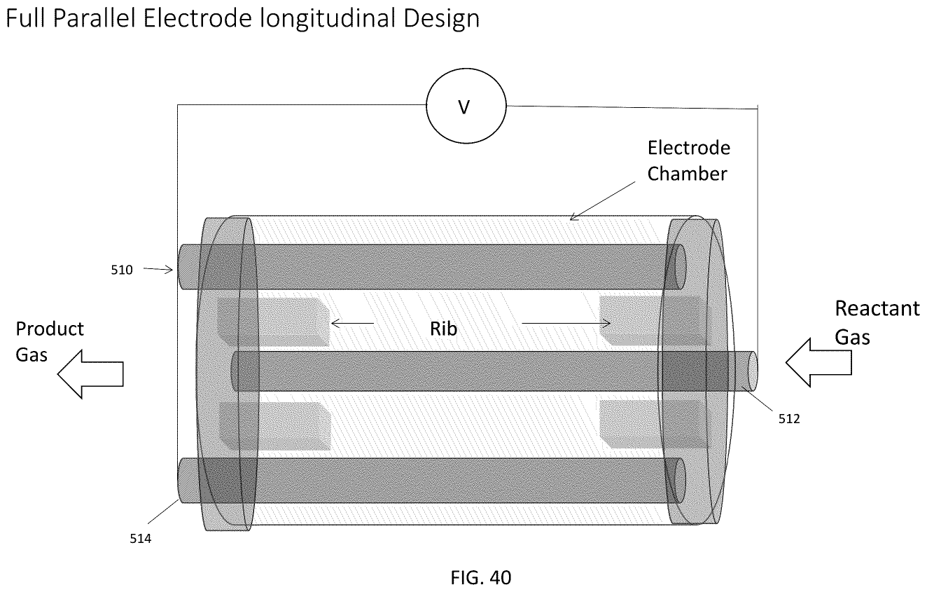

FIG. 40 depicts an embodiment of a full parallel design with three electrodes 510, 512, 514 oriented along the direction of reactant gas flow.

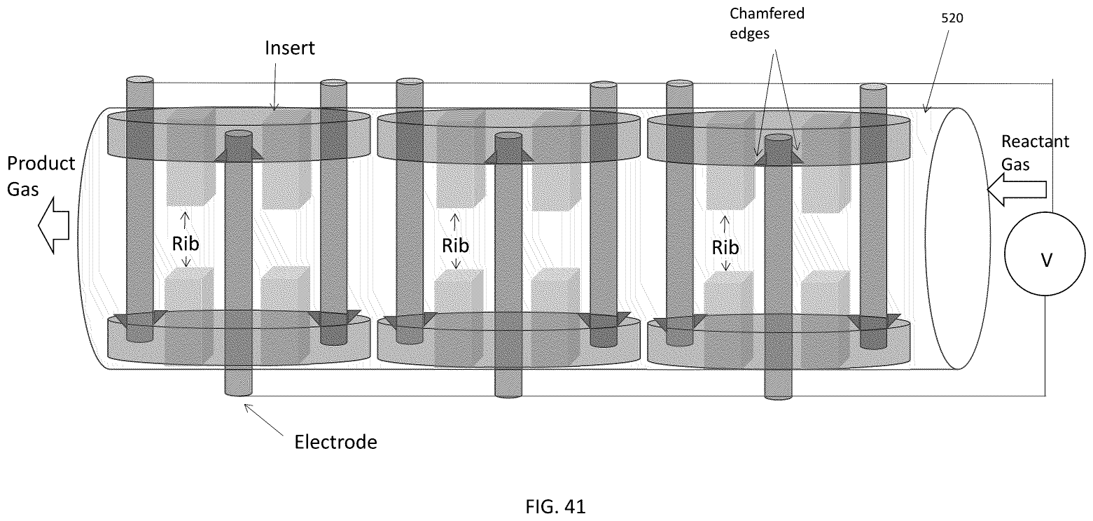

FIG. 41 depicts an embodiment of an electrode chamber 520 having a 3-electrode full parallel design in an array configuration. This design provides six electrode gaps from 9 electrodes for increased service life.

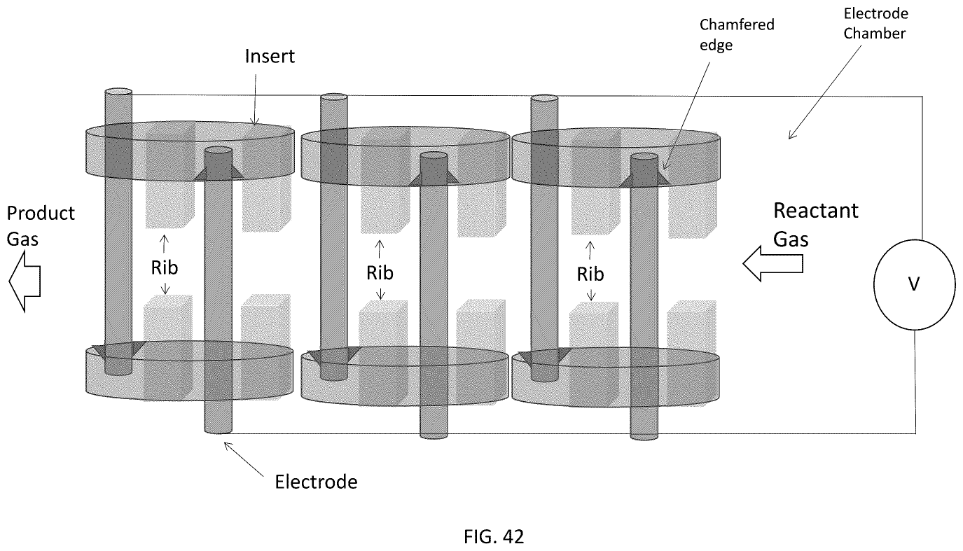

FIG. 42 depicts an embodiment of full parallel electrodes with six electrodes providing 5 electrode gaps.

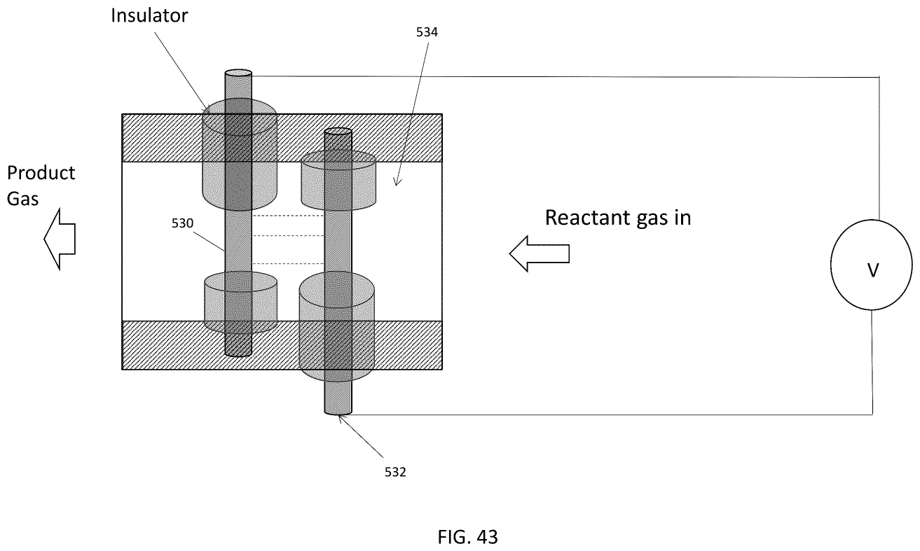

FIG. 43 depicts an embodiment of full parallel electrodes. Electrodes 530, 532 are in inserted from adjacent (not shown) or opposite (shown) faces of the plasma chamber 534. The inserted end of the electrode mates with the opposite inner wall of the chamber. An insulator on the inserted end of the electrode prevents triple junction effect on the electrical field to mitigate against arcs emanating from the insertion point. The other end of the electrodes has an insulator on the outer diameter to prevent arcing. In some embodiments, the insulator(s) are pressed into the walls of the plasma chamber. This electrode design controls where arcing occurs by only exposing electrode surfaces where arcing is desired. By controlling arc location and length, NO production becomes more stable and predictable. Dashed lines depict exemplary electrical arcs between the electrodes.

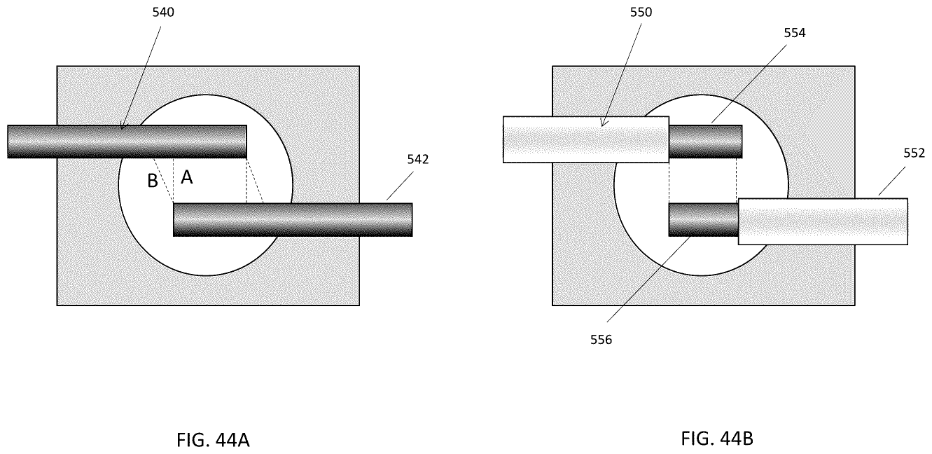

Overlapping parallel electrodes break down from the end of one electrode to the side of another electrode. The electrical discharge that occurs can occur at various angles between the electrodes. FIG. 44A depicts an embodiment of a pair of overlapping parallel electrodes 540, 542 in a plasma chamber. Gas flow through the plasma chamber is orthogonal to the figure. Arcing paths between a pair of parallel electrodes are depicted as dashed lines. When arcing is orthogonal to the long axis of the electrodes (marked with letter A), the arc length is shortest resulting a first amount of NO production. Arcing can also occur between the end of an electrode and another location on the side of the electrode resulting in a longer arc length (marked with letter B). Experimentally, differences in arc length can result up to 12% variation in NO production.

FIG. 44B depicts an embodiment of a parallel electrode design that includes insulators 550, 552 around the electrodes 554, 556. The insulators prevent arcing between the electrodes except in the orthogonal direction. This approach can improve the consistency of NO production with a parallel electrode design.

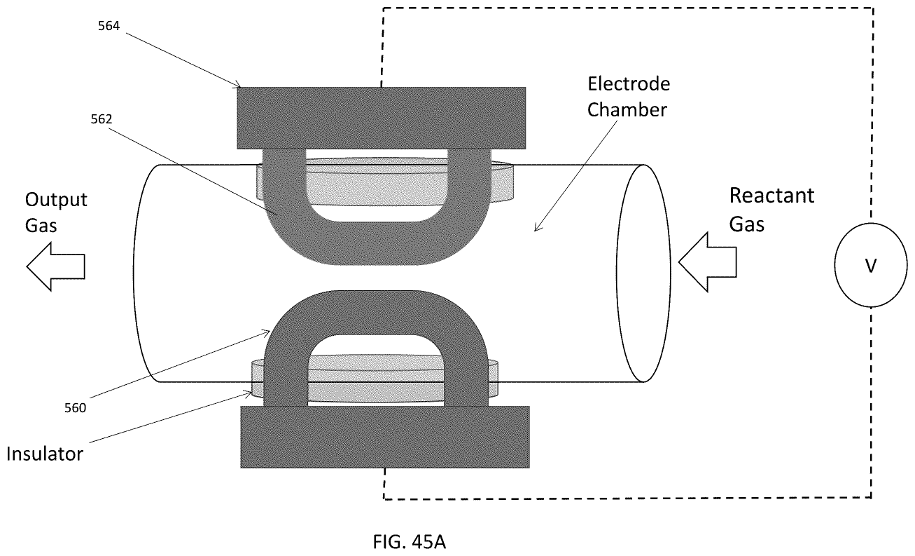

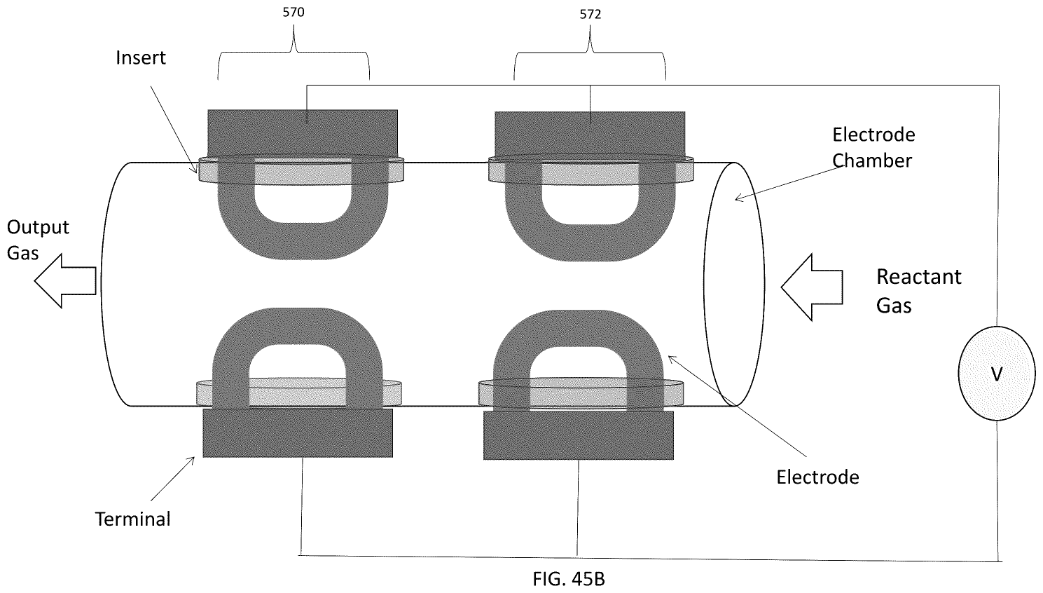

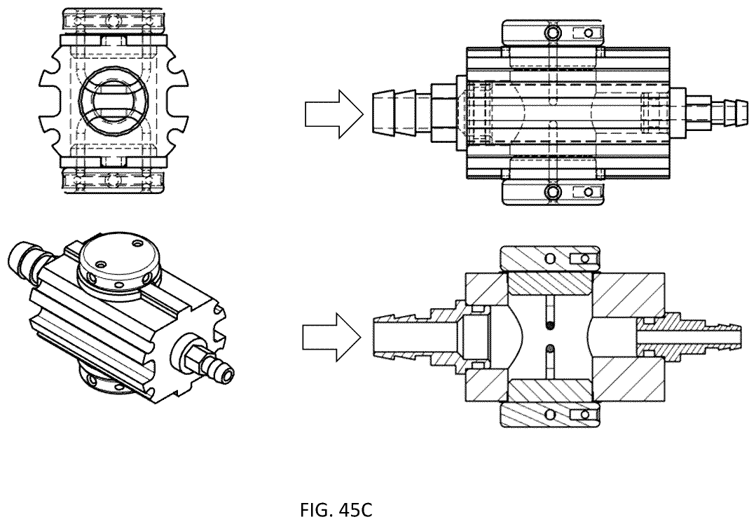

FIG. 45A depicts an embodiment of an electrode configuration referred to as “bent electrodes.” The electrodes 560, 562 consist of a cylindrical shape (e.g. a rod) that enters the plasma chamber, bends 90 degrees, travels along the plasma chamber, then bends another 90 degrees to exit the plasma chamber. In some embodiments (not shown), the electrode travels orthogonal to the direction of gas flow. Tent electrode design creates a high electrical field between the opposed electrodes to reduce the voltage level required for electrical breakdown of the reactant gas. In addition, the bent electrodes have a large electrode surface area facing the gap to prolong electrode longevity. Additionally, the radius of the 90-degree electrode bends are gentle to prevent arcing in that region. The bent electrodes are installed into insulating plugs that interface with the plasma chamber walls. On the outside of the chamber, the electrodes are optionally connected to a terminal 564 and are electrically connected to a high voltage supply.

FIG. 45B depicts an embodiment of a plasma chamber housing two pairs of bent electrodes 570, 572. The electrodes are electrically connected to a common high voltage source. When breakdown occurs between two electrodes, the voltage drops, preventing additional plasma discharges from occurring. In some embodiments, the plasma chamber includes one or more nozzles, diffusers (e.g. mesh) or flow straighteners (not shown) to ensure consistent gas velocity at each electrode pair to ensure consistent NO production independent of breakdown location. In some embodiments, the plane of the U-shaped electrode is orthogonal with the direction of gas flow. In some embodiments, the cross-section of the plasma chamber is sufficiently large to result in uniform or nearly uniform gas velocity across the width of the plasma chamber. This improves repeatability of NO production between the multiple electrode pairs within the chamber.

FIG. 45C depicts an exemplary design of a plasma chamber housing bent electrodes. The plasma chamber is constructed from a non-electrically conductive material (e.g. ceramic, polymer, glass). The outer surface of the plasma chamber features grooves that increase the surface distance between to electrical terminals to minimize the potential for electrical creepage. The terminals are made from an electrically conducive material and serve as mounts for the bent electrodes as well as seals for the plasma chamber. The ends of the electrode rods are buried within the terminals, thereby preventing arcing from potentially sharp edges. In the depicted embodiment, the plasma chamber exit is in line with the plasma chamber inlet to increase the potential for smooth, non-turbulent flow.

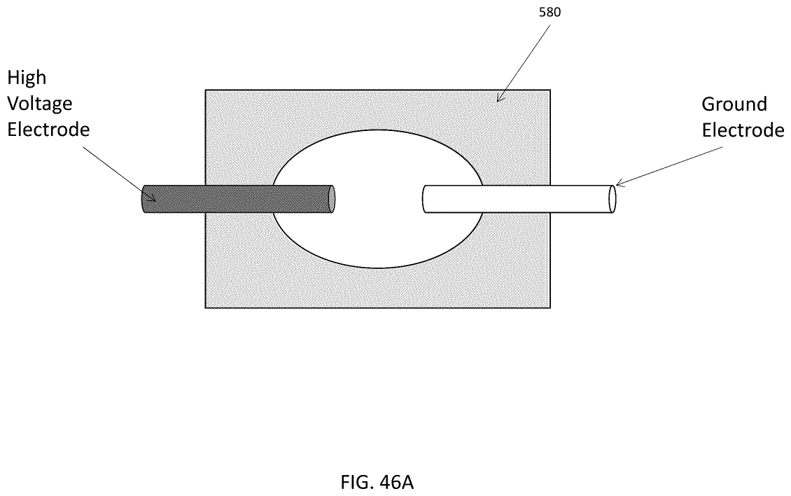

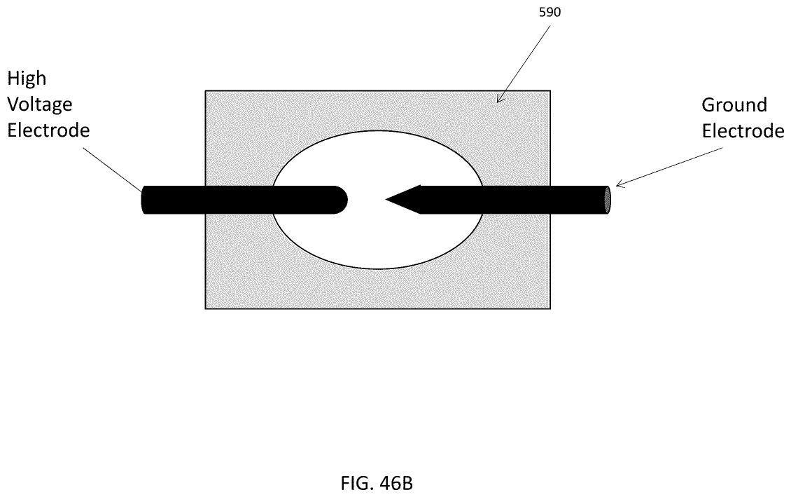

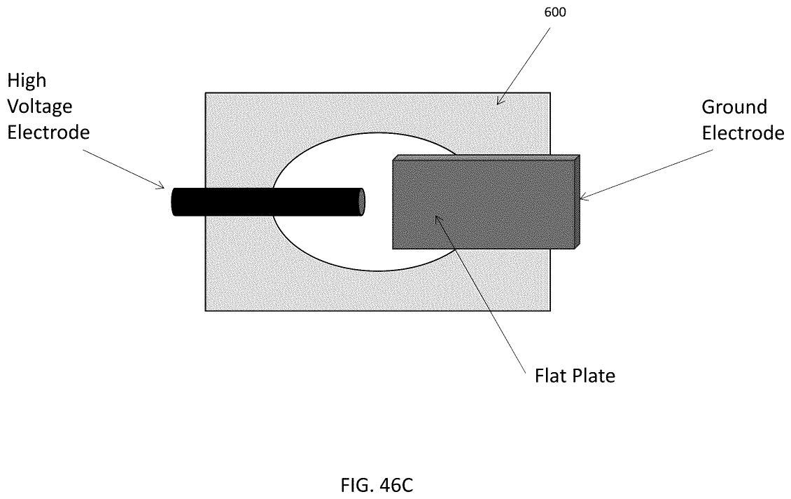

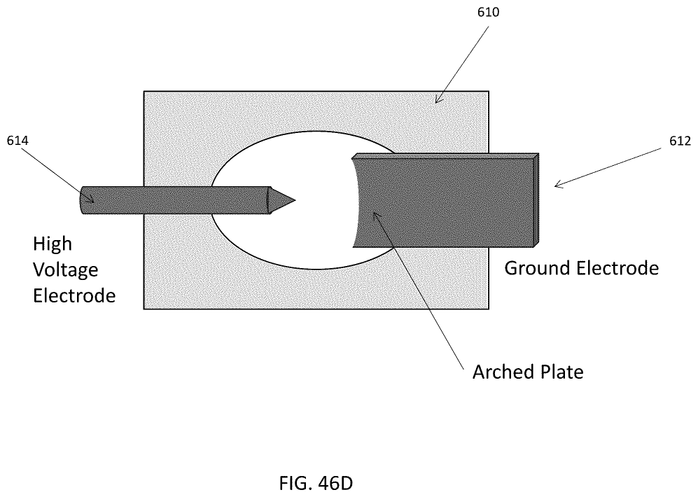

FIG. 46A depicts an exemplary plasma chamber 580 with electrodes made from different materials. In some embodiments, one electrode erodes faster than the other electrode(s) due to the polarity of the voltage applied. In some embodiments, only the faster-wearing electrode is replaceable. In some embodiments, the faster-eroding electrode is the ground electrode. In some embodiments, the faster-wearing, high voltage electrode is made from a more robust material than the ground electrode to make wear of both electrodes more even. This approach can reduce the cost of an electrode assembly without compromising the life of the electrode pair.

FIG. 46B depicts an embodiment of a plasma chamber 590 with two electrodes of different geometry. In some embodiments, one electrode erodes faster than the other electrode due to electrode geometry. For example, one electrode can have a domed end and is expected to wear slowly. The other electrode is pointed, which will increase electric field strength at the tip to facilitate electrical breakdown between the electrodes but will also wear more quickly. In some embodiments, the electrode that wears more rapidly can be individually replaced. It should be understood that this same concept can be applied to plasma chambers that incorporate more than two electrodes whereby some electrodes are expected to wear more quickly than other electrodes.

FIG. 46C depicts an embodiment of a plasma chamber 600 that increases electrical field strength between the electrodes by geometry. It is known that point to line or point to plane geometries require less voltage to breakdown than point to point geometries. In the depicted embodiment, one electrode is larger than the other, resembling a point to plane/line geometry. In some embodiments, the larger electrode is made from an inexpensive material (e.g. stainless steel) and the smaller electrode is made from a more expensive material (e.g. iridium). Less expensive materials typically have lower melting points are prone to wear faster. The larger surface area of the electrode on the right provides additional surface area to wear as well as improved thermal conduction of heat out of the electrode. In some embodiments, a single electrode may be replaceable. In the depicted embodiment, the surface of the right electrode as it faces the gap is a straight line/flat surface. In some embodiments, the gap between the high voltage electrode and the ground electrode is less than the width of the ground electrode to ensure that arcing does not contact the corners of the ground electrode.

FIG. 46D depicts an embodiment of a plasma chamber 610 with dissimilar electrodes. In this embodiment, a first electrode 612 has a concave surface facing the electrode gap. A second electrode 614 is made has a conical shape pointing towards the gap. In some embodiments, the tip of the pointed electrode is located at the center of curvature for the arc-shaped electrode, providing a consistent gap between the left electrode and right electrode. As the arc-shaped electrode erodes, the gap remains consistent owing to the larger surface area, thereby improving the consistency of arc lengths and related NO production. In other embodiments (not depicted), the surface is arched in a convex orientation which increases electrical field strength over flat and concave designs while retaining the improved longevity and reduced cost of having a large electrode made from an inexpensive material.

### Plasma Vortex

A plasma vortex electrode assembly, or plasma vortex, operates by applying a magnetic field to an annular electrode gap. As electrons travel across the gap during an electrical discharge, they experience a lateral force imparted by the magnetic field in accordance with Fleming's left hand rule. The force is proportional to ((vector)I×(vector)B), using the left-hand rule. Since the current vector points from the center outward to the outer electrode, for the force to rotate the plasma about the pin electrode, (vector)B must be parallel to the direction of gas flow. In some embodiments, a plasma vortex is operated with a DC voltage applied to the electrodes (central rod and outer ring) and a permanent magnetic field (e.g. with a permanent magnet or electromagnet). In some embodiments, an AC current is applied to a plasma vortex electrode with a permanent magnetic field. If the magnetic field is sufficiently powerful, the force on the plasma is strong enough to induce one or more rotations around the central electrode before the plasma pulse terminates. The outer ring may be constructed from a solid or a plated material. In some embodiments, the electrode consists of a iridium-plated substrate. In some embodiments, the outer electrode is magnetic (this could eliminate the need for the outer magnets). The outer electrode wears slowly, owing to the large surface area and constantly moving arc which distributes the heat. In some embodiments, a plasma vortex electrode design is modular such that the center electrode is replaceable. In some embodiments, the center electrode is withdrawn from a plasma chamber and replaced. Upon insertion, the center electrode is inserted until a positive stop is contacted to ensure electrode gap alignment. In some embodiments, a plasma calibration is performed after center electrode replacement.

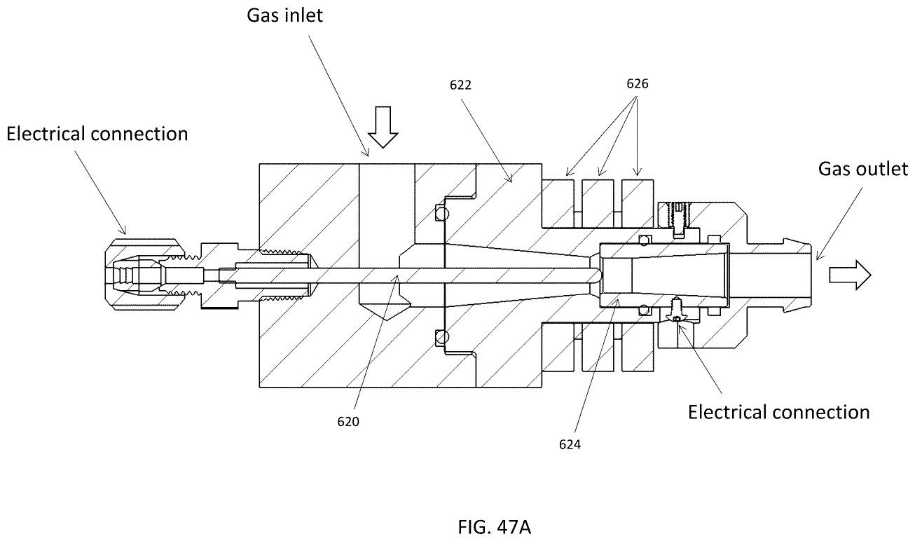

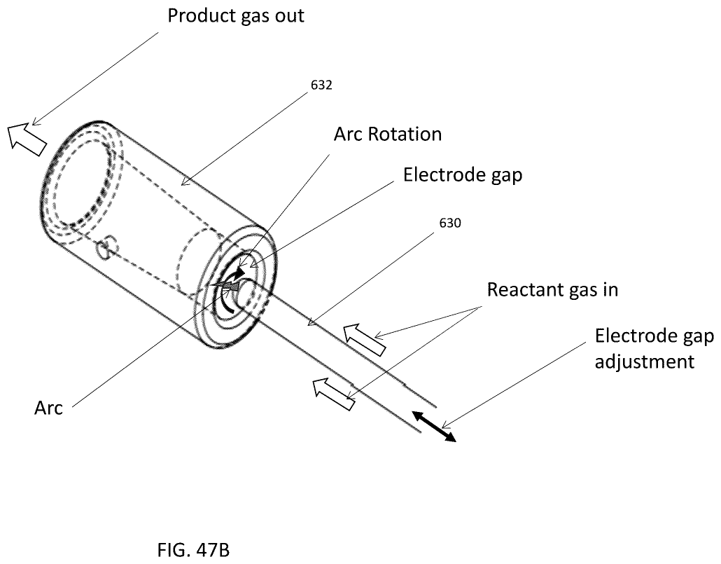

FIG. 47A depicts an exemplary embodiment of a plasma vortex electrode assembly. An electrical connection provides high voltage to a center electrode 620. Reactant gas enters the plasma chamber 622 and travels parallel to the center electrode. The center electrode terminates at or near the end of an annular electrode 624. One or more magnets 626 (3 are pictured) are located around the plasma chamber to create a magnetic field that is parallel with the direction of reactant gas flow. The annular electrode is connected to high voltage through an electrical connection as well. The electrode gap is typically between 0.5 mm and 10 mm. As depicted, there is a smooth transition in plasma chamber diameter to annular electrode diameter. The length of the chamber before the electrode gap is sufficient to establish laminar flow after the 90-degree bend. The direction of gas flow can be in either direction. In the direction depicted, there can be an advantage in maintaining arcing when reactant gas flows are high because the arc can touch down deeper into the annular electrode.

FIG. 47B depicts exemplary electrodes of a plasma vortex electrode assembly. The terminus of a center/pin electrode 630 is aligned with an end of an annular electrode 632 forming an electrode gap. Arcing within the electrode gap rotates within the annular gap about center electrode. Reactant gas flows from the center electrode to the annular electrode. The center electrode can be translated (e.g. by a screw thread) to adjust the electrode gap. In some embodiments, the center electrode is pulled away from the annular electrode to increase the gap. Moving the center electrode in this direction can be advantageous because arcing continues to occur from the edge of one electrode the edge of the other electrode, thereby creating consistent arc lengths for tight control of NO production.

In some embodiments, the translational position of the center electrode is adjusted to compensate for wear at the end of the center electrode. In some embodiments, the center electrode position relative to the annular electrode is adjusted as a degree of freedom for varying NO production (in addition to power, frequency, duty cycle, dithering, etc.). In some embodiments, the location of the center electrode with respect to the annular electrode is determined by the NO production level (e.g. 5000 ppm.lpm) required. In some embodiments, the center electrode location is automatically adjusted by a NO generation controller (e.g. by turning a motor that turns a screw). In other embodiments, the center electrode location is manually adjusted by a user.

In some embodiments, an AC current is applied to a plasma vortex electrode while an electromagnet is utilized to vary the magnetic B field in phase with the AC plasma current. This approach can achieve consistent lateral motion of the electrons, an electro-magnet is utilized to generate the magnetic field. The electromagnet is supplied with AC voltage that is synchronized with the AC electron current such that the magnetic field reverses when the electron direction of travel reverses, thereby maintaining a lateral force on the electrons in a consistent direction. This approach results in a plasma vortex arc rotating in a consistent direction despite being utilized with an AC current. Consistent motion of the plasma arc around the annular gap ensures even heating of electrode surfaces, even wear of electrode surfaces and more consistent creation of NO within the reactant gas flow. In some embodiments, the electromagnetic magnet is wired in series with the high voltage circuit so that the magnetic field reverses in unison with electron direction within the electrode gap as the AC voltage reverses. In another embodiment, the electromagnet is wired in parallel with the electrodes. Selection of how to wire the electromagnet is decided, at least in part, by choosing the option that poses the least impact to the secondary impedance as that would have an impact on resonant frequency. In another embodiment, the electromagnet is part of the primary high voltage circuit. In other embodiments, the electromagnet is driven of a dedicated secondary winding on the transformer high voltage transformer.

A plasma vortex electrode assembly design for NO generation allows for a large surface area for spreading out heat and a reduction in electrode wear. This can enable an NO generation system to operate at higher duty cycles without overheating electrodes. Furthermore, the sweeping motion of the electric arc improves the gas/plasma interaction in a reactant gas flow when compared to static arcs.

Arcing within a plasma vortex electrode assembly occurs from the edge of the center, pin electrode to the edge of the outer, annular electrode. When the center pin electrode edge is concentric with the annular electrode edge, the electrode gap is at its minimum, which can facilitate initiating electrical discharge. In one embodiment, the pin electrode can be translated along its axis to increase the electrode gap and thereby increase NO production. In some embodiments, arcing within a plasma vortex electrode assembly is pulsed (e.g. with a duty cycle). In other embodiments, the plasma vortex is energized continuously.

### Scrubbing Technology

It should be understood that there are many materials that can be used to remove NO₂ from a gas stream, including but not limited to soda lime, ascorbic acid, metal-organic frameworks (MOF), and TEMPO. Reference to a “scrubber” in this document is inclusive of all of these types of chemistries.

### Reducing Agent Scrubbers

Reducing agents have the ability to strip an oxygen atom from NO₂ to convert it into NO. Examples of reducing agents include ascorbic acid (vitamin C), Vitamin E, potassium iodide, and ferulic acid. In some embodiments, a scrubber includes an additional acid to reduce pH to a level that the reducing agent can operate (e.g. ascorbic acid requires a pH<4). Examples of the added acid include hydrochloric acid and sulfuric acid in appropriate amounts to achieve a particular range of pH. In some embodiments of a NO generation system (e.g. electric, NO-donor molecule, etc.), product gas containing NO and NO₂ is passed through a reducing scrubber that converts NO₂ to NO. When utilized in an electric-NO generation system, a reducing scrubber provides many benefits. Typical electrical NO generators generate an amount of NO₂ along with NO. The portion of gas that is NO₂ can vary from 5 to 50%, for example, depending on electrode material, electrode condition, reactant gas mixture, reactant gas flow rate, applied voltage, frequency of discharges, electrode temperature, and other factors. When a reducing agent is utilized as a scrubber, it converts the NO₂ generated in the plasma chamber in NO. Converting the NO₂ to NO enables the system to operate at a lower energy level to maintain a particular loop concentration, thus reducing the amount of wear and power draw. In some embodiments, a plasma chamber is calibrated for NOx (i.e. NO+NO₂) production, instead of only NO production since a large portion (nearly all) of the NOx will be converted to NO in a reducing scrubber. In some embodiments, fully hydrated silica gel beads are added to the ascorbic acid in the scrubber. The beads act as a reservoir of moisture as the ascorbic acid powder dries out, increasing the potential service life of the scrubber. In another embodiment, silica gel beads, with dense mesh (i.e. small diameter, e.g. 0.2 to 2 mm bead diameter), are used to increase the effective surface are to come in contact with the product gas containing NO₂. The beads would initially be hydrated with highly saturated ascorbic acid solution, thus coating the surface inside the silica mesh with ascorbic acid. In some embodiments, the silica gel beads serve as a substrate for a coating of ascorbic acid. In other embodiments, the hydrated silica gel beads are interspersed in a matrix with granules containing ascorbic acid.

When a reducing scrubber is utilized in a system with a recirculation loop architecture, additional benefits can be realized. Product gas circulating within a recirculation loop ages and a portion of the product gas oxidizes into NO₂. When a reducing scrubber is utilized in a recirculation architecture, any NO₂ that is formed by oxidation within the recirculation loop is converted back to NO. This process greatly decreases the amount of NO lost within the system and can eliminate the need for one or more of a product gas NO sensor and a NO loss calculation in some embodiments.

### Sheet Scrubber

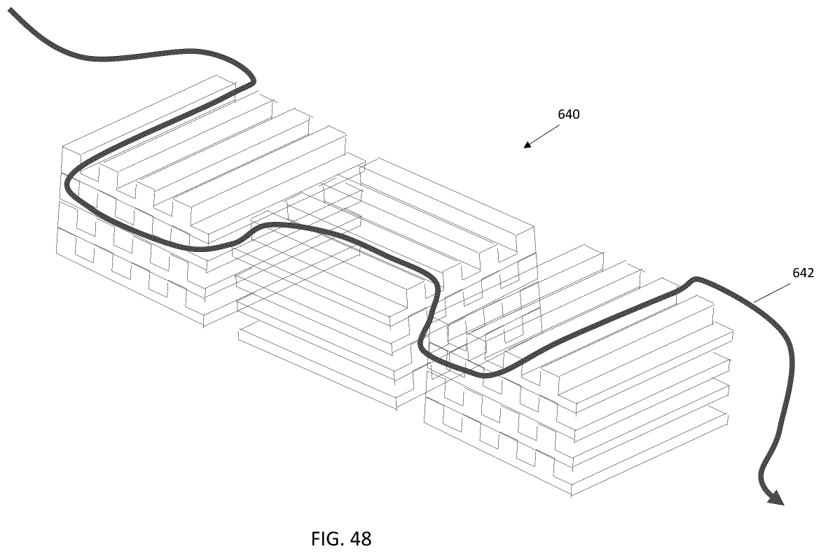

Efficient scrubbing of a gas stream requires an interaction between the gas and scrubbing material. Turbulent flow and tortuous flow paths improve the interaction between gas and scrubbing material. In applications where grooved sheets of scrubber material are utilized, a tortuous path is created by stacking the sheets into cubes and staging the cubes in series such that the gas channels do not align between sections. FIG. 48 depicts an exemplary embodiment of a scrubber 640 having 3 stacks of grooved scrubber sheet material 4 layers tall. The stacks are oriented so that the grooves are not parallel, thereby requiring gas flow to follow a tortuous path, as shown by a curved arrow 642. The tortuous path promotes gas mixing and scrubber/gas interaction. In some embodiments, the sections are rotated 90 degrees from each other.

### Metal Organic Framework (MOF)

In some embodiments, a metal organic framework (MOF) material, such as UiO-66, UiO-66-NH2, and/or MFM-520, is utilized as a NO₂ scrubber material. Metal organic frameworks (MOF) are specific class of material consisting of a metal ion within a cluster of organic molecules. The structure is repeated in a three-dimensional lattice to form a crystalline structure that can be used to attract and store gases. In some embodiments, a MOF is used as a NO₂ scrubbing material in an electric NO generator.

In some embodiments, the MOF material is made from fibers of MOF (e.g. a woven structure, or amorphous structure). In some embodiments, the MOF material is in the form of a membrane or sheet. Membranes can be rolled into spirals, twisted into a helix within a tube, or stacked. Some membrane embodiments are permeable to gas flow, enabling designs that direct gas flow through the membrane.

In some embodiments, the MOF material is in the form of granules (uniform or random size) that can be packaged in a canister through which product gas is flowed.

### Granular Scrubber Material Compaction

When a volume is filled with granular scrubber material, the material settles to the bottom with void space between the granules. The void space provides a pathway for gases to permeate the bed of granules and interact with the scrubber material. When granule packing is left to gravity, settling of granules can be uneven and low-resistance channels can exist which become the preferred pathway for gas flow. Channeling, as this phenomenon is referred to, results in reduced scrubber bed service life because scrubber material lining the channels is exhausted first. Channeling also results in reduced scrubber efficiency due to a decrease in gas/scrubber interaction.

One approach to reducing the potential for channeling and increasing gas/scrubber interaction is compaction of scrubber granules. In some embodiments, granules are added to the scrubber housing by weight. Then, the granules are compacted by a specific distance. Then, additional granules are added and compacted. Additional layers of compacted granules can be added, as needed. Compaction of a granule bed improves the consistency of scrubbing efficiency and service life across multiples of scrubbers. This approach can be utilized with soda lime, MOF, molecular sieve, activated carbon and other types of granules.

### Variable Scrubbing

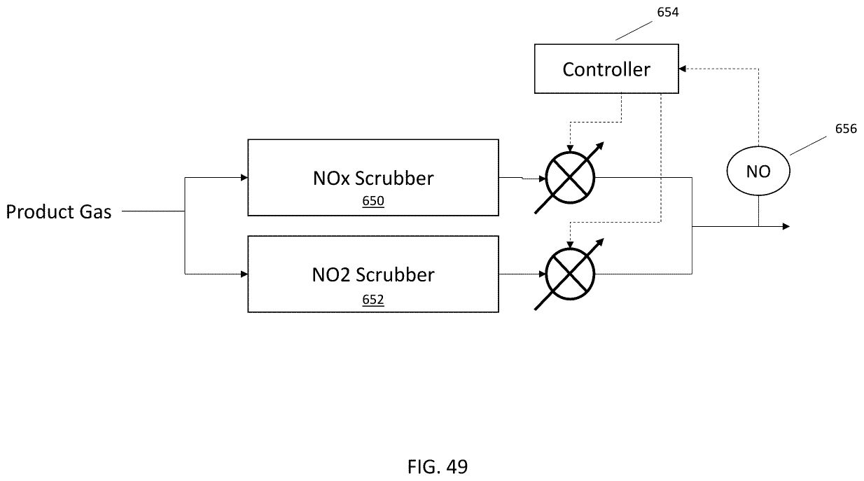

In some embodiments of a NO generation device, low levels of NO production can be difficult to achieve. One solution to this issue is to partially scrub the product gas to remove a known amount of NO. The amount of NO intentionally removed from the product gas can be modulated by the controller in multiple ways. In some embodiments, the ratio of NO-scrubbed vs. non-scrubbed product gas is modulated by varying the flow through two respective flow paths. In some embodiments, the efficiency of the NO scrubber is varied to modulate the amount of NO intentionally removed from product gas. In some embodiments, product gas is aged a varying amount of time in order to lose a variable amount of NO to oxidation and the resulting NO₂ is scrubbed from the product gas. FIG. 49 depicts an exemplary embodiment of a variable scrubbing system to partially and controllably scrub a product gas flow to achieve a target NO concentration. Product gas enters the system, and the flow bifurcates into a flow path that removes NO₂ using an NO₂ scrubber 650 and a flow path that removes both NO and NO₂ (i.e. NOx) using an NOx scrubber 652. The flow rate through each of the paths is controlled by one or more flow controllers at the entrance (not shown) or exit (shown) of the scrubber. The flow controllers are controlled by a process controller 654. An optional NO sensor 656 measures the concentration of NO in the partially scrubbed product gas to provide feedback to the process controller. In some embodiments, the NO₂ scrubber consists of one or more of MOF, TEMPO, and soda lime. In some embodiments, the NOx scrubber comprises one or more of MOF, TEMPO, soda lime, potassium permanganate, a reducing agent (e.g. ascorbic acid, vitamin E), and activated carbon.

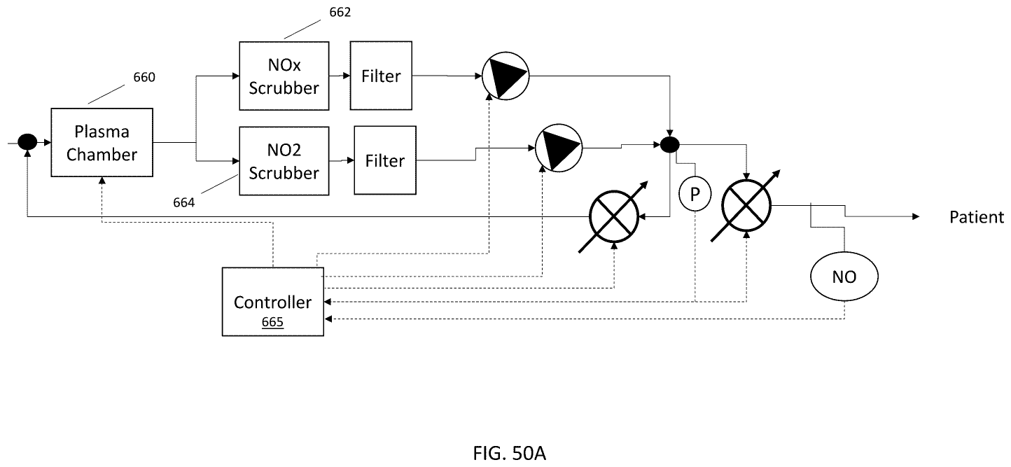

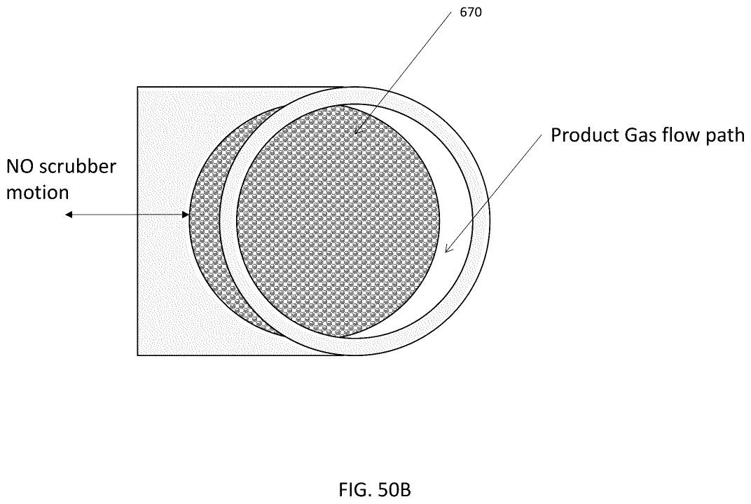

In some embodiments, variable scrubbing is utilized in a recirculation architecture, as depicted in FIG. 50A. The flow of gas exiting a plasma chamber 660 bifurcates. A portion of the product gas passes through a NOx scrubber 662 with the balance passing through a NO₂ scrubber 664. The distribution of flow through each pathway is controlled by a device controller 665 by varying the flow rate through each channel via pump effort. A person skilled in the art can envision other methods to control the flow, such as using a single pump and two flow controllers. The two gas flows join at or before the node of constant pressure. In some embodiments, the concentration of NO is measured at the node (not shown), or closer to the patient (shown). The NO concentration can be measured in the product gas or in diluted inspiratory gas. In some embodiments, when the NO concentration is measured in the inspiratory gas, the controller utilizes the dilution ratio to convert the measured NO concentration to the product gas concentration. In some embodiments, the controller simply controls the degree of product gas NO scrubbing based on the NO measurement at the patient. This approach can be particularly useful in neonate NO applications, when very low levels of NO are required.

FIG. 50B depicts an embodiment of a variable product gas NO scrubber 670. A NO scrubber is translated left and right to variably occlude a NO product gas flow path. With higher degrees of occlusion, higher levels of NO pass through the scrubber and are removed from the gas stream. The scrubber overlap with the flow path can be controlled by the device controller. In one embodiment, a motor (e.g., linear, rotary, etc.) is driven by the controller to achieve a target level of occlusion of the product gas pathway with a NO scrubber.

### Scrubber Moisture Management

Some NO₂ scrubber materials (e.g. soda lime) have water content that is essential to scrubber performance. As product gas passes through the scrubber, scrubber hydration can decrease. In many cases, scrubber hydration limits scrubber service life more than the quantity of any other material. Hence, various approaches to maintaining scrubber moisture have been contemplated to prolong the service life of a scrubber.

In some embodiments, water is wicked from a reservoir into the soda lime to keep it moist. When the reservoir is empty, it can either be refilled by a User or replaced. In some embodiments, the water reservoir is part of a disposable gas conditioning cartridge. In some embodiments, water is pulled into the scrubber via capillary action. In some embodiments, water from a gas sample line water trap is wicked into a scrubber. The wicking material may be made from a multitude of materials, including but not limited to cotton, polyurethane, and EPTFE fibers.

In some embodiments, water condensed during the drying process for incoming reactant gas is utilized to wet the soda lime scrubber. In some embodiments, water is transferred from the drying stage to the scrubber via a wick. In some embodiments, a web of wicking fibers is placed over soda lime granules to distribute water over a volume of soda lime.

Soda lime can be used as a desiccant in a gas stream. In some embodiments, a NO generation system includes two soda lime scrubbers. A first soda lime scrubber is used to remove humidity (i.e. water) from incoming air while the second soda lime scrubber is used to remove NO₂ from product gas. When the second soda lime scrubber shows decreased scrubbing efficiency, or after a set period of time, the role of the two scrubbers is swapped. Then, the dry scrubber is utilized to remove ambient humidity and in doing so gains water content. The hydrated scrubber is utilized to scrub NO₂. The system requires pneumatic plumbing to rout product gas and incoming air to the appropriate scrubber. In some embodiments, the scrubbers are manually interchanged by a user. In some embodiments, the scrubbers are in a common cartridge and the user changes the scrubber roles in the system by manipulating (rotating, sliding, inserting upside down, etc.) the scrubber cartridge. In some embodiments, the NO generation system automatically controls the role of each scrubber. In some embodiments, the role of scrubbers is changed based on a measurement from a humidity sensor. In some embodiments, the role of scrubbers is changed based on a time measurement.

### Foam Scrubber

Many scrubber materials are available in granular form. Granules require packing to prevent channeling as well as fillers or batting to prevent relative motion. Granules can erode, introducing particulate to a gas stream as well. Granules require manufacturing steps to measure and load them into a cartridge. In one embodiment, an open cell foam is generated with scrubber and/or desiccant material. The open cell foam permits gas flow through it and interaction between the gas stream and embedded material. The foam can be formed in a shape that drops into a cavity of a scrubber cartridge housing.

When a foam of soda lime is used, it can be inserted into a cavity within a housing in a dry state. Then, the housing is one or more of submerged in water or exposed to high humidity so that water enters the foam and the foam swells. As it swells, it increases in volume to eliminate gaps between the foam and housing that could create dead spaces where NO₂ forms.

In some embodiments, the density of an open cell foam varies along the length of a cavity within the scrubber housing. The density of foam is higher at the entry end of the cavity for increased NO₂ scrubber. Towards the exit end, the density is less (higher porosity) to facilitate rapid release of NO gas (e.g. pulsed delivery applications).

In some embodiments, a foam gas scrubber is constructed by mixing a molten polymer (e.g. polyethylene, polypropylene, etc.), scrubber particles, and a soluble material (e.g. table salt soluble in water). The mixture is cooled to enable the polymer to solidify. Then, the solid is placed in solvent (e.g. water) to dissolve the soluble material out of the solid, leaving voids for gas flow.

### TEMPO Coating

In some embodiments, a substrate is coated with a nitroxide radical containing compound (e.g. TEMPO) by dissolving the compound into a solution. In one embodiment, TEMPO is dissolved into ethanol, for example. The substrate structure (e.g. a tube, fiber, foam, housing) is then dipped into the solution. The solvent is then driven from the structure via heat and/or gas flow and/or time leaving a substrate coated with scrubber material.

### NO Loss Within a Scrubber

In some embodiments, a portion of NO within a product gas stream is lost as it passes through a NO₂ scrubber. In some instances, NO loss to a scrubber exceeds the amount that would be expected from oxidation alone. In some scrubber chemistries (e.g. soda lime), the amount of NO lost can be as much as one to two times the amount of NO₂ scrubbed, as measured in moles. The amount of NO lost to the scrubber can also vary over the service life of the scrubber.

In some embodiments, a NO generation system predicts the amount of NO lost to oxidation and to a scrubber in order to accurately predict the concentration of NO being delivered to a patient. NO loss prediction is particularly useful in systems that do not measure NO concentration downstream of the scrubber. In some embodiments, the NO device calculates the amount of NO loss as a function the chemistry of a soda lime scrubber. In some embodiments, the NO device calculates the amount of NO loss as a function of how the scrubber chemistry (e.g. calcium hydroxide availability, sodium hydroxide availability, water content) varies over time. This time-based assessment of NO loss can be a function of one or more sensor measurements and calculations made by the controller, such as elapsed time of scrubber use, scrubber inlet humidity, downstream humidity, quantity and concentration of NO gas passed through the scrubber, quantity and concentration of NO₂ gas passed through the scrubber, volume of total gas passed through, scrubber temperature, and other factors.

In some embodiments, the amount of NO lost to the scrubber varies as a function of water content in the scrubber. In one embodiment, the amount of remaining water content within the scrubber is calculated as a function of scrubber inlet humidity (measured or known) and scrubber outlet humidity (typically measured).

### Fault Detection: Resonant Frequency

An electric NO generation system that utilizes AC voltage to form the plasma can measure the resonant frequency of the high voltage circuit. In some embodiments, the resonant frequency is determined by measuring the electrical current through the high voltage circuit at a range of frequencies. The resonant frequency is identified as the frequency associated with maximum current.

The resonant frequency can be identified at various times. In some embodiments, the resonant frequency is identified during one or more of power-on, power-off, before a patient, at specific time intervals (e.g. daily), at the request of a user, and other times.

In some embodiments, a change in the resonant frequency of the resonant circuit is interpreted by the control software as a fault in the system. For example, a faulty electrical connection to an electrode would manifest as a difference from the expected resonant frequency. In another example, a pair of electrodes that have eroded over time will have a different resonant frequency than when they were new.

A sudden change in resonant frequency after a period of normal operation at the nominal resonant frequency would be indicative of a failure in the system (e.g. a short in the transformer secondary windings). The transformer secondary windings operate at high voltages, which can break down insulation over time and lead to a short.

Small changes in resonant frequency may also be used to detect electrode wear and remaining life, as the capacitance of the circuit can change as the electrodes wear through use. In some embodiments, the controller measures the resonant frequency and/or the capacitance of the high voltage circuit and requires service with those measurements cross a threshold.

By knowing the resonant frequency, a NO device can adjust the AC waveform that excites the high voltage circuit for optimal performance. This can compensate for drifts in system performance over the life of a device. For example, the resonant frequency can be updated in response to changed due to electrode wear. It should be noted that some systems operate at a frequency near but not equal to the resonant frequency. The resonant frequency is a point of peak production with low stability. By operating at a frequency to one side or the other of the resonant frequency, small changes in the AC signal produce small deviations in NO generation (i.e. are more stable).

### Fault Detection: Gas Measurement

In some embodiments, a NO generation and/or delivery system has the capability to compare two or more measurements of a parameter to confirm proper functioning. In some embodiments, two independent methods of determining NO concentration can be compared, for example, by calculation and by measurement. In some embodiments, a NO generation controller compares two or more of the following measurements: the product gas concentration, the inhaled gas concentration, and the predicted gas concentration based on plasma activity and expected gas losses (i.e. oxidation and scrubber NO loss calculations) to confirm proper functioning of the system. For example, if the controller commands a duty cycle of plasma within the plasma chamber and NO is not detected by one or both of the product gas NO sensor and the inhaled NO sensor, then the system shall generate a fault condition. In another example, the controller can compare the product gas NO sensor readings with expected NO levels within a recirculation loop as product gas ages. In the event that the product gas measurement and the NO concentration prediction differ by a threshold amount, the controller can respond by one or more of generating an alarm, automatically switching to a backup NO generator, ceasing NO production, and ceasing NO delivery.

### Inspiratory Limb Characterization

NO is often delivered to an inspiratory limb of a ventilation circuit. Ventilation circuits vary by length, diameter, presence/absence of a humidifier, and other factors. These variations alter the dead volume and compressibility of the gas within the circuit. The dead volume of the inspiratory circuit is directly related to the transit time for NO to travel from the location of introduction to the inspiratory limb (i.e., injection) to the patient. In some embodiments, a NO device generates a signal in the NO gas flow so that the transit time from device to patient can be quantified. In some embodiments, the signal includes an intentional variation in the NO delivery rate (e.g., a square wave, or a brief pulse or pause in NO delivery) at a known point in time and then detects that event through gas sensors located near the patient. The signal is generated by the controller by manipulating one or more flow control devices (e.g. pumps, valves, flow controllers) to achieve the desired signal. The elapsed time between generating the signal and detecting the signal is a function of the timing between NO generator and patient as well as gas sensor response time. When the signal is generated by the device controller, the controller concomitantly begins a time counter to track the elapsed time until the signal is detected at the gas sensors. Gas sensor response time and transit time from the inspiratory limb to the gas sensors can be characterized a priori and subtracted from the time to the signal, as needed, to derive the transit time to the patient. In some embodiments, the timing of the signal arrival to the gas sensors is determined to occur when the gas sensor signal exceeds or falls below a threshold. For example, a patient is being treated with 20 ppm NO. The device determines the inspiratory limb transit time by delivering 1 second of 0 ppm NO. The transit time is determined as the elapsed time from when the 1 second event began until when the gas sensor signal drops below 18 ppm. In this example, the transit time takes 20 seconds. the t90 of the sensors is 10 seconds and the transit time of the gas sample from the inspiratory limb to the gas sensors is 2 seconds. Thus, the transit time from the NO device to the gas sample collection point (a point in close proximity to the patient) is 20-10-2, or 18 seconds.

In some embodiments, a NO delivery system measures the transit time of NO through the inspiratory limb and knows the oxygen concentration in the inspiratory gas (either by direct measurement, default parameters, or communication with the ventilation device). The NO delivery system can then calculate the amount of NO that would oxidize to NO₂ as a function of the transit time, pressure, temperature, oxygen concentration and NO concentration in the inspiratory limb. This enables the system to compensate for oxidation that occurs during the transit time as the NO is delivered to the patient. In some embodiments, the transit time and O₂ concentration are utilized to determine the effects of oxidation on NO levels in the absence of a NO sensor.

In some embodiments, a NO delivery device is utilized to maintain a target NO concentration within a closed-circuit anesthesia system. The system measures the NO concentration within the circuit and adds NO as needed to achieve a target inspired concentration. In some embodiments, the inhaled NO measurement is made immediately after the anesthesia system CO₂ scrubber so that any NO loss to the scrubber is accounted for.

### NO Measurement

Various methods can be utilized within a NO generation system to measure NO levels in the product gas and inhaled gas including but not limited to electrochemical, chemiluminescent, photo-ionization detection, and spectroscopic. In some embodiments, electrochemical sensors with solid electrolyte are ideal due to their long service life and low cross-sensitivity to water.

### NO Measurement Error Detection

In some NO generation systems, there are three types of NO measurements that can be made: 1) predicted NO concentration based on plasma and reactant gas properties, 2) measured product gas concentration, and/or 3) measured inhaled gas concentration. In some embodiments, a NO generation and delivery system controller will compare two or more of these measurements and ensure that they are aligned within acceptable tolerances. For example, the mathematical difference between the indicated NO concentration from two independent or redundant NO measurement techniques should be within a particular limit (e.g. 5%) or the system can infer that there is an error. When the values between two or more assessments (i.e. calculations or measurements) do not align, the system can do one or more of change NO production channels or alarm. Some alarm conditions (e.g. faulty plasma plant) prompt the user to replace the NO generation/delivery system. Some alarm conditions prompt an NO production channel change (e.g., automatic channel change, or manual change by a User), while other alarm conditions prompt the user to check the system (e.g., for leaks, disconnected pneumatic fittings, clogged gas sampling filter, etc.).

### Calibration

Gas sensors within a NO delivery device require periodic calibration. Calibration is often performed by a user using a bottle of calibration gas on a periodic or as-need basis. This adds burden to the user which can be avoided if the NO delivery system is able to self-calibrate. In some embodiments, the NO delivery device includes a cylinder of calibration gas in it so that it can self-calibrate. In some embodiments, the NO delivery system flows calibration gas through one or more gas sensors to check or adjust calibration. Calibration can occur at multiple times, including but not limited to before use, periodically during treatment and at the request of a user. In some embodiments, the calibration gas is a mixture of more than one gas (e.g. a combination of N₂, NO and NO₂). In some embodiments, the NO device includes more than one calibration gas cylinder. The calibration gas is typically stored in a high-pressure cylinder. The gas is regulated down to a known pressure via a pressure regulator. Flow is modulated with a binary or analog flow controller.

In some embodiments, a NO delivery device houses a calibration gas cylinder that lasts the entire interval between periodic servicing (e.g. 1 year). In some embodiments, the NO device receives gas cylinders that are single use for a single calibration session. In some embodiments, the gas cylinders interface with the NO device through a threaded interface, the threads being unique for a specific type of gas (e.g. NO/NO₂ and O₂ mixtures) so cylinders are inserted in the correct location. In some embodiments, the gas cylinder is punctured upon insertion into the NO delivery device.

In some embodiments, the calibration gas cylinders include information that is conveyed to the NO delivery device, such as gas lot number, certified concentration level, gas type, gas manufacturer, expiration date, date of manufacture, etc. This information can be communicated to the NO device via a variety of means including but not limited to optical (bar codes), magnetic strip, and radiofrequency (RFID, blue tooth, etc.).

In some embodiments, a NO generation device includes a separate NO generator that is utilized to generate a calibration gas. The calibration gas generator is utilized periodically when calibration is required. Owing to the fact that the calibration gas generator is seldomly used, it remains in calibration producing a acceptable accurate concentration of calibration gas for a period of time.

### Product Gas NO Measurement for Inspired Gas Sensor Calibration

In some embodiments of a NO generation system, a product gas NO sensor is utilized as a reference sensor to calibrate inspiratory gas NO and NO₂ sensors. This approach, despite having some experimental error, can be acceptable because the accuracy requirement for inhaled NO sensors is somewhat forgiving (e.g. FDA requires +/−20% accuracy for inhaled NO). The reference gas sensor (e.g. NO, NOx) is typically located immediately downstream of a NO₂ scrubber and/or NO₂ reducer (e.g. ascorbic acid) so that the product gas is essentially devoid of NO₂. In some embodiments, the product gas with known concentration is, in turn, flowed to secondary gas sensors utilized for measuring the concentration of NO and NO₂ in the inhaled gas. In some embodiments, the system generates a low concentration product gas that can be directly used to calibrate an NO sensor. In some embodiments, the NO product gas is diluted a known amount to produce a concentration of gas that is compatible with the inhaled gas NO sensor. In some embodiments, the system controller that orchestrates the calibration process takes into account the NO lost between the time of measurement at the reference NO sensor and the time of measurement at the inspired gas NO sensor, thereby lowering the reference NO concentration to which the inspired NO gas is calibrated to. In some embodiments, the controller calculates the amount of NO lost as a function of one or more of NO concentration, residence time, pressure, oxygen concentration, humidity level, and temperature. In some embodiments, the product gas is diluted with air. In some embodiments, the product gas is diluted with a nitrogen-rich (i.e. nitrogen concentration >79%) gas that is either externally sourced or generated (e.g. pressure-swing adsorption method) to decrease oxidation rate of the product gas as it travels to the inspiratory gas sensor(s) to be calibrated.

In another embodiment, product gas of known NO concentration is used to calibrate an inspiratory NO₂ sensor within the system. In this method, product gas is generated by the NO generator and one or more of a) scrubbed of NO₂ (e.g., soda lime) and b) reduced to all NO (e.g., ascorbic acid) prior to NO measurement with a reference NO sensor. In another embodiment, the product gas is optionally scrubbed/reduced and a reference NOx sensor is utilized to measure the sum of NO and NO₂ within the product gas. This provides the device controller with a reference gas concentration measurement. The product gas is then permitted to age within the system.

In some embodiments, the gas is partially aged prior to subjecting the gas to the inhaled gas sensors. In order to obtain an accurate estimate of NO₂ levels in the product gas after partial aging, the controller calculates the amount of NO that converted to NO₂ prior to exposure to the inhaled gas sensors as a function of one or more of time, product gas humidity level, product gas temperature, product gas O2 concentration, product gas NO concentration, and product gas pressure.

In some embodiments, the product gas is permitted to fully oxidize (i.e. fully or nearly-fully convert to NO₂) prior to optional dilution and use as a reference gas for calibration of one or more inspiratory gas NO₂ sensors. In one embodiment, product gas aging is accomplished by transferring the gas through a dedicated gas aging tube of sufficient length that the transit time is sufficient that all of the remaining NO within the gas converts to NO₂ (i.e. the product gas is fully oxidized). In another embodiment, product gas is either circulated around a recirculating loop or aged in a chamber for sufficient time that all NO converts to NO₂ prior to utilizing the NO₂ gas for calibration. In some embodiments, the product gas aging is hastened by subjecting the product gas to elevated pressure. Given that NO₂ is stable in air, there are no timing concerns with complete aging of oxidized product gas and the system can err on the side of excessive NO aging time. After sufficient aging time (e.g., several minutes), all of the NO within the product gas oxidizes to form NO₂. The NO₂ gas can then be utilized to subject the inspiratory NO₂ sensor to a known concentration of NO₂ gas for calibration purposes. In some embodiments, the NO₂ gas is diluted prior to being used to calibrate the inspiratory NO₂ gas sensor. In some embodiments, the product gas is diluted before aging to NO₂, however this results in longer oxidation time due to the slower oxidation rate. In some embodiments, the product gas is held at elevated pressure (e.g., greater than atmospheric pressure) during gas aging to accelerate the oxidation process.

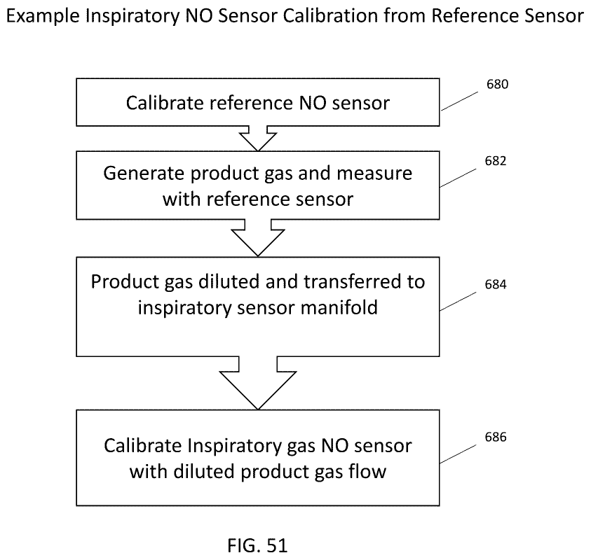

FIG. 51 depicts an exemplary process whereby an electric NO generator utilizes a reference NO concentration measurement to calibrate one or more inspired NO gas sensors. The reference NO sensor is typically either calibrated (step 680) at the time of system manufacturing or during periodic maintenance intervals. When the system is in the field, the system generates NO product gas at a constant rate and measures with a reference sensor at step 682 (e.g., constant reactant gas flow rate, constant plasma activity). In some embodiments, the NO production parameters (i.e. reactant gas flow rate, plasma duty cycle, plasma frequency, plasma dithering, etc.) are controlled in a closed-loop manner based on the reference NO concentration measurement to maintain consistent product gas for calibration purposes. The product gas can be diluted and transferred to a manifold (step 684), and the inspiratory gas NO sensor can be calibrated with diluted product gas flow (step 686). Errors in the calibration process stem from errors in the original reference sensor calibration (from step 680) as well as errors in the estimates for NO loss and dilution of product gas. For example, errors in step 682 can include errors in the product gas NO measurement with the reference sensor and an assumption that NO₂ is zero after scrubbing, and errors in step 684 can include errors in dilution ratio accuracy and errors in estimation of NO loss between the reference measurement and the inspiratory sensor, including accuracy of aging time (e.g., flow rate/volume) and errors in flow rate accuracy. Despite the stacking of multiple sources of error, the accuracy of the inspiratory NO sensor can be within the accuracy requirements cited by the US FDA (i.e., +/−20%).

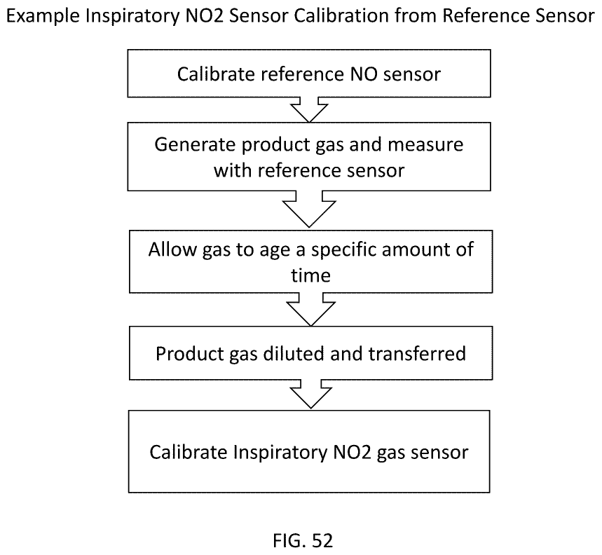

FIG. 52 depicts an exemplary process whereby an electric NO generator utilizes a reference NO concentration measurement to calibrate one or more inspired NO₂ gas sensors. Errors in the inspired NO₂ gas sensor calibration stem from the accuracy of the reference NO sensor calibration and dilution accuracy. Additional sources of error include those from product gas NO measurements with a reference sensor, assumption that NO₂ is zero after scrubbing, the accuracy of the aging volume based on tube length, the accuracy of the aging volume based on flow rate/volume, and errors in dilution ratio accuracy.

In some embodiments, the inspired gas NO and/or NO₂ sensors are calibrated during patient treatment. A NO generation system with redundant NO generators can utilize one NO generation channel to treat a patient while the other NO generation channel is utilized to calibrate the inspired NO and/or NO₂ sensors. While the inspiratory gas sensors are being calibrated, the user interface typically does not show any NO or NO₂ measurements. In addition, the user interface may show a message such as “gas sensor calibration in process.” Once the calibration process is completed, some embodiments of the inspiratory gas calibration system purge the product gas pathway to the inspiratory gas sensors to prevent long-term storage of NO₂ gas within the system.

In some embodiments, a product gas NO sensor (e.g. electrochemical or solid state) is replaceable by a user. In some embodiments, this eliminates the need for field calibration of the NO sensor. In some embodiments, replacement of the NO sensor is prompted based on a timing interval, detected drift in the sensor, or the amount of cumulative sensor use. In one embodiment, the NO sensor is included in a scrubber cartridge for frequent replacement.

In some embodiments, a gas concentration sensor (e.g. NO, NO₂) is packaged with a finite source of calibration gas. In one embodiment, when the gas sensor is installed, calibration gas is released, exposing the sensor to a known concentration of gas for calibration purposes.

In some embodiments, a gas sensor is packaged with calibration gas located in the sensing region of the sensor. In some embodiments, during installation, the sensor is first electrically connected to the system so that the sensor reading from the calibration gas is captured. Then, the calibration gas is released from the sensor either manually by the user or passively as the sensor is inserted into the system. The sensor is then exposed to a second gas (e.g. air) with known concentration of the target gas to provide the second point in a two-point gas calibration.

### Gas Sensor Signal Processing

Despite delivered NO dose having a wide tolerance of +/−20% of target, user expectations are that a NO delivery system delivers exactly the concentration that was requested. For example, delivering a dose of 19 ppm when the user requests 20 ppm, may not be acceptable despite being acceptably accurate from a regulatory perspective. Gas concentration measurements can vary over time as well, causing fluctuations in the indicated NO concentration. In some embodiments, a NO delivery system utilizes a heavy filter when the gas sensor data are stable. If there is a rapid change in the value, then the system changes/removes the filter so that the rapid change in values is indicated to the user on the user interface. This eliminates the risk of small fluctuations in the signal from distracting the user.

### Method to Hasten Gas Sensor Calibration

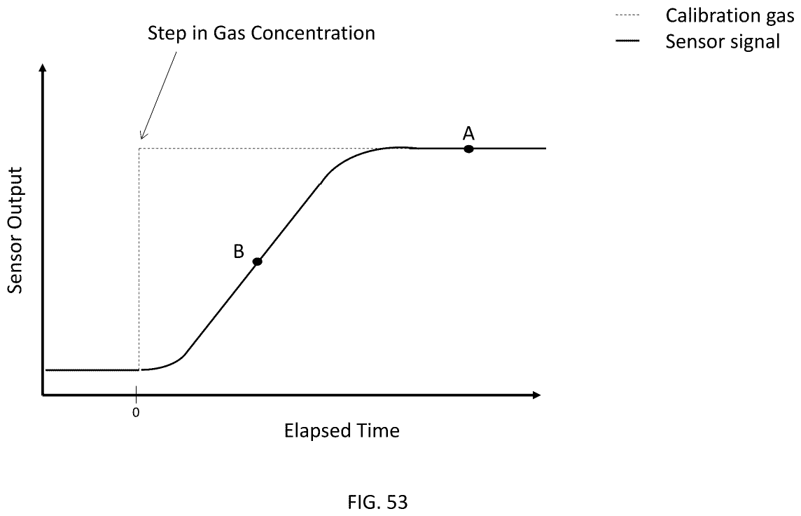

Electrochemical gas sensors are typically calibrated by recording their signal in two known conditions and then updating the offset and gain accordingly. This can be done by initially exposing a sensor to a baseline value followed by a calibration gas of known concentration and waiting for the signal to stabilize. Once the sensor signal has stabilized, the appropriate gain and offset for the calibration can be determined so that the sensor output indicates the true contents of the gas. In some embodiments, this process is expedited by characterizing sensors beforehand for their response to a step change in gas concentration. A particular type of sensor will respond to the step change over time in a predictable and repeatable manner, as shown in the exemplary graph of FIG. 53. The sensor is exposed to an initial concentration of gas (e.g. 0 ppm NO or 210,000 ppm oxygen) and then a step change in concentration is applied. The sensor responds to the step change in concentration by increasing the sensor output signal over time. The calibration technician can wait until the sensor signal stabilizes, depicted at Point B, however this could take 30 seconds or more. An alternative approach is to analyze the slope of the sensor signal at an earlier time point depicted as point B. Given that the sensor is expected to respond the same way to similar inputs, the final signal output can be predicted by the magnitude and slope of the signal a set amount of time after the step gas concentration change. In some embodiments, this information is stored in a look-up table or a mathematical function that the device controller uses to set the gain and offset values for the next period of use.

### Clinical Applications

### Organ Transplant Application

In some embodiments, harvested organs are perfused with gaseous NO and/or NO liquid solution (e.g. nanobubble solution) during transport and storage to preserve them prior to implantation. The NO is expected to dilate blood vessels to improve perfusion of the organ. In some embodiments, the NO additionally prevents infection in the organ.

In a specific example, gaseous NO is used to prevent lung transplant rejection. When an organ is harvested, the rejection process begins immediately. There is a biomarker that provides a quantitative measure of the level of rejection taking place. NO effects of vasodilation, angiogenesis and anti-infection are expected to prolong the viability of the transplanted lung. In practice, harvested lung is filled with a gaseous mixture containing NO to prevent lung collapse.

Progressive loss of lung function is a common malady that occurs following lung transplants. The etiology is understood to be a combination of rejection by the host's immune system as well as infection (e.g., from airborne microbes). In some embodiments, inhaled NO is administered to lung transplant patients prophylactically to prevent infection from taking root within a transplanted lung. In some embodiments, inhaled NO is administered to lung transplant patients once lung rejection and/or infection has been detected. The inhaled NO, when dosed in appropriate concentrations can provide benefit to oxygen uptake as well as kill pathogens within the lung. NO therapy may be delivered continuously or intermittently, depending on the severity of the patient condition and the concentration of the NO gas.

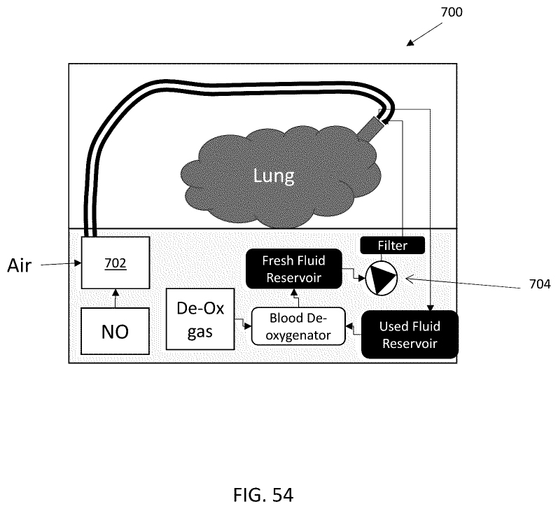

FIG. 54 depicts an exemplary embodiment of a lung transport device 700 that perfuses the lung with de-oxygenated fluid (e.g., blood) while ventilating the lung with positive pressure to keep it open. A pressure source 702 provides a flow of pressurized gas containing one or more of air, oxygen and nitric oxide. In some embodiments, the gas flow is continuous. In other embodiments, the gas flow is intermittent and cyclical like typical respiratory motion. In some embodiments, the concentration of NO is roughly 20 ppm throughout the treatment. In some embodiments, high dose NO (i.e., concentrations of NO greater than 150 ppm) are utilized either continuously or intermittently to prevent infection of the lung. A PEEP is maintained on the lung at all times to prevent lung collapse. The vasculature of the lung is perfused by fluid (e.g., de-oxygenated blood) to pick up and remove oxygen and waste from the lung tissue. A pump 704 moves fluid from a reservoir, through a blood oxygenator, past a NO nanobubble generator, through the organ and to a storage reservoir.

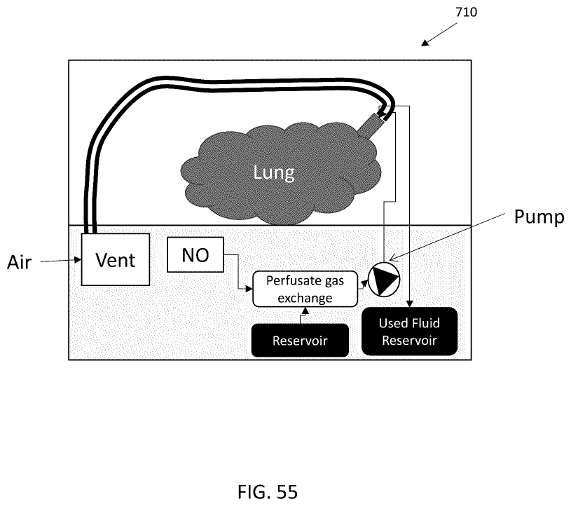

FIG. 55 depicts an exemplary embodiment of a lung transport device 710. The lung is ventilated with air. Perfusate is drawn from a reservoir and passes through a perfusate gas exchange device (e.g. membrane gas exchanger) where NO is added to the perfusate. The perfusate is then pumped through the lung to collect oxygen and carbon dioxide. The NO dilates the blood vessels within the lung, reducing the flow restriction through the lung and maximizing recruitment of lung blood vessels. Used perfusate is collected within a second reservoir. In other embodiments (not shown), the perfusate is de-oxygenated and reused.

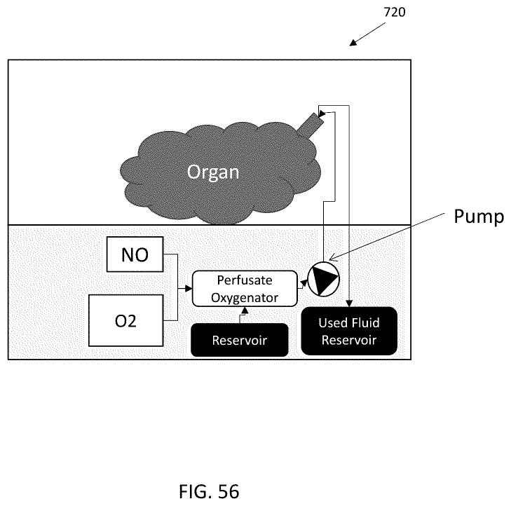

FIG. 56 depicts an exemplary embodiment of an organ transport device 720. In this embodiment, NO is introduced to the perfusate within the oxygenator. The NO dissolves in the perfusate and relaxes smooth muscle tension within the organ. This improves perfusion of the organ and decreases the potential for blockage of the blood vessels within the organ. In this embodiment, the perfusate is not re-used to prevent methemoglobin build up within the perfusate. The NO can be sourced from a variety of NO sources, including but not limited to gas cylinder, electric generator, N₂O₄-derived NO, NO donor molecule, and other approaches.

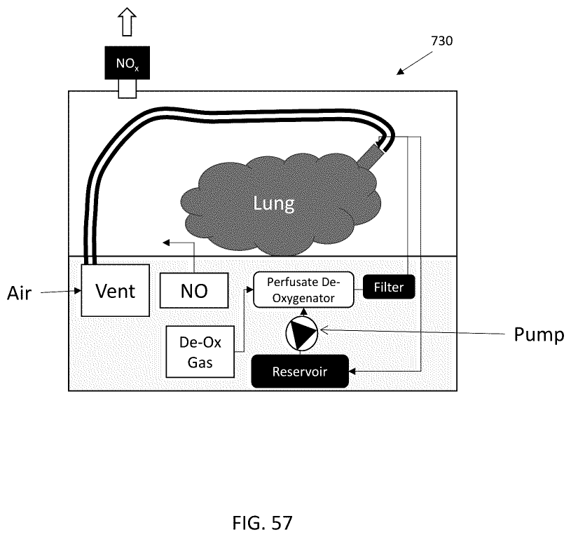

FIG. 57 depicts an exemplary embodiment of an organ transplant device 730 wherein the chamber housing the organ is filled with an NO-containing gas. The NO containing gas prevents microbes from growing on the external surfaces of the organ. In some embodiments, the chamber housing gas is a mixture of NO and N₂. In some embodiments, the concentration of NO within the chamber is between 20 ppm and 100,000 ppm NO in a balance of N₂. Gas exits the chamber through a NOx scrubber that removes NO and/or NO₂ from the gas. The perfusate flows in a closed loop, propelled by a pump. The organ in this case is a lung that is ventilated with oxygen-containing gas (e.g., air). The perfusate (e.g., blood) adsorbs oxygen as it flows through the lung. A de-oxygenator removes oxygen from the blood so the blood can be reused. A filter after the pump removes leukocytes, emboli and other potentially harmful materials.

### Intestinal Oxygen Exchange

In some embodiments, a gaseous mixture containing oxygen and NO is delivered to the rectum/colon/large intestine/small intestine of a patient to promote oxygen exchange across the gastrointestinal wall. Gas is introduced to the gastrointestinal tract, through a lumen in a device inserted either orally or anally. In some embodiments, the device consists of one or more of an endoscope, colonoscope, catheter, nasogastric tube, laparoscopic device, or other device. The NO acts on the smooth muscle within the blood vessel walls within the intestine, making the smooth muscles relax. This increases blood flow through the intestines, helping increase oxygen uptake from the oxygen-containing gas. This level of oxygen uptake through the gastrointestinal tract walls can supplement or in some instances replace the oxygen uptake normally performed by the lungs. In one embodiment, GI-oxygenation is utilized during a surgical procedure (e.g., lung transplant, heart transplant, heart surgery) to improve patient oxygenation. In some embodiments, GI oxygenation is utilized continuously by a patient to improve systemic oxygenation.

In some embodiments, the concentration of NO and/or oxygen during GI-oxygenation is modulated by the device controller based on a patient SpO₂ measurement by a sensor in the system.

In some embodiments, gas flow can be continuous. In some embodiments, gas flow can be intermittent (for example, 5 minutes every hour, only when the patient is sleeping, etc.). In some embodiments, gas is introduced at a deepest location within the patient and migrates out of the patient along the gastrointestinal tract. In some embodiments, a filter at the outlet of the patient (e.g., mouth, anus) removes one or more of VOCs, NO, and NO₂ from the gas stream to prevent contamination of the patient environment. In some embodiments, the scrubber contains one or more of soda lime, activated carbon, potassium permanganate, TEMPO, and metal-organic framework material.

In some embodiments, the GI-oxygenation device includes a pressure sensor to measure the gas pressure within one or more of the gas delivery lumen and location of gas delivery. In some embodiments, the controller uses the pressure sensor measurement as feedback to modulate the gas flow to a tolerable level for the patient. In some embodiments, the controller will generate a fault and/or stop treatment if the gas pressure exceeds a threshold.

The NO gas source for a GI oxygenation system can be in the form of a NO tank, electric NO device, N₂O₄-derived-NO device, NO-donating molecule device or other source.

### Laparoscopy

In some embodiments, the peritoneal cavity is inflated with gas containing oxygen and nitric oxide to improve oxygen uptake into the blood stream. In some embodiments, insufflation gas used during laparoscopic procedures is comprised in part of NO. The NO gas combats infection and increases O₂ uptake. In some embodiments, cancerous lesions within the peritoneal cavity are treated with high concentration NO gas (for example, up to 100,000 ppm) by insufflating the peritoneal cavity with gas. In some embodiments, the insufflation gas is devoid of oxygen to prevent NO oxidation into NO₂.

### Ultrasonic Energy

Ultrasonic energy can be utilized to quantify blood flow rates (e.g. Doppler method) and the geometry of 3d structures within the body via ultrasonic imaging. Typically, ultrasound is applied to a patient with a hand-held probe for acute measurements. Recent advancements in ultrasound technology have enabled the creation of a compact sensing device that can be attached to the body for chronic use (e.g. a patch configuration). The device is applied is specific locations to analyze target internal organs. Visualization of the thorax requires placement of the sensor between the ribs. The sensor sends and receives ultrasonic waves and can reconstruct the sensed information into static and dynamic information. Simultaneous use of multiple sensors (e.g. an array) can analyze activity within the entire thoracic cavity.

Current state of the art medicine involves placing a Swan Ganz cardiac output catheter within the pulmonary artery of a patient to measure cardiac output. A noninvasive, real time approach to cardiac output measurement can greatly simplify the cardiac output measurement procedure and reduce the patient safety risk. In one embodiment, an ultrasonic cardiac output measurement device is utilized to create a dose-response relationship for a specific patient. This information can be utilized to identify patients that do not response to NO and determine the minimum effective dose in patient that do respond. This approach can be applied in a clinical setting by a doctor when prescribing NO to a patient. The same approach can be utilized to determine the level of need for supplemental oxygen for a given patient.

In some embodiments, a patient receiving respiratory therapy (e.g. nitric oxide, ventilation, supplemental oxygen), wears an ultrasonic device that analyzes the thorax over time (e.g. hours, days). In some embodiments, the ultrasound device quantifies the volume of the heart over time. Heart volume measurements are then input into a processor that determines cardiac output based on the changes in volume over time with each heart contraction. In other embodiments, the volume of the lung is quantified using data from the ultrasound sensor. This information provides insight into whether or not a particular region of the lung is ventilated or not ventilated (e.g. obstructed, collapsed). Changes in lung volume are indicative of respiration and can be used to calculate the tidal volume and respiratory rate. In some embodiments, a respiratory therapy device titrates the patient treatment to maximize cardiac output and/or lung perfusion. For example, a NO delivery device determines that lowest NO dose that will still maximize lung perfusion. A ventilator device can vary one or more of inspiratory pressure, volume, flow rate, positive end expiratory pressure (PEEP) and respiratory rate to maximize lung perfusion. In one example, optimum PEEP is varied to optimize the balance of ventilation and hemodynamics.

In some embodiments, a NO generation and/or delivery system includes one or more integrated ultrasound sensors. The ultrasound sensors communicate with the NO generation system controller (e.g. wired, or wirelessly) either directly or after data processing into volume and flow data. In some embodiments, the ultrasound sensor(s) are attached to the patient's thorax. In other embodiments, the ultrasound sensor is attached to one or more of a patient's neck, abdomen, or extremity. In some embodiments, the ultrasound sensor reports a blood flow rate within a blood vessel (e.g. carotid, aortic, pulmonary artery, etc.). In some embodiments, the sensor reports a volume measurement (e.g. total heart, one or more heart chambers). In some embodiments, the NO device utilizes the blood flow and/or volume measurements as feedback to a NO dosing algorithm. Higher concentrations of inhaled NO induce greater vascular relaxation which reduces pulmonary resistance resulting in improved cardiac output and subsequently reducing the stress in the right heart. In some embodiments, the NO device monitors lung perfusion in one or more regions of the lung based on lung volume measurements from the one or more ultrasonic sensors. In some embodiments, when lung perfusion is detected to be low or decreasing, the NO generation system can do one or more of the following: notify a ventilator to increase ventilation and increase NO delivery. In some embodiments, optimal patient positioning (e.g. prone, vertical, supine) is determined based on ultrasonic visualization and respiratory assessment.

In some embodiments, cardiac output data are utilized by a NO generation and/or delivery system as feedback during a weaning process. In some embodiments, the NO generation system decreases the inhaled NO concentration and monitors cardiac output. If cardiac output is not affected by the decrease in inhaled concentration, the NO device continues at the new inhaled concentration level. If cardiac output decreases, then the NO device will increase the inhaled concentration. In some embodiments, the weaning process can be automated in real time.

### Anesthesia

Nitric oxide is often delivered to an anesthesia circuit. Anesthesia circuits are typically a closed circuit to maintain anesthetic materials. Carbon dioxide from the patient is absorbed by a soda lime scrubber in the circuit. When NO is delivered to an anesthesia circuit, there is a risk of NO accumulating within the circuit resulting in a higher delivered dose than targeted. In some embodiments, the anesthesia circuit is operated in an open or partially open mode whereby a portion of the circulating anesthetic gas is released from the system and replaced with fresh air. In some embodiments, a NO delivery system measures the flow of fresh gas entering the anesthesia circuit. NO is then delivered to the fresh gas entering the anesthesia circuit in proportion to the fresh gas flow to maintain a target NO concentration within the anesthesia circuit. In one embodiment, the dosed fresh gas enters the circulating flow in the same location or immediately after the location of gas expulsion. This improves consistency of NO concentration throughout the anesthesia circuit.

### ECG Analysis

In some embodiments, a NO delivery system can measure the degree of pulmonary hypertension in a patient with an ECG measurement. The system uses this measurement as an input to the NO dose control system. In some embodiments, as the pulmonary hypertension increases, as indicated by the EKG, the system automatically increases the NO dose to the patient within a pre-defined range of acceptable doses. In some embodiments, the NO delivery system decreases the amount of NO delivered as pulmonary hypertension decreases.

### CO₂ Waveform Analysis

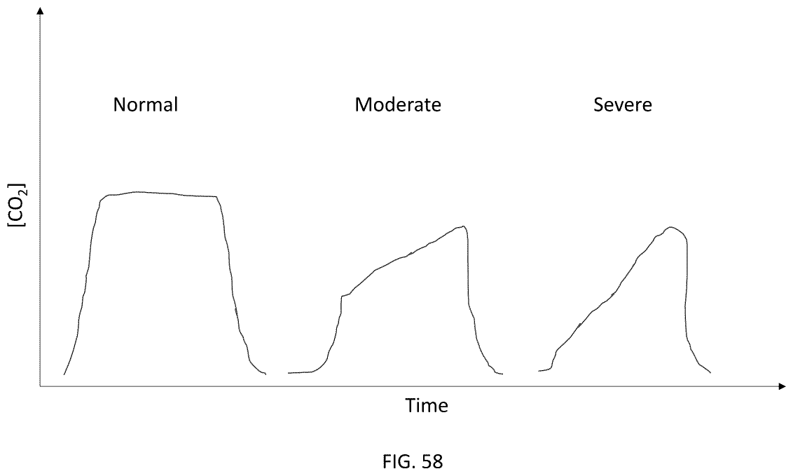

In some embodiments, an NO delivery device analyzes the CO2 waveform of a patient to assess the degree of pulmonary obstruction. Higher levels of obstruction result in a slower rise in CO2 levels during exhalation, as depicted in the exemplary graph shown in FIG. 58. A NO delivery device can utilize capnography (i.e. CO2 waveform) data to assess the effect of NO on the patient. A patient experiencing elevated obstruction based on the capnograph is administered additional NO (i.e. a higher dose) to improve gas exchange within the patient.

In some embodiments, the CO2 measurement is made at periodic intervals (e.g., every 3rd minute, or every 5 minutes). In some embodiments, NO delivery is halted during the CO₂ waveform data collection. The CO₂ waveform can be measured in multiple ways including but not limited capnography and thermopile. Furthermore, periodic monitoring of the CO2 waveform enables clinicians using the device to track disease progression.

In some embodiments, CO2 sample collection is done through a dedicated lumen in the NO delivery device. In other embodiments, NO is delivered through the same lumen that CO2 is sampled from. The NO is delivered to inhalation while the CO2 is sampled during exhalation with the two events being out of phase with each other.

In some embodiments of a NO delivery device with capnography capability, the system utilizes a common lumen for NO delivery and CO2 analysis. The system pushes a bolus of NO to the patient which may or may not be facilitated with a non-NO containing purge gas. After delivering the NO pulse, the device reverses flow within the delivery lumen to draw exhaled gas from the patient to the device. Exhaled gas is analyzed for CO2 content. This approach, commonly known as side stream capnography, purges the cannula of NO/NO₂ between breaths.

A system that samples CO2 from the patient allows for the capnograph (i.e., CO2 signal) to be utilized to detect the beginning of inhalation. When CO2 falls to near zero (atmospheric levels are roughly 420 ppb), inhalation has begun.

### Methemoglobin

Methemoglobin (metHb) is an oxidized form of the hemoglobin molecule that can be formed in the presence of NO. Methemoglobin is unable to deliver O₂ to the tissue presenting a problem clinically referred to as “methemoglobinemia” when its quantities are elevated, potentially leading to serious adverse events. Methemoglobin reductase is an enzyme that can metabolize methemoglobin and reduce levels in-vivo. When a patient is treated with nitric oxide (e.g., inhaled NO), ideally the rate of methemoglobin formation remains moderate as methemoglobin reductase metabolizes methemoglobin. If there is a deficiency of methemoglobin reductase, the kinetics of methemoglobin formation are accelerated, and high levels can ensue. When the methemoglobin formation rate exceeds the reduction rate, the patient ultimately displays markedly elevated methemoglobin, i.e. a large portion of their hemoglobin has been converted to methemoglobin and they are unable to deliver sufficient oxygen.

Clinically, measurements of nitrate, nitrite, methemoglobin and/or cyclic GMP in the blood can be utilized to assesses biomarkers that indicate exposure to NO. These markers are a direct reflection of exposure and vary in nearly real time with NO exposure. In one embodiment, more than one measurement of one or more of nitrate, nitrite, metHb and cyclic GMP are made at a known time interval(s). Using kinetic modeling, the time course of formation of these compounds in the blood can be ascertained. This modeling can be done manually by a clinician or automatically within the controller of an NO delivery device. By understanding the rate of change over time of one or more of nitrate, nitrite, metHb or cyclic GMP, a clinician or system can understand the relative magnitude of the methemoglobin clearance rate versus the metabolism rate of the nitrate, nitrite, metHb or cyclic GMP which informs the clinician and/or device whether or not the current treatment levels lead to adequate bioavailability in the lungs. This approach can be patient-specific and directly relates to a patient's own ability to metabolize nitrate, nitrite, metHb and cyclic GMP.

Considering safety, one or more of nitrate, nitrite, methemoglobin or cyclic GMP can be used as safety parameters. In one method, a patient with greater than 4% methemoglobin cannot be discharged from the hospital. When MetHb is >4%, the patient's ability to metabolize metHb is limited. The ability to look at a time history of nitrate, nitrite, methemoglobin and/or cyclic GMP enables a clinician to monitor bioavailability and to provide safety markers.

### No Mask Delivery

Nitric oxide is commonly delivered non-invasively to a patient through a nasal cannula or a facemask. These delivery systems are not perfectly sealed against the patient, hence allowing some NO and NO₂ to be emitted into the ambient air around the patient. In one embodiment of a NO delivery device, the patient wears a face mask in fluid communication with an inspiratory gas source and an NO source. When the patient inhales, they breathe in inspiratory gas. NO is delivered as a pulse either into the mask or through a nasal cannula when breath is detected, thereby ensuring that NO enters the patient. In some embodiments, the controller detects inspiratory events with a respiratory sensor (e.g. a pressure, temperature, humidity, or flow sensor) in fluid communication with either the mask, NO delivery lumen or inspiratory gas lumen. In other embodiments, respiratory events are detected by the NO delivery device controller by a sensor within the NO delivery device that is in fluid communication with the patient. In some embodiments, a NO pulse is delivered in response to a detected respiratory event. In some embodiments, a NO pulse is delivered at an instant in time predicted based on the timing of one or more prior respiratory events.

In some applications, patients require positive pressure in their airway during respiration (e.g. CPAP). FIG. 59 depicts an exemplary embodiment of a NO delivery device 740 that utilizes 742 a blower to pressurize an airway and deliver gas (e.g., air, air+O₂ mixture) to a patient. An optional sensor 744, labeled “S,” can be configured to measure one or more of flow rate, pressure, humidity and oxygen level of the inspired gas. The device includes a source of NO (e.g. a tank, NO generator). In some embodiments, NO is introduced to the inspiratory flow at or near the NO delivery device (no shown). This can result in elevated NO oxidation into NO₂ due to exposure to elevated O₂ levels and longer transit time. In some embodiments (shown), NO is delivered through a separate lumen and introduced to the inspiratory flow at or near the patient. In some embodiments, NO is delivered through a nasal cannula beneath the mask (not shown) to direct the NO into the patient. In the case depicted, the patient breathes through a mask. In some embodiments, NO is delivered continuously. In other embodiments, NO is delivered in pulses that are synchronized with the breathing cycle.

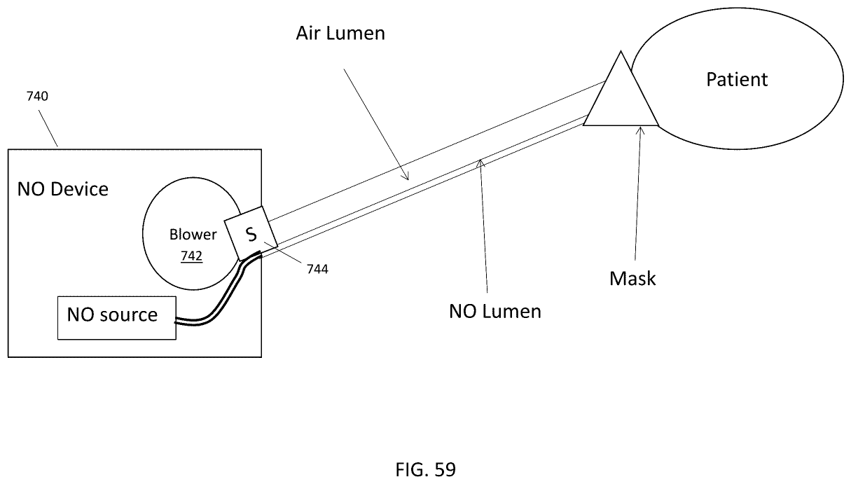

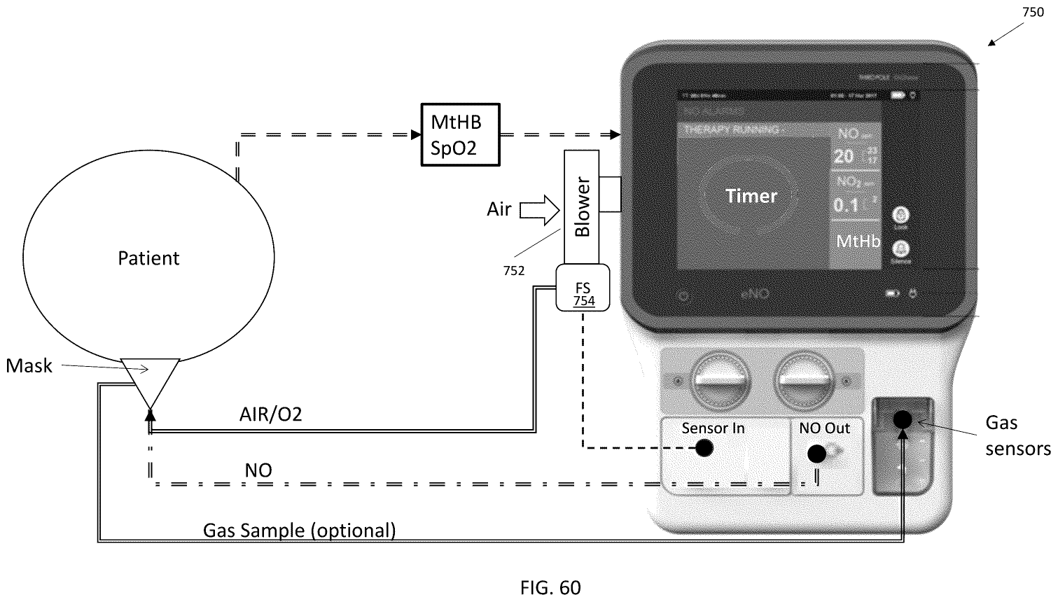

FIG. 60 depicts an embodiment of an electrical NO generation and delivery system 750 configured for delivery of inspiratory gas and NO to a patient. The system includes a blower 752 that provides pressurized gas flow to the patient. A flow sensor 754 between the blower and patient measures inspiratory flow. NO is introduced to the inspiratory air flow in proportion to the inspiratory air flow rate to achieve a target dose. In some embodiments, the blower and flow sensor are an accessory to a NO generation system that can be removably attached. A gas sample from the patient mask can be optionally flowed to gas sensors within the NO generation system for gas analysis (e.g. NO, NO₂, O₂).

One or more patient parameters are measured by the NO delivery system to monitor patient status, titrate the level of NO delivered to a minimal but effective level, and to detect potential alarm conditions. In some embodiments, one or more of patient methemoglobin and SpO2 levels are measured with a sensor and input into the control system through a wired (shown) or wireless (not shown) connection. In the presence of unacceptably high methemoglobin levels (e.g. >7%), the system controller may decrease the NO dose, pause NO delivery for a period of time, or cease NO delivery altogether. In the presence of low SpO2 levels, the system controller may increase the inhaled concentration of NO within a pre-programmed acceptable range of NO concentrations until a maximal SpO2 response is detected. In some embodiments, once a maximum SpO2 value has been reached, the system decreases the NO dose (e.g. in a step-wise manner) to identify the minimum dose that provides maximum SpO2. This cycle of increasing and decreasing NO dose can be done automatically by a NO device controller to ensure minimum NO dose for maximum oxygenation effect. It should be understood that every patient will respond differently to inhaled NO and not all patients respond at all. Hence, a titration approach of gradually increasing the NO dose until the SpO2 has reached a maximal value can provide clinical benefit while minimizing the potential for elevated methemoglobin levels.

In some embodiments, a user can customize the automated NO dose selection method by setting method parameters (e.g. NO incremental step size, NO decremental step size, time period between SpO2 checks, time period between dose increments, time period between dose decrements, quantity of SpO2 readings to average over, filter setting for results, initial NO dose setting, maximum NO dose setting, minimum acceptable SpO2 level, SpO2 alarm limits, etc.). In one embodiment, the User specifies a minimum acceptable SpO2 value to be 92%, the sampling period to be 2 minutes, the initial NO dose to be 20 ppm and the NO dose step size to be 10 ppm. The NO delivery device controller begins delivery of NO at 20 ppm, waits 2 minutes and reads the SpO2. If the SpO2 is greater than 92%, the NO delivery device controller maintains the current NO dose level for the time period between dose decrements. If the SpO2 is less than 92%, the NO delivery device increases the NO dose by the NO dose step size.

In some embodiments, a patient receiving inhaled NO therapy wears a wearable cardiac imaging device. The wearable cardiac imaging device provides data to the NO device controller pertaining to cardiac output and pulmonary resistance. In some embodiments, the NO delivery device varies the amount of NO delivered to the patient in response to cardiac imaging data. In one specific example, if the cardiac imaging data indicate that cardiac output has decreased, the dose of NO is increased to compensate. In another specific example, if the cardiac imaging data indicate that cardiac output has reached a maximal level, the NO dose is either held constant or decreased in an effort to titrate to a minimal but effective dose of NO.

In some embodiments, a NO CPAP system is utilized to treat lung and/or airway infection. The positive pressure aids in inflating the lungs to ensure adequate exposure throughout the lung. Inhaled NO doses can range from low (1 ppm) to high (1000 ppm). Duration of treatment can be prescribed or dependent on patient methemoglobin levels with the system decreasing or stopping NO dose when methemoglobin levels reach a threshold. Methemoglobin level is reported on the screen of the NO generation and/or delivery device. In some embodiments, the system includes alarms that initiate when methemoglobin levels exceed a threshold (e.g. 7%). In some embodiments, the user interface includes a treatment timer that indicates remaining treatment time (e.g. a timer that counts down).

The NO generation and delivery system can also be used to improve patient oxygenation. In some embodiments, the system starts at a low dose of NO (20 ppm) and increases the dose over time until SpO₂ levels have reached a maximum value. Then, the NO dose is sustained while methemoglobin values are monitored for safety.

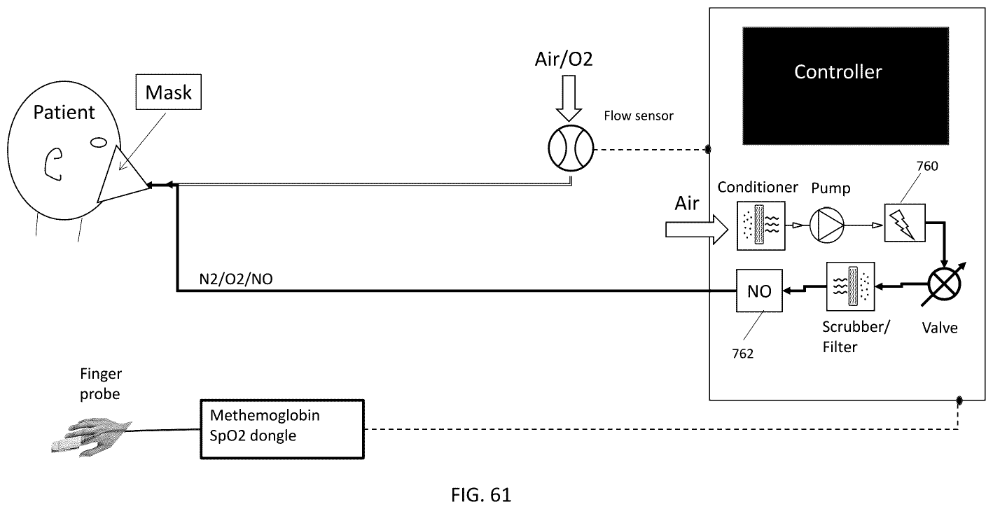

FIG. 61 depicts an embodiment of a NO generation and delivery system. Inspiratory gas passes through a flow sensor connected to the device controller. The controller pulls reactant gas (i.e. room air) through a gas conditioner (e.g. one or more of particle filter, VOC scrubber, water management component (e.g. desiccant)) with a pump. The gas then passes through a plasma chamber 760 where nitrogen and oxygen molecules in the reactant gas are divided and recombined, at least in part, into nitric oxide and nitrogen dioxide. The gas then passes through flow controller, a NO₂ scrubber, and a particle filter. The product gas is optionally measured with a sensor 762 for NO and or NO₂ prior to delivery to the patient. It should be noted that there are various architectures that can provide NO product gas and this invention is not limited to the depicted architecture. For example, in some embodiments, product gas is stored in a reservoir between the plasma chamber and the flow controller prior to delivery to a patient. In some embodiments, a gas reservoir contains NO₂ scrubber material. In some embodiments, the pump is located after the plasma chamber to decrease pressure within the plasma chamber as well as transit time under pressure of the product gas. The controller can modulate the dose delivered to the patient by varying the flow rate and concentration of the product gas. In some embodiments, the product gas delivered is proportional to the amount of gas inspired by the patient. In the depicted embodiment, the methemoglobin level in the patient's blood is measured with a finger probe (or equivalent). In the event that methemoglobin levels increase beyond a threshold, the controller will one or more of terminate treatment, decrease the delivered dose, generate an alarm, prolong the treatment at a lower concentration level, or deliver a quantity of methylene blue.

Automatic Conversion from Proportional to Constant Dosing

Consistent and accurate NO concentration within an inspiratory stream can be achieved by dosing the inspiratory stream with a proportional amount of constant concentration NO gas. Inspiratory flows that vary (e.g. ventilator flows) typically require continuously varying, proportional NO flows in order to achieve acceptably accurate NO dose consistency. Inspiratory flows that have a constant flow rate can be dosed with a proportional and constant NO gas flow to achieve acceptably accurate NO dose consistency. There is a certain level of variation in the inspiratory flow that can occur and still be accurately dosed by a constant flow of NO. The maximal amount of variation of inspiratory flow that can be acceptably dosed with a constant flow of NO gas is related to the NO dose accuracy acceptance criteria, the magnitude of fluctuation in the inspiratory flow rate, the volume of inspiratory limb between the NO injection point, the frequency of oscillation in the inspiratory flow rate, the patient/gas sampling location, and the degree of gas mixing that can occur between the NO injection pint and the patient/gas sampling point. As an example, a high frequency oscillatory ventilation (HFOV) treatment operating at hundreds of breaths per minute can be accurately dosed with a constant flow of NO-containing gas.

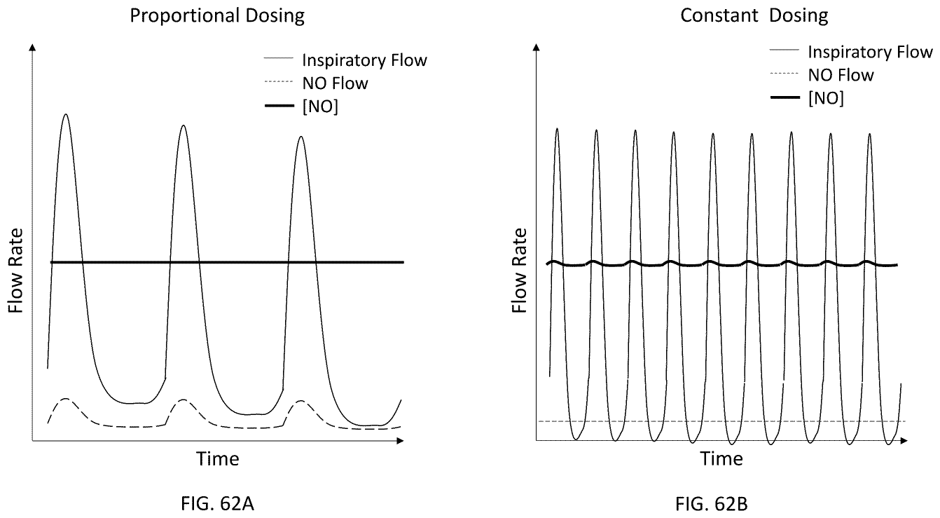

In some embodiments, a NO device automatically changes from a dynamic proportional dosing methodology to a static (i.e. constant flow) proportional dosing methodology, depending on the inspiratory flow characteristics (e.g. peak flow rate, tidal volume, inspiratory time, product gas transit time). FIGS. 62A and 62B depict an example of proportional dosing of constant concentration NO into a dynamic inspiratory flow stream in FIG. 62A. The resulting inhaled NO concentration is constant. FIG. 62B depicts delivery of a constant flow of NO into a higher frequency dynamic inspiratory flow. The inhaled concentration may include minor fluctuations in inhaled NO concentration, as shown, but is acceptable for clinical use.

In one embodiment, the system converts from dynamic proportional dosing to static proportional dosing when the respiratory rate exceeds a threshold value (e.g. 40 breaths/minute). In some embodiments, the constant NO flow rate is proportional to the minute volume of the dynamic inspiratory flow.

In some embodiments, the threshold for transitioning between dynamic and static proportional dosing is based on the relationship between inspired volume and bias flow. In one example, a neonate breathes at 50 ml breaths at 40 breaths per minute with 2 lpm bias flow. The inspired volume per minute is 2 liters (50 ml×40 bpm) which is equal to the bias flow resulting in an inspired to bias flow ratio of 1. In some embodiments, a NO delivery system changes to a static NO flow methodology when the inspired volume to bias flow volume falls below a threshold (e.g. a ratio=1). The inspired volume and bias flow volumes are calculated by the NO device controller based on inspiratory flow measurements either made by the NO delivery device or supplied from an external device.

Some modes of ventilation, such as high frequency oscillatory ventilation (HFOV) and jet ventilation, involve applying a high frequency “breath” cycle to the patient. The frequency of these treatments greatly exceeds the normal breath rates of a patient. Whereas patient respiratory rates rarely exceed 50 breaths per minute, HF treatments can involve breath rates of 300 to 900 per minute and jet ventilation can involve breath rates of 240 to 660 breaths per minute. In one embodiment, a NO delivery system controller determines the NO flow methodology (i.e. dynamic vs. static) based on the breath rate within the inspiratory flow. The breath rate is sensed by the controller in any number of ways including but not limited to counting the number of pressure pulses per unit time or counting surges in inspiratory flow rate per unit time. In one embodiment, the NO delivery device controller calculates the breath rate as the reciprocal of the breath period, the breath period being measured as the duration of time between repeated respiratory events (e.g. peak inspiratory flow, end of inspiration, beginning of exhalation, etc.), the respiratory events being detected by one or more sensors readings (e.g. inspiratory pressure sensor, inspiratory flow rate sensor, expiratory temperature sensor, etc.). In one embodiment, a NO delivery system uses a Fast Fourier Transform (FFT) analysis of inspiratory waveform to quantify the breath rate and determine the NO delivery methodology to apply. In another embodiment, FFT analysis is utilized by a NO delivery device controller to identify ventilation type. In some embodiments, the NO delivery mode is selected based on the ventilation type (e.g. conventional invasive ventilation vs. HFOV). Some ranges of respiratory frequencies are treated with constant flow NO and others are dosed proportionally.

### High Frequency Jet Ventilation

High Frequency Jet Ventilation (HFJV) is a method of invasive patient ventilation that utilizes rapid, low-volume breath pulses to oxygenate patient lungs. Most commonly used in neonates, this technology involves pulsed gas delivery to a pressurized ventilation circuit. The jet ventilator operates by pressurizing (e.g., up to 20 psig) and humidifying respiratory gas (e.g., air, oxygen) and releasing the gas in a pulsatile manner at frequencies of roughly 200 to 7000 “breaths” per minute. The low volume pulses induce gas exchange within the lungs without the risk of over pressurizing and potentially damaging the lung tissue.

Conventional, tank-based systems, introduce nitric oxide to a HFJV circuit in the high-pressure portion of the jet ventilator before the humidifier. The NO mixes with the medicinal gas and is metered out of the high-pressure portion of the system through a flow controller (AKA “flow interrupter”). Adding nitric oxide to a roughly 20 psig gas flow within the jet ventilator is straight forward when sourcing gas from a high pressure (e.g. 2000 psig) tank. NO generation systems, however typically operate at much lower internal gas pressure.

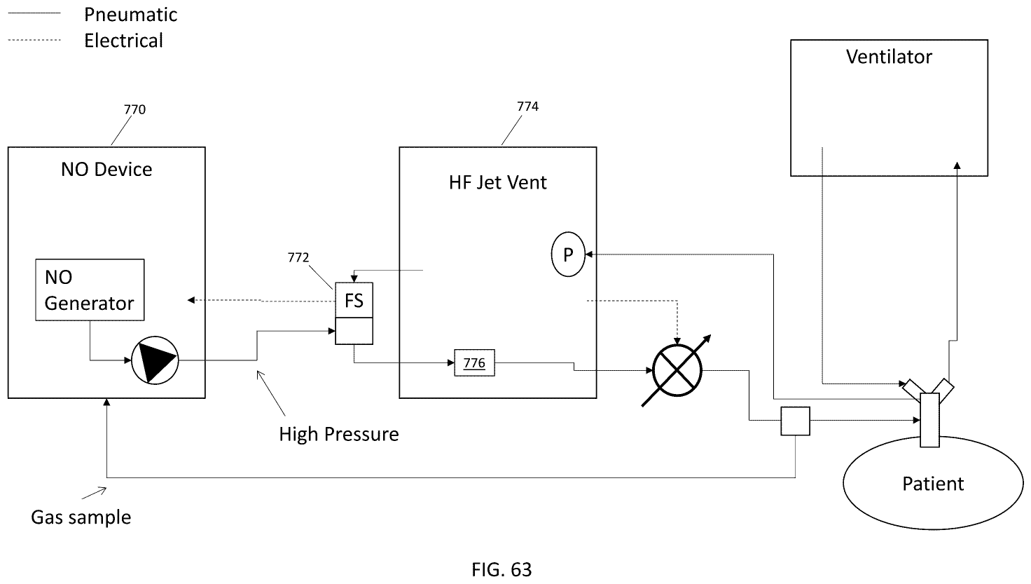

FIG. 63 depicts an embodiment of a NO generation system that delivers NO to the high pressure gas within a HFJV system. NO is generated within a NO device 770. The NO product gas pressure is then increased to a level that exceeds the pressure within the HFJV system 774 to ensure NO flow. A mass flow sensor 772 placed in the high pressure flow path of the HFJV system reports mass flow of the gas within the HFJV system to the NO device controller. This location for flow measurement can be advantageous because gas flow is less pulsatile than flow after the flow interrupter. Furthermore, the added volume of the flow sensor is upstream of the NO injection location, thereby not adding dead volume and time to the NO transit post-injector. The NO generation system releases a NO mass flow in proportion to the HFJV flow, according to the target NO dose. In some embodiments, the NO generation system releases NO at a constant mass flow into the HFJV flow. The controller determines the constant flow rate by measuring the Jet Vent flow rate over time and calculating an average volumetric flow rate (e.g. slpm). The controller then introduces NO to the HFJV circuit at a constant flow rate that is proportional to the average HFJV flow rate. In some embodiments, NO is introduced to the HFJV flow in a pulsatile fashion, in unison with the HFJV flow. In some embodiments, pulses of NO are introduced to the Jet flow immediately after the HFJV flow. This reduces the pressure that the NO system requires to deliver NO. The quantity of NO delivered to the HFJV system can be varied by the NO device controller by varying the product gas NO concentration and the product gas mass flow rate introduced to the HFJV system. In some embodiments, the mass flow sensor also reports a pressure measurement of the gas flow within the HFJV system. In some embodiments, the NO delivery system varies the product gas pressure according to the pressure measured within the HFJV system. In other systems, the NO delivery system operates at a consistent high pressure for all applications. While operating at a consistently high pressure can be simpler in ensuring that NO will enter the HFJV circuit, more NO can be expected to be lost to oxidation due to the high pressure. A gas sample is collected from the low-pressure portion of the HFJV flow to confirm that one or more gas concentrations (e.g., NO, NO₂, O₂, He) are within acceptable limits.

After injection of NO into the HFJV gas stream, the gas mixture can travel through a humidifier 776 and be released to the patient ventilator circuit through a flow controller controlled by the HFJV controller. A ventilator (depicted on the right side of the figure) provides a positive pressure to keep patient lungs inflated and a steady bias flow to sweep exhaled carbon dioxide out of the system. Inspiratory pressure is measured by the HFJV device, which modulates either pressure or flow within the HFJV system to maintain a target positive inspiratory pressure (PIP).

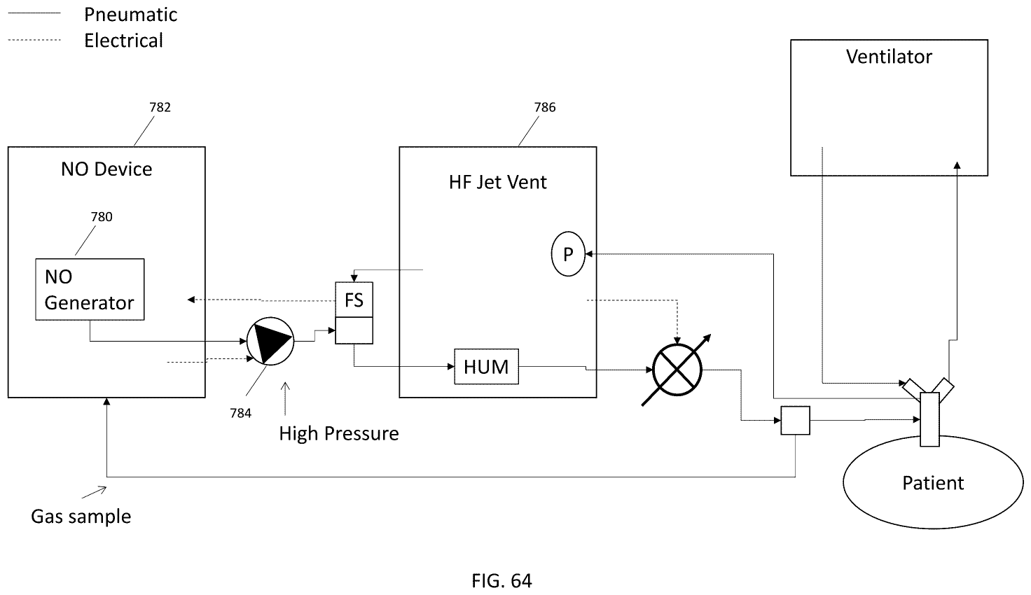

FIG. 64 depicts an exemplary embodiment of a NO generation system installed in a HFJV system. Low pressure NO product gas exits the NO generator 780 of the NO device 782. An external pump accessory 784 pressurizes the NO product gas to a pressure that exceeds the internal pressure within the HFJV system 786 to ensure positive flow of the product gas. Mass flow of the product gas is measured to ensure proportional dosing based on a mass flow measurement within the HFJV gas flow. This allows the external pump to act as an accessory to the NO generation system, only utilized in HFJV applications. The external pump accessory includes electrical and pneumatic connections to the main NO generation device. In some embodiments (not shown), one or more of the pump, mass flow sensor, and injector are combined into a single module to make the design more compact and reduce use steps in setting up the system. Similarly, the electrical and pneumatic connections for the pump and flow sensor can be combined to reduce use steps. Non-pulsatile (e.g., piezoelectric) or minimally pulsatile pumps are preferred so as to not disrupt the pressure controls within some HFJV systems. In some embodiments, the pump fills a reservoir that serves to attenuate pump vibrations (not shown). In some embodiments, the reservoir is at least partially filled with a NO₂ scrubbing or reducing material. In some embodiments, the outflow from the reservoir is controlled by a flow controller (not shown).

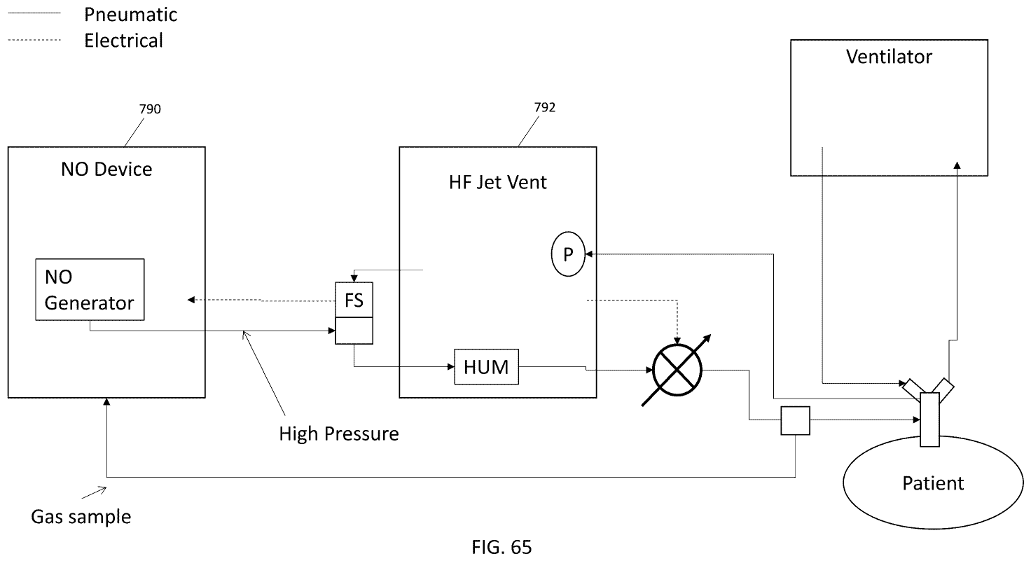

FIG. 65 depicts an exemplary embodiment of a NO generation system 790 that connects directly to a HFJV system 792. In this embodiment, the NO generation system increases its internal operating pressure to exceed that of the HFJV system. For example, a NO generation system operating with a recirculation loop architecture can increase the pre-injector gas pressure (i.e. the product gas pressure within the recirculation loop) to ensure positive gas flow into the HFJV system. A NO generation system that utilizes a pressured scrubber architecture can pressurize the scrubber to a pressure that exceeds the pressure within the HFJV system.

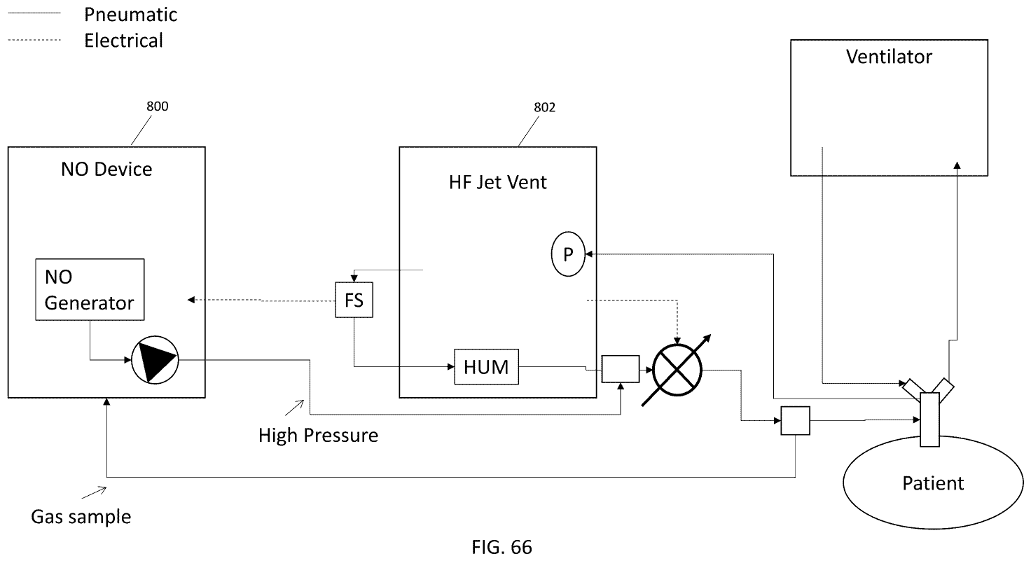

FIG. 66 depicts an exemplary embodiment of a NO generation system 800 that delivers high pressure NO gas to the HFJV system 802 after the humidifier and before the HFJV flow controller. The HFJV flow controller controls the flow of a mixture of HFJV gas and NO product gas. This configuration allows the NO to have a shorter transit time before reaching the patient resulting in lower inhaled NO₂ concentration, as the gas within the HFJV system is at high pressure and NO oxidation rates are proportional to pressure. The shorter transit time is most apparent when the HFJV gas is 100% oxygen. In some embodiments (shown), the NO device measures the flow rate of Jet ventilator gas within the high-pressure region of the jet ventilator upstream of the jet ventilator humidifier. In some embodiments (not shown), mass flow of the HFJV flow is measured between the humidifier and HFJV flow controller as well.

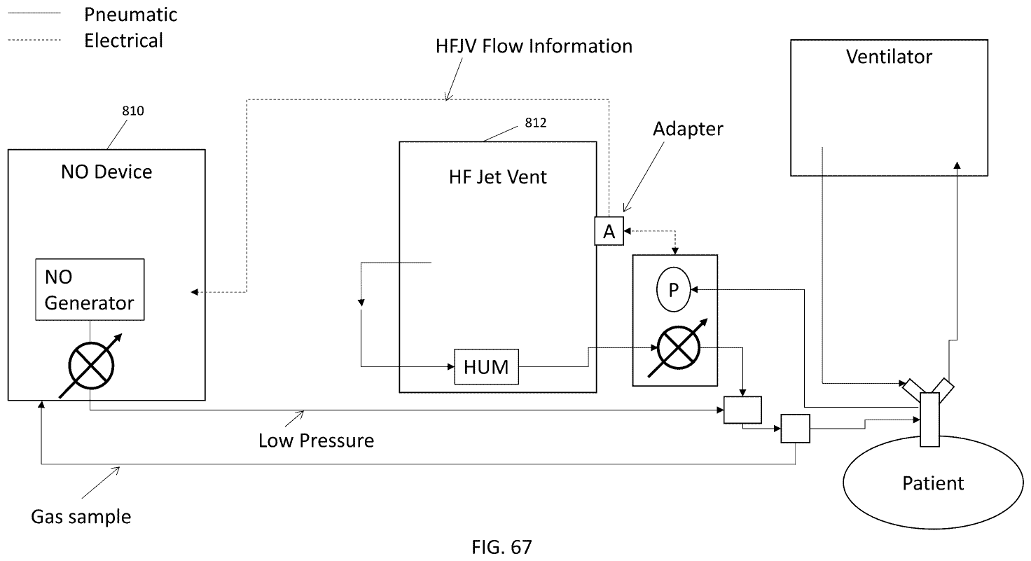

FIG. 67 depicts an exemplary embodiment of a NO generation system 810 that delivers NO to the low-pressure portion of a HFJV system 812. NO is delivered through an injector module inserted into the HFJV flow after the HFJV flow controller. In the embodiment depicted, the HFJV flow controller module includes a pressure transducer that measures pressure within the endotracheal tube. The external HFJV flow control module electrically connects to the HFJV controller through an adapter (labeled with an “A”). The adapter is part of the NO generation system that is inserted in series between the external HFJV flow controller and the HFJV controller. The adapter enables the NO generation device to know the timing of the HFJV pulses. This timing information is utilized to time the flow of NO pulses in unison with the HFJV pulses to proportionally and accurately dose the low pressure HFJV flow. In other embodiments (not shown), the NO device non-invasively detects the activity of the HFJV flow controller (e.g. capacitively, acoustically, vibrationally) using an appropriate transducer (e.g. capacitive sensor, microphone, accelerometer, respectively). NO flow is controlled within the NO device with a flow controller (shown). In some embodiments, the NO flow controller is located remotely, at or near the point of NO injection into the low-pressure side (not shown).

In some embodiments (not shown), one or more of the pressure and mass flow of the HFJV high pressure region is measured by the NO generation system. When pressure is measured, the NO generation system relies on the pressure/flow relationship of the HFJV controller to quantify the mass flow of gas exiting the HFJV flow controller. When mass flow of the HFJV is measured directly, it is equivalent to the mass flow downstream of the HFJV flow controller and can be used directly in calculating the proportional NO flow required. For example, if the mass flow rate at the high-pressure portion of the HFJV system is 3 slpm, the target dose is 20 ppm, the target NO production level is 3 slpm×20 ppm, or 60 ppm.slpm. If the product gas concentration is 600 ppm, then the required product gas flow rate is 60 ppm/slpm/600 ppm, or 0.1 slpm.

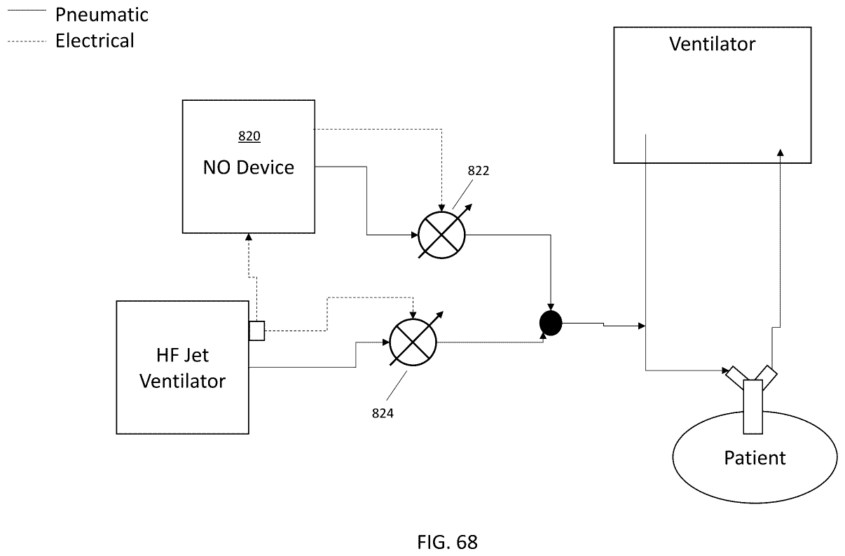

FIG. 68 depicts another exemplary embodiment of a NO device 820 that monitors activity on a HFJV flow controller line. The NO device operates a NO flow controller 822 in synchrony with the HFJV flow controller 824 to proportionally match gas flow rates to accurately dose the HFJV flow.

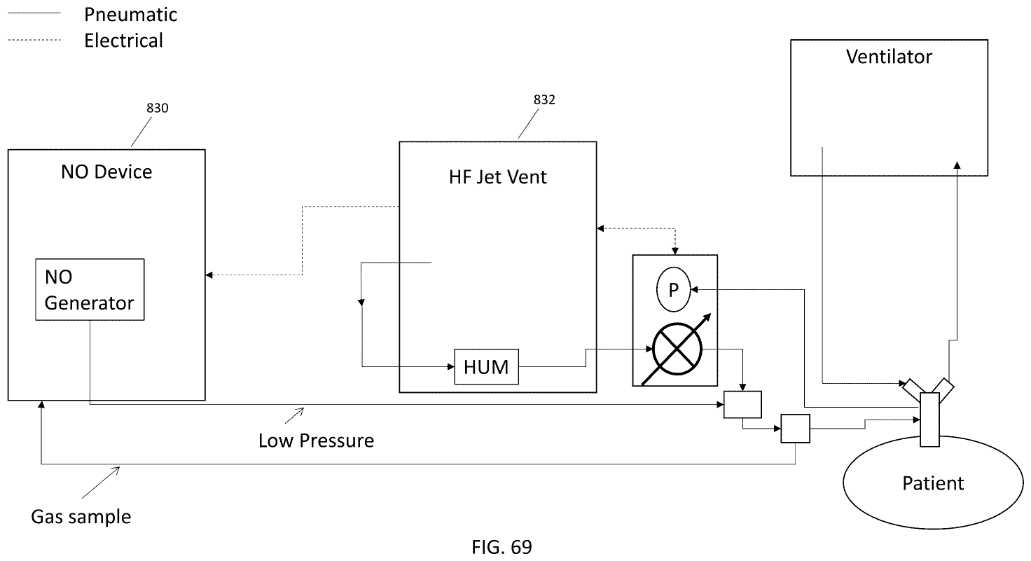

FIG. 69 depicts another exemplary embodiment of a NO generation system 830 dosing a HFJV system 832 wherein the HFJV system communicates via wired (shown) or wireless (not shown) means one or more of the HFJV mass flow rate, the HFJV system pressure, and the HFJV flow controller pulse timing to enable the NO generation device to accurately dose the flow from the HFJV device.

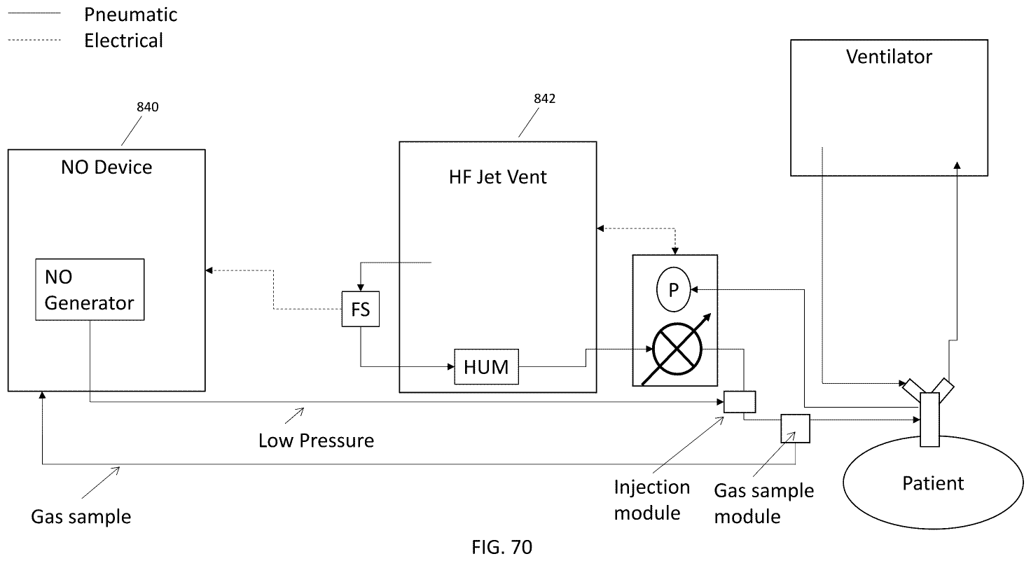

FIG. 70 depicts an exemplary embodiment of a NO generation system 840 dosing a HFJV 842 where in the NO generation device detects mass flow level and perturbations in the higher-pressure portion of the HFJV system. The NO generation system controller analyzes the flow within the HFJV system to determine the timing of gas pulses delivered to the patient. In turn, the NO generation system delivers pulses of NO, synchronized and in proportion with the HFJV pulses to accurately deliver the patient inspiratory flow with NO.

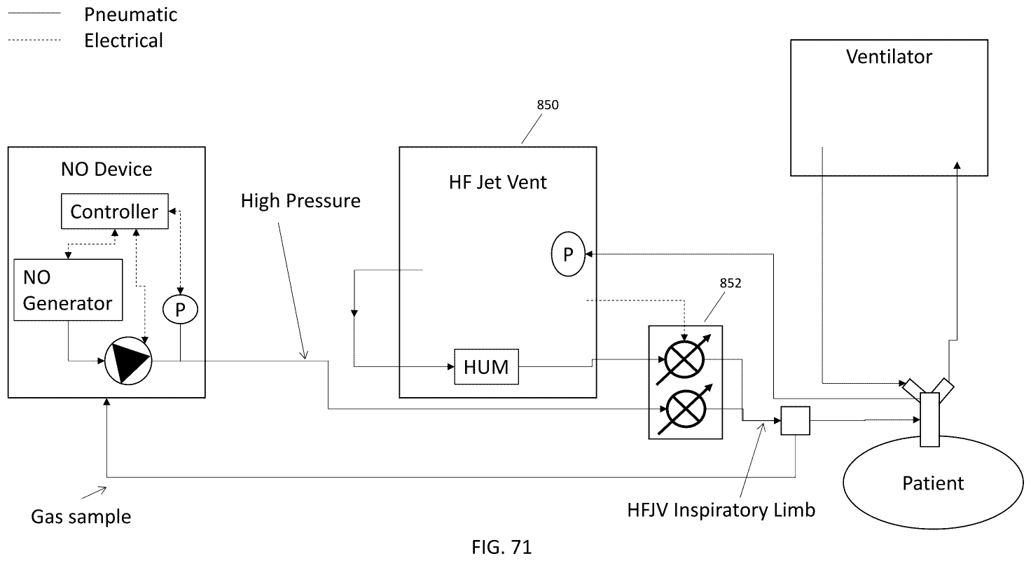

FIG. 71 depicts an exemplary embodiment of a HFJV system 850 that maintains separation between HFJV gas and NO gas until the HFJV flow controller 852. In some embodiments (not shown), NO is sourced from a NO generator built into the HFJV ventilator. In other embodiments (shown), NO is sourced from an external source (bottle, generator, etc.). Pressurized NO is provided to the HFJV flow controller. The HFJV flow controller blends the HFJV gas (e.g. air, oxygen) with the NO gas as the two gases are released into the patient inspiratory circuit. In the depicted embodiment, the NO is sourced from a NO generator. NO product gas is pressurized to a target pressure indicated by a pressure sensor under the control of the NO generation device controller. The pressure within the NO product gas must exceed the pressure within the HFJV inspiratory limb for NO product gas to flow toward the patient. In one embodiment, the pressure at the HFJV inspiratory limb is measured by a pressure sensor within the NO device (not shown) through the gas sample lumen. In some embodiments (not shown), the NO generator and HFJV devices are combined into a single device to save space and reduce complexity and use steps. In some embodiments, combined systems share one or more of the user interface, power supply, controller (e.g. microprocessor), inlet gas filter, and inlet gas scrubber.

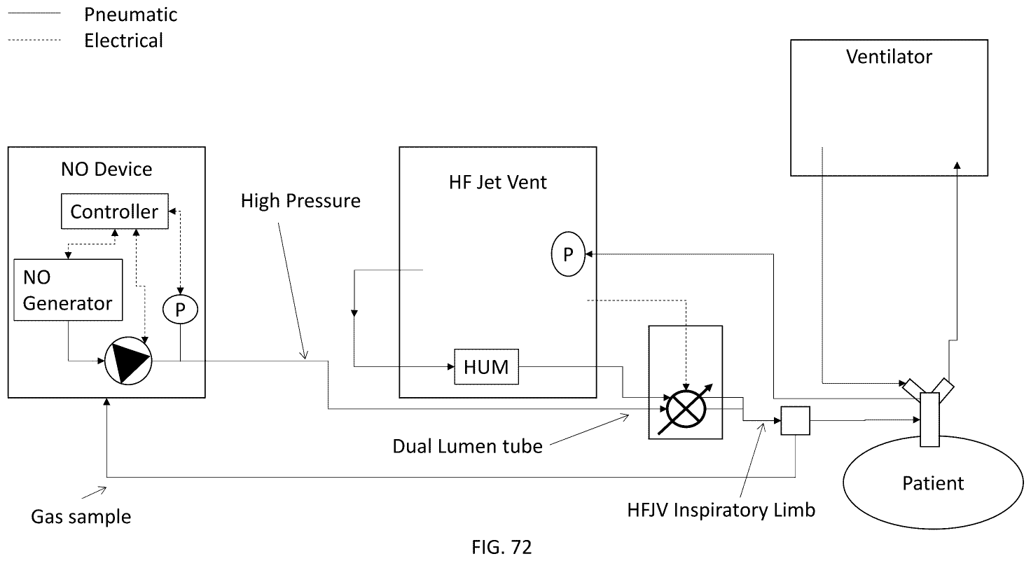

FIG. 72 depicts an embodiment that utilizes a dual-lumen tube to carry NO product gas and HFJV gas in independent lumens. A first lumen flows HFJV gas (e.g. air, oxygen). A second lumen flows NO product gas. The dual-lumen tube is placed in a common flow interrupter (e.g. a pinch valve), controlled by the HFJV controller. When in the closed position, the flow interrupter interrupts flow in both lumens at the same time. In this embodiment, the flow of NO product gas and HFJV gases are synchronized in time.

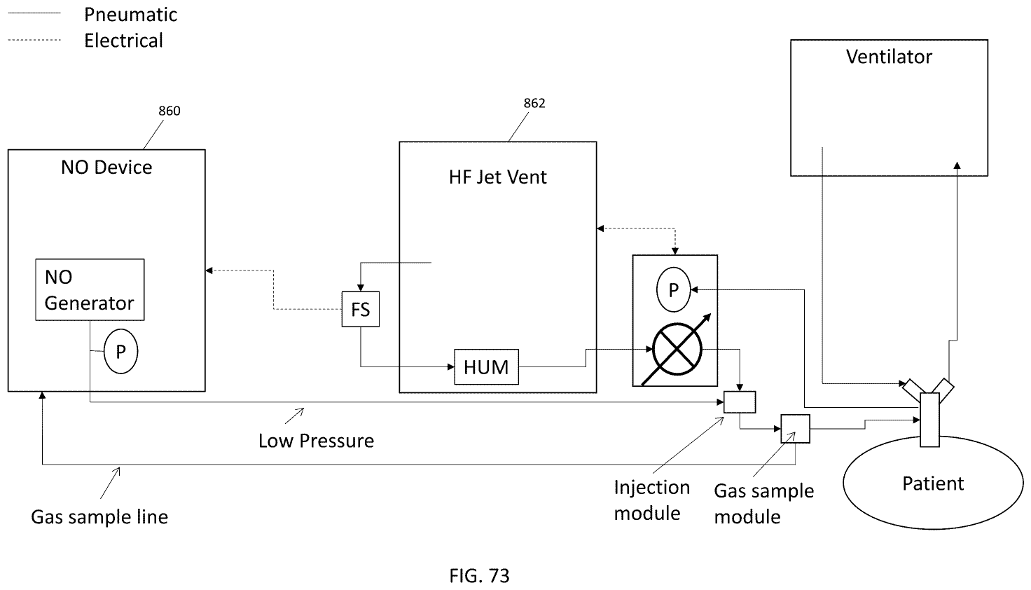

FIG. 73 depicts an exemplary embodiment of a NO delivery system 860 operating with a HFJV system 862. The NO delivery system quantifies gas flow through the HFJV system by one or more of measurement with a flow sensor (shown) or measurement of a pressure (not shown). When a pressure is measured, the pressure is utilized along with a characterization of the HFJV flow controller to determine the flow rate through the system. In some embodiments, the NO delivery system utilizes pulsations in the high pressure flow or pressure signal to determine timing of the pulsations of the HFJV flow. In some embodiments, the NO delivery system detects the timing of the HFJV pressure signal by a pressure sensor in fluid communication with the NO delivery tube (shown). The NO delivery system then delivers NO in a pulsatile pattern that proportionally matches that of the HFJV flow. In some embodiments, the NO delivery system, based on known characteristics of the NO delivery tube (e.g. flow restriction) intentionally operates with a phase delay so that the NO pulses exiting the NO delivery tube are in sync with the HFJV pulses.

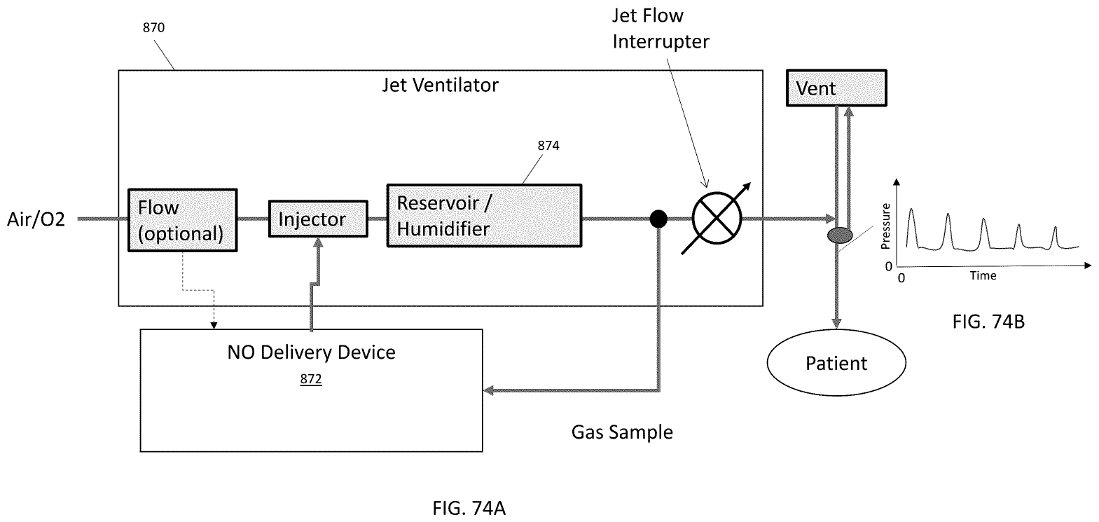

Jet ventilation is a non-invasive method for providing oxygen and other gases to a patient that requires elevated peek end expiratory pressure (PEEP). In this method, a ventilator is utilized to maintain PEEP while a jet ventilator is utilized to provide high-frequency (up to 900 “breaths” per minute) pulses of inspiratory gas to the patient. FIG. 74A depicts an exemplary embodiment of a jet ventilation system 870 in combination with an NO delivery system 872. Inspiratory gas (e.g., O₂, air) is introduced to the jet ventilator. In some embodiments (shown), input gases are provided under pressure. In some embodiments (not shown), the jet ventilator includes an internal pump to bring the gases up to a target internal pressure. Pressurized gas is stored in a reservoir 874 and optionally humidified. An optional flow sensor located in the input gas stream (before or after the pump) can inform the NO device of the mass flow of gas to be dosed with NO. NO is introduced to the input gas flow at the appropriate flow rate to achieve a target NO concentration within the jet ventilator gas, as indicated by NO concentration measurements made from a sample of gas withdrawn before the jet ventilator flow controller. This provides closed-loop control of NO concentration within the Jet ventilator based on measurements by the NO sensor. Jet ventilator gas containing NO is introduced to the patient limb in a pulsatile fashion. Using this approach, it will be noted that the flow measurement of the jet ventilator gas is optional, thereby simplifying the system while ensuring accurate NO delivery with the assumption that all of the patient inspiratory volume is provided by the jet ventilator and the ventilator is only utilized to provide PEEP. FIG. 74B depicts an exemplary graph of the pulsed inspiratory pressure within the patient during treatment.

Delivery of NO During Manual Breathing (i.e. Bagging)

In some embodiments of an NO delivery system, NO gas is delivered in a pulsatile manner to one or more breaths. In some embodiments, the user sets one or more of the following parameters in the system: NO pulse duration, pulse flow rate, frequency (e.g. every breath, alternative breaths, time period between pulses, etc.), NO concentration (within the concentrated pulse or at the patient), and NO dose (e.g. mg/hr). The system then delivers NO according to these settings, independent of patient respiratory rate, tidal volume, and inspiratory flow rate. In some embodiments, the NO delivery system monitors inspiratory flow with a flow sensor and delivers a pulse of NO during inspiratory events.

In some embodiments, the bag gas flow measurements are analyzed by the NO delivery device. For example, in one embodiment, the system quantifies the number of bag squeezes, and/or the duration of bag squeezes. The system can then use this information to quantify the volume of gas entering the patient and the NO dose delivered. The duration of a single squeeze of a bag relates to the quantity of gas that came out of the bag.

In some embodiments, the mixture of gas entering the bag is analyzed by a NO delivery device. In some embodiments, this is done by directing a sample gas flow from the bag gas flow to a gas analyzer. In some embodiments, the gas analyzer is integrated into the NO delivery system. The delivery device controller can utilize the gas mixture information (e.g. oxygen concentration) to adjust the bag mass flow sensor calibration for improved accuracy. Improved bag flow measurement accuracy results in improved NO dose accuracy.

### Patient Monitoring

In some embodiments, a NO generation and/or delivery device monitors patient respiratory events. In some embodiments, the device monitors the patient by one or more of measuring pressure within a tube that is in fluid communication with a patient airway, monitoring sound levels (i.e. a microphone) near a patient and monitoring a flow rate within a gas flow tube in fluid communication with a patient. In one embodiment, the device can identify cough events. In one embodiment, the device aggregates cough data into metrics (e.g. quantity of coughs per unit time, magnitude of the cough). In some embodiments, the number of coughs per unit time and/or cough magnitude are utilized as a measure of patient health. In some embodiments, cough data are presented to a user directly or through a wired or wireless communication method.

### Infection Treatment

High dose NO (i.e. >80 ppm, >160 ppm, >300 ppm, >500 ppm) can be used to treat infection. In some embodiments, a system with multiple NO generators generates a high dose of NO by operating more than one NO generator simultaneously and injecting their product gas streams into an inspiratory flow. The risk associated with lack of NO generation redundancy (i.e. a backup NO generator) for a high-dose NO treatment can be acceptable because high dose NO treatment is not life support. Hence, if one or both of the NO generators fail, a back-up generator can be sourced to continue treatment without risk to the patient. In an example of combining the output of multiple NO generators, a NO generation system includes two NO generators, each capable of generating 4800 ppm.lpm of NO. Each system can inject up to 3 lpm of product gas at 1600 ppm into an inspiratory limb flowing at 20 lpm. When the maximum production of a first NO generator is introduced to the inspiratory flow, the resulting inhaled NO concentration and flow rate are 209 ppm and 23 lpm, respectively. When the product gas from both generators are introduced to the inspiratory flow, the resulting inhaled NO concentration and flow rate are 369 ppm and 26 lpm, respectively.

In some embodiments, high dose NO (i.e. NO concentrations >160 ppm) is delivered with helium gas. The lower viscosity of the helium gas enables lower breathing effort and improves NO gas penetration into obstructed regions of the lung to improve infection treatment.

In some embodiments of a NO delivery system, a patient is treated for a first condition (e.g. hypoxia) at a first dose of NO (e.g. 20 ppm, 4 mg/hr, etc.). The system periodically doses the patient at a second dose (e.g. 300 ppm) to prevent and/or treat an infection of the lungs or airway. In one specific example, a patient is delivered pulsatile doses of NO to a plurality of breaths over time amounting to a delivery rate of 4 mg/hr to treat pulmonary hypertension. Periodically (e.g. every hour), the device increases the inhaled concentration to an anti-infection level (e.g. >160 ppm, depending on the microbe to kill), for a period of time (e.g. a set number of minutes or breaths) to treat the patient for infection.

Electrical generation of NO can be used directly, without additional gas mixing, when delivering high doses of NO because the NO product gas contains oxygen. At therapeutic levels of NO to treat infection (e.g. 160 ppm, 300 ppm, 600 ppm), the amount of oxygen within the product gas is essentially unaltered from atmospheric 21,000 ppm. In contrast, high dose treatment with NO sourced from a gas cylinder presents a risk of low FiO₂ levels, requiring supplemental oxygen. For example, a patient treated with 300 ppm NO from a cylinder of 900 ppm NO in a balance of N₂, would require ⅓ of the gas they breath to come from the NO cylinder, resulting an FiO₂ of 14%. With a 5000 ppm NO tank, a treatment of 300 ppm NO would dilute inspiratory air from 21% to 20%.

### Bidirectional Communication

In some embodiments, a NO generation and/or delivery system is capable of bidirectional communication with an external device. Examples of external devices include but are not limited to ventilators, patient monitors, CPAP machines, and anesthesia machines. In some embodiments, the NO generation and/or delivery device receives signals and/or data (e.g. oxygen saturation, pulmonary artery pressure, systolic blood pressure, diastolic blood pressure, heart rate, respiratory rate, tidal volume, airway pressure, inspiratory flow rate). In some embodiments, information received from an external device is presented on a trending screen or stored in a data file. In some embodiments, information received from an external device serves as an input to a NO dose control algorithm. For example, ventilator flow information can be utilized to quantify the amount of gas flowing within an inspiratory circuit that requires NO dosing. In another example, when the SpO₂ is low, as indicated by an external device, a NO delivery system can increase the NO dose delivered to the patient automatically. Typically, automated NO dose control is performed within User-selected limits for NO concentration and NO concentration rate of change. In some embodiments, automated NO dose control is referred to as “physiologic closed loop control.”

In some embodiments, the NO generation and/or delivery device is also capable of sending information to an external device. Examples of information sent from the NO generation and/or delivery device to an external device include but are not limited to inhaled NO concentration, scrubber status, battery status, and gas measurements (e.g. NO, NO₂, O₂, He). In some embodiments, the external device displays information from the NO generation and/or delivery device on its user interface. For example, a ventilator can display inhaled NO concentration on the ventilator user interface screen. In some embodiments, the user interface of the external device includes controls for the NO generation and/or delivery device enabling a user to control the NO generation and/or delivery device remotely.

In some embodiments, a NO generation and/or delivery device provides clinical decision support. Users can obtain information related to a treatment in the form of trending tables, trending plots, sensor measurement displays from the NO generation and/or delivery device. In some embodiments, a NO generation and/or delivery device follows a weaning protocol to wean a patient from NO. In some embodiments, the weaning protocol (or other treatment protocols) is entered by a user or their institution into the NO device. In some embodiments, the NO generation and/or delivery device follows the weaning protocol automatically. In some embodiments, the NO generation and/or delivery device prompts the user for approval to advance to one or more of the steps in a weaning process. In some embodiments, a NO generation and/or delivery device holds multiple, pre-programmed weaning protocols for a user to select from. An example of another protocol that can be entered by a user into a NO device is a treatment onset protocol. An institution sets the initial NO dose for a particular therapy (or in general) and optionally sets the time period and step increment for NO that the system follows based on patient condition (e.g. SpO₂, cardiac output, pulmonary artery pressure, etc.)

In some embodiments, a NO delivery device has a “wean” button (physical or virtual on touch screen). When pressed, the system decreases the current inhaled NO concentration by an amount. In some embodiments, the amount of dose decrease is set by the user in the device settings. In some embodiments, the dose is decreased by 50% when the “wean” button is pressed.

In some embodiments, the user interface of a NO generation and/or delivery system displays a “pick list” of pre-programmed values for data entry. For example, a user can select a box on a touch screen for treatment duration and select from a menu of durations. In some embodiments, a NO generation and/or delivery system includes an oxygen index calculator, whereby the system calculates the oxygen index from one or more of user-entered data, received data from an external device, and parameters the device measured directly. The oxygen index is defined as the mathematical product of FIO2 (%), mean airway pressure (cmH20) and 100 divided by the PAO2 (mmHg). In some embodiments, a NO generation and/or delivery system includes one or more of a PFratio (i.e. PaO2/FIO2 ratio) calculator and a SFratio (i.e. SpO₂/FIO2) calculator that makes the calculation based on one or more of User-entered data, received data from an external device, and parameters measured by the device directly. User-entered values are entered using the user interface. Calculated values are one or more of displayed on the user interface, logged in the data file, and displayed on trending screens.

### Sterile Field Applications

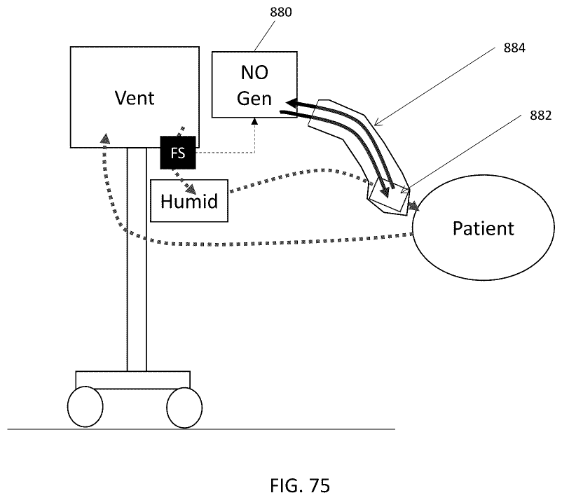

In some embodiments, NO can be utilized in additional types of treatments, including surgeries and other treatments that require a sterile field. Providing a sterile surface to the sterile field presents a challenge with respect to device cost. Device components must either be disposable or sterilizable for re-use. FIG. 75 depicts an exemplary NO delivery system 880 setup applied to a ventilator circuit. NO injection and gas sample measurement are delivered proximal (close) to the patient. Given the proximity to the patient, it is desirable for all surfaces to be sterile. In this embodiment, the injection module 882 is connected to the inspiratory limb. The injection module includes a sterile sleeve 884 that extends and covers the portion of the NO lines that are in the sterile field at a minimum. In some embodiments (not shown), the NO injection module includes a gas sampling feature to collect inspiratory gas samples post-NO injection. In some embodiments, inspiratory gas samples are analyzed for one or more of nitrogen dioxide, nitric oxide, oxygen and helium.

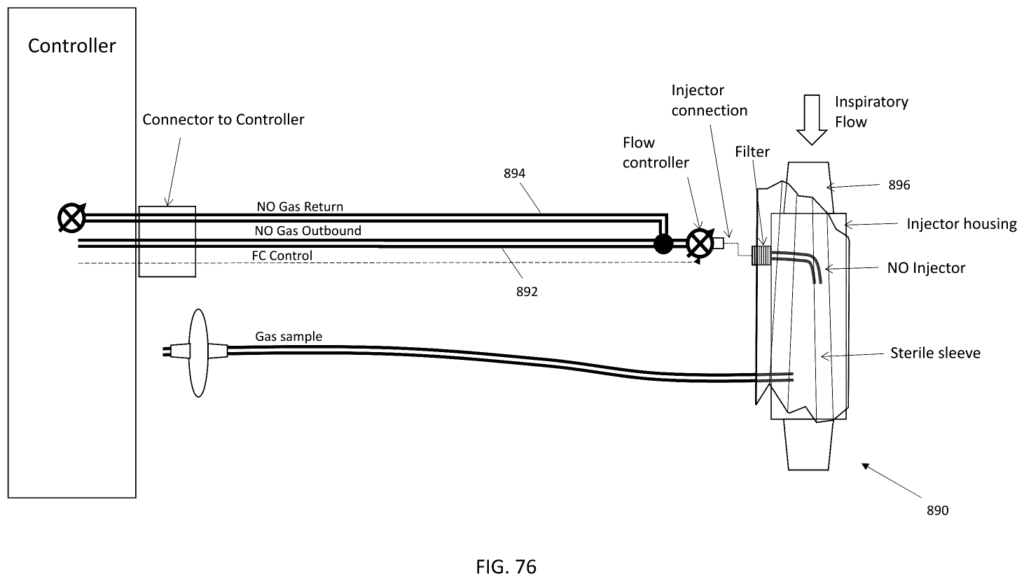

FIG. 76 depicts an exemplary embodiment of external portions of a NO generation and delivery system. A NO generation assembly connects to a remote NO injection module 890 with one or more pneumatic or electrical connections (single connector shown). The assembly includes a NO delivery lumen 892 (also known as an “outbound lumen”), an NO return lumen 894 and 2 or more electrical conductors for actuating a flow control valve. In some embodiments, there are two or more of the outbound lumens and return lumens (not shown) for fault-tolerant, continuous NO delivery in the event that an outbound lumen is kinked or occluded. In some embodiments, the gas lumens have structures within them (not shown) to resist complete occlusion during kinking (e.g. ribs). The flow control valve within the injection module connects to an optional filter and NO injector. When included in the design, the filter protects the valve and NO lumens from potential contamination from the inspiratory limb. The injector is mounted in a housing that connects to the inspiratory limb with standard conical connectors 896. The injector housing may include one or more features to promote mixing between the NO-containing gas and inspiratory gas (e.g. static mixer, multiple-orifice injection). In some embodiments, as shown, a gas sample of the mixed gas is optionally collected from the housing downstream of NO injection. The optional gas sample lumen includes a sterile filter. The sterile filter protects gas sensors within the NO device from contamination from the inspiratory limb such as nebulized drugs. The gas sample filter is located near the NO delivery device and is typically user-replaceable. In one embodiment (shown), a sterile sleeve is provided on the injector housing. After the NO injector is connected to the injector housing filter, the sterile sleeve is extended over the NO delivery lumens and gas sample line, rendering the entire assembly sterile on the outer surfaces.

Given that the NO injection assembly shown requires user-established connections in the NO delivery and NO return lumens, some NO delivery systems perform a leak test on the NO delivery assembly prior to use to ensure that the system does not leak. In one embodiment, the NO device controller closes the NO injection flow controller to create a closed volume. Then, the NO delivery system introduces gas (e.g., air) to the delivery system to increase the pressure to a specific level. The device controller then performs a leak-down test by monitoring the pressure within the closed system. If the pressure falls faster than a pre-established acceptable rate, it is indicative of a leak in the NO delivery assembly and/or connections. In the event that the leak test fails, the controller does one or more of notify the user to replace the NO delivery assembly, instruct the user to check connections on the NO delivery assembly, and automatically repeat the test for confirmation. In some embodiments, the leak test is conducted immediately and automatically after connection of the NO delivery assembly. In some embodiments, the device controller uses detection of electrical connection with the NO injection flow controller as an indication that the NO delivery assembly has been connected. In some embodiments, calibration information (e.g., flow vs. voltage information) for the NO injection flow controller is stored in a memory device within the NO delivery assembly connector.

In some embodiments, product gas returning from the remote injection module is merged with incoming reactant gas and passed through the plasma chamber again, forming a recirculation loop. In other embodiments, product gas that is not injected into the inspiratory flow is released into the atmosphere. In some embodiments, product gas is released into the atmosphere at the remote injection module. In other embodiments, the product gas is released into the atmosphere between the remote injection module and the controller. In other embodiments, the product gas is released into the atmosphere at or within the controller. In some embodiments, product gas is scrubbed for one or more of NO and NO₂ prior to release into the atmosphere.

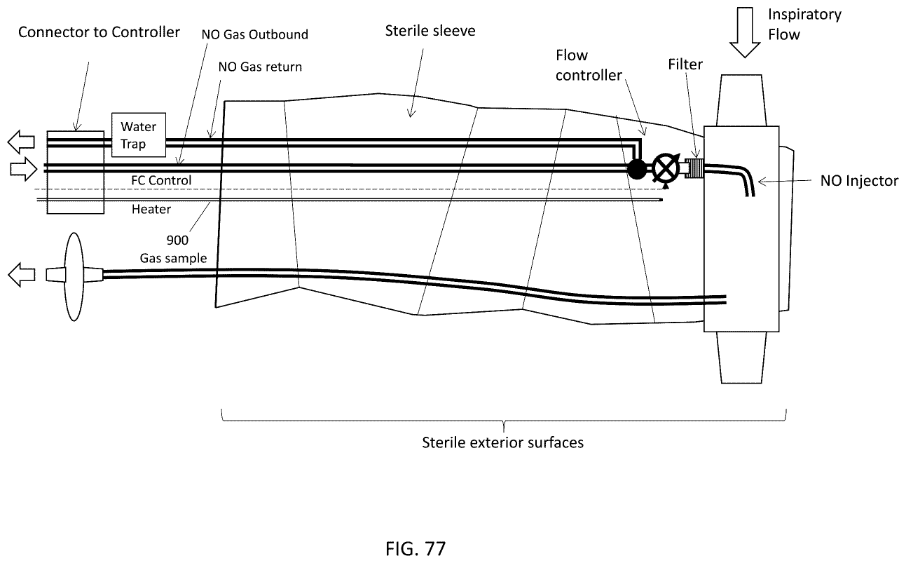

FIG. 77 depicts an exemplary embodiment of a NO delivery and gas sampling assemblies with the sterile sleeve deployed. In some embodiments, the NO delivery assembly is sterilizable (e.g., EtO, autoclave). A heater circuit 900 (e.g. nichrome wire loop) can be used to keep one or more pneumatic pathways warm to prevent product gas condensation within the external gas pathways (recirculation paths, injection module). In some embodiments, the heater circuit is controlled by the controller using a temperature measurement (e.g. ambient temperature, line temperature, injection module temperature) as a control input. In some embodiments, the heater is energized in all use scenarios to simplify the system controls. In some embodiments, an optional water trap (shown) collects water in the returning product gas flow to prevent liquid water from affecting internal components of the NO generation system.

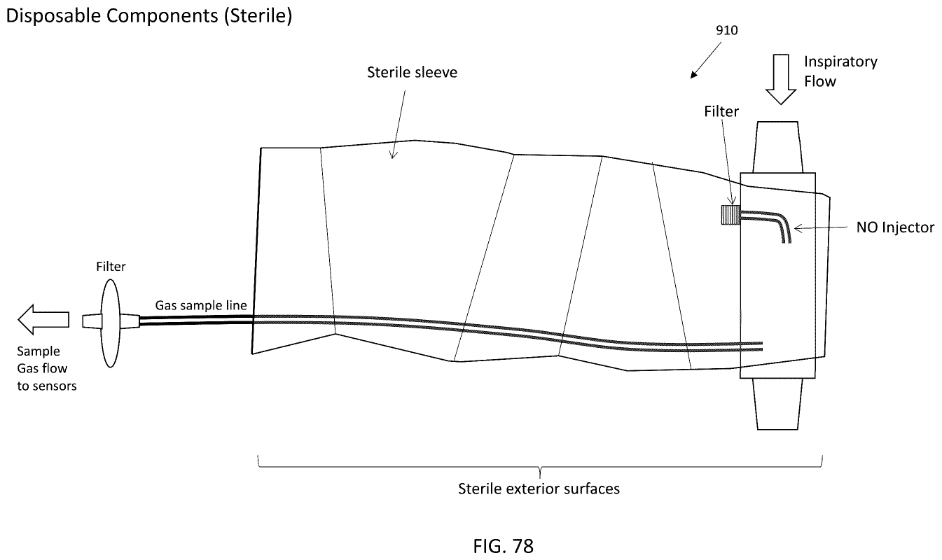

FIG. 78 depicts an exemplary embodiment of the NO injector, gas sample and sterile sleeve assembly 910. In some embodiments, this assembly is single-patient use and disposable. In other embodiments, portions of the assembly can be re-sterilized and other portions (e.g. the filters) are single use and disposable.

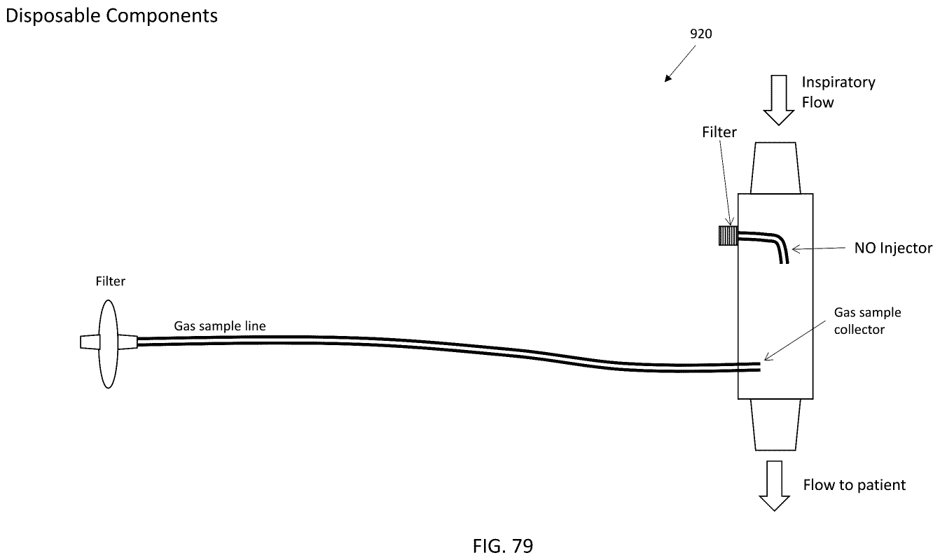

FIG. 79 depicts an exemplary embodiment of a NO injector and gas sample assembly 920 provided without the sterile sleeve. This assembly can be appropriate for patient treatments that do not require a sterile field. In some embodiments, the product gas is actively or passively mixed into the inspiratory gas flow between the NO injector and gas sample collector.

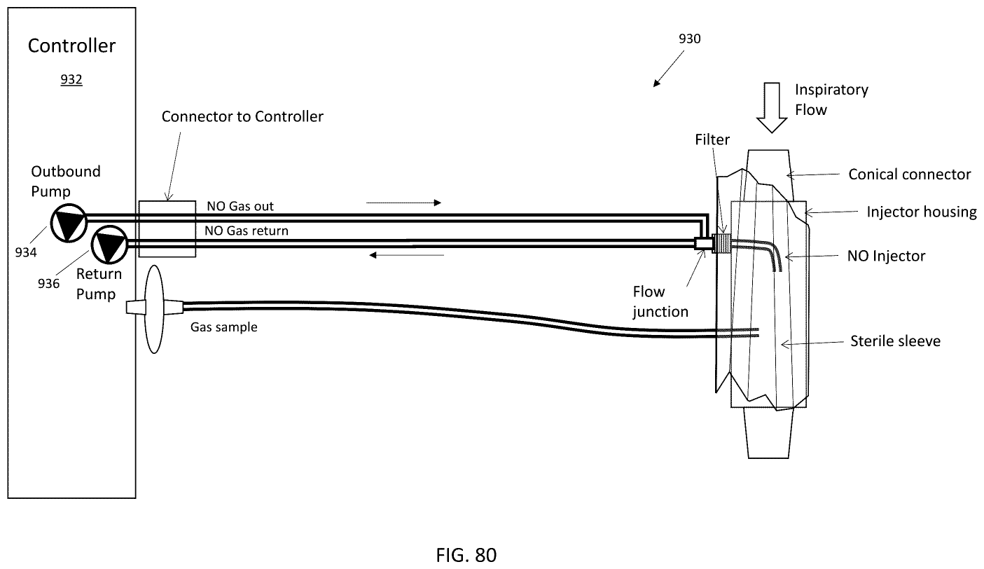

FIG. 80 depicts an exemplary embodiment of a NO injector and gas sample assembly 930 that utilizes flow control within a controller 932. The outbound flow pump/controller 934 provides a constant flow rate of NO product gas through the injector assembly. The return flow pump/controller 936 modulates the return flow. When the return flow controller slows or restricts return flow, pressure builds within the injector assembly and flow is directed towards the patient inspiratory flow. When the return flow pump matches the outbound flow, NO injection into the inspiratory flow is at or near zero. This allows for external recirculation lines do not include any electronics, thereby reducing cost and complexity.

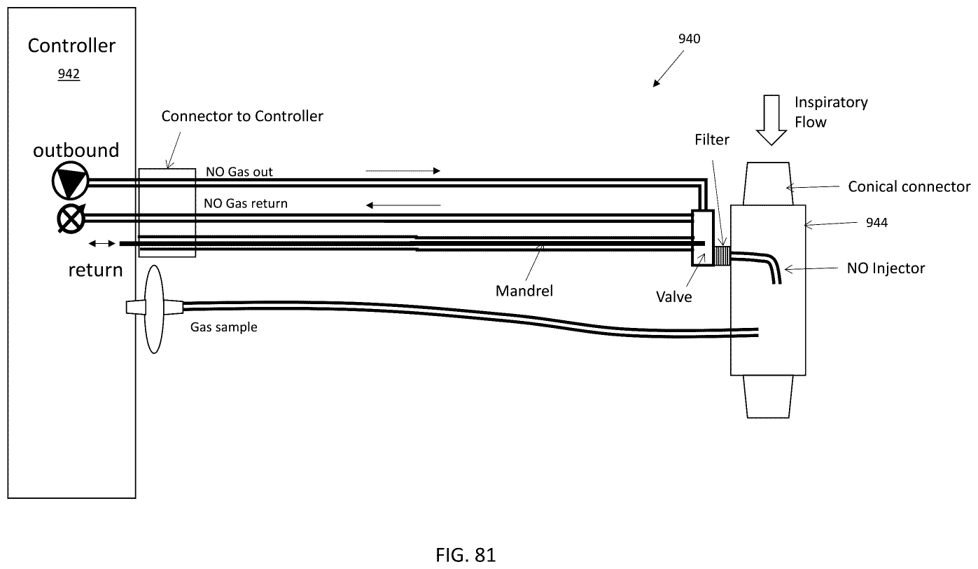

FIG. 81 depicts an exemplary embodiment of a NO injector assembly 940. Three lumens extend from a controller 942 to the injector housing 944. The first lumen delivers NO product gas to the injector housing. A second lumen permits NO product gas to return to the controller. The third lumen houses a mandrel that can translate within the lumen. Translation of the mandrel is controlled by an actuator within the controller (e.g. linear actuator, screw mechanism, solenoid, rotational motor, etc.). When the mandrel is fully pushed into the third lumen, it bottoms out at the injector to block flow of NO product gas into the inspiratory flow. As the mandrel is retracted back towards the controller, a flow path opens enabling product gas to flow into the inspiratory flow. The mandrel valve can be utilized to modulate product gas flow in proportion to inspiratory limb flow. In some embodiments, the controller initially bottoms out the mandrel into the third lumen to “zero” the system and records the position of the mandrel associated with zero flow. Position of the mandrel is measured by measuring the position or orientation of the positioning component within the controller. The zero position can be utilized to vary the y-offset in the valve flow characterization equation defining the pressure/flow/mandrel position relationship. In some embodiments (not shown), mandrel translation is achieved by rotating the mandrel about its long axis so that it travels along screw threads (i.e. like a rotary needle valve). In some embodiments, the mandrel is actively pushed and actively pulled by the controller. In another embodiment, the mandrel is biased to one position (e.g. by a spring) and actuated the other direction by the controller moving the mandrel. This approach allows for a NO delivery assembly that is very simple, reducing cost and enabling the assembly to be disposable. It can also be constructed without metallic components, which allows for the NO injector to be located close to the patient during magnetic resonance imaging (MRI) procedures.

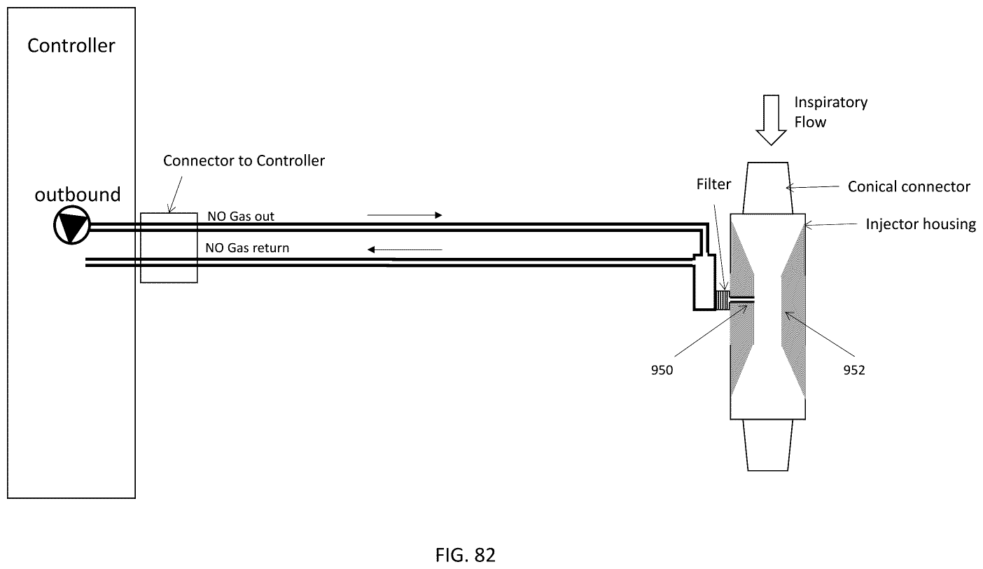

FIG. 82 depicts an embodiment of an NO injector 950 that utilizes a venturi 952 to draw NO product gas into the inspiratory flow. The inspiratory flow is directed through a venturi which increases the velocity of the inspiratory gas. The venturi includes a NO product gas supply line connecting within the high velocity region. The higher velocity of the product gas induces suction on the NO line, drawing NO into the inspiratory flow. The quantity of NO added to the inspiratory flow increases with increased inspiratory flow.

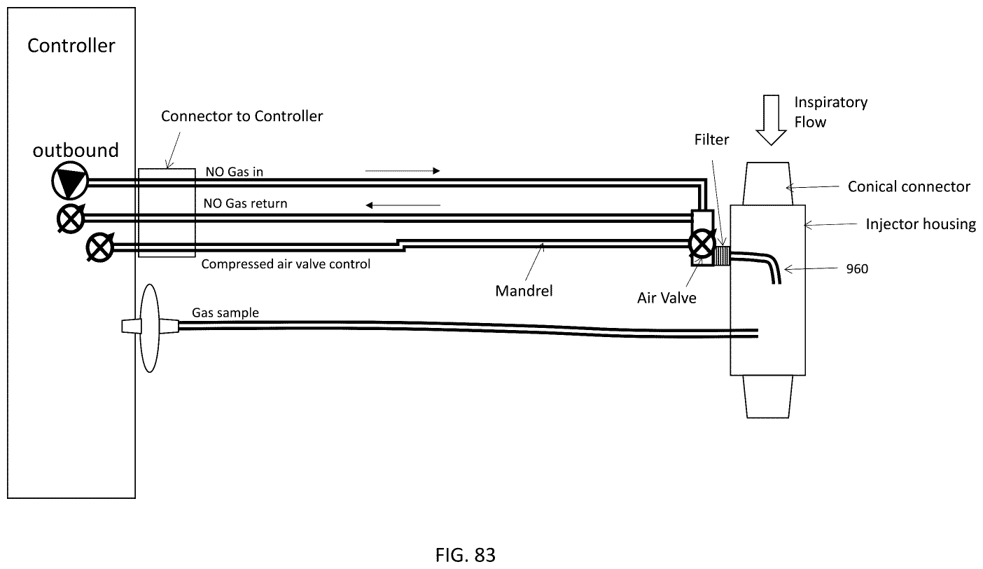

FIG. 83 depicts an embodiment of a remote NO injector 960 that utilizes a compressed gas-actuated valve to modulate NO injection into the inspiratory flow. Gas (e.g. NO product gas, air, etc) is compressed within the controller or comes from an external source. The gas pressure within the valve control lumen is controlled by a flow controller or pump within the NO delivery controller. In some embodiments, as the pressure increases in the valve control lumen, the valve closes. In some embodiments, as the pressure increases in the valve control lumen, the valve opens. This approach has no electrical conductors within the remote NO delivery subassembly, thereby reducing artifact and heating associated with magnetic fields during MRI. In some embodiments, the valve is constructed entirely of non-ferrous, non-electrically conductive materials to prevent heating and MRI image artifact.

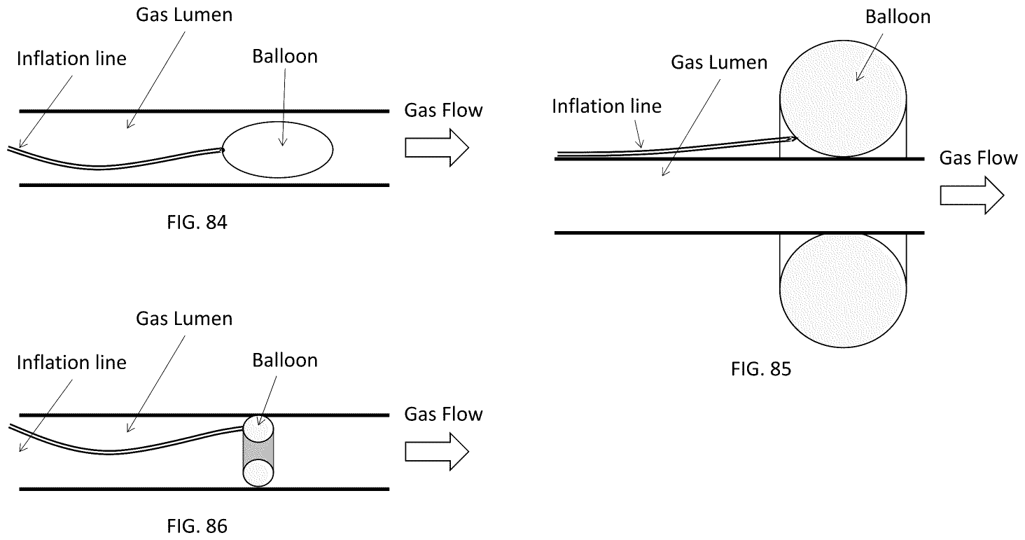

In some embodiments involving remote gas injection (i.e. injection of NO into an inspiratory gas stream external to the NO delivery device), the gas flow is modulated by a flow controller within the NO delivery device. In other embodiments, the gas flow controller is located at the injection location (i.e. external to the NO delivery device). External gas flow controllers can take many forms including but not limited to a balloon with a lumen that variably occludes gas flow (FIG. 84), an annular/torroidal balloon that squeezes a gas flow tube (FIG. 85), and an annular/toroidal balloon that variably occludes gas flow (FIG. 86). In some embodiments, the fluid utilized to actuate the flow controller is a liquid. In other embodiments, the fluid utilized to actuate the flow controller is a gas.

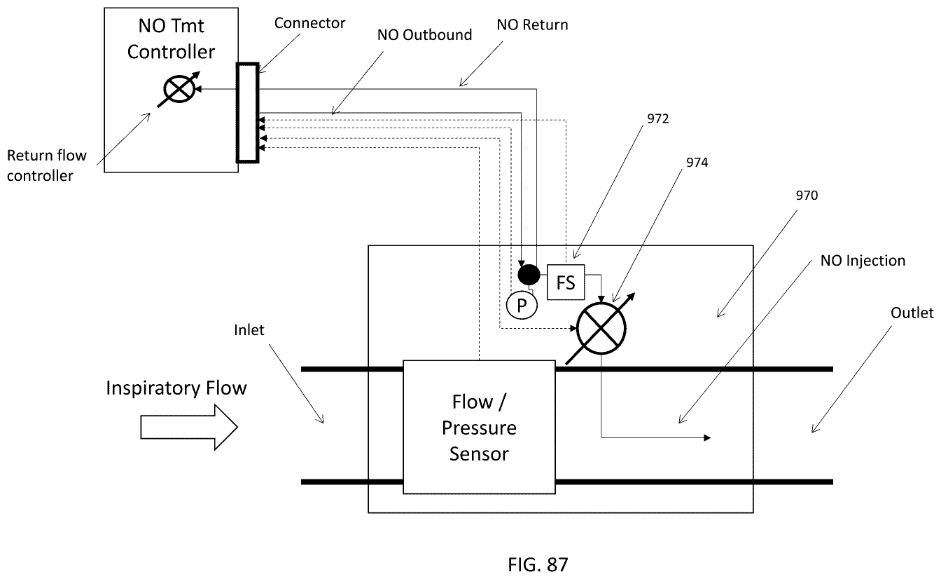

FIG. 87 depicts an exemplary embodiment of a remote injection module 970 for a NO delivery system with an external recirculation loop. NO-containing gas exits the controller and travels to the injector module mounted in an inspiratory line. In some embodiments, the NO gas is delivered at a constant flow rate from the controller to the external recirculation loop. The flow path bifurcates at the injection module, where NO gas will either pass through a flow sensor 972, a flow controller 974 and into an inspiratory flow or pass through a return leg. Returning gas reenters the NO treatment controller and passes through a return flow controller. In some embodiments, the NO treatment controller maintains a constant pressure within the outbound NO flow based on a measurement from a pressure sensor in fluid communication with the output gas flow. The NO injection flow controller is controlled by the NO treatment controller through a wired connection. The NO injection module also includes an inspiratory flow sensor to inform the NO treatment controller of how much NO to add to the inspiratory limb in accordance with set dilution ratio.

The injection module can include an inspiratory flow inlet and a dosed inspiratory flow outlet. In some embodiments, pneumatic and electrical connections are established with the NO treatment controller through a single connector (shown). In some embodiments, pneumatic and electrical connections are made with 2 or more connectors.

### Remote Injection Module Leak Test

In some embodiments, the NO treatment controller tests the external recirculation loop for leaks by closing the inspiratory injection flow controller and return flow controller and pumping the contents of the external recirculation loop up to a specific pressure. The pressure is then monitored for a decay (aka a leak down test).

### Remote Injection Module Calibration

In some embodiments, the calibration and control of the flow controller located in the remote injection module is checked by the NO generation controller in one or more ways. In some embodiments, the NO generation controller closes the injection and return flow controllers and pressurizes the external recirculation loop by running the pump. When the external recirculation loop and remote injector are installed correctly and in good condition, the pressure within the closed system increases in a predictable manner, indicating that there are no leaks and the system was assembled correctly. Continuing with the calibration/self-test, the system controller opens the injection flow controller a known amount. The injection flow controller is determined to be within calibration when the rate of pressure reduction within the external recirculation loop and/or the duration of time that it takes for pressure within the recirculation loop to drop from a first known value to a second known value (e.g. atmospheric pressure), as indicated by a pressure sensor in fluid communication with the external recirculation loop, matches an expected pressure reduction rate or duration, respectively, within a specified tolerance.

In some embodiments, the flow controller within the injection flow control module is a mass flow controller. In some embodiments, calibration of the injection flow controller within a remote injection module is checked by closing the flow controller on the return leg of the external gas circuit, sending a known mass flow of gas into the outbound leg (e.g. running the pump at a known speed, or measuring a mass flow rate of gas leaving the device controller) and confirming that the external gas flow controller reports a similar gas flow through it. Calibration and leak tests are typically performed with non-NO containing gas (e.g. air).

In some embodiments, the gas flow through a remote NO injection flow controller in an external recirculation loop can be determined as the difference between an outbound flow rate and a return flow rate within the recirculation loop. In one embodiment, the system controller first closes the injection flow controller and measures the outbound and return gas flow rates to ensure that they are matching or nearly matching. The remote NO injection flow controller is then opened by controlled amounts, often in a step-wise manner. For each step in the calibration, the difference between outbound and return flow rates is used as the actual flow rate through the injection flow controller for that flow controller setting and pressure head across the flow controller. The relationship between pressure head, flow controller setting and mass flow is recorded in a way that it can be utilized to control product gas injection during treatment. For example, calibration results may be recorded as coefficients to an algorithmic equation or as cells in a look-up table.

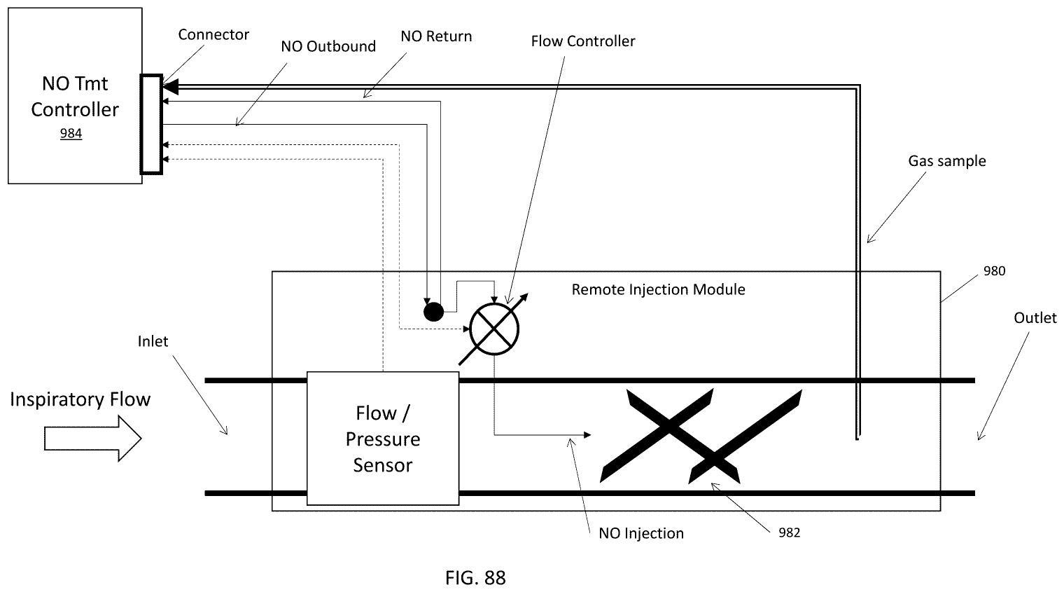

FIG. 88 depicts another exemplary embodiment of an injection module 980 with external product gas recirculation. This embodiment includes a mixing element 982 that mixes injected NO into the inspiratory gas stream. Various types of mixers have been contemplated, including static and dynamic. In some embodiments (not shown), NO product gas is introduced to the inspiratory gas flow through more than one orifice to encourage mixing of the NO product gas. After the mixer, a sample flow of inspiratory gas is collected and returned to the NO treatment controller 984. In some embodiments (not shown), the gas sample line includes one or more of a particle filter, hydrophobic filter, and water trap. For example, a combination injection/gas sampling module can be mounted near the patient in the inspiratory limb. This location is often after a humidifier. As a result, flow sensors and other gas contacting components are selected for moisture compatibility.

In some embodiments, an external injection module is disposable. In some embodiments, an external injection module is reusable and either cleanable or sterilizable. In some embodiments, injection modules are sized for specific applications (e.g. adult ventilation, neonate ventilation, pediatric ventilation, manual bagging, high frequency ventilation, jet ventilation, etc.). For each application, the length and diameter of tubing is optimized for the flow rates, NO concentration, and oxygen concentration for their target treatment conditions. In some embodiments, the sensors (e.g. flow, pressure) within an injector module are specified for the target treatment cases of an injection module.

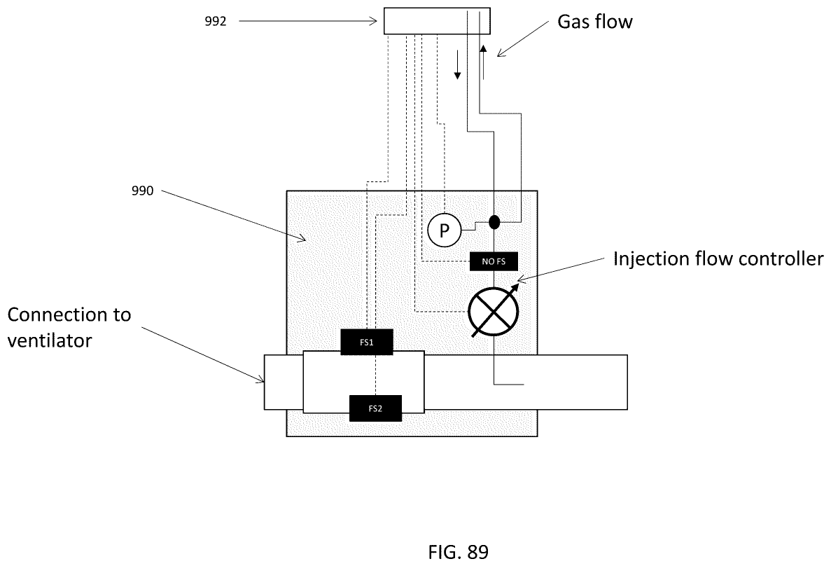

FIG. 89 depicts an embodiment of a remote injection module 990. The module connects to a NO device controller through a single, electronic/pneumatic connector 992. Electrical connections are established for one or more pressure sensors, flow sensors, and flow controllers (e.g. valves, mass flow controllers, pumps, etc.). In the depicted embodiment, gas flows into the connector and down to the remote injection module. In some embodiments (not shown), the product gas is scrubbed for NO₂ at or near the remote injection module. In some embodiments, the remote injection module includes a NO₂ scrubber (not shown). In some embodiments, the NO₂ scrubber at a remote injection module is replaceable.

Within the remote injection module, the gas path bifurcates with a portion of the gas returning to the NO generator and a portion flowing through an injection flow controller. Pressure within the product gas pre-injection flow controller is measured by a pressure sensor and reported back to the NO generation controller. Product gas flow through the injection flow controller is measured by a product gas flow sensor. In some embodiments (not shown), the injection flow sensor is located downstream of the injection flow controller.

It should be apparent to the reader that product gas flow into the remote injection module does not directly match injected product gas flow because a portion of the product gas returns to the device controller. The recirculation flow rate within the NO generation system can vary from 2 lpm to 10 lpm or more. In one embodiment, the outbound flow rate to the remote injection module is in the range of 3 to 5 lpm.

Inspiratory flow in the remote injection module is measured by one or more inspiratory flow sensors. In the depicted embodiment, two inspiratory flow sensors are utilized for redundancy. The length of cable and tubing between the connector and the remote injection module can vary from 0.5 to 4 m, with a 2 m length being typical. In some embodiments, the remote injection assembly is cleanable and/or sterilizable. In other embodiments, the remote injection assembly is disposable.

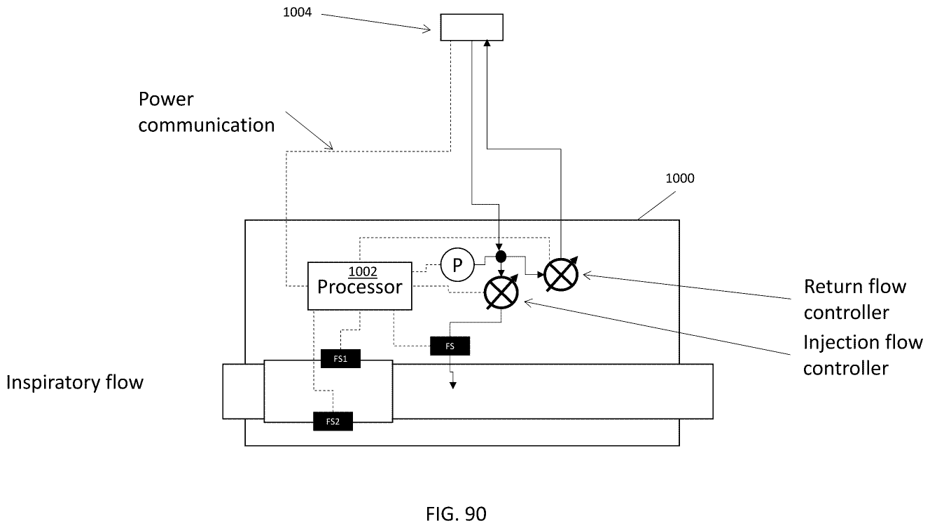

FIG. 90 depicts an embodiment of a remote injection module 1000 with a built-in processor 1002 (e.g. microprocessor, Field Programmable Gate Array (FPGA), Complex Programmable Logic Device (CPLD), Programmable Intelligent Computer (PIC), etc.). The injection module may also include random access memory (RAM), non-volatile memory, a power source (e.g. a battery), and power electronics (e.g. for actuating the flow controller). In some embodiments, the non-volatile memory stores one or more of sensor calibration, valve calibrations, fault conditions (e.g. sensor out of range, inspiratory flow sensor not connected), use data, and sensor data. When connected to a main unit using a connector 1004 and powered on, the remote injection module processor executes a hand-shake protocol to confirm communication has been established. The processor receives inspiratory flow measurements from one or more inspiratory flow sensors. The processor utilizes the inspiratory flow measurements to identify one or more of the peak inspiratory flow, inspiratory minute volume, inspiratory event timing, inspiratory event period, and inspiratory pressure. Based on the inspiratory flow information, the processor selects a dilution ratio and calculates a target product gas injection flow rate. The processor then controls the product gas injection flow rate to proportionally dose the inspiratory flow. In one embodiment, the processor within the remote injection module receives one or more of a target dose, a product gas concentration, a target dilution ratio, and a target dilution factor from the main NO generation device and it determines the control parameters for NO injection with additional information collected from inspiratory flow, product gas flow and product gas pressure sensors. In some embodiments, the information potentially sent from the main unit is not required because it is derived from the other parameters and/or a known value stored within the injection module processor memory. For example, a NO injection module that always delivers the same inhaled NO concentration (e.g. 300 ppm) to treat infections does not require the target NO concentration from the main unit. In one embodiment, the module receives a dilution ratio from the main unit and determines the target and controls NO flow through the injection flow controller based on vent flow measurements. In another embodiment, the injection module controller receives one or more of a target dose and product gas concentration from the main unit and selects a dilution ratio based on one or more of the target NO dose, product gas NO concentration, peak observed inspiratory flow rate, recirculation and flow rate.

In the depicted embodiment, a secondary flow sensor modulates the flow of product gas returning to the NO controller. The return flow controller is modulated by the processor to maintain a target pre-injection pressure, as measured by a pressure sensor within the injection module. It should be understood that the components presented herein can be located in various locations with similar performance. For example, the return flow controller can be located within the main NO gas production device and not within the injection flow controller. One of the advantages of including a processor within the remote injection module is that it decreases the number of electrical conductors required between the main NO generation device and the remote injection module. In its simplest form, there are only three to four wires that convey power and communications between the main unit and remote injection module. Another advantage is that sensor measurements made within the remote injection module travel less distance to a processor, thereby being less susceptible to EMI.

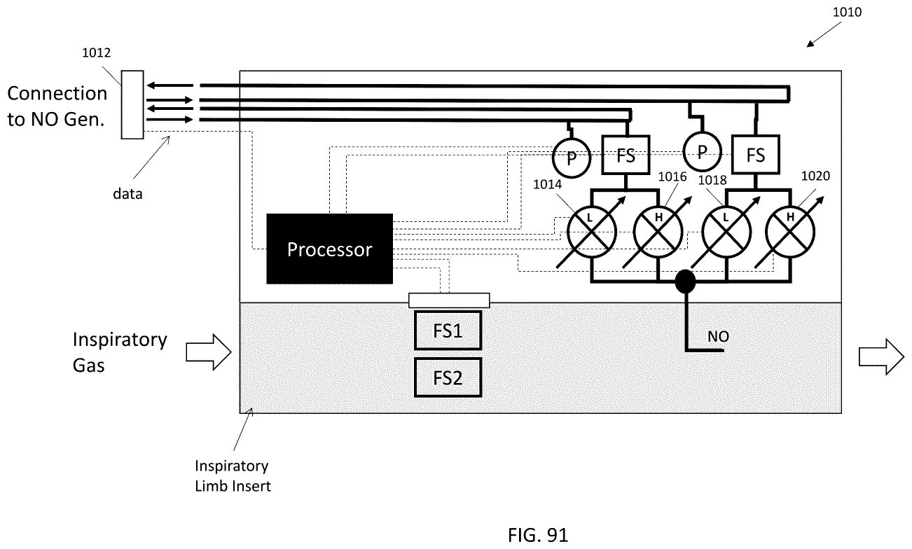

FIG. 91 depicts an embodiment of a remote injection module 1010 with full pneumatic and sensing redundancy. Pneumatic and electrical connections to the main controller are established with one or more connectors 1012 (one shown). Two independent product gas flow control circuits are depicted. Each flow control circuit includes one or more injected gas flow sensors (one shown), one or more injected gas flow controllers, and one or more product gas pressure sensors. In the depicted embodiment, each channel of product gas includes low flow controllers 1014, 1018 and high flow controllers 1016, 1020 for improved flow accuracy. In some embodiments, the measured injection flow rate is utilized by the controller as feedback in a closed-loop control scheme for the injection valve to achieve a target amount of NO product gas flow into the inspiratory limb. In some embodiments, the measured injection flow rate is utilized by the processor to detect an error condition in the injection flow controller. Error conditions may be reported and/or indicated to the User directly through user interface elements (e.g. a display, buzzer, indicator lights) on the injection module or indirectly by communicating to the main controller which, in turn, reports the error condition to the user. The two redundant product gas injectors are controlled by a common processor. In other embodiments (not shown), the processor is redundant as well. The processor(s) receive one or more flow signals from flow sensors mounted to an inspiratory limb insert. The inspiratory limb insert includes the inspiratory flow sensors and NO injector. The inspiratory limb insert is detachable from the remote flow injection module. In some embodiments, the inspiratory limb insert is disposable. In other embodiments, it is cleanable and/or sterilizable. One advantage to this design is that the injection module does not contact inspired gas and therefore doesn't require to be replaced, disinfected or sterilized between patients. The inspiratory limb insert is typically either replaced, cleaned, or sterilized before the NO delivery system is utilized with a subsequent patient.

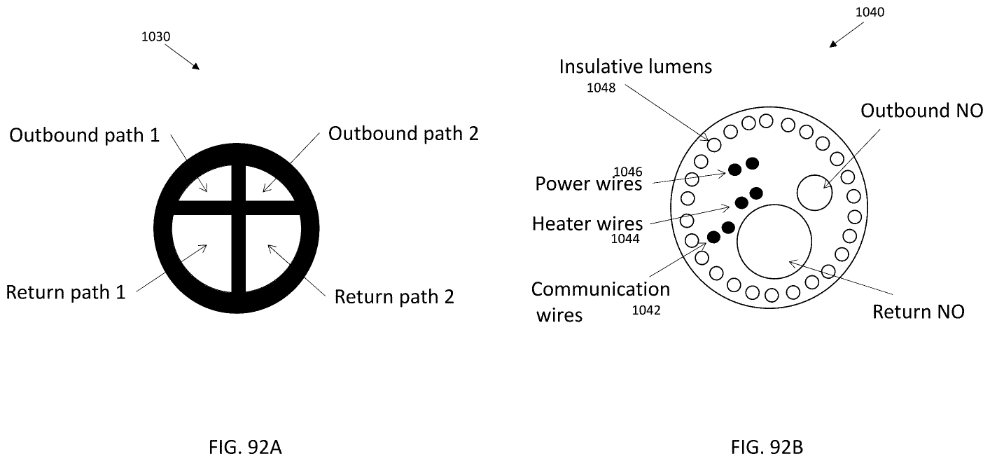

Systems that have multiple gas flow lumens between the NO generator and remote injection module can utilize a multi-lumen extrusion to reduce complexity for the user and mitigate the potential of tangling. FIG. 92A depicts the cross-section of a multi-lumen extrusion 1030 for redundant product gas delivery to a remote injection module. The outbound lumen cross sections are less than the return cross sections to hasten product gas delivery and decrease return flow resistance.

FIG. 92B depicts the cross section of a multi-lumen extrusion 1040 for delivering and retrieving product gas from a remote injection module. The extrusion includes wires 1042 for communication (e.g., sensor data, processor to processor communication, etc.), wires 1044 for heating the structure to prevent condensation, and wires 1046 for delivering power to the remote injection module. Insulative lumens 1048 add cross-sectional moment of inertia which reduce the risk of kink and reduce thermal losses. In some embodiments, the insulative layer consists of a foam that is either co-extruded with the extrusion or added as a jacket. In some embodiments, the extrusion is made from an elastomeric material with excellent NO and NO₂ compatibility (e.g. silicone, PVC). In some embodiments, one or more outbound lumens measure 1 mm to 3 mm in diameter and one or more return lumens measure 2 mm to 6 mm in diameter, for example.

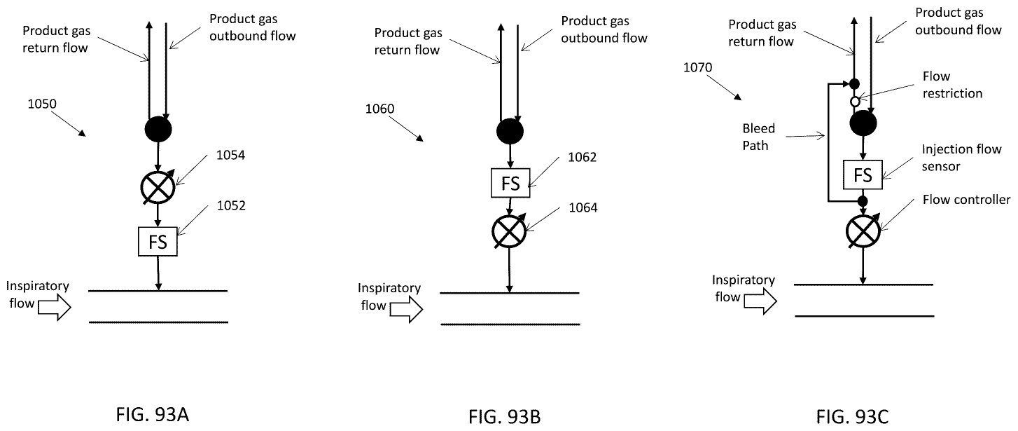

The following product gas injection designs are applicable to product gas injectors that are housed within the main enclosure of a NO delivery device and remote injector applications. FIG. 93A depicts an embodiment of a product gas injector 1050 with the injection flow sensing element 1052 downstream of the flow control element 1054. FIG. 93B depicts a product gas injector 1060 with an injection flow sensing element 1062 before the flow control element 1064. This approach can reduce dead volume between the injection flow controller to the inspiratory flow for reduced transit time and improved dose accuracy. Placing the flow sensing element upstream of the flow control element increases the dead volume between the recirculation loop flow and the injector. Stagnant or slow-moving product gas in this dead volume can age, increasing NO₂ levels within the product gas. This is particularly a concern when product gas flow is completely stopped, as is the case when inspiratory flow rates go to zero. FIG. 93C depicts an embodiment of a product gas injector 1070 that provides a bleed flow path that slowly purges the pre-injection flow controller dead volume to reduce stagnation and product gas aging pre-injection flow controller. In some embodiments (shown), bleed gas flows through the bleed pathway to bypass a flow restriction in the product gas return gas pathway. In some embodiments, the bleed gas is vented to atmosphere. In some embodiments, the bleed gas is scrubbed for one or more of NO and NO₂ prior to release into the atmosphere. The amount of flow through the bypass is typically trivial (e.g. <1 ml/min), but sufficient to maintain fresh product gas at the flow controller. When the bypass flow is trivial, no compensation is required in the injection flow rate measurement. In embodiments, where the bypass flow could impact overall dose accuracy, the bypass flow rate is subtracted from the injected product gas flow sensor measurement to obtain the actual product gas flow rate. In some embodiments, the bleed gas flow rate is measured by a flow sensor (not shown) so that the controller can determine the injected product gas flow rate as the product gas flow measurement minus the bleed gas flow rate.

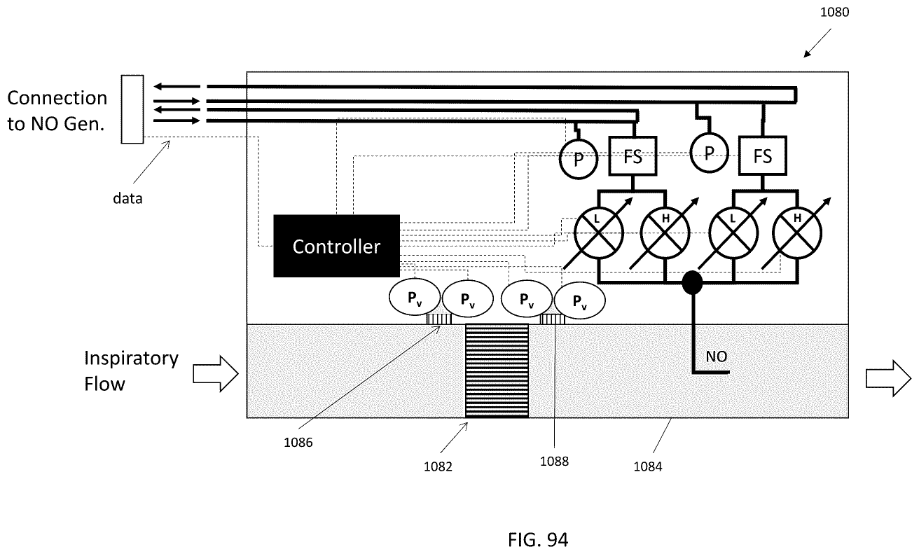

FIG. 94 depicts another embodiment of a remote injection module 1080 that utilizes reusable pressure sensors within the injection module to measure the pressure difference across a flow restriction 1082 within the inspiratory limb insert 1084. Sterile filters 1086, 1088 between the inspiratory flow and the pressure sensors prevent migration of pathogens into the pressure sensors. This design can reduce the cost of the inspiratory limb insert.

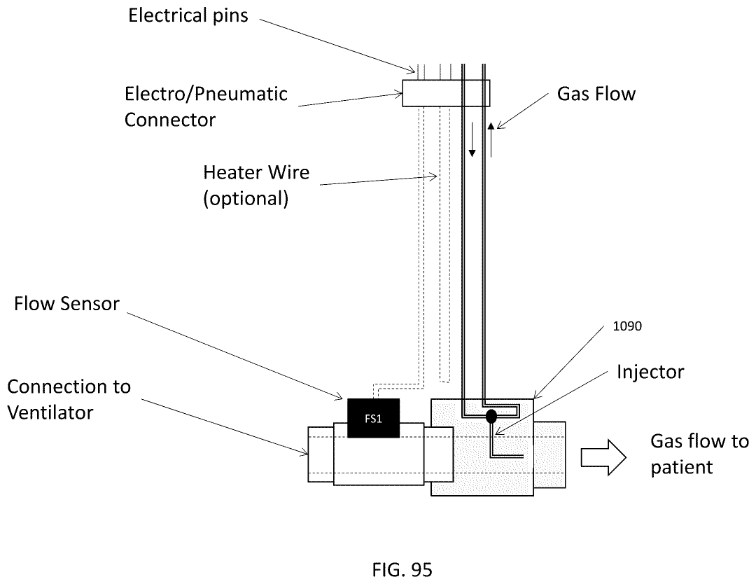

FIG. 95 depicts a remote injection module 1090 with no active flow control elements. The relationship between injected flow and return flow are controlled by the device controller via flow control elements with the main NO generation device. In some embodiments, the outbound product gas flow rate and concentration are constant. The controller modulates the amount of product gas injected into the inspiratory limb by varying a flow controller on the return flow path. The pressure within the return leg of the push-pull recirculation loop is often negative (i.e. vacuum) to pull excess product gas back to the NO generator from the NO injector. In some embodiments, the pressure/vacuum of the outbound flow and/or the inbound flow are varied with ambient pressure (e.g. altitude fluctuation) to maintain a constant pressure within the plasma chamber. In some embodiments, the pressure within the ventilator stream is locally measured in order that this dynamic pressure can be used to improve valve flow accuracy and response time for dynamic dose control. The remote injection module connects to the downstream end of an inspiratory flow sensor. In the depicted embodiment, the flow sensor electrical conductors and NO outbound and return pneumatic pathways are grouped into a single connector that connects to the main device. This decreases complexity and use steps. In other embodiments, one or more of the conductors/pathways and/or connectors are separate. Also included in this design is an optional resistive wire loop that heats the pneumatic lines to prevent condensation and/or freezing. In some embodiments, an insulative layer (not shown) is placed around the external recirculation pathway to prevent ambient temperature from inducing condensation or freezing within the product gas flow. In some embodiments, a check valve (not shown) in the injector ensures forward product gas flow into the inspiratory limb.

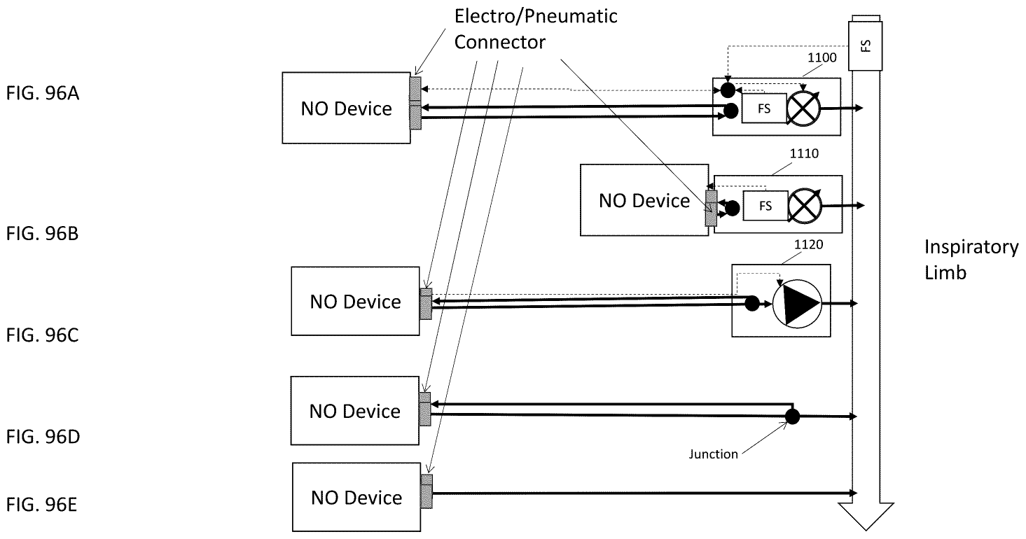

FIGS. 96A-96E depict five embodiments of a remote injection module. Each embodiment interfaces with the NO treatment controller with one or more electrical and pneumatic connections. In some embodiments, each external module interfaces with the same electrical/pneumatic connector. Measurement of inspiratory flow is depicted for the top embodiment, but it should be understood that inspiratory flow can be measured for each of the depicted embodiments.

FIG. 96A depicts an external recirculation system with a remote injection module 1100. The remote injection module includes one or more product gas flow sensors and injection flow controllers. The length of tubing between NO delivery device and remote injection module can vary in length up to several meters.

FIG. 96B depicts a short injection module assembly 1110. This provides reduced inhaled NO₂ levels when it can be used, owing to the short transit time from the NO Controller to the injection module. It is used for applications where the NO device can be located closer to the point of NO delivery (e.g. inspiratory limb, patient mask, etc.).

FIG. 96C depicts an external injection module 1120 with boost pump. Gas is passed from the NO device through the connector and a length of tubing to the injection module. An optional return gas path permits the mass flow of gas out to the injection module to be more than the mass flow injected into the inspiratory limb, thereby reducing transit time and inhaled NO₂ levels. The pump draws gas from the outbound NO flow line and pressurizes the gas to a level that exceeds the pressure within the destination flow. In one embodiment, the destination flow is a high flow nasal cannula system with a pressure of up to 25 psi.

FIG. 96D depicts an external injection module with a push/pull configuration. In one embodiment, gas is pushed out of the NO delivery device as a constant mass flow rate such that the NO production (i.e. the mathematical product of product gas flow rate and product gas concentration) exceeds the peak production of the destination gas flow. The return gas line operates under vacuum to suck back a variable quantity of product gas from the junction. In one embodiment, the return gas line flow rate is inversely proportional to the flow rate within the destination gas flow resulting in proportional introduction of product gas into the destination gas flow. Destination gas flow is typically measured directly with a flow sensor (not shown).

FIG. 96E depicts how the same electrical/pneumatic connection can be utilized to deliver NO through a long tube. In some embodiments, NO is delivered through a long tube in proportion to the flow rate in the destination flow. In other embodiments, NO is delivered at a constant flow rate through the long tube. A recirculation architecture can be utilized for long tube delivery by not drawing back product gas.

In some embodiments, the NO treatment control system can detect the type of external injection module connected (e.g. via memory chip). In one embodiment, the treatment controller utilizes the length of the injection line as an input into the NO loss model. When the injection line is shorts, there is less NO loss to oxidation than when the NO injection line is long. Hence, when the injection line is short, the controller can set the plasma parameters (i.e. frequency and duration) to a lower level to achieve a target concentration of NO at the patient. In another embodiment, the controller changes from a proportional flow to a constant flow mode, depending on the type of injection module attached.

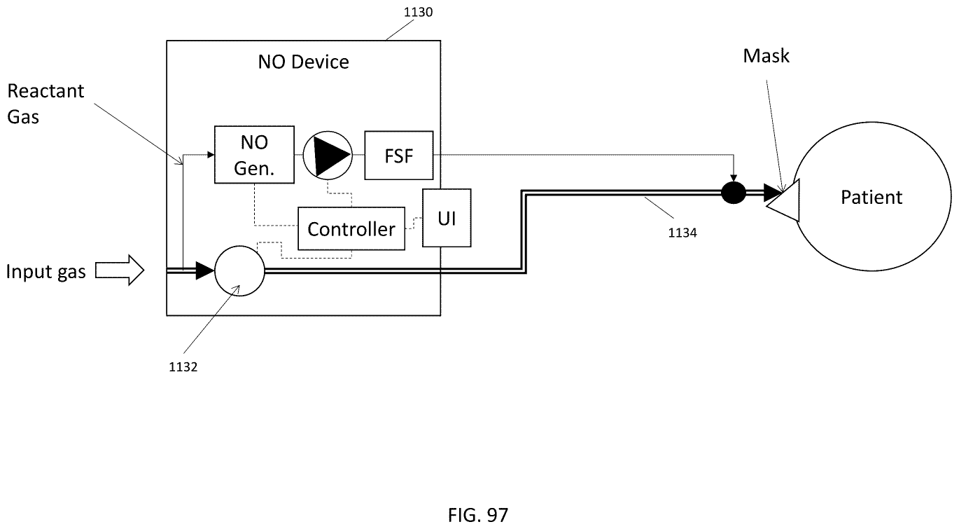

FIG. 97 depicts an exemplary embodiment of a NO delivery device 1130 that provides NO and inspiratory gas to a patient. Input gas entering the system is sourced from one or more of external compressed gas source(s) or ambient air. In some embodiments, input gas is simply air. In some embodiments, the system receives compressed nitrogen, oxygen, helium, and/or air. Gas entering the system passes through a pump 1132 (e.g. a blower) that delivers inspiratory gas to the patient. In embodiments where inspiratory gas is sourced from a compressed source, the pump is optional.

A NO generator within the NO delivery device sources reactant gas either from the input gas stream (shown) or an independent gas entry port (not shown), if required. The NO generator generates NO by one or more of electrical discharge, conversion of NO₂ gas, release of NO from a donor molecule or other means. NO-containing product gas passes through a pump and a filter-scrubber-filter assembly prior to traveling through an independent lumen to a point at or near the patient where the two gas streams (inspiratory and NO) merge. It should be noted that the sequence of NO generator, pump, filters, and scrubber can vary with different designs is not constrained to the arrangement depicted. In some embodiments, the system includes multiple plasma chambers in series or parallel to generate sufficient NO in the product gas for high dose (>80 ppm) treatments. In some embodiments, a system designed with redundant NO generators for safety can utilize both NO generators simultaneously for high dose treatments and still be considered safe because immediate cessation of treatment does not present a harm to a high-dose patient (i.e. a patient being treated for an infection). When using this design, a patient can receive an inhaled dose of NO from a single device (i.e. no additional gas delivery device (e.g. ventilator, CPAP) required). NO and inspiratory gas are kept separate for as long as possible to minimize formation of NO₂ in the inspired gas. In some embodiments, the NO and inspiratory gas pass through a dual-lumen tube 1134. In some embodiments, NO product gas is delivered through a high flow nasal cannula, face mask or CPAP system with separate air and NO lumens.

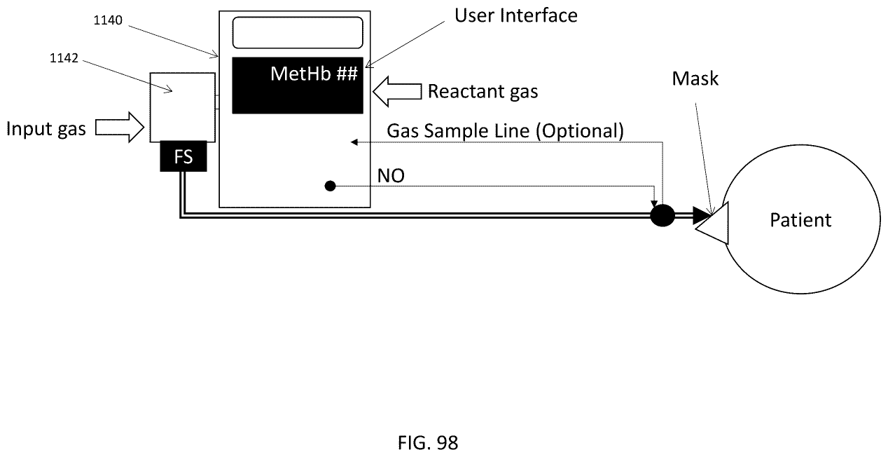

FIG. 98 depicts an exemplary embodiment of a NO generation device 1140 with a blower accessory 1142. The blower removably attaches to the NO device, establishing electrical connections. The NO generation device provides one or more of power and command signals to the blower and receives flow measurements from a flow sensor. In some embodiments (not shown), the blower attachment includes a processor that modulates the blower flow rate based on flow sensor measurements to achieve a target gas flow rate to the patient requested from the NO device. Other permutations of these key components are also possible.

An inspiratory limb consists of independent air and NO lumens with an optional gas sampling lumen. The blower sources air from the environment and delivers air to the patient at a flow rate (typically 5 to 60 lpm). In one embodiment, the NO device provides NO in proportion to the blower flow rate to achieve a target NO concentration within the inhaled gas. In one application, this type of system is used to delivery high concentration NO, i.e. concentrations >80 ppm. In some embodiments, a NO device that contains redundant NO generators utilizes more than one NO generator at a time to achieve a target NO production level. Although the redundant NO generator is typically reserved as a backup during NO treatments, high dose NO delivery is different because a disruption in NO delivery does not present immediate harm to a patient.

NO can be delivered either continuously or in pulses to the inspiratory line or mask. Pulsed delivery can be triggered by breath detection within the NO delivery lumen, the gas sampling lumen, or an additional lumen (not shown). In some embodiments, NO pulses are chased with an inert gas (e.g. air) to prevent the NO delivery lumen from holding high concentration NO between breaths. This minimizes the amount of NO formation between breaths. Inhaled NO concentrations can be as high as 1000 ppm or more during high dose NO treatments.

In one embodiment, a clinician inputs treatment parameters (e.g. target inhaled NO concentration, treatment duration, portion of breaths to dose) into the NO device. In some embodiments, the patient receiving treatment is unable to change the treatment parameters. In one embodiment, the user interface includes a progress bar and/or timer to indicate remaining treatment time.

### Wastegate Valve

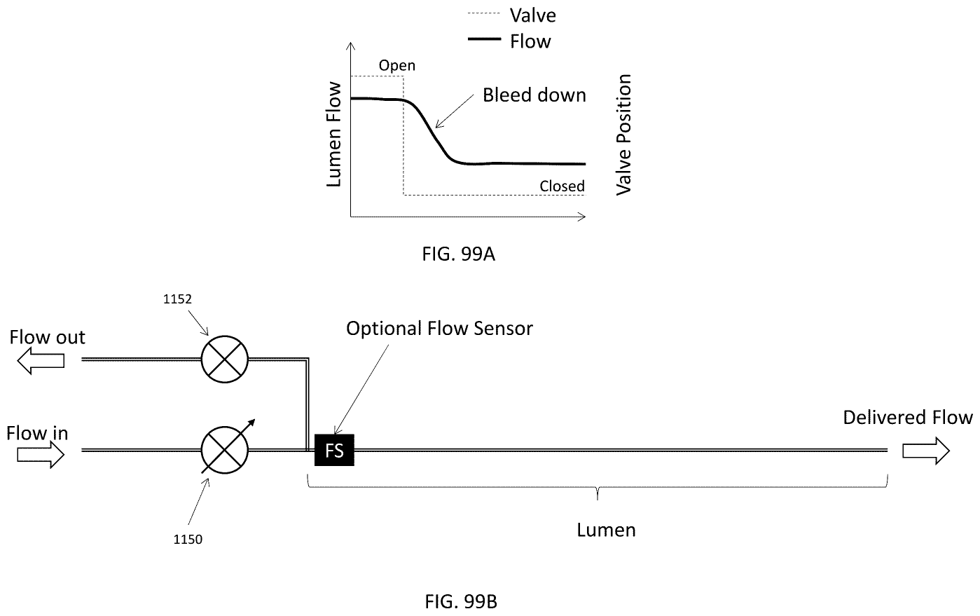

When gas is delivered through a long lumen to another flow, pressure is required to push the gas through. When an upstream flow controller closes (partially or fully) to decrease gas flow, residual pressure within the lumen decreases over time, resulting in a slow change in the flow rate within the lumen. FIG. 99A depicts an exemplary graph showing how the flow controller can close, but flow out the end of the lumen continues for up to hundreds of milliseconds, depending on the pressure, lumen diameter, lumen length, and temperature. This effect can result in excessive NO being dosed into an inspiratory flow, resulting in higher than target NO concentration in the merged flow.

FIG. 99B depicts an embodiment of a system that enables a NO delivery system to rapidly slow or arrest the gas flow through a lumen. Flow through the lumen is controlled by an upstream flow controller 1150. A waste gate valve 1152 is located at the upstream end of the lumen, in fluid communication with the upstream flow controller. When the upstream flow controller is partially or fully closed, the device controller simultaneously opens the waste gate valve to release some or all of the upstream pressure. In some applications, the waste gate valve is closed again when the upstream pressure reaches the downstream lumen pressure (hence, no flow through the lumen). In some embodiments, the upstream waste gate is closed again when the flow rate through the lumen reaches a new target flow rate. In some embodiments (not shown), gas flow rate through the lumen is measured buy an optional flow sensor.

In some embodiments, gas flow through a waste gate valve is released into the atmosphere. In some embodiments, gas flow released through a waste gate valve is scrubbed prior to release. In some embodiments, gas released from a waste gate valve is merged with another gas flow within an NO generation system (e.g. reactant gas, product gas). In some embodiments, the gas released from a waste gate valve is merged with a product gas flow within a recirculation loop.

### Remote Injection Timing

The volume of the inspiratory limb can vary with inspiratory tube diameter, length, and presence/absence of auxiliary respiratory devices (e.g. humidifier, nebulizer, gas sample ports, etc.). These variations in inspiratory limb set-up create variation in the limb compressibility and gas transit time. In some embodiments of a NO delivery system, the mass flow rate of the inspiratory flow is measured at a different location than where the NO is injected. This can cause the NO injection pattern to be out of phase with the inspiratory flow pattern due to lags in the NO generation system as well as compressibility of inspiratory gas. In some embodiments, the NO delivery system characterizes the phase shift and/or transit time between inspiratory flow measurement location and NO delivery location. In some embodiments, the phase shift is quantified by introducing an acoustic pulse (e.g. sound or ultrasound produced with a speaker, buzzer, etc.) to a first location and detecting the pulse at a second location. The transit time from the first location to the second location is indicative of the distance between the two locations. In some embodiments, the NO injection signal is delayed by the acoustic pulse transit time in order to inject NO in phase with the inspiratory pattern. In some embodiments, the properties and conditions of the inspiratory limb gas are also measured and/or entered by the user. Properties including but not limited to the oxygen content, humidity and temperature of a gas enable a more accurate estimate of the gas density which affects the compressibility and propagation of sound.

### Pulsed Delivery

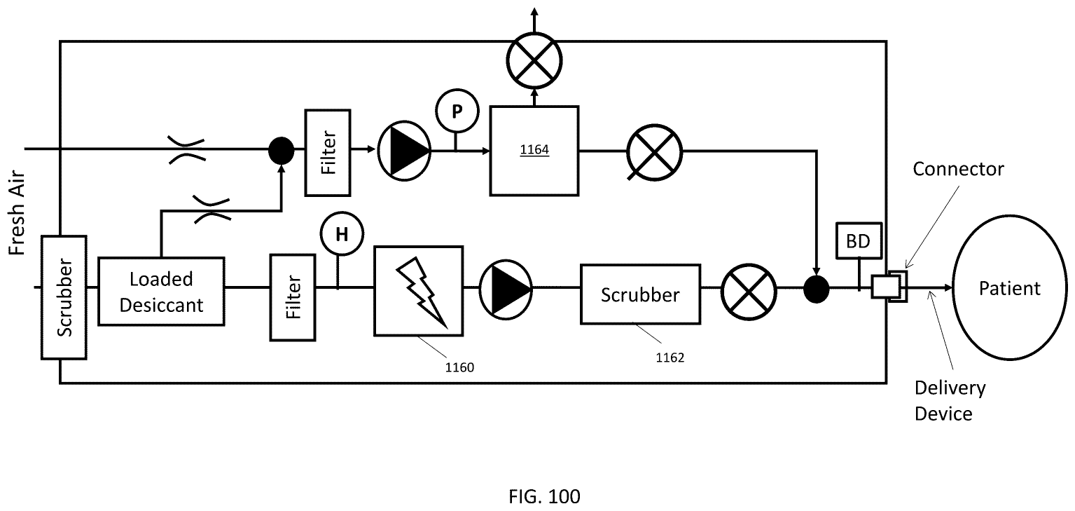

FIG. 100 depicts an exemplary embodiment of a NO generation and delivery system for pulsed NO delivery. The system includes two parallel gas paths. A first gas path passes through a plasma chamber 1160 for formation of NO product gas that accumulates in a pressurized scrubber 1162. A second gas path is used to accumulate purge gas in a reservoir 1164 for purging the system and delivery system. In some embodiments, the pumps for each flow path operate at a constant flow rate to enable the use of the smallest possible pumps and to minimize vibration, noise, and variation in reactant gas flow. Sometimes during the operation of such NO generation system, the purge gas pump is unable to sufficiently pressurize the purge reservoir (i.e. unable to reach a target pressure threshold) prior to the next detected breath. This most commonly can occur during fast respiratory rates (e.g. >40 breaths/minute). In this case, the NO delivery system can skip a breath and not deliver NO into in order to provide additional time for the pump to bring the purge reservoir to pressure. In some embodiments, additional NO is delivered in one or more subsequent breaths to account for the skipped breath and maintain a target dosing rate (e.g., 6 mg·hr).

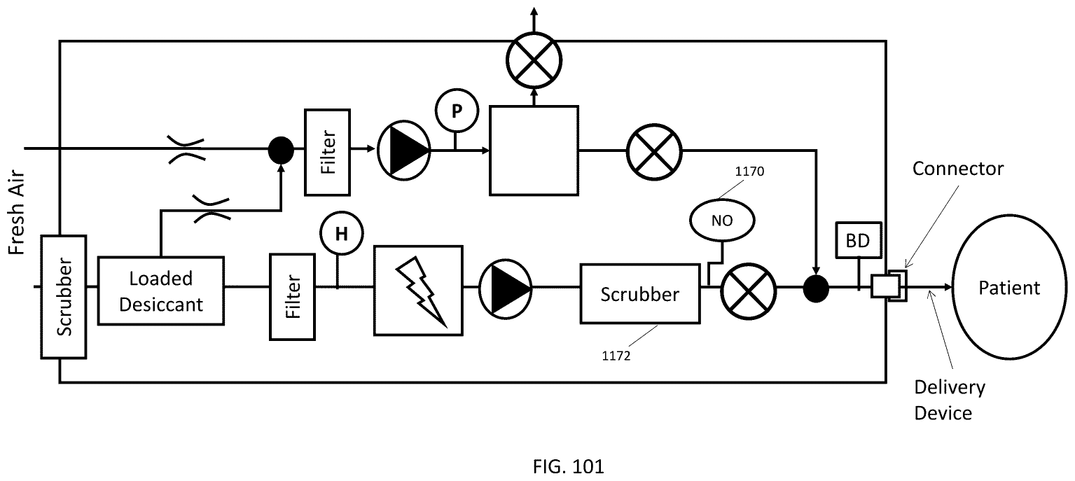

FIG. 101 depicts another exemplary embodiment of a similar NO generation system shown in FIG. 100 with a NO concentration sensor 1170 in fluid communication with a scrubber 1172. The NO concentration sensor enables the system to know the NO concentration being delivered. Knowing the NO concentration, the device controller can adjust the NO pulse flow rate and duration to deliver a target quantity of NO to the patient in each pulse.

In some embodiments, the amount of NO delivered from a NO source to an inspiratory flow can be a function of the NO gas concentration and gas flow rate. Smaller diameter tubes decrease the transit time of gas flowing a particular flow rate at the cost of higher pressure and higher NO loss to oxidation. For every patient inspiratory flow rate and dose, there is an optimum NO concentration and product gas flow rate. In some embodiments, a NO delivery tube has two or more lumens. In some embodiments, the two lumens are different sizes with a small diameter for low flow rates and a large diameter for high flow rates. In some embodiments, the diameters are the same. A single lumen is utilized for low flow rates and more than one lumen is used for higher flow rates. By utilizing more than one lumen, a NO delivery system can minimize NO transit time to reduce NO₂ formation during transit. In one specific example, a ventilated patient has low minute volume and low bias flow and a low NO dose. In this example, the No delivery system would use the smallest lumen to deliver NO because the quantity of NO to deliver is low. In another example, a patient is being treated with 30 lpm of 80 ppm NO gas. The system delivers NO through a larger lumen or multiple lumens to enable a higher flow rate of lower concentration NO to delivery target NO dose.

In some embodiments, a NO delivery lumen is purged (i.e. flowed with non-NO containing gas) during treatment. In some embodiments, one or more lumens in an array of lumens is purged so that NO does not oxidize with the lumen. Purging of an injection line is done with non-NO gas (e.g. air). In some embodiments, the system purges one or more NO lumens at the end of an inspiratory event. In some embodiments, the timing of the end of the inspiratory event is determined by timing of the end of prior inspiratory events. In some embodiments, the end of inspiratory event is determined based on the inspiratory flow rate crossing a threshold as the inspiratory flow rate slows down.

Similarly, in some embodiments, the NO delivery lumen can be primed with NO prior to the next inspiratory event based on timing of one of more prior inspiratory events. In some embodiments, the volume of the NO delivery lumen is sufficiently small and/or the NO flow rate is sufficiently fast that priming prior to inspiration is not necessary for acceptably accurate NO delivery (i.e. the timing delay due to priming the NO delivery lumen does not have a significant impact on overall dose consistency and/or accuracy).

### Electrical Impedance Tomography

In some embodiments, electrical impedance tomography (EIT) is utilized to monitor patient respiration and/or observe regions of the lung that have gas exchange (i.e. air distribution within the lung). In some embodiments, a NO delivery system delivers NO to one or more portions of a patient inspiration based on the state of the respiratory cycle, as indicated by EIT. Improvements to lung ventilation resulting from NO delivery can be quantified in terms of cross-sectional area or lung volume. In some embodiments, NO dose is varied to obtain maximal lung ventilation. For example, in one embodiment, EIT data are collected before NO is delivered. The patient is then administered inhaled NO at a low dose and corresponding changes in EIT area are quantified. NO dose is then increased in a step-wise fashion, holding at each step for 30 seconds to several minutes to provide time for the higher dose to have an effect. There will come a point where an incremental step in NO dose has negligible to no effect on lung recruitment. When this occurs, the NO dose is left at that that level. In some embodiments, the NO dose is left at a level one or more steps before the maximal level. This process can be repeated periodically (e.g. hourly) as more and more of the lung is recruited and becomes functional.

In some embodiments, EIT is utilized in combination with EKG to capture cross-section of the chest, heart rate, respiratory cycle and blood flow. The blood flow is a ripple (i.e. higher frequency content) in the signal that can be determined via filtering and/or data post-processing. The amplitude of the blood flow ripple varies with pulmonary hypertension. This measurement approach is such that the readings are independent of how the patient is feeling and other factors that affect a 5-minute walk test. In some embodiments, measurements made at time intervals (e.g. monthly) can help ascertain trends in disease progression or reversal. In some embodiments, NO treatment parameters (e.g. dose, concentration, timing within the breath) are varied by the NO delivery system to determine optimum settings based, at least in part, on maximizing the blood flow ripple in the EKG waveform.

### Extended Ventilator Cartridge

When a NO generation and delivery system is added to an active ventilator circuit, it is desirable to add the NO system quickly to minimize disruption to patient ventilation. In addition, it is desirable to add as little volume to the inspiratory limb as possible to prevent the ventilator from requiring an inspiratory limb compliance test (i.e. an assessment of inspiratory limb volume, compressibility, flow restriction, and leak) as this would present a significant delay in patient ventilation and require manual ventilation in parallel.

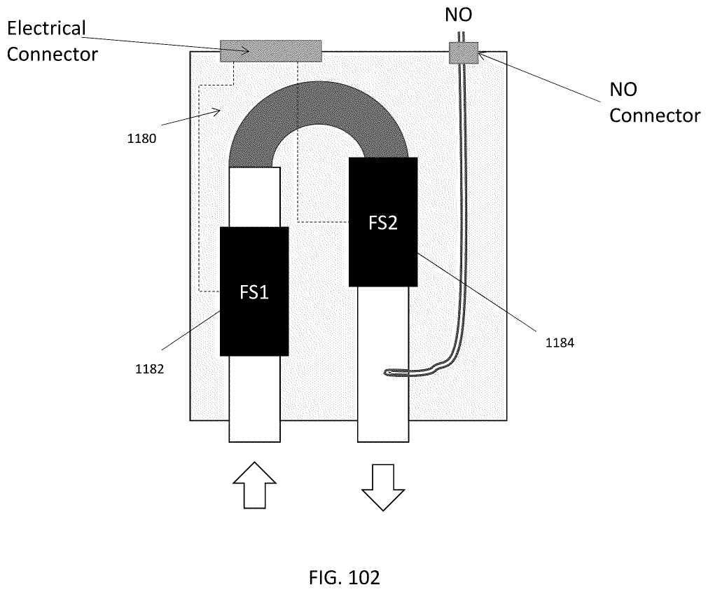

In some embodiments, a NO generation system utilizes a ventilation cartridge to interface with a ventilator circuit. A ventilator cartridge can be useful because it introduces NO into the ventilator circuit as quickly as possible (i.e. minimal transit time). FIG. 102 depicts an exemplary embodiment of a ventilator cartridge 1180. Inspiratory gas enters the cartridge and flows through a first flow sensor 1182. The gas flows around a U-turn and through a second flow sensor 1184. After the second flow sensor, NO is delivered to the inspiratory flow and the gas flow exits the cartridge. In some embodiments, the connections to the inspiratory limb are standard, ISO conical connections. In some embodiments, there is a single flow sensor. In some embodiments, there are three flow sensors so there can be a voting scheme to identify an errant flow sensor. In some embodiments, a single flow sensor housing includes multiple sensing elements to reduce the size of the sensor and place all sensors in a similar flow stream (i.e. on the same side of the u-turn). The ventilator cartridge interfaces to the rest of the NO generation via one or more pneumatic (e.g. NO) and electrical (e.g. flow sensor power and data) connections. One caveat of a ventilator cartridge, however, is that plumbing a ventilator cartridge into a ventilator circuit can involve adding significant tubing (i.e. volume) to a ventilator circuit during installation. For example, in one application, the addition a of a ventilator cartridge requires adding the volume of the ventilator cartridge and a tube from the ventilator cartridge to the humidifier in a circuit.

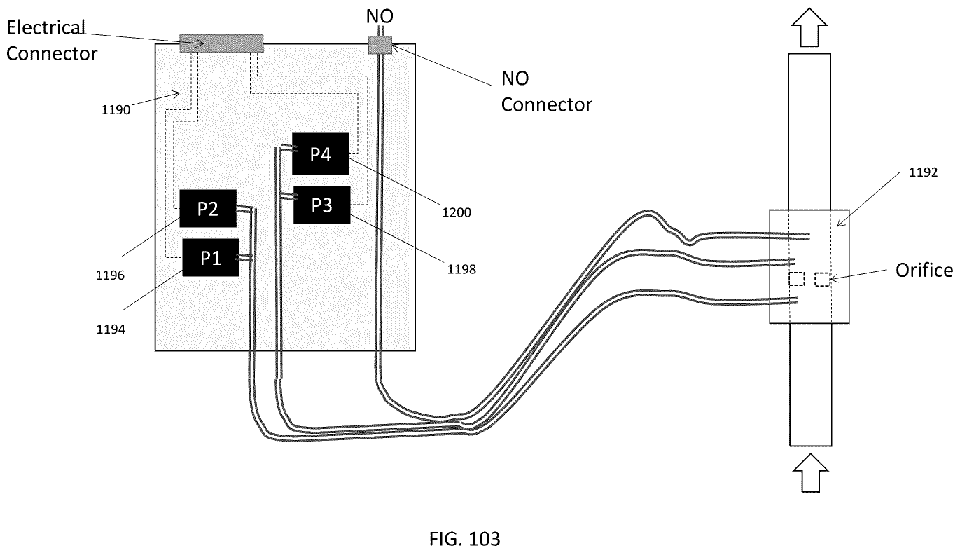

In some embodiments, a NO generation device delivers NO remotely through a long tube such that the NO injector can have small volume and be inserted in many locations within the inspiratory limb of a ventilator setup. FIG. 103 depicts an exemplary embodiment of a remote ventilator cartridge 1190 with similar interface as the design depicted in FIG. 102, so that the ventilator cartridges are interchangeable. A 3-lumen extrusion is utilized to remotely deliver NO and measure inspiratory flow. Inspiratory flow is quantified by measuring the delta-pressure across a known flow restriction in an injector body 1192. Pressure sensors 1194, 1196 (P1 and P2) measure the upstream, high pressure value. Pressure sensors 1198, 1200 (P3 and P4) measure the pressure downstream of the flow restriction which is lower. In some embodiments, the 2 or more sensors measuring the same value are the same sensor for redundancy. In some embodiments, the two sensors are utilized to measure specific ranges of pressure values to broaden the range of flow rates that can be detected by the system.

In some embodiments, the injector body is specific for the size of tubing that the vent cartridge is intended for (e.g. neonate, pediatric, adult). In some embodiments, the pressure sensors and/or flow restriction are specific for the intended range of flow rates, patient types, or treatment conditions.

In some embodiments (not shown), flow measurement is made with one or more remote flow sensors located within the injector body. In some embodiments, wires to power and communicate with the remote flow sensors are bundled with the NO delivery lumen. In some embodiments, the wires are separate. In some embodiments, the remote flow sensors are battery powered and communicate wirelessly with the NO generator.

### Post-Injector Inspiratory Limb Volume Determination

The volume of various system components in an NO generator and inspiratory circuit can affect the NO dose accuracy. For example, the volume of an inspiratory circuit affects the transit time of NO from the NO delivery system to the patient. This, in turn, affects the amount of NO that is lost to oxidation as it is delivered to the patient. In some embodiments, a NO delivery device determines the post-injector inspiratory limb volume by generating a known signal in the NO time history (e.g. a step change in NO concentration, a spike in NO, or a lapse in NO) at a known point in time. The system then looks for that signal in the NO gas sensor data stream.

In some embodiments, the controller of a NO delivery system marks the time when NO is first introduced to an inspiratory limb (i.e. a step change in NO). The controller then marks the time that NO is first detected in gas sensors that sample inspiratory gas. The volume of the pathway from the injection point to the gas sensors is a function of transit time (t_{detection}−tₛₜₐᵣₜ), the inspiratory limb flow rate, the NO flow rate, the gas sample flow rate, the length of the gas sample path (a known value), and the sensor t90 response time.

Neglecting delays attributed to the gas sensor and gas sample flow, the volume of the inspiratory limb can be calculated as a function of the inspiratory limb flow rate and the transit time.

$${V}_{insp} = {\int }_{0}^{{t}_{r} - {l}_{s} - {t}_{s}} Qdt$$

Where, Vinsp=the inspiratory limb volume, tr=the elapsed time that the signal appears in the gas sensor data, 1 s is the lag time of the sensor (e.g. t90), ts is the transit time of sample gas to the gas sensor (i.e., sample line volume/sample flow rate), and Q is the inspiratory flow.

By knowing the post-injector inspiratory limb volume and the inspiratory gas oxygen level, a NO delivery device controller can predict the amount of NO oxidation that will take place as it transits from the injection location to the patient. The controller can then increase the amount of NO injected into the inspiratory limb to compensate for anticipated losses in order to improve overall delivered dose accuracy.

### Pressurized Volume Determination

In a NO generation system with a pressurized reservoir and/or pressurized scrubber. The system can determine the dead volume of the reservoir or scrubber by pumping or releasing a known volume of gas and measuring the change in pressure. For example, the dead volume within scrubbers can vary with type of scrubber media used and manufacturing variance. In some embodiments, the NO generator pressurizes the scrubber with a known amount of gas. The dead volume of the scrubber is then calculated as follows:

$${V}_{d} = \frac{{P}_{0}}{{P}_{f}} {\int }_{0}^{{t}_{f}} Qdt$$

Where Vd=dead volume, P0=initial pressure of the volume, Pf=final pressure of the volume, tf=the time at which pressure reaches final pressure and Q=the flow rate into the volume.

In some embodiments, a NO device reads a dead volume (e.g. from a bar code or memory device) or measures a dead volume of a scrubber component prior to using that component. When the dead volume is read from the scrubber, the dead volume was measured during manufacturing. In some embodiments, the system varies NO generation parameters (e.g. NO concentration, reactant gas flow rate, etc.) in response to the dead volume measurement to achieve a known delivered concentration of NO.

In some embodiments, a NO device sweeps through acoustic frequencies to determine the resonant frequency of a volume. The resonant frequency coincides with a particular volume.

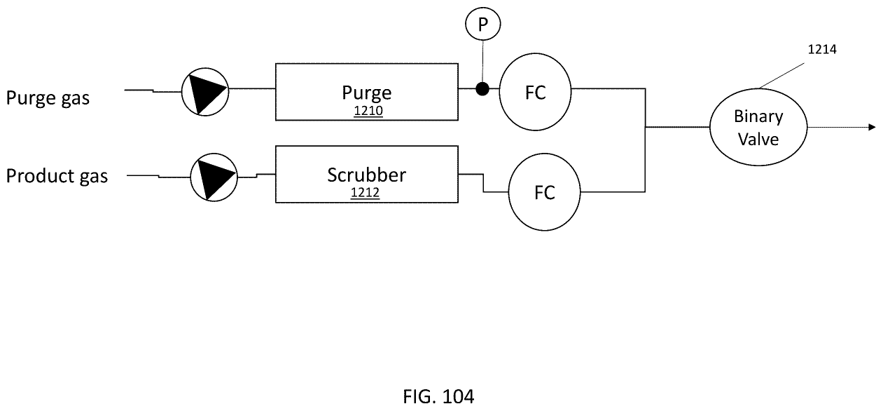

In some embodiments, an unknown volume is filled with a known volume of gas. The change in pressure within the unknown volume enables the calculation of the size of the unknown volume using Boyle's law, P1V1=P2V2. FIG. 104 depicts an embodiment of a system that is configured to determine the size of an unknown volume (a scrubber in this case). The system consists of a purge volume 1210 of known size that is filled with gas (e.g., air) by a pump. The system also includes a scrubber component 1212 with unknown internal volume. Gas from each volume flows through a respective flow controller and merge. The merged flow passes through a binary (on/off) valve 1214.

For example, the device controller can determine the unknown volume of the scrubber as follows. First, the controller opens the binary valve to ensure that the scrubber and purge reservoir are at atmospheric pressure. The controller then closes the binary valve and both flow controllers. The controller then enables the purge pump to pressurize the purge reservoir to a known pressure. In one embodiment, the pressure is quantified by a pressure sensor in fluid communication with the purge reservoir (not shown). The controller then turns off the purge pump and opens up both flow controllers. Gas from within the purge reservoir pressurizes the scrubber and the pneumatic pathway between the purge reservoir and scrubber. The resulting pressure (P2) is recorded. The gas volume of the purge reservoir, pneumatic path and scrubber (V2) is calculated as P2=P1V1/V2, or P2=(initial purge reservoir pressure)*(purge reservoir volume)/(final pressure). In some embodiments (not shown), pressure within the scrubber is measured directly. In this case, either the purge pressure sensor or the scrubber pressure sensor can be utilized to measure P2. When pumps that do not prevent backflow are utilized, binary valves (not shown) may be necessary to prevent retrograde flow through the pumps during the pressure test.

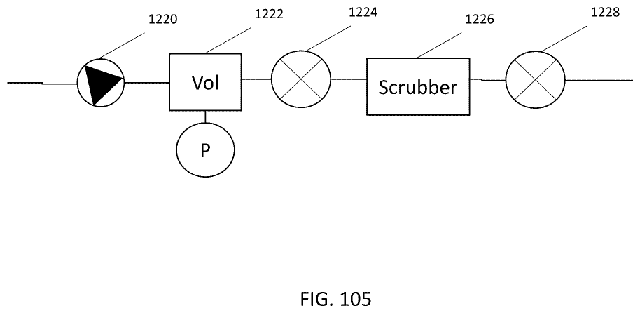

FIG. 105 depicts another exemplary embodiment of a system architecture that can be utilized by a NO generation and/or delivery system to quantify an unknown volume. The system comprises of a gas inlet through which gas is drawn into the system by a pump 1220. After a pump is a cavity 1222 of known volume followed by a valve 1224. After the valve is a cavity 1226 of unknown volume (e.g., a scrubber). After the scrubber is another valve 1228 (e.g. binary valve, flow controller). The controller of the system (not shown) initially opens both valves to reach a known pressure (e.g. atmospheric pressure, ambient pressure). In some embodiments, a port to ambient pressure with an additional valve provides fluid communication with the atmosphere (not shown). After zeroing the pressures, the controller closes both valves and operates the pump. The pump is operated until the known volume pressure reaches a target value, as indicated by the pressure sensor. The controller then opens the valve between the known volume and the unknown volume. Gas within the known volume flows into the unknown volume until a consistent pressure is achieved in both volumes. The size of the unknown volume is calculated using Boyle's lay, as above.

### Dead Volume Compensation

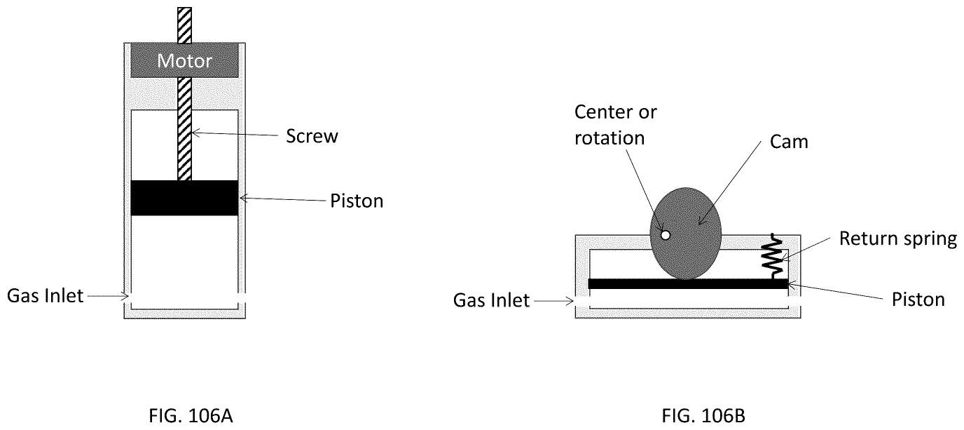

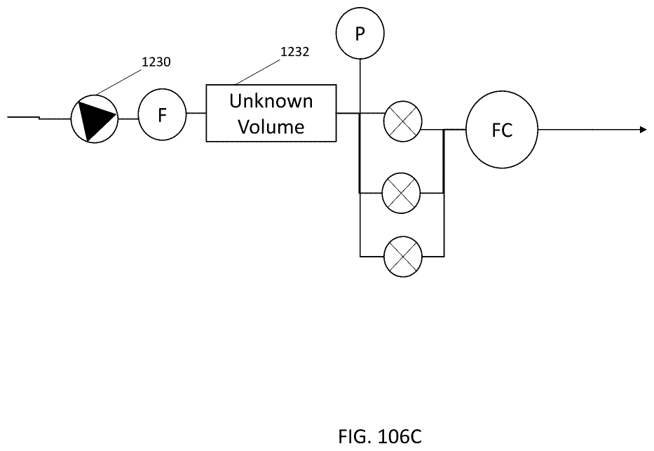

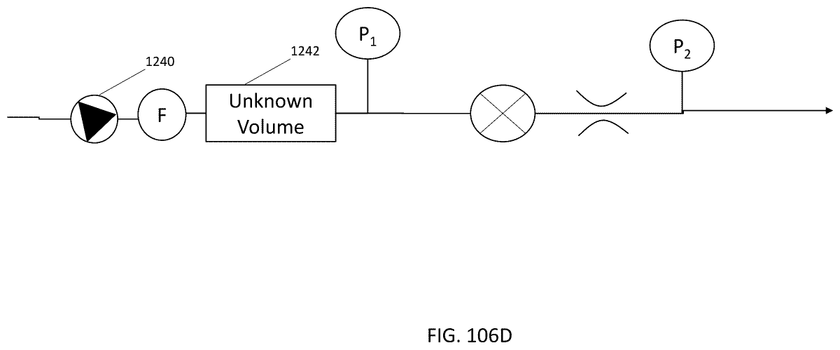

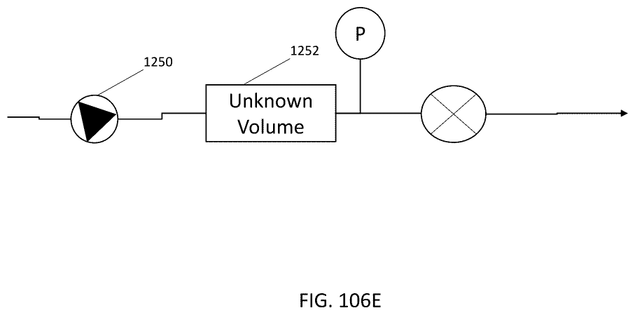

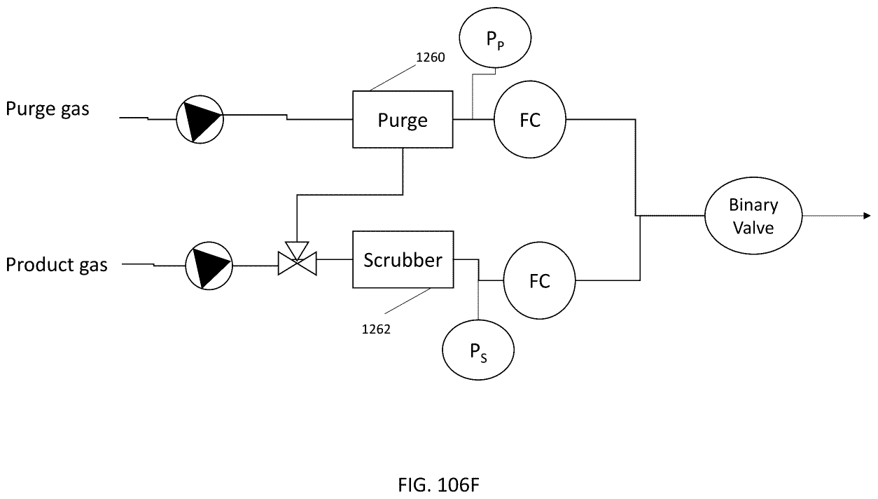

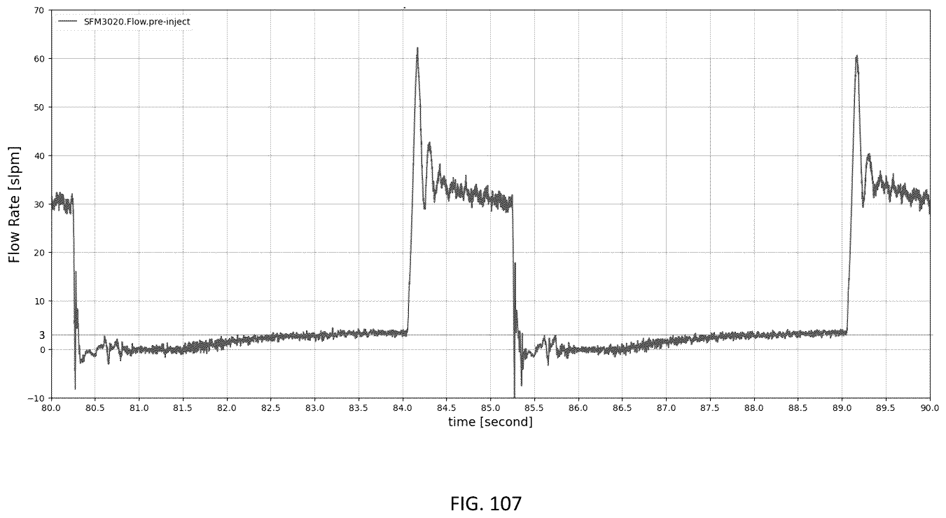

In some embodiments, the system adjusts a position of a volume displacement component (e.g., a piston) within the system to achieve a target dead volume of the system. The volume displacement component is adjusted according to the measured or read dead volume of the system, including scrubber. In one embodiment, the volume compensation is achieved with a syringe pump. FIG. 106A depicts an embodiment of a volume displacement component that varies the position of a piston in a cylinder with a motor and ball-screw. FIG. 106B depicts an embodiment of a volume displacement component that varies a dead volume with a piston driven by a motor-driven cam and return spring.

FIG. 106C depicts another exemplary embodiment of an approach to compensating for a variable dead volume. The system includes a pump 1230 that flows gas through a flow sensor and pressurizes a volume 1232 of variable size (e.g. a scrubber filled with granular scrubber particles). Flow exiting the volume is regulated by a flow controller (far right side of the image). Multiple pathways of varying length connect the unknown volume to the flow controller. The unknown volume is determined by filling the volume to a known pressure as indicated by a pressure sensor with a known volume of gas (integral of data from a flow sensor). In some embodiments, the device controller software compensates for variation in volume by adjusting one or more of NO concentration, peak pressure, and NO pulse duration to achieve target NO delivery. In other embodiments (shown), the system selects a pathway from volume to flow controller that achieves a combined volume that most closely approximates the target volume for the system. Flow through each of the pathways is controlled by a valve. The valve is left open for the flow path that achieves the target dead volume for the system.

FIG. 106D depicts another exemplary embodiment of an architecture for determining the volume of an unknown cavity. The controller begins by ensuring that the pump 1240 is off and the downstream valve is open so that the unknown volume 1242 reaches atmospheric pressure. Then the controller closes a downstream valve and enables the pump to fill the cavity to a known pressure, as indicated by a pressure sensor (P1). The downstream valve is then opened so that the cavity depressurizes to atmospheric pressure. The exiting gas flow passes through a fixed orifice. In one embodiment, the pressure downstream of the fixed orifice is measured by a pressure sensor (P2). The difference in pressure is utilized to calculate a flow rate. The flow rate is integrated over time to determine the volume of gas released from the volume. The volume of unknown cavity is determined with Boyle's law (V1=P2V2/P1), where V1=the cavity volume, P2=the peak pressure in the cavity, V2=the volume of gas measured through the orifice, and P1 is the baseline pressure (typically atmospheric pressure). In some embodiments, if the downstream pressure is known (e.g. it always atmospheric pressure), then the P2 sensor is optional.

FIG. 106E depicts another exemplary embodiment of an architecture for determining the volume of an unknown cavity. A pump 1250 is utilized that is very well characterized for mass flow rate at a range of pressures. The controller enables the pump to fill the cavity 1252 to a known pressure. The flow rate of the pump at each time point is determined based on the pump characterization and cavity pressure. In some embodiments, (e.g. when the pump inlet is not atmospheric pressure), the pump inlet pressure is measured by an additional pressure sensor (not shown) to more accurately determine pressure across the pump and actual mass flow rate. The mass flow rate between the time points is integrated over time to determine a volume of gas added to the cavity. Then, the controller uses Boyle's law is utilized to calculate the volume of the cavity.

FIG. 106F depicts an exemplary embodiment of a system with an unknown scrubber dead volume. The controller initially opens the flow controllers to permit the purge reservoir and scrubber to equilibrate to atmospheric pressure. Then, the flow controllers are closed. A three-way valve is utilized to direct gas flow from the scrubber pump (lower pump) to the purge gas reservoir 1260. The purge reservoir 1260 is pressurized up to a peak pressure, as indicated by the purge reservoir pressure sensor. The pressure measurements in the purge reservoir 1260 and known volume of the purge reservoir are utilized to characterize the mass-flow rate of the scrubber pump at various pressures. Then, the 3-way valve is set to direct gas flow from the scrubber pump to the scrubber 1262. The pump is utilized to fill the scrubber to a known pressure, as indicated by the scrubber pressure sensor. The volume of gas added to the scrubber to achieve the final pressure is determined by integrating the pump flow over time during the filling time period, based on the prior pump characterization. Then, Boyle's law is utilized to determine the dead volume (i.e. void space) within the scrubber.

### Proportional No Delivery at the Patient Y-Fitting

In some embodiments, NO is delivered to the inspiratory limb at or near the patient Y-fitting. Inspiratory flow is measured with a flow sensor near the Y-fitting to ensure accurate quantification. The NO delivery and/or generation device controller determines the NO concentration and flow rate to be injected into the inspiratory limb based on the target dose, product gas concentration, and inspiratory flow rate.

Injecting NO at the patient Y-fitting avoids NO product gas having to travel through the inspiratory limb and humidifier, which can take several seconds and often involves elevated oxygen levels. This decreased transit time can result in a significant reduction in inhaled NO₂ levels. In addition, the gas is injected into the inspiratory limb in phase with patient breathing. Unlike NO injection near the ventilator flow, where the actual subset of gas inhaled by the patient is uncertain, injection at or near the patient Y eliminates uncertainty about which gas will be inhaled. In turn, knowing which gas will be inhaled enables to cease NO delivery during exhalation and between breaths. This results in less wear on an NO generation system, prolonging the life of electrodes, scrubbers, pumps, valves, and other components.

### Zero Bias Flow

Some ventilators bring inspiratory flow rates to zero between breaths. Any NO injected into the inspiratory limb during a period of zero bias flow will create a volume of gas within the inspiratory limb that is overdosed with NO. By injecting NO near the patient or at the patient-Y fitting, overdosed gas during a zero bias flow period is swept into the expiratory limb when the bias flow resumes and is not inhaled by the patient.

FIG. 107 depicts an exemplary graph showing exemplary inspiratory flow data from a Hamilton T−1 ventilator circuit. Inspiration begins at 84 seconds and ends at 85.25 seconds. The inspiratory flow is equal to zero slpm for more than 1 second until time equals 86.5 seconds. Then, the inspiratory flow ramps up to roughly 3 slpm over the next second. The bias flow continues at 3 slpm until the next inspiratory event.

When NO product gas is introduced to the inspiratory limb during a period of zero bias flow, the concentration within the inspiratory limb near the injector becomes the concentration of the product gas. While there can be some mixing along the length of the inspiratory limb, this effect can be small. Depending on several factors including but not limited to the inspiratory flow rates, the inspiratory limb volume, bias flow rate, and patient breath volume, the overdosed volume of gas may be inhaled by a patient.

In some embodiments of a NO delivery system, the system controller varies NO flow rate proportionally to the inspiratory limb flow rate all the way down to zero slpm. Inspiratory flow measurement can be very challenging at very low flow rates, so some embodiments will utilize an inspiratory flow threshold whereby NO flow is proportional to inspiratory flow above the threshold and equal to zero when the inspiratory limb flow rate goes below the threshold. In one specific embodiment, NO flow is set to zero when inspiratory flow goes below 0.5 slpm, for example. In another embodiment, the NO flow is set to zero when the flow rate tolerance (i.e. error) in NO injection flow would result in greater dose error than the error associated with not injecting NO.

In some embodiments, the threshold for setting NO flow to zero is calculated based on the ventilator flow profile. In some embodiments, the volume of gas not dosed with NO is calculated by integrating the inspiratory flow rate over the time that the inspiratory flow rate is below the threshold. Then, the volume of gas dosed with zero NO is compared to the tidal volume of the patient. For example, FDA Regulations permit 10% of the inspired volume to have zero NO. Hence, a patient breathing 300 ml tidal volume, could have 30 ml of gas dosed with zero NO. In one embodiment, the threshold for arresting NO flow is determined by selected the inspiratory flow rate threshold that results in the acceptable underdosed volume (30 ml in this example) to occur.

### Pulsed Delivery at the Patient Y-Fitting

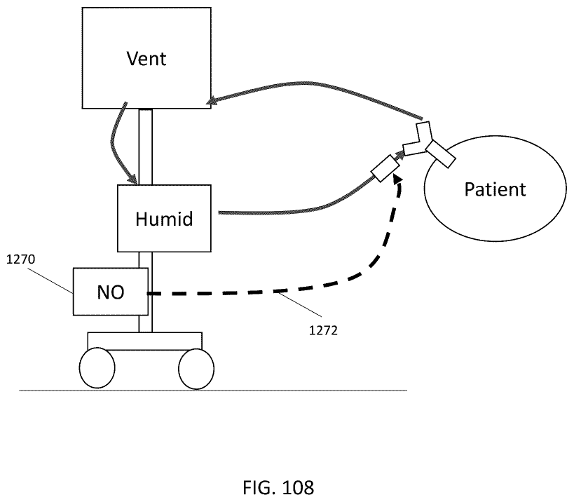

In some embodiments, NO is delivered in pulsatile form to a patient on a ventilator through a delivery tube. The tube connects at or near the patient Y. In some embodiments, it connects to the endotracheal tube. FIG. 108 depicts an embodiment of a system with NO delivered from a NO generator 1270 through a tube or delivery device 1272 that is independent of the inspiratory limb. In some embodiments, pulses of NO are delivered only during an inspiratory event. Pulsed NO should be delivered very close to the Y fitting so that the NO is delivered to the beginning of the breath to treat the deep lung. Any dead volume between the injection point and Y fitting will enter the patient without NO.

In the embodiment shown, the NO device detects inspiratory events based on pressure changes within the NO delivery lumen. A person skilled in the art will understand that there are multiple approaches to inhalation detection that could be applied, including but not limited to flow measurement in the inspiratory limb and communication with the respiratory equipment (the ventilator).

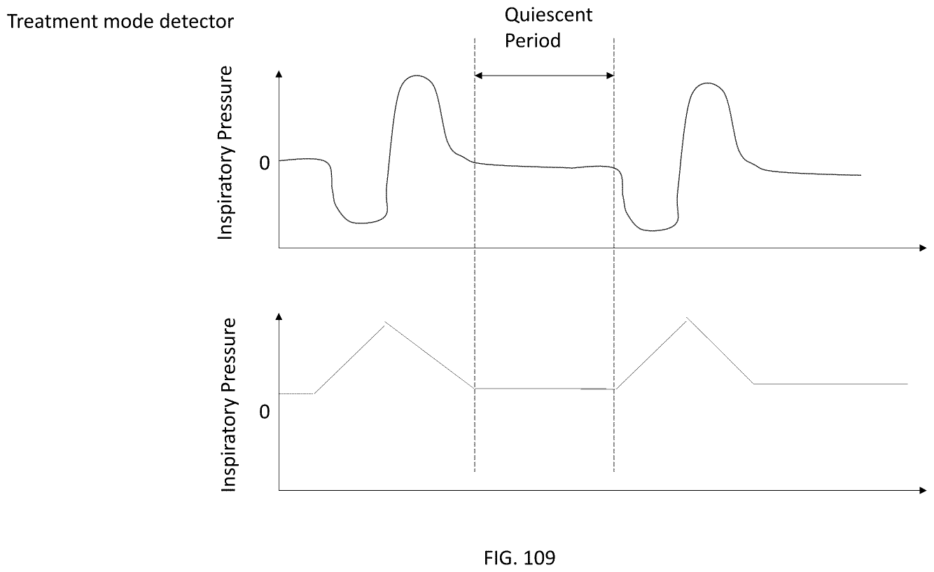

The triggering event for detecting inspiration varies with treatment modality. For example, a spontaneously breathing patient pulls a vacuum when their diaphragm contracts, drawing air into their lungs. Contrastingly, pressure within the airway increases when respiration is driven by an external device such as a ventilator. There can also be a shift in the baseline pressure within the respiratory system, such as when a CPAP system is used to maintain an elevated pressure within the lungs. An automatic treatment mode controller can detect the type of treatment by the breath signals. FIG. 109 depicts an exemplary graph of a method that can be used by a NO delivery system to detect treatment type. The system identifies the quiescent period that takes place after exhalation and before inhalation. This period lasts hundreds of milliseconds to several seconds. The event that follows the quiescent period is inhalation. In a spontaneously breathing patient, inhalation involves pulling vacuum on the delivery system. In a ventilated patient, inhalation is associated with increased pressure in an inspiratory limb. By observing the slope of the inspiration signal (i.e. positive=ventilation, negative=spontaneously breathing), the system can determine when inhalation occurs and provide pulsatile NO at a target time point in the respiratory cycle.

In some embodiments, the NO device communicates with the treatment device to be notified of the timing of inspiratory events.

Patients receive a finite amount of NO with a portion or all of their breaths. The quantity of NO is tracked in units of mg NO to deliver a target dosing rate in units of mg/hr.

The NO device is set to deliver a specific amount of NO per unit time (e.g., 6 mg/hr). The user can also select what portion of the breath should receive NO (e.g. all, first 50%, middle 50%, etc.). The portion of the breath that is dosed is related to the region of the airway and/or lung that receives the NO. For example the portion of the breath that is dosed can differ, depending on whether an airway infection or a deep lung infection is being treated.

Using pulsed NO delivered to a location near the patient, the NO is not exposed to elevated oxygen levels within the inspiratory limb which can hasten the formation of NO₂. The NO can also be delivered rapidly from a storage location (e.g., a pressurized scrubber) to the patient so that the transit time is extremely brief (tens of milliseconds) Pulsed delivery can also allow for prolonged exposure to NO₂ scrubber material ensuring high levels of NO₂ scrubbing (e.g., >99% removal of NO₂).

When electrically-derived NO is used with pulsed delivery, the oxygen level within the product gas is typically atmospheric (21%) and there is very little time for NO₂ to form before the gas reaches the patient. This can change the risk profile for NO₂ exposure, NO dose uncertainty and FiO₂ changes. With pulsed NO delivery, a lack of NO₂ measurement at the patient can be justified because it is essentially zero. In addition, a lack of NO measurement at the patient can be justified because the NO concentration at the patient is essentially the same as it was tens of milliseconds before when it was in its storage vessel. In some embodiments, the concentration of NO in the storage vessel is measured by a sensor and/or determined based on an open-loop control scheme. Pulsatile NO delivery also results in negligible dilution of the inspiratory oxygen stream, making the measurement of O₂ at the patient unnecessary because the O₂ measurement at the O₂ delivery device (e.g. ventilator) will be accurate.

Elimination of gas sensing at the patient enables significant simplification of the NO delivery system and its deployment steps. A gas collection apparatus, sample line, sample line filter, water trap and other components specific to gas measurement at the patient can be eliminated from the system. This reduces the use steps for deploying a NO delivery device along with the potential failure modes that those use steps can cause.

In some embodiments, the dose for a patient in mg/hr is selected based, in part, on the ideal body weight of the patient. In some embodiments, the dose is selected based, in part, on the stature (AKA height) of the patient. The clinical purpose of the NO treatment is also considered when determining the NO dose. For example, NO treatment to dilate the blood vessels of the lung and airway (e.g. 0 ppm to 80 ppm) are much lower than doses for killing infectious organisms (e.g. 80 ppm to 1000 ppm).

### NO Delivery at the Endotracheal Tube

In some embodiments, a pulse of NO is delivered to an ET tube during the quiescent period after exhalation and before inspiration. During the next inspiration, the patient draws the NO within the ET tube into their lungs.

### Injected NO Mixing

State of the art NO delivery systems require introduction of NO to the inspiratory limb at a location that is 2-feet to 6-feet away from the patient. This is because it takes time for injected NO to mix with inspiratory gases to form a homogenous mixture. Without a homogeneous gas mixture, the concentration of NO within various regions of the lung and airway would be inconsistent.

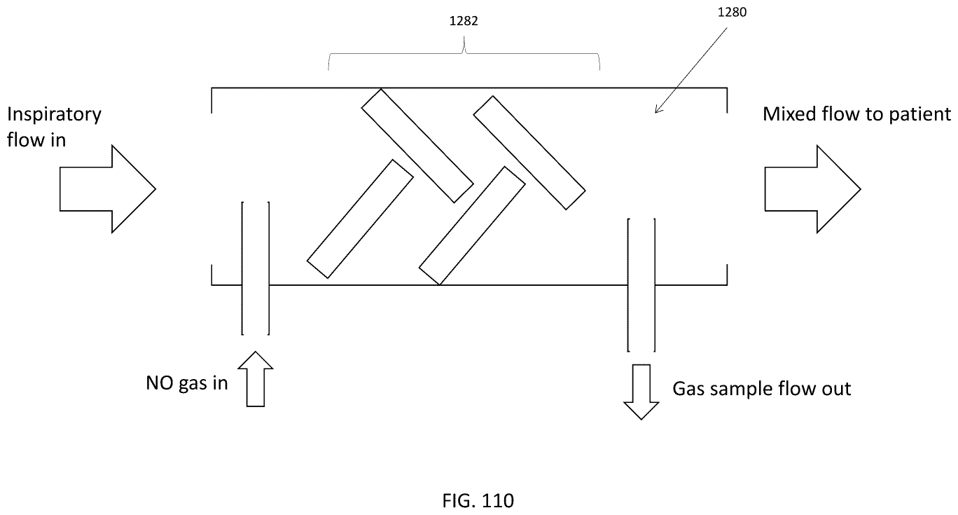

Delivery of NO proximal to the patient, whether by pulse or continuous delivery, can provide benefits in lower NO₂ levels but presents a challenge in gas mixing. FIG. 110 depicts an exemplary embodiment of a NO injection module 1280 that is inserted into an inspiratory limb. The module 1280 includes an opening to receive an inspiratory gas flow. NO is injected into the inspiratory gas flow. The inspiratory gas flow and NO gas pass through a mixer. Either static or dynamic mixer can be utilized. A static mixer 1282 with multiple flow interrupters that induce turbulence is shown. An example of a dynamic mixer is a rotating fan within the gas stream. After the gas is mixed, a gas sample is pulled from the flow for measurement of the concentration of one or more of NO, NO₂, O₂, and He. The gas mixture exits the right side of the figure and is delivered to the patient.

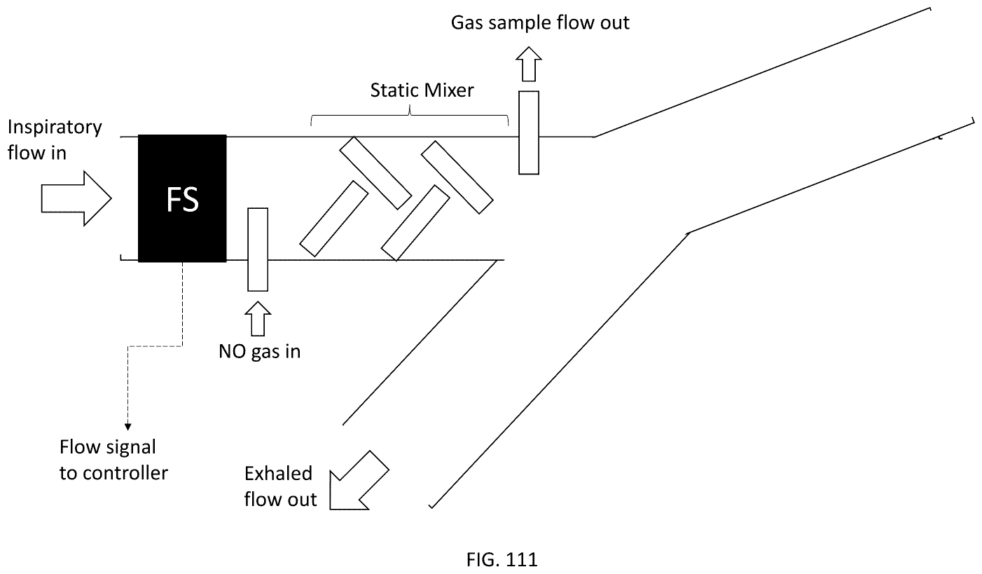

FIG. 111 depicts an embodiment of an NO injection module that is built into a patient Y fitting. This embodiment includes an optional flow sensor as well. In one embodiment (not shown), a one-way valve is included between the gas sampling location and the patient expiratory flow path to prevent the gas sampling system from sampling from the expiratory flow. In another embodiment (not shown), the distance between the NO sampling location and the inspiratory/expiratory flow intersection is elongated to prevent exhaled gas from entering the gas sampling tube.

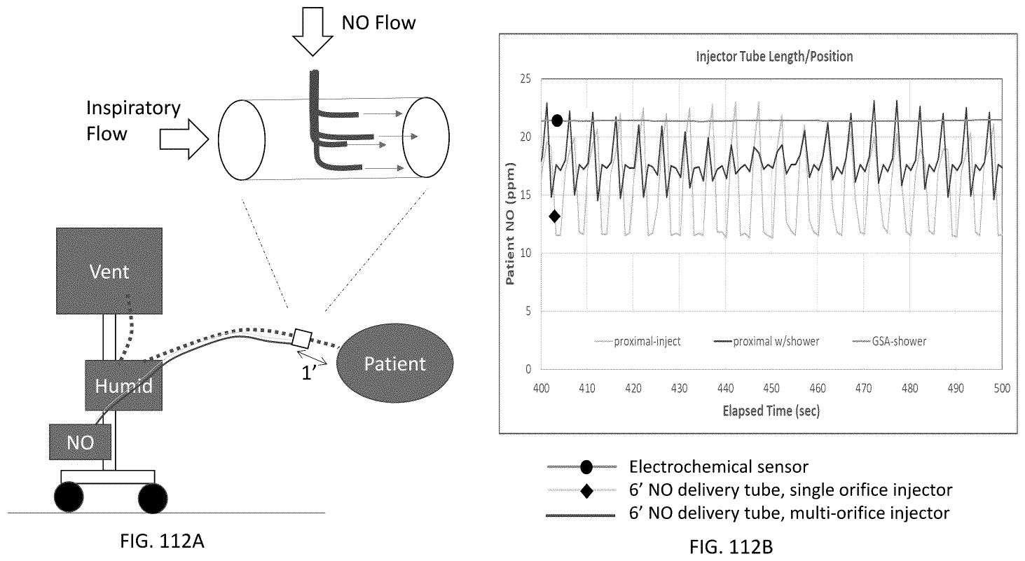

FIG. 112A depicts an embodiment of a NO injection module that promotes mixing of NO into the inspiratory flow. NO is introduced to the inspiratory flow through multiple orifices that are distributed across the inspiratory flow. The multiple orifices comprise multiple tubes connected to a common NO inlet. By introducing the NO to multiple locations across the inspiratory flow, the NO gas mixes more quickly with the inspiratory flow, enabling the introduction of NO at locations more proximal to the patient and with less NO transit time in potentially high inspiratory concentrations of oxygen. FIG. 112B illustrates an exemplary graph that demonstrates the improved homogeneity of the gas mixture when a multiple orifice injector is utilized. These data were collected with a rapid NO analyzer (Sievers) that could capture variations in concentration within the inspiratory gas stream. Gas samples were collected one foot downstream of the NO injection location. For reference, the data from a slow electrochemical sensor, industry standard on NO delivery systems, is provided for reference. The slow response time of the electrochemical sensor results in an indication of constant NO concentration within the inspiratory limb. The rapid analyzer, however, is able to distinguish between the variation in concentration with and without a multi-orifice NO injector.

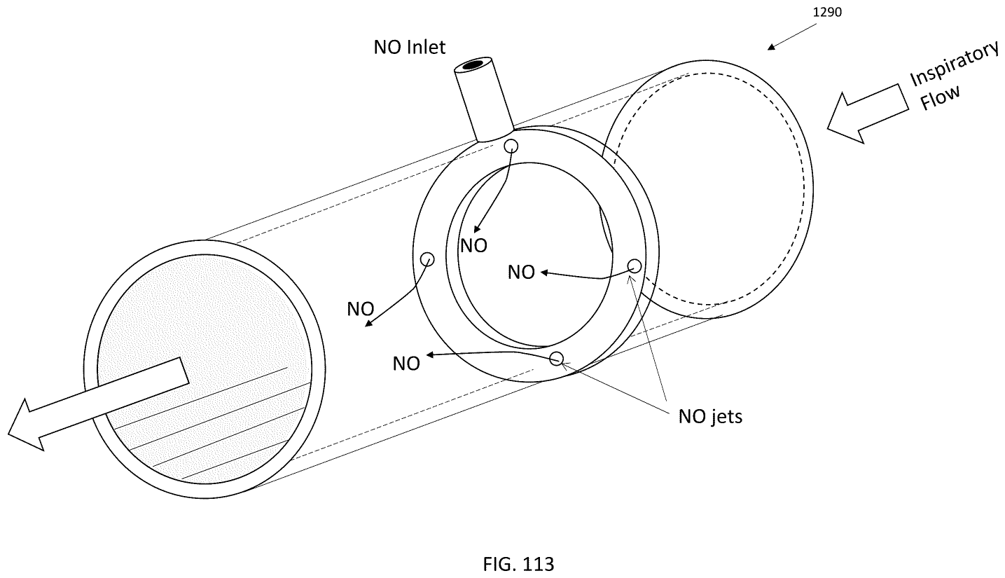

FIG. 113 depicts an embodiment of a NO injection module 1290. NO is introduced to an anulus. The annulus includes one or more orifices that release NO into the inspiratory gas stream. In some embodiments the NO is introduced in a direction that is oblique to the direction of inspiratory gas flow (i.e. not parallel) to promote mixing. In some embodiments (not shown), NO gas flow is introduced in a retrograde direction (opposite or partially opposite the inspiratory flow direction) to promote gas mixing.

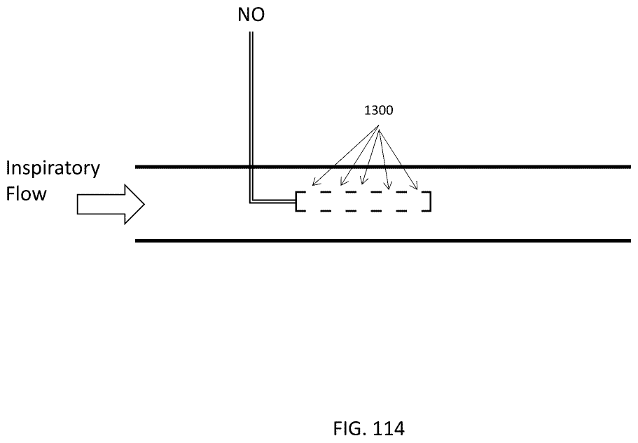

FIG. 114 depicts an embodiment of an inspiratory flow path in which NO is introduced to an inspiratory flow through an array of orifices 1300 oriented along the length of the inspiratory flow path. In some embodiments, the size of the orifices varies with the upstream orifices having smaller diameter than the downstream orifices to promote more even flow through all of the orifices (i.e. to account for pressure drop within the injector body). By injecting NO along the length of the inspiratory flow, the NO is effectively volume-averaged into the inspiratory flow, smoothing out inaccuracies in the dose delivery. In some embodiments, the injector is in the form of a hollow needle with an array of radial holes. This design can distribute NO evenly into the inspiratory flow with small volume, to minimize transit time of the NO through the injector, thereby minimizing NO₂ formation.

### Alternative Embodiments

### Pole Mounted

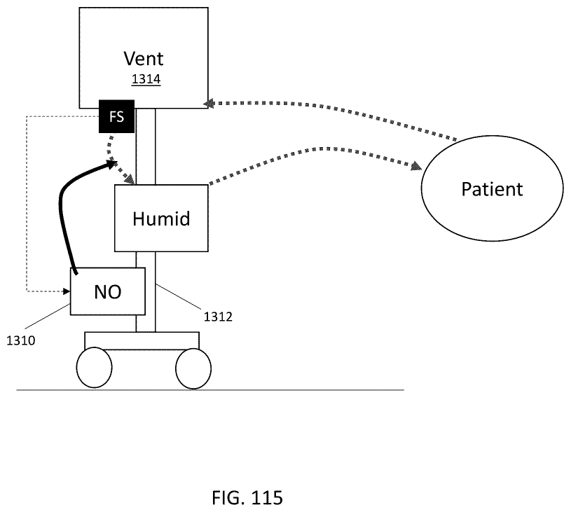

FIG. 115 depicts an embodiment of a NO generation and delivery system 1310 that is mounted to a ventilator stand 1312. Inspiratory gas exits a ventilator 1314 and passes to a humidifier that is also mounted on the ventilator stand. The NO device delivers NO to the dry side of the inspiratory limb (i.e. upstream of the humidifier). An optional flow sensor (labeled “FS”) is mounted to the inspiratory limb to provide the NO generator is inspiratory mass flow rate measurements. In some embodiments, the ventilator communicates inspiratory flow rate information to the NO device via wired or wireless means. Mounting the NO system on the same stand as the ventilator can reduce the number of stands/poles/devices that are required to treat a patient. This decreases the footprint of the ventilator and NO device as well as simplifies the process of transporting a patient.

### Remote GUI

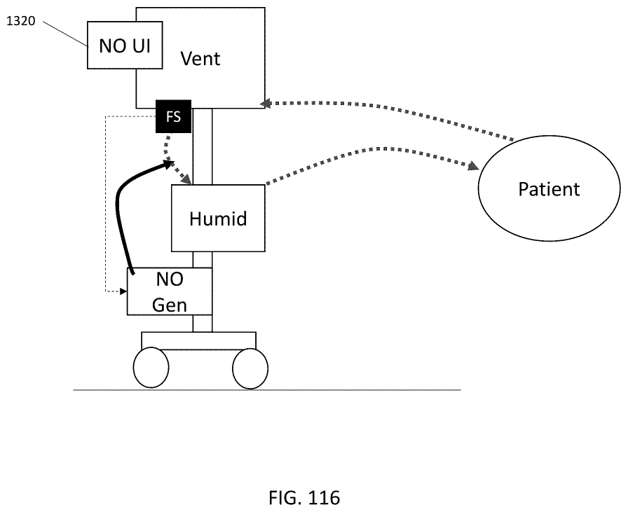

Placing the NO device at the bottom of a ventilator stand can make it difficult to view the NO device user interface and adjust NO treatment settings. FIG. 116 depicts an embodiment of a system with a remote user interface 1320. In some embodiments, the user interface consists of a remote screen with tactile controls (e.g. buttons, knobs, levers) and/or a touchscreen. The user interface displays one or more of current treatment dose setting, gas sensor measurements, alarm conditions, treatment elapsed time, weaning settings, calibration information, trending data, system set-up instructions, and system tear-down instructions. The user interface communicates with one or more wired and wireless means with the NO generation device and/or respiratory device (e.g. ventilator, CPAP machine).

### Voice Prompts

In some embodiments, the UI of a NO generation device provides voice prompts and/or videos to instruct a user how to one or more of set up a system, use a system, respond to system faults, replace disposable components, calibrate a system, and tear down a system. In some embodiments, the system detects various actions of the user (e.g., introducing calibration gas, inserting a scrubber cartridge, pressing a button) and advances to the next step of instruction automatically.

### Home No Generation System

In some embodiments, a stationary NO generator is utilized to fill tanks of NO in a balance of NO₂ for use with a respiratory device. In some embodiments, the stationary NO generator in the form of a cradle for a portable NO delivery device, typically placed on a nightstand overnight. When a portable NO delivery device is docked, the stationary NO generator does one or more of fills an onboard NO tank within the NO delivery device, charges the delivery device battery, cleans an NO₂ scrubber (e.g. MOF scrubber), downloads data from the device (e.g. performance data, faults, patient data), and calibrates sensors onboard the NO delivery device (e.g. flow, NO concentration, pressure, etc.). In some embodiments, the stationary NO generator is in the form of a nightstand or a device that sits atop a nightstand such that a NO delivery device can be docked with the NO generator while the patient sleeps.

In some embodiments, a stationary NO generator is utilized to fill one or more gas cylinders with a mixture of NO and nitrogen gas. In some embodiments, the gas cylinders are large cylinders used for hospital applications. In some embodiments, the gas cylinders are removably attached to a portable NO delivery device. A user receives NO from the cylinders through the portable delivery device and replaces the NO cylinders when they are at or near empty.

In some embodiments, the stationary NO generator first generates NO in an air mixture (e.g. electrically, with an NO donor molecule, reducing N₂O₄ gas, etc.). The NO/air mixture is then passed through a pressure-swing adsorption process whereby molecular sieve material with an affinity for nitrogen and NO separates oxygen from the gas mixture, leaving NO in nitrogen. In some embodiments, the NO in nitrogen is scrubbed for NO₂ (e.g. soda lime) prior to storage in a gas cylinder for use. In some embodiments, a membrane is utilized to separate at least some of the nitrogen from the NO to increase the concentration of the NO gas mixture prior to storage in a cylinder. In other embodiments, a membrane that can separate NO from N₂ and/or NO₂ is utilized to increase NO concentration in the gas mixture within an NO delivery device prior to NO delivery to a patient with the balance of nitrogen being released into the atmosphere. In some embodiments, concentrated oxygen exiting the pressure-swing adsorption process is also stored in gas cylinders for later use.

### NO Generation Systems

In some embodiments, water is permitted to condense out of product gas at the point of highest pressure within the system. In some embodiments, the location of highest pressure is at or before a scrubber. In some embodiments, the location of highest pressure is within or after a pump. Condensed water is collected in a water trap. In some embodiments, the water trap is periodically drained by a user. In other embodiments, the water collected from reactant/product gas is merged with water separated from an inspiratory gas sampling stream into a common reservoir that can be drained or replaced by a user. In other embodiments, water is introduced to the gas flow that convectively cools the overall device, the elevated temperature convection gas having a greater capacity to hold water than ambient air.

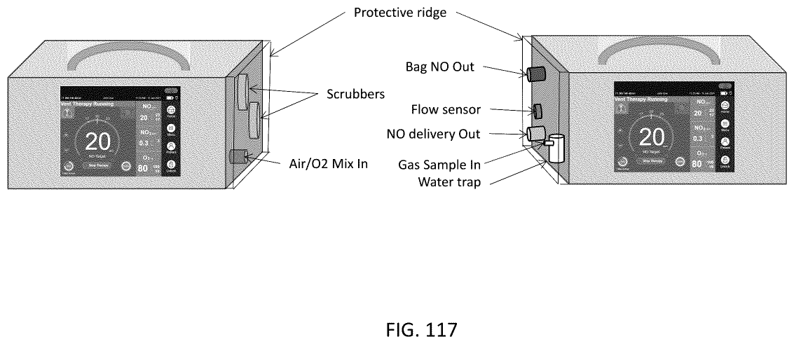

FIG. 117 depicts an exemplary embodiment of a NO generation and delivery system. The system includes an enclosure that houses a processor, pumps, valves, sensors, and other components. The device is short in stature to fit on a helicopter skid. Patient connections including inspiratory flow sensor, NO delivery, bag NO delivery, and gas sample line (through water trap) are all on one side of the device. This facilitates establishing patient connections and minimizes the length of patient-related electrical cables and pneumatic tubes. The opposite side of the device includes other, non-patient connections including one or more of replaceable NO₂ scrubbers and incoming air preparation components (humidity, VOC, and/or particulate). External power and replaceable battery connections (not shown) are typically located on the rear panel of the device. The system optionally receives external communication (e.g., RS232) from a ventilator as well. In some embodiments (shown), the enclosure includes a protective ridge that protects electrical and pneumatic connections to the system from being contacted during device transport.

### Docking Station

In some embodiments, a portable and/or wearable NO generation and delivery device is periodically docked at a docking station. In some embodiments, the docking station battery charging. In some embodiments, the docking station is utilized to reset an NO₂ scrubber (e.g. apply one or more of temperature/vacuum/gas flow to reset a metal organic framework (MOF) material. In another embodiment, a docking station can hydrate a soda lime to prolong the scrubber service life. In another embodiment, a docking station connects to one or more of the internet, local area network, Wi-Fi network, etc. for one or more of data transfer, software update, remote control, and system status reporting. In some embodiments, a docking station includes one or more cannisters of calibration gas (e.g. NO, NO₂, O₂) for calibration of sensors in the NO delivery and/or generation device. In another embodiment, a docking station includes a NO sensor that is utilized to calibrate the NO production/delivery from a NO generation and/or delivery device. In one embodiment, the NO generation and/or delivery device sends product gas generated by the device to the docking station through a pneumatic connection. The docking station measures the NO concentration within the product gas with a sensor. If the product gas is accurate to within acceptable limits, the docking station communicates (wired or wirelessly) with the NO delivery/generation device that calibration is complete. If the NO product gas is not within calibration, the docking station does one or more of the following: Communicates the error to the NO generation/delivery device, records the failure in a log file, communicates the failure with a user, communicates the failure with an entity (e.g. manufacturer, doctor, etc.) through a communications network. In some embodiments, the NO generation/delivery device adjusts NO production/delivery settings (e.g. plasma frequency, plasma duty cycle, plasma power, plasma AC waveform, reactant gas flow rate) based on the feedback from a failed calibration test. In some embodiments, the NO generation/delivery device repeats the NO production procedure one or more times in order achieve a passing result.

### User Interface

In some embodiments of a NO generation and/or delivery system, the user interface includes a display of patient methemoglobin level. In some embodiments, the measurement depicted comes from a methemoglobin sensor that is part of the NO generation and/or delivery system. In some embodiments, the methemoglobin measurement is sourced in a wired or wireless way from an external methemoglobin analyzer. In some embodiments, the NO delivery system alters or stops NO delivery when methemoglobin levels reach or cross a threshold. In other embodiments, the NO delivery system generates an alarm when methemoglobin levels reach or cross a threshold.

In some embodiments, a NO delivery device user interface displays the rate of NO drug delivery (e.g. mg/hour). In other embodiments, the user interface displays a timer. The timer is used for one or more of displaying remaining treatment time, and time until the next dose of NO is to be delivered.

In some embodiments, a NO delivery device user interface displays a measurement of cardiac output. In some embodiments, a NO delivery device displays a measurement of lung cross-sectional area or volume (e.g. EIT data). In some cases, a NO delivery device displays a change in lung cross-sectional area or volume with respect to a baseline measurement. This information can be used by a user to quantify and optimize the effectiveness of nitric oxide with a subject patient.

### Separation of Flow Measurement and No Injection

In some embodiments, inspiratory flow is measured in a different location within the inspiratory limb than NO injection. This approach can be useful because NO injected into the inspiratory limb has very short transit time to the patient; an issue that is most significant when oxygen levels within the inspiratory limb are elevated. This approach also minimizes the complexity of the NO injection module and components near the patient, which is preferred by respiratory therapists. A further benefit is that it keeps the inspiratory flow sensor upstream of the humidifier, in a dry environment, which can improve sensor readings and longevity. In some embodiments, an NO injector is combined with a gas sampling module.

In some embodiments, the NO generation system measures the inspiratory limb flow rate in, at or near the ventilator (e.g., pre-humidifier) and injects NO downstream, closer to the patient. As the ventilation flow waveform propagates down the inspiratory limb, the waveshape changes due to one or more of gas compression, back pressure, turbulence, and inspiratory limb volume. This difference in inspiratory flow waveshape can result in errors in inspiratory gas concentration because the inspiratory flow being dosed at the point of NO injection does not have the same profile as the inspiratory flow waveform that was measured and served as input into the dosing algorithm (e.g., proportional flow algorithm).

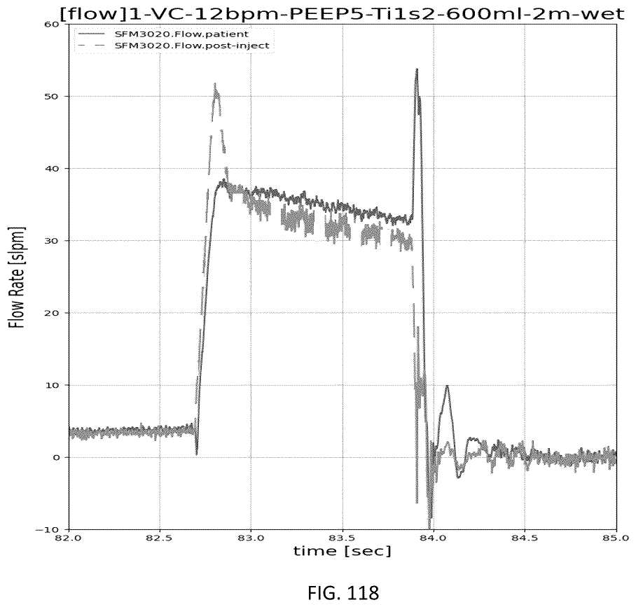

FIG. 118 depicts exemplary flow data collected from a ventilator circuit near the ventilator (dashed line) and near the patient (solid line) in real time. The flow signal at the patient is delayed due to pressure propagation through the length of the inspiratory limb at the speed of sound (roughly a 6 msec delay). In addition, sharp features of the initial inspiratory flow waveform soften as they travel through the inspiratory limb. The measurement near the patient also includes a spike in flow rate at the end of inspiration due to expiratory flow beginning in the expiratory limb. In some embodiments, the flow rate of NO delivery is concomitantly spiked to dose the expiratory event. In other embodiments, the expiratory flow spike is not dosed since it is at the end of inhalation and the specific gas molecules passing through the injector at that time are unlikely to enter the patient. In some embodiments, the NO delivery device includes a flow sensor in the expiratory limb of the patient that indicates when exhalation begins (not shown). In another embodiment, the ventilator communicates the timing and/or flow profile of exhalation to the NO delivery device for the NO delivery device to know the timing of the exhalation artifact in the inspiratory flow.

In some embodiments, the amount of error introduced by measuring flow and injecting NO in different locations is small enough that the system can meet dose accuracy requirements without correction and can therefore be ignored. In some embodiments, there is sufficient mixing downstream of the NO injector (e.g. by a length of tubing or a static mixer) that variations in concentration stemming from mismatch between the actual flow rate at the injector and injector flow that is proportional to upstream flow measurements become acceptable before they reach the patient. In some embodiments, the NO delivery system applies a transfer function (e.g. an equation) to the measured inspiratory flow to transform the measured flow signal into one that would be more representative of the flow at the injector. In some embodiments, the transfer function applied to measured inspiratory flow includes one or more of a delay and a low pass filter.

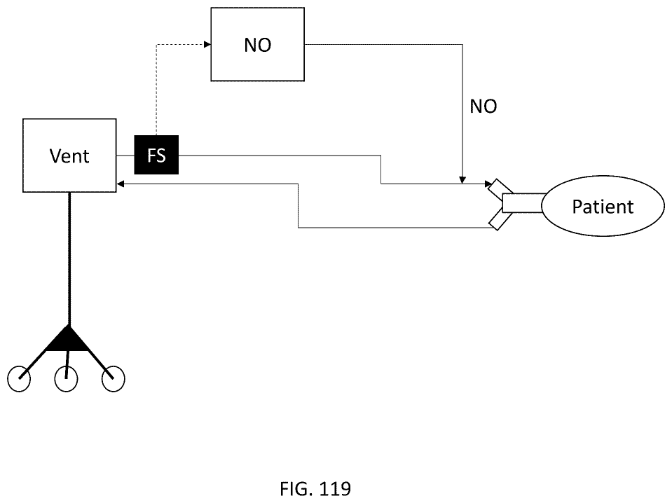

FIG. 119 depicts an embodiment of a NO delivery system connected to the inspiratory limb of a ventilation circuit. The device controller takes the inspiratory flow signal and modifies it to resemble the inspiratory flow at the injector. In some embodiments, the modification involves applying a low pass filter. In some embodiments, the inspiratory circuit type is known, detected or entered by the user and the NO delivery system applies the appropriate transfer function for that tubing set to the measured inspiratory flow signal. In some embodiments, inspiratory pressure is also measured and serves as an input to the inspiratory flow transfer function.

In some embodiments, the system can characterize the inspiratory limb to ascertain the transfer function. This may be done before or during NO treatment.

### Humidifier Effect on NO Concentration

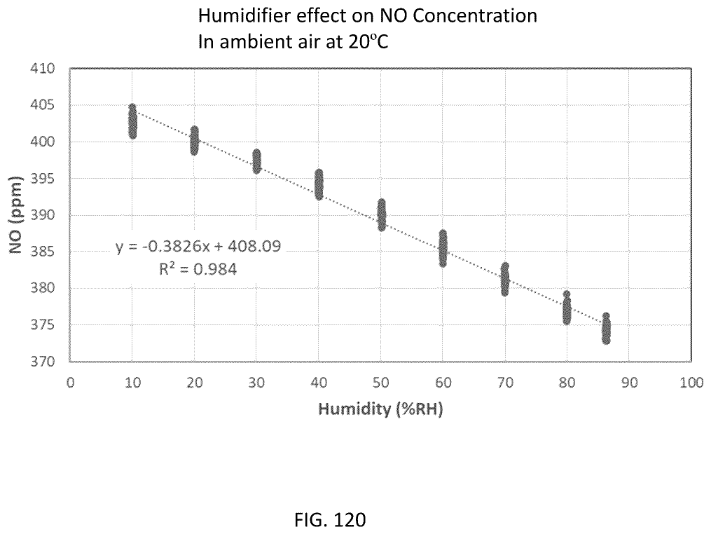

A NO delivery system typically measures the flow rate of inspiratory gas in the dry portion of an inspiratory circuit (i.e. before the humidifier) and delivers NO according to the target dose setting to the dry inspiratory air. As the gas passes through a humidifier, the gas absorbs water from the humidifier. The water molecules are added to the inspired gas, thereby decreasing the concentration of NO within the inspired gas. FIG. 120 depicts an exemplary graph showing the effect of adding humidity to a 403 ppm NO gas stream at room temperature. At 85% relative humidity, the concentration of the gas has decreased to 375 ppm, or an 7% decrease. At physiologic temperature (e.g. 37 deg C.), the gas can hold even more water with a more dramatic decrease in NO concentration. This difference between delivered dose and actual inspired concentration can result in under dosing of a patient. In some NO delivery systems, the system is calibrated by measuring the diluted NO concentration downstream of a humidifier. In other embodiments, a NO delivery utilizes gas measurements downstream of a humidifier as input to a closed-loop dose controller to avoid inaccuracies in delivered dose arising from humidity effects.

### Sterilization

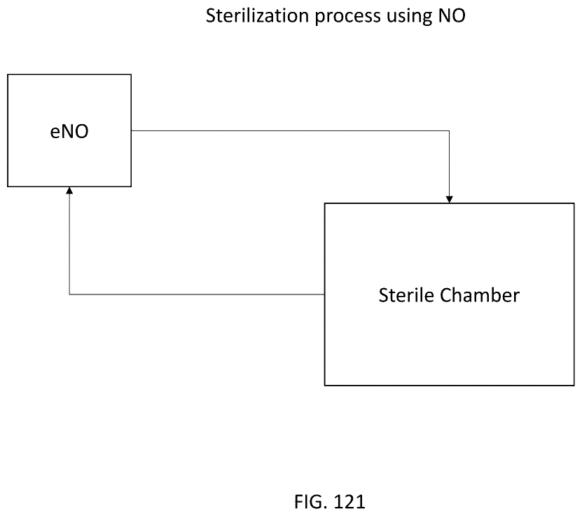

In some embodiments, an electrical NO generation system provides high concentration NO gas (>80 ppmNO) to a sterilization chamber. FIG. 121 depicts an exemplary electric NO system for sterilization. Product gas containing one or more of NO and NO₂ from the electric NO generator are introduced to the sterilization chamber and flow through it. In some embodiments, gas exits the chamber and returns to the NO generator to form a recirculation loop (shown). In other embodiments (not shown), product gas exits the sterilization chamber and is exhausted from the system. In some embodiments, released product gas is scrubbed for one or more of NO, NO₂, NOx prior to release from the system. This can prevent the accumulation of NOx gases in the local environment.

### Internet of Things

In some embodiments, a NO generation and/or delivery system is connected to the internet. Through the internet connection, the device can provide data to a remote clinician or health care provider. These data can enable remote observers to monitor the status of the device, the patient and the patient's conformance to the prescribed treatment. In some instances, the data collected trigger action like a reminder to the patient to use the device, automatically sending a replacement NO device when the current one is showing signs of nearing the end of service life, or prompting a shipment of replacement scrubbers based on usage data. In some embodiments, the device can also be remotely controlled by a remote clinician or healthcare provider.

In some embodiments, a medical device generates a code (e.g. bar code, 2D bar code, QR code, code number, etc.) on the user interface for communication of information. In some embodiments, a medical device projects a code on a graphical user interface. In some embodiments, the code is read by a user utilizing a scanning device (e.g. a cell phone or other optical device (e.g. a code scanner)) to read the code. In one embodiment, the scanning device utilizes address (e.g. world wide web URL, IP address, data transfer phone number) information embedded within the code to connect with a server. The scanning device is utilized to one or more of process (e.g. decode, transform, calculate results from) information from the medical device, display to a user the decoded information, or deliver information embedded within the code to a secondary device (e.g. a cloud-based server). Examples types of information that can be communicated in this way include but are not limited to status of consumables (e.g. quantity of gas used), cumulative system run time, system fault conditions, alarm conditions, system use information (e.g. number of patients treated), current system configuration (e.g. calibration data, software settings, software version), disposables information (e.g. lot #, serial #, expiration date, duration of use, etc.), environmental conditions, billing information (e.g. amount owed for service provided by the medical device), and the occurrence of any drop/impact events.

In one example, a medical device (e.g. a ventilator) detects that an inlet filter is due for replacement. The medical device displays a 2D bar code on the user interface screen. A user uses their cell phone to scan the 2D bar code. The 2D bar code directs the cell phone to a web page that instructs the user on how to replace the inlet filter. In another example, the cell phone runs an application that transfers information embedded in the 2D bar code (e.g. hours of use) to a cloud-based server using the cellular network for billing purposes. This approach removes the need for the medical device to connect to a local, hospital network. It also provides a paperless means to inform the owner/manager of said medical equipment use information that can inform the billing process.

It is common practice for care givers to scan a bar code on a drug vial to identify the drug administered and time of delivery for a Hospital Information System (HIS). In one embodiment, a NO generation and/or delivery device provides an optical code on the user interface that can be scanned by a care giver. The code provides information to the HIS related to one or more of the patient (e.g. their ID number), the drug administered (i.e. nitric oxide) and the dose (e.g. inhaled concentration, cumulative drug delivered, etc.).

In some embodiments, a code on the user interface of a medical device can also be scanned by a user to initiate a voice or text-based chat with service regarding the particular unit. In some embodiments, the unit also sends diagnostic information to the service team in parallel with the interaction with service personnel to provide additional information for diagnosis and instruction (e.g. device settings, sensor readings, accessory installation detection, fault conditions, etc.).

What is claimed is:

1. A nitric oxide (NO) delivery system, comprising:
one or more pairs of electrodes configured to ionize a reactant gas into an NO-containing product gas;
a delivery line configured to deliver at least a portion of the product gas into an inspiratory flow of gas; and
at least one controller, the at least one controller configured to control an amount of NO in the product gas generated by the one or more pairs of electrodes using one or more parameters as input to the at least one controller;
wherein one of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

2. The system of claim 1, wherein the dilution value is a dilution factor, the dilution factor being a function of an injected inspiratory flow and a pre-injection inspiratory flow.

3. The system of claim 1, wherein the dilution value is a dilution ratio, the dilution ratio being a function of an injected inspiratory flow and a flow of inspiratory gas downstream of the NO injection.

4. The system of claim 1, wherein the dilution value is variable.

5. The system of claim 4, wherein the controller is configured to select the dilution value to minimize a level of inhaled NO₂.

6. The system of claim 4, wherein the controller is configured to select the dilution value such that the flow controller operates within an acceptable operating range.

7. The system of claim 4, wherein the controller is configured to select the dilution value such that the NO concentration of the product gas is compatible with materials in a product gas flow pathway.

8. The system of claim 4, wherein the controller is configured to select the dilution value to minimize a dilution of inspiratory flow and corresponding inhaled oxygen levels.

9. The system of claim 1, wherein the controller is configured to select a high dilution value to purge one or more product gas pathways to reset the product gas concentration within the one or more pathways to one or more known conditions.

10. The system of claim 9, wherein at least one of the one or more known condition is a known product gas concentration.

11. The system of claim 9, wherein the controller is configured to initiate a purge at specific time intervals.

12. The system of claim 9, wherein the controller is configured to initiate a purge when an expected product gas NO concentration and a measured product gas NO concentration differ by a threshold amount.

13. The system of claim 1, wherein the flow rate of product gas injected into the inspiratory flow has a variable flow rate.

14. The system of claim 13, wherein the controller is configured to convert the variable flow rate of product gas injected into the inspiratory flow to a constant flow rate of product gas when a detected breath frequency increases beyond a threshold.

15. A nitric oxide (NO) delivery system, comprising:
one or more pairs of electrodes configured to ionize a reactant gas into an NO-containing product gas;
a scrubber configured to remove NO₂ from the product gas;
a flow controller configured to deliver at least a portion of the product gas into an inspiratory flow of gas and at least a portion of the product gas upstream of the scrubber; and
at least one controller, the at least one controller configured to control an amount of NO in the product gas generated by the one or more pairs of electrodes using one or more parameters as input to the at least one controller;
wherein one of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

16. A nitric oxide (NO) delivery system, comprising:
one or more pairs of electrodes configured to ionize a reactant gas into an NO-containing product gas; and
at least one controller, the at least one controller configured to control an amount of NO in the product gas generated by the one or more pairs of electrodes using one or more parameters as input to the at least one controller;
wherein one of the parameters is a dilution value derived as a function of an inspiratory flow rate and a target inspiratory gas NO concentration level, the dilution value being used by the at least one controller to set a flow rate of the product gas injected into the inspiratory flow and to determine a target concentration of NO in the product gas.

17. The system of claim 16, further comprising a delivery line configured to deliver at least a portion of the product gas into an inspiratory flow of gas.

18. The system of claim 16, further comprising a flow controller configured to deliver at least a portion of the product gas into an inspiratory flow of gas and at least a portion of the product gas upstream of the one or more pairs of electrodes.

19. The system of claim 18, wherein the flow controller is in electrical communication with the controller such that the controller is configured to regulate the amount of the product gas delivered to the inspiratory flow and upstream of the one or more pairs of electrodes.

20. The system of claim 16, wherein the dilution value is a dilution factor, the dilution factor being a function of an injected inspiratory flow and a pre-injection inspiratory flow.

21. The system of claim 16, wherein the dilution value is a dilution ratio, the dilution ratio being a function of an injected inspiratory flow and a flow of inspiratory gas downstream of the NO injection.
